(19) United States
(12) Patent Application Publication
(10) Pub. No.: US 2018/0212159 A1
(43) Pub. Date: Jul. 26, 2018

(54) ORGANIC ELECTROLUMINESCENT ELEMENT

(71) Applicant: IDEMITSU KOSAN CO., LTD., Tokyo (JP)

(72) Inventors: Masahiro KAWAMURA, Sodegaura-shi, Chiba (JP); Hideaki NAGASHIMA, Sodegaura-shi, Chiba (JP); Thomas SCHAEFER, Liestal (CH)

(73) Assignee: IDEMITSU KOSAN CO., LTD., Tokyo (JP)

(21) Appl. No.: 15/745,687

(22) PCT Filed: Sep. 14, 2016
(86) PCT No.: PCT/JP2016/077194

§ 371 Date: Jan. 17, 2018

(30) Foreign Application Priority Data
Sep. 18, 2015 (JP) 2015-185526

(51) Int. Cl. H01L 51/00; C07D 519/00; C09K 11/02; C07D 487/04; C07D 251/24; C07D 401/04; C07D 213/16; C07D 239/26; C07D 403/14; C07D 401/14; C07D 403/10; C07D 495/04; C07D 491/048; C09K 11/06

(52) U.S. Cl. CPC H01L 51/0072; H01L 51/5016; C09K 11/025; H01L 51/0073; C07D 487/04; H01L 51/0067; C07D 251/24; H01L 51/0054; C07D 401/04; C07D 213/16; C07D 239/26; H01L 51/0056; H01L 51/0052; C07D 403/14; C07D 401/14; C07D 403/10; C07D 495/04; H01L 51/0071; C07D 491/048; C09K 11/06; H01L 51/0085; C09K 2211/185; C09K 2211/1029; C09K 2211/1007; C07D 519/00

(57) ABSTRACT

An organic electroluminescence device comprising an anode, a cathode and an emitting layer between the anode and the cathode, wherein the emitting layer comprises as a host material a compound represented by the following formula (1) and a compound represented by the following formula (2):

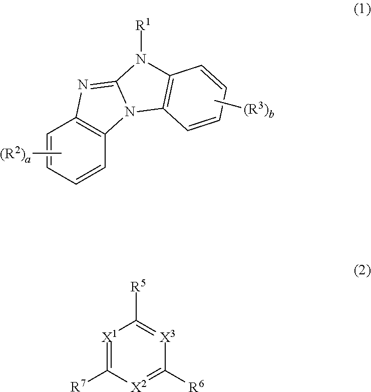

### TECHNICAL FIELD

The invention relates to an organic electroluminescence device.

### BACKGROUND ART

Lowering in driving voltage, improvement in luminous efficiency and prolongation of lifetime of a phosphorescent organic EL device is a universal object, and various materials have been developed in order to attain this object.

It is known that a compound having a 4H-imidazo[1,2-a]imidazole skeleton can be used as a host material for a phosphorescent emitting material in an electroluminescence device (Patent Documents 1 to 4).

### RELATED ART DOCUMENTS

### Patent Documents

Patent Document 1: WO2012/130709

Patent Document 2: WO2014/009317

Patent Document 3: WO2013/068376

Patent Document 4: WO2011/160757

### SUMMARY OF THE INVENTION

An object of the invention is to provide a phosphorescent organic electroluminescence device that can be driven at a lower voltage.

According to one aspect of the invention, the following organic electroluminescence device and the electronic appliance are provided.

The organic electroluminescence device according to one aspect of the invention is an organic electroluminescence device comprising an anode, a cathode and at least an emitting layer therebetween,

wherein the emitting layer comprises as a host material a compound represented by the following formula (1) and a compound represented by the following formula (2):

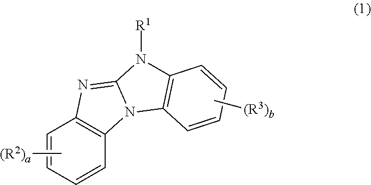

wherein in the formula (1),

R¹ to R³ are independently a group represented by —(B¹)ₒ—(B²)ₚ—(B³)_{q}—(B⁴)ᵣ—R⁴,

o, p, q and r are independently 0 or 1,

B¹ to B⁴ are independently a substituted or unsubstituted arylene group including 6 to 24 carbon atoms that form a ring (hereinafter referred to as “ring carbon atoms”) or a substituted or unsubstituted heteroarylene group including 5 to 30 atoms that form a ring (hereinafter referred to as “ring atoms”),

R⁴ is a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of an alkyl group including 1 to 25 carbon atoms and an aryl group including 6 to 24 ring carbon atoms, a cyano group or a —P(═O)R⁹R¹⁰ group,

R⁹ and R¹⁰ are independently a substituted or unsubstituted alkyl group including 1 to 25 carbon atoms, a substituted or unsubstituted aryl group including 6 to 18 ring carbon atoms or a substituted or unsubstituted heteroaryl group including 5 to 18 ring atoms, and

a and b are independently an integer of 0 to 4,

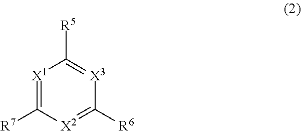

wherein in the formula (2),

X¹ to X³ are independently CR¹¹ or N,

R⁵ to R⁷ and R¹¹ are independently —(B⁵)ₛ—(B⁶)ₜ—(B⁷)ᵤ—(B⁸)ᵥ—R⁸,

s, t, u and v are independently 0 or 1,

B⁵ to B⁸ are independently a substituted or unsubstituted arylene group including 6 to 24 ring carbon atoms or a substituted or unsubstituted heteroarylene group including 5 to 30 ring atoms,

R⁸ is a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of an alkyl group including 1 to 25 carbon atoms and an aryl group including 6 to 24 ring carbon atoms, a cyano group or a —P(═O)R¹⁴R¹⁵ group,

adjacent substituents on R⁸ may be bonded with each other to form a ring, and

R¹⁴ and R¹⁵ are independently a substituted or unsubstituted alkyl group including 1 to 25 carbon atoms, a substituted or unsubstituted aryl group including 6 to 18 ring carbon atoms or a substituted or unsubstituted heteroaryl group including 5 to 18 ring atoms.

An electronic device according to one aspect of the invention is provided with the organic electroluminescence device according to one aspect of the invention.

According to one aspect of the invention, a phosphorescent organic electroluminescence device that can be driven at a low voltage is provided.

### Mode for Carrying out the Invention

A. Organic Electroluminescence Device

The organic electroluminescence device according to one aspect of the invention (hereinbelow, the “electroluminescence” may often be abbreviated as the “EL”) is an organic electroluminescence device comprising an anode, a cathode and an emitting layer between the anode and the cathode,

wherein the emitting layer comprises as a host material a compound represented by the following formula (1) and a compound represented by the following formula (2):

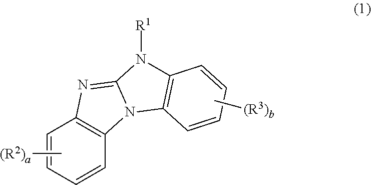

wherein in the formula (1),

R¹ to R³ are independently a group represented by —(B¹)ₒ—(B²)ₚ—(B³)_{q}—(B⁴)ᵣ—R⁴,

o, p, q and r are independently 0 or 1,

B¹ to B⁴ are independently a substituted or unsubstituted arylene group including 6 to 24 ring carbon atoms or a substituted or unsubstituted heteroarylene group including 5 to 30 ring atoms,

R⁴ is a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of an alkyl group including 1 to 25 carbon atoms and an aryl group including 6 to 24 ring carbon atoms, a cyano group or a —P(═O)R⁹R¹⁰ group,

R⁹ and R¹⁰ are independently a substituted or unsubstituted alkyl group including 1 to 25 carbon atoms, a substituted or unsubstituted aryl group including 6 to 18 ring carbon atoms or a substituted or unsubstituted heteroaryl group including 5 to 18 ring atoms, and

a and b are independently an integer of 0 to 4,

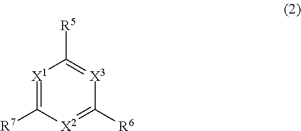

wherein in the formula (2),

X¹ to X³ are independently CR¹¹ or N,

R⁵ to R⁷ and R¹¹ are independently —(B⁵)ₛ—(B⁶)ₜ—(B⁷)ᵤ—(B⁸)ᵥ—R⁸,

s, t, u and v are independently 0 or 1,

B⁵ to B⁸ are independently a substituted or unsubstituted arylene group including 6 to 24 ring carbon atoms or a substituted or unsubstituted heteroarylene group including 5 to 30 ring atoms,

R⁸ is a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of an alkyl group including 1 to 25 carbon atoms and an aryl group including 6 to 24 ring carbon atoms, a cyano group or a —P(═O)R¹⁴R¹⁵ group,

adjacent substituents on R⁸ may be bonded with each other to form a ring, and

R¹⁴ and R¹⁵ are independently a substituted or unsubstituted alkyl group including 1 to 25 carbon atoms, a substituted or unsubstituted aryl group including 6 to 18 ring carbon atoms or a substituted or unsubstituted heteroaryl group including 5 to 18 ring atoms.

The organic EL device according to one aspect of the invention is characterized in that the compound represented by the above formula (1) and the compound represented by the above formula (2) are used in the emitting layer in combination.

The compound represented by the formula (1) has a 4H-imidazo[1,2-a]imidazole skeleton represented by the following formula. It is supposed that, since this skeleton has strong electron-donating property (donar property), hole-injection property is improved.

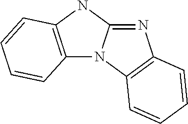 [SMILES: c1ccc2c(c1)nc1[nH]c3ccccc3n12]

The compound represented by the formula (2) has any of hetero-aromatic six-membered ring skeletons represented by the following formula that has 1 to 3 nitrogen atoms as the ring-forming atom. It is supposed that, since this skeleton has strong electron-accepting property (acceptor property), electron-injection property is improved.

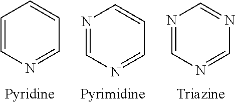

Here, the compound represented by the formula (1) is a material having a higher hole-injecting property than the compound represented by the formula (2). For example, it has an ionization potential of 5.0 eV to 6.0 eV, preferably 5.3 eV to 5.8 eV. Further, it is preferred that the electron-injection property of the compound represented by the formula (1) be smaller than the electron-injecting property of the compound represented by the formula (2), and that the affinity (Af) value of the compound represented by the formula (1) be smaller than 2.0 eV. In order to have an affinity value smaller than 2.0 eV, it is preferred that the compound represented by the formula (1) do not have an electron-deficient (i.e. having a strong electron-accepting property) nitrogen-containing six-membered ring skeleton (pyridine, pyrimidine, triazine, etc.).

The compound represented by the formula (2) is a material having a higher electron-injecting property as compared with the compound represented by the formula (1). The compound has an affinity (Af) value of preferably 2.0 eV to 2.6 eV, more preferably 2.2 eV to 2.5 eV.

The ionization potential (Ip) may be measured in the atmosphere by using a photoelectron spectroscopic device (AC-3: manufactured by Riken Keiki Co., Ltd.). Specifically, a measurement is conducted by a method in which a compound as a target is irradiated with light, and the amount of electrons generated by carrier separation at that time is measured.

The affinity (Af) value may be measured with reference to the statement in Forrest et al, Org. El. 2005, 6, 11-20.

By using in combination the compound represented by the formula (1) having a higher electron-donating property (donar property) as compared with a conventional carbazole compound which is a conventional host material and the compound represented by the formula (2), that serves as a phosphorescent host, that has a nitrogen-containing six-membered ring skeleton and exhibits a high luminous efficiency and a high electron-accepting property (acceptor property) in a single emitting layer, hole-injection property to the emitting layer is improved while retaining a high luminous efficiency. As a result, an advantageous effect of allowing an organic EL device to be driven at a lower voltage even if the luminous efficiency and the lifetime that are equal to those of conventional technologies can be obtained.

In order to allow two host materials to be contained in a single emitting layer, a method in which co-deposition is conducted from two deposition sources, a method in which materials are mixed in advance and the mixture is deposited, etc. can be given.

In the present specification, a hydrogen atom includes isomers differing in number of neutrons, i.e. protium, deuterium and tritium.

In the present specification, the number of “ring carbon atoms” means the number of carbon atoms among atoms constituting a ring of a compound in which atoms are bonded in the form of a ring (for example, a monocyclic compound, a fused ring compound, a cross-linked compound, a carbocyclic compound or a heterocyclic compound). When the ring is substituted by a substituent, the carbon contained in the substituent is not included in the number of ring carbon atoms. The same is applied to the “ring carbon atoms” mentioned below, unless otherwise indicated. For example, a benzene ring includes 6 ring carbon atoms, a naphthalene ring includes 10 ring carbon atoms, a pyridinyl group includes 5 ring carbon atoms, and a furanyl group includes 4 ring carbon atoms. When a benzene ring or a naphthalene ring is substituted by an alkyl group as a substituent, for example, the number of carbon atoms of the alkyl group is not included in the number of ring carbon atoms. When a fluorene ring is bonded with a fluorene ring as a substituent (including a spirofluorene ring), for example, the number of carbon atoms of the fluorene ring as the substituent is not included in the number of ring carbon atoms.

In the present specification, the number of “ring atoms” means the number of atoms constituting a ring of a compound having a structure in which atoms are bonded in the form of a ring (for example, monocycle, fused ring, ring assembly) (for example, a monocyclic compound, a fused ring compound, a cross-linked compound, a carbocyclic compound or a heterocyclic compound). It does not include atoms which do not form a ring or atoms contained in a substituent when the ring is substituted by the substituent. The same is applied to the “ring atoms” mentioned below, unless otherwise indicated. For example, a pyridine ring includes 6 ring carbon atoms, a quinazoline ring includes 10 ring atoms, and a furan ring includes 5 ring atoms. Hydrogen atoms respectively bonded with a carbon atom of a pyridine ring or a quinazoline ring or atoms constituting a substituent are not included in the number of ring atoms. When a fluorene ring is bonded with a fluorene ring as a substituent (including a spirofluorene ring), for example, the number of atoms of the fluorene ring as a substituent is not included in the number of ring atoms.

In the present specification, the “XX to YY carbon atoms” in the “substituted or unsubstituted ZZ group including XX to YY carbon atoms” means the number of carbon atoms when the ZZ group is unsubstituted. The number of carbon atoms of a substituent when the group is substituted is not included.

In the present specification, the “)0(to YY atoms” in the “substituted or unsubstituted ZZ group including XX to YY atoms” means the number of atoms when the ZZ group is unsubstituted. The number of atoms of a substituent when the group is substituted is not included.

In the present specification, the “unsubstituted” in the “substituted or unsubstituted” means bonding of a hydrogen atom, not substitution by the substituent mentioned above.

An explanation will be given on specific examples of each group and the substituent in the “substituted or unsubstituted” in the formulas mentioned above and mentioned below.

As examples of the alkyl group, a methyl group, an ethyl group, a propyl group, an isopropyl group, a n-butyl group, a s-butyl group, an isobutyl group, a t-butyl group, a n-pentyl group, a n-hexyl group, a n-heptyl group, a n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxpropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group or the like can be given.

A preferable alkyl group is selected from a methyl group, an ethyl group, a propyl group an isopropyl group, a n-butyl group, a s-butyl group, an isobutyl group, a t-butyl group, a n-pentyl group and a n-hexyl group.

The number of carbon atoms of the alkyl group is preferably 1 to 50, more preferably 1 to 25, and further preferably 1 to 10.

An alkenyl group is a group having a double bond in the above-mentioned alkyl group. The number of carbon atoms of an alkenyl group is 2 to 50, preferably 2 to 25, and more preferably 2 to 10. A preferable alkenyl group is a vinyl group.

An alkynyl group is a group having a triple bond in the alkyl group. The number of carbon atoms of an alkynyl group is 2 to 50, preferably 2 to 25, and more preferably 2 to 10. A preferably alkynyl group is an ethynyl group.

As examples of the cycloalkyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, a 2-norbornyl group or the like can be given.

The number of ring carbon atoms of a cycloalkyl group is 3 to 50, preferably 3 to 25, more preferably 3 to 10, further preferably 3 to 8, and particularly preferably 3 to 6.

The alkoxy group is a group represented by —OY¹⁰. As examples of Y¹⁰, the same groups as those mentioned above with reference to the alkyl group and the cycloalkyl group can be given.

The alkylthio group is a group represented by —SY¹⁰. As examples of Y¹⁰, the same groups as those mentioned above with reference to the alkyl group and the cycloalkyl group can be given.

The number of carbon atoms of the alkylthio group is 1 to 50, preferably 1 to 25, and more preferably 1 to 10.

As the halogen atom, fluorine, chlorine, bromine, iodine or the like can be given, with a fluorine atom being preferable.

The haloalkyl group is a group in which one or more hydrogen atoms in the alkyl group and the cycloalkyl group are substituted by the halogen atom.

The alkylcarbonyloxy group is a group represented by —O—(C═O)—Y¹⁰, and Y¹⁰ is as mentioned above.

As examples of an aryl group, a phenyl group, a 1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-pyrenyl group, a 2-pyrenyl group, a 4-pyrenyl group, a 6-chrysenyl group, a 1-benzo[c]phenanthryl group, a 2-benzo[c]phenanthryl group, a 3-benzo[c]phenanthryl group, a 4-benzo[c]phenanthryl group, a 5-benzo[c]phenanthryl group, a 6-benzo[c]phenanthryl group, a 1-benzo[g]chrysenyl group, a 2-benzo[g]chrysenyl group, a 3-benzo[g]chrysenyl group, a 4-benzo[g]chrysenyl group, a 5-benzo[g]chrysenyl group, a 6-benzo[g]chrysenyl group, a 7-benzo[g]chrysenyl group, a 8-benzo[g]chrysenyl group, a 9-benzo[g]chrysenyl group, a 10-benzo[g]chrysenyl group, a 11-benzo[g]chrysenyl group, a 12-benzo[g]chrysenyl group, a 13-benzo[g]chrysenyl group, a 14-benzo[g]chrysenyl group, a 1-benzo[a]anthryl group, a 2-benzo[a]anthryl group, a 3-benzo[a]anthryl group, a 4-benzo[a]anthryl group, a 5-benzo[a]anthryl group, a 6-benzo[a]anthryl group, a 7-benzo[a]anthryl group, a 8-benzo[a]anthryl group, a 9-benzo[a]anthryl group, a 10-benzo[a]anthryl group, a 11-benzo[a]anthryl group, a 12-benzo[a]anthryl group, a 13-benzo[a]anthryl group, a 14-benzo[a]anthryl group, a 1-triphenyl group, a 2-triphenyl group, a 1-fluorenyl group, a 2-fluorenyl group, a 3-fluorenyl group, a 4-fluorenyl group, a benzofluorenyl group, a dibenzofluorenyl group, a 2-biphenylyl group, a 3-biphenylyl group, a 4-biphenylyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, a fluoranthenyl group, or the like can be given.

Among these, a phenyl group, a 1-naphthyl group, a 2-naphthyl group, a 1-phenanthryl group, a 2-phenanthryl group, a 3-phenanthryl group, a 4-phenanthryl group, a 9-phenanthryl group, a 1-fluorenyl, a 2-fluorenyl group, a 3-fluorenyl group, a 4-fluorenyl group, a 5-benzo[c]phenanthryl group, a 4-benzo[a]anthryl group, a 7-benzo[a]anthryl group, a 1-triphenyl group, a 2-triphenyl group and a fluoranthenyl group are preferable.

The ring carbon atoms of the aryl group is 6 to 50, preferably 6 to 24, more preferably 6 to 20, and further preferably 6 to 18.

The arylene group is a divalent group Y²¹ in which one hydrogen atom or one substituent is further removed from the aryl group.

An aralkyl group is represented by —Y¹¹-Y²⁰. As examples of Y¹¹, a divalent group (alkylene group or cycloalkylene group) in which one hydrogen atom or one substituent is further removed from those mentioned above as the alkyl group and the cycloalkyl group can be given. As examples of Y²⁰, the aryl group mentioned above can be given.

An aryloxy group is represented by —OY²⁰. As examples of Y²⁰, the same groups as those mentioned above as the aryl group can be given.

An arylthio group is represented by —SY²⁰. As examples of Y²⁰, the same groups as those mentioned above as the aryl group can be given.

An arylcarbonyloxy group is represented by —O—(C═O)—Y²⁰. Y²⁰ is as mentioned above.

A substituted carbonyl group having a substituent selected from an alkyl group and an aryl group is represented by —(C═O)—Y¹⁰ or —(C═O)—Y₂₀, and Y¹⁰ and Y²⁰ are as mentioned above.

As examples of the heteroaryl group, a 1-pyrrolyl group, a 2-pyrrolyl group, a 3-pyrrolyl group, pyrazinyl group, a 2-pyridinyl group, a 3-pyridinyl group, a 4-pyridinyl group, a pyrimidinyl group, a triazinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a 1-dibenzofuranyl group, a 2-dibenzofuranyl group, a 3-dibenzofuranyl group, a 4-dibenzofuranyl group, a 1-dibenzothiophenyl group, a 2-dibenzothiophenyl group, a 3-dibenzothiophenyl group, a 4-dibenzothiophenyl group, a carbazolyl group, a benzocarbazolyl group, an azadibenzofuranyl group, an azadibenzothiophenyl group, a diazadibenzofuranyl group, a diazadibenzothiophenyl group, a diazaphenanthryl group, a 1-naphthobenzofuranyl group, a 2-naphthobenzofuranyl group, a 3-naphthobenzofuranyl group, a 4-naphthobenzofuranyl group, a 1-naphthobenzothiophenyl group, a 2-naphthobenzothiophenyl group, a 3-naphthobenzothiophenyl group, a 4-naphthobenzothiophenyl group, a 2-benzothiophenyl group, a 3-benzothiophenyl group, a 4-benzothiophenyl group, a 5-benzothiophenyl group, a 6-benzothiophenyl group, a 7-benzothiophenyl group, a 1-carbazolyl group, a 2-carbazolyl group, a 3-carbazolyl group, a 4-carbazolyl group, a 9-carbazolyl group, a 1-benzocarbazolyl group, a 2-benzocarbazolyl group, a 3-benzocarbazolyl group, a 4-benzocarbazolyl group, a 9-benzocarbazolyl group, a quinolyl group, a 3-quinolyl group, a 4-quinolyl group, a 5-quinolyl group, a 6-quinolyl group, a 7-quinolyl group, a 8-quinolyl group, a 1-isoquinolyl group, a 3-isoquinolyl group, a 4-isoquinolyl group, a 5-isoquinolyl group, a 6-isoquinolyl group, a 7-isoquinolyl group, a 8-isoquinolyl group, a 2-quinoxalinyl group, a 5-quinoxalinyl group, a 6-quinoxalinyl group, a 2-quinazolinyl group, a 4-quinazolinyl group, a 5-quinazolinyl group, a 6-quinazolinyl group, a 7-quinazolinyl group, a 8-quinazolinyl group, a 1-phenanthridinyl group, a 2-phenanthridinyl group, a 3-phenanthridinyl group, a 4-phenanthridinyl group, a 6-phenanthridinyl group, a 7-phenanthridinyl group, a 8-phenanthridinyl group, a 9-phenanthridinyl group, a 10-phenanthridinyl group, a 1-acridinyl group, a 2-acridinyl group, a 3-acridinyl group, a 4-acridinyl group, a 9-acridinyl group, a 1,7-phenanthrolin-2-yl group, a 1,7-phenanthrolin-3-yl group, a 1,7-phenanthrolin-4-yl group, a 1,7-phenanthrolin-5-yl group, a 1,7-phenanthrolin-6-yl group, a 1,7-phenanthrolin-8-yl group, a 1,7-phenanthrolin-9-yl group, a 1,7-phenanthrolin-10-yl group, a 1,8-phenanthrolin-2-yl group, a 1,8-phenanthrolin-3-yl group, a 1,8-phenanthrolin-4-yl group, a 1,8-phenanthrolin-5-yl group, a 1,8-phenanthrolin-6-yl group, a 1,8-phenanthrolin-7-yl group, a 1,8-phenanthrolin-9-yl group, a 1,8-phenanthrolin-10-yl group, a 1,9-phenanthrolin-2-yl group, a 1,9-phenanthrolin-3-yl group, a 1,9-phenanthrolin-4-yl group, a 1,9-phenanthrolin-5-yl group, a 1,9-phenanthrolin-6-yl group, a 1,9-phenanthrolin-7-yl group, a 1,9-phenanthrolin-8-yl group, a 1,9-phenanthrolin-10-yl group, a 1,10-phenanthrolin-2-yl group, a 1,10-phenanthrolin-3-yl group, 1,10-phenanthrolin-4-yl group, a 1,10-phenanthrolin-5-yl group, a 2,9-phenanthrolin-1-yl group, a 2,9-phenanthrolin-3-yl group, a 2,9-phenanthrolin-4-yl group, a 2,9-phenanthrolin-5-yl group, a 2,9-phenanthrolin-6-yl group, a 2,9-phenanthrolin-7-yl group, a 2,9-phenanthrolin-8-yl group, a 2,9-phenanthrolin-10-yl group, a 2,8-phenanthrolin-1-yl group, a 2,8-phenanthrolin-3-yl group, a 2,8-phenanthrolin-4-yl group, a 2,8-phenanthrolin-5-yl group, a 2,8-phenanthrolin-6-yl group, a 2,8-phenanthrolin-7-yl group, a 2,8-phenanthrolin-9-yl group, a 2,8-phenanthrolin-10-yl group, a 2,7-phenanthrolin-1-yl group, a 2,7-phenanthrolin-3-yl group, a 2,7-phenanthrolin-4-yl group, a 2,7-phenanthrolin-5-yl group, a 2,7-phenanthrolin-6-yl group, a 2,7-phenanthrolin-8-yl group, a 2,7-phenanthrolin-9-yl group, a 2,7-phenanthrolin-10-yl group, a 1-phenazinyl group, a 2-phenazinyl group, a 1-phenothiadinyl group, a 2-phenothiadinyl group, a 3-phenothiadinyl group, a 4-phenothiadinyl group, a 10-phenothiadinyl group, a 1-phenoxadinyl group, a 2-phenoxadinyl group, a 3-phenoxadinyl group, a 4-phenoxadinyl group, a 10-phenoxadinyl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 1-benzoimidazoly group, a 2-benzimidazolyl group, a 4-benzimidazolyl group, a 5-benzimidazolyl group, a 6-benzimidazolyl group, a 7-benzimidazolyl group, a 2-imidazo[1,2-a]pyridinyl group, a 3-imidazo[1,2-a]pyridinyl group, a 5-imidazo[1,2-a]pyridinyl group, a 6-imidazo[1,2-a]pyridinyl group, a 7-imidazo[1,2-a]pyridinyl group, a 8-imidazo[1,2-a]pyridinyl group, a benzimidazol-2-on-1-yl group, a benzimidazol-2-on-3-yl group, a benzimidazol-2-on-4-yl group, a benzimidazol-2-on-5-yl group, a benzimidazol-2-on-6-yl group, a benzimidazol-2-on-7-yl group or the like can be given.

Among these, a pyrazinyl group, a 2-pyridinyl group, a 3-pyridinyl group, a 4-pyridinyl group, a pyrimidinyl group, a triazinyl group, a quinazolinyl group, a diazadibenzofuranyl group, a diazadibenzothiophenyl group, a 1-dibenzofuranyl group, a 2-dibenzofuranyl group, a 3-dibenzofuranyl group, a 4-dibenzofuranyl group, a 1-dibenzothiophenyl group, a 2-dibenzothiophenyl group, a 3-dibenzothiophenyl group, a 4-dibenzothiophenyl group, a 1-naphthobenzofuranyl group, a 2-naphthobenzofuranyl group, a 3-naphthobenzofuranyl group, a 4-naphthobenzofuranyl group, 1-naphthobenzothiophenyl group, a 2-naphthobenzothiophenyl group, a 3-naphthobenzothiophenyl group, a 4-naphthobenzothiophenyl group, a 1-carbazolyl group, a 2-carbazolyl group, a 3-carbazolyl group, a 4-carbazolyl group, a 9-carbazolyl group, a 1-benzocarbazolyl group, a 2-benzocarbazolyl group, a 3-benzocarbazolyl group, a 4-benzocarbazolyl group or a 9-benzocarbazolyl group are preferable.

The number of ring atoms of the heteroaryl group is 5 to 50, preferably 5 to 30, more preferably 5 to 24, and further preferably 5 to 18.

As the ring atom other than a carbon atom of the heteroaryl group, a nitrogen atom, an oxygen atom or a sulfur atom is preferable.

The heteroarylene group is a divalent group Y³¹ in which one hydrogen atom or one substituent is further removed from the heteroaryl group.

The mono-substituted amino group having a substituent selected from an alkyl group and an aryl group is represented by —NH(Y¹⁰) or —NH(Y²⁰), and Y¹⁰ and Y²⁰ are as mentioned above.

The di-substituted amino group having a substituent selected form an alkyl group and an aryl group is represented by —N(Y¹⁰)₂, —N(Y²⁰)₂ or —N(Y¹⁰ )(Y²⁰), and Y¹⁰ and Y²⁰ are as mentioned above. When two Y¹⁰s or two Y²⁰s are present, they may be the same or different.

The mono-substituted silyl group having a substituent selected from an alkyl group and an aryl group is represented by —SiH₂(Y¹⁰) or —SiH₂(Y²⁰).

The di-substituted silyl group having a substituent selected from an alkyl group and an aryl group is represented by —SiH(Y¹⁰)₂, —SiH(Y²⁰)₂ or —SiH(Y¹⁰)(Y₂₀).

The tri-substituted silyl group having a substituent selected from an alkyl group and an aryl group is represented by —Si(Y¹⁰)₃, —Si(Y²⁰)₃, —Si(Y₁₀)²(Y²⁰) or —Si(Y¹⁰)(Y²⁰)₂. Y¹⁰ and Y²⁰ are as mentioned above, and when plural Y¹⁰s or plural Y²⁰s are present, they may be the same or different.

The substituted sulfonyl group selected from an alkyl group and an aryl group is represented by —S(═O)₂—Y₁₀ or —S(═O)₂—Y²⁰, and Y¹⁰ and Y²⁰ are as mentioned above.

The di-substituted phosphoryl group having a substituent selected from an alkyl group and an aryl group is represented by —O—P(═O)(Y¹⁰)₂, —O—P(═O)(Y²⁰)₂ or —O—P(═O)(Y¹⁰)(Y20). Y¹⁰ and Y²⁰ are as mentioned above, and when two Y¹⁰s or two Y²⁰s are present, they may be the same or different.

The alkylsulfonyloxy group having an alkyl group is represented by —O—S(═O)₂(Y¹⁰). Y¹⁰ is as mentioned above.

The arylsulfonyloxy group having a substituent selected from aryl groups is represented by —O—S(═O)₂(Y²⁰). Y²⁰ is as mentioned above.

In the present specification, as the substituent in the “substituted or unsubstituted”, in addition to all groups exemplified above, a carboxy group, a hydroxyl group, an amino group can be given. Among these, an alkyl group, an alkoxy group, a halogen atom, a cyano group, a substituted silyl group, an aryl group and a heteroaryl group are preferable.

The substituents mentioned above may be bonded with each other to form a single ring or a fused ring. In another embodiment, no ring is formed.

### <Preferable Embodiment of the Compound Represented by the Formula (1)>

In one embodiment of the invention, the compound represented by the formula (1) is preferably a compound represented by the following formula (3):

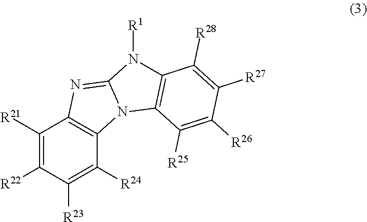

wherein in the formula (3),

R¹ is as defined in the formula (1), and

R²¹ to R²⁸ are independently a hydrogen atom, a substituted or unsubstituted alkyl group including 1 to 25 carbon atoms, a substituted or unsubstituted aryl group including 6 to 24 ring carbon atoms or a substituted or unsubstituted heteroaryl group including 5 to 30 ring atoms.

It is preferred that the compound represented by the formula (3) be a compound represented by the following formula (4):

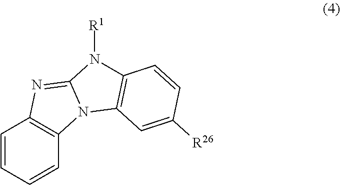

wherein in the formula (4),

R¹ and R²⁶ are as defined in the formula (3).

In one embodiment of the invention, in the formula (1), it is preferred that B¹ to B⁴ be independently a group selected from the following group:

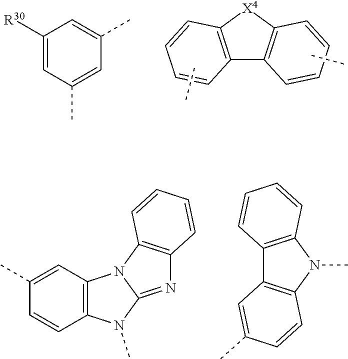

wherein in the formula, X⁴ is an oxygen atom, a sulfur atom or NR²⁰,

R²⁰ is a substituted or unsubstituted aryl group including 6 to 24 ring carbon atoms or a substituted or unsubstituted heteroaryl group including 5 to 30 ring atoms,

R³⁰ is a hydrogen atom or a group selected from the following group:

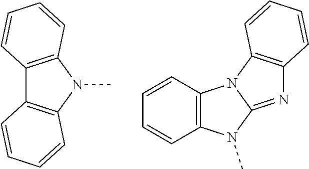

[in the formula, the dotted line indicates an atomic bonding]

In the formula (1), it is preferred that R⁴ be a group selected from the folloing group:

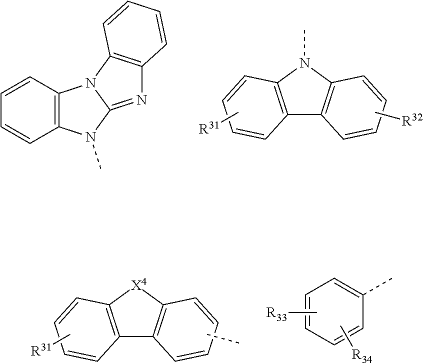

wherein in the formula,

R³¹ and R³² are independently a hydrogen atom or a group selected from the following group:

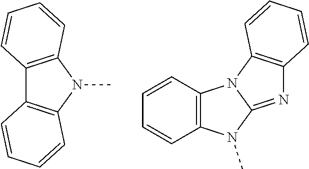

R³³ and R³⁴ are independently a hydrogen atom or a substituted or unsubstituted heteroaryl group including 5 to 30 ring atoms.

[in the formula, the dotted line indicates an atomic bonding]

In another embodiment, in the formula (1), it is preferred that B¹ to B⁴ be the above-mentioned groups and R⁴ be the above-mentioned group.

In another embodiment, in the formula (1), it is preferred that B¹ to B⁴ be independently a group selected from the following group:

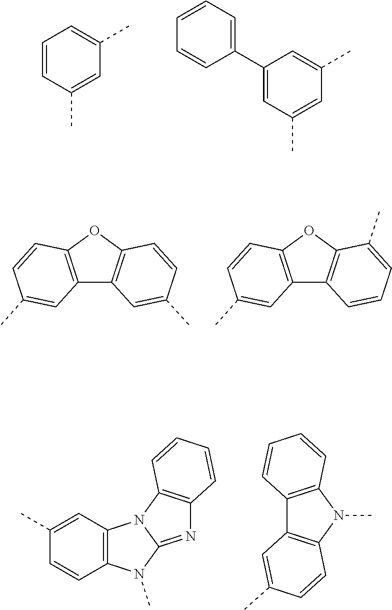

[in the formula, the dotted line indicates an atomic bonding]

In the formula (1), it is preferred that R⁴ be a group selected from the following group:

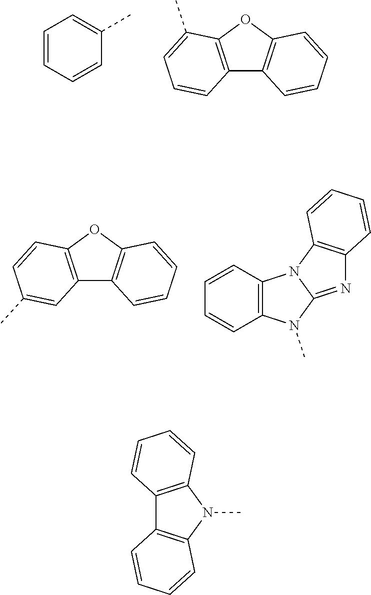

[in the formula, the dotted line indicates an atomic bonding]

In another embodiment, in the formula (1), it is preferred that B¹ to B⁴ be the above-mentioned group and R⁴ be the above-mentioned group.

In another embodiment, in the formula (1), when R¹ is a group represented by —B¹—((B²)ₚ—(B³)_{q}—(B⁴)ᵣ—R⁴, it is preferred that R⁴ be a group selected from the following group:

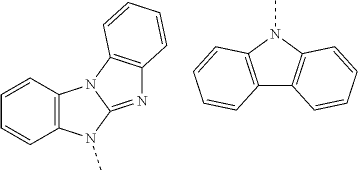

[in the formula, the dotted line indicates an atomic bonding]

In another embodiment, in the formulas (1) to (4), it is preferred that R¹ be a group selected from the following group:

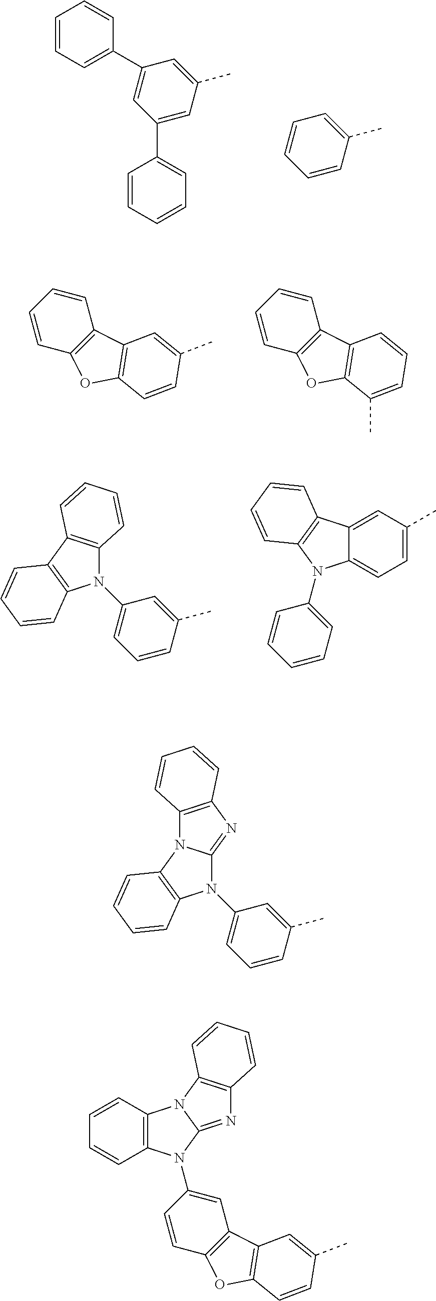

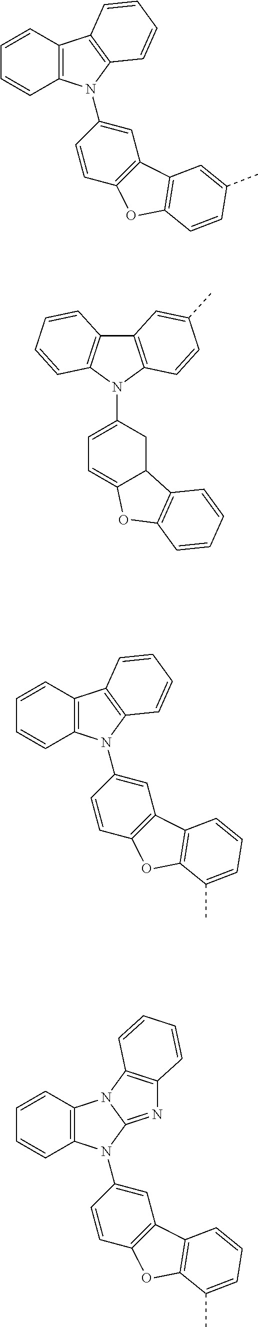

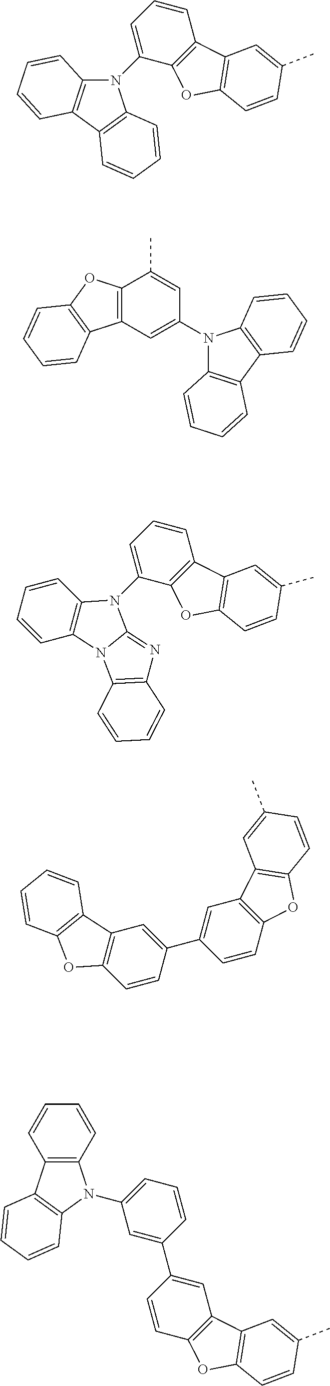

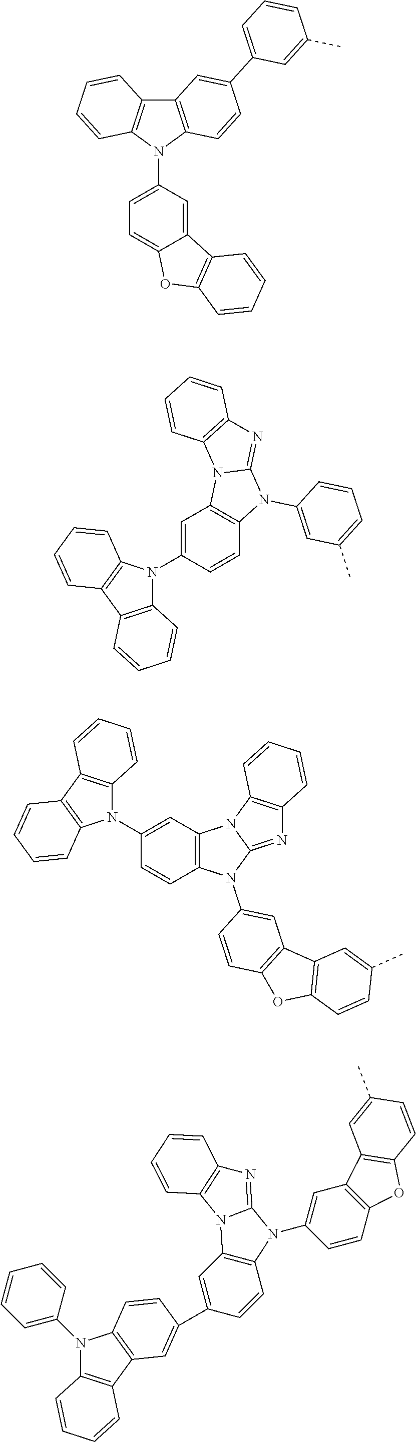

[in the formula, the dotted line indicates an atomic bonding]

In another embodiment, in the formulas (3) and (4), it is preferred that R²⁶ be represented by —(B^{1a})ₒ—(B^{2a})ₚ—(B^{3a})_{q}—(B^{4a})ᵣ—R^{4a}, o to r are as defined in the formula (2), and

B^{1a} to B^{4a} are independently a group selected from the following group:

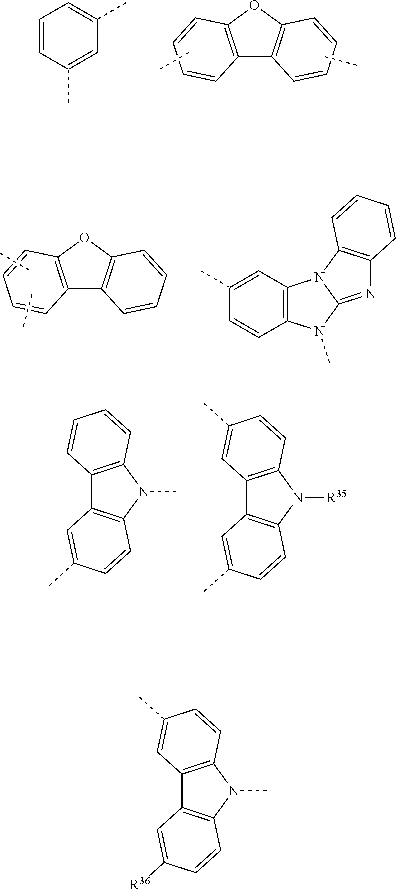

[in the formula, the dotted line indicates an atomic bonding]

In the formulas (3) and (4), it is preferred that R^{4a} be a group selected from the following group:

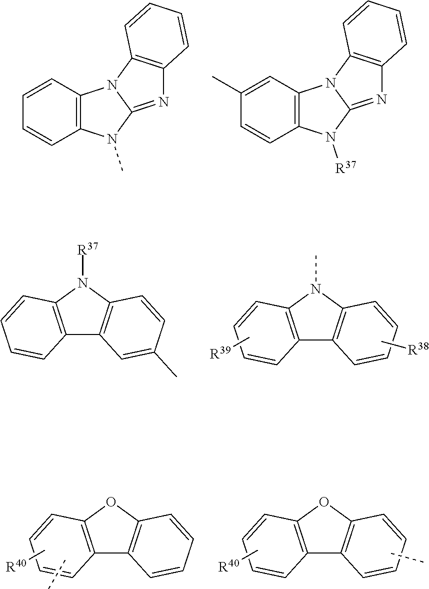

wherein in the formula,

R³⁵ and R³⁷ are independently a group selected from the following group:

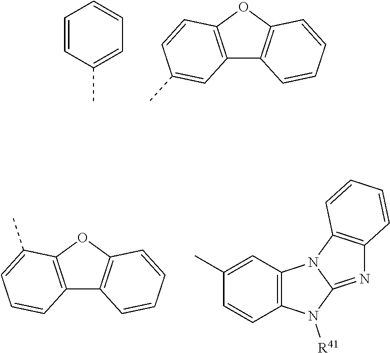

R³⁶, R³⁸, R³⁹ and R⁴⁹ are independently a group selected from the following group:

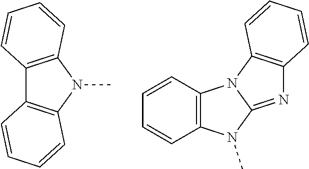

It is preferred that R⁴¹ be a group selected from the following group:

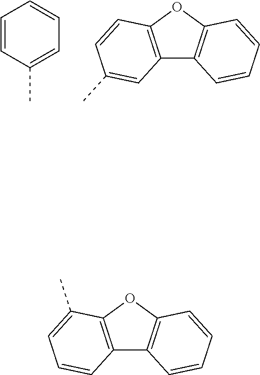

[in the formula, the dotted line indicates an atomic bonding]

In another embodiment, in the formulas (3) and (4), it is preferred that B^{1a} to B^{4a} be a group represented by the above formula, and R^{4a} be a group represented by the above formula.

In another embodiment, in the formulas (3) and (4), it is preferred that B^{1a} to B^{4a} be a group selected from the following group:

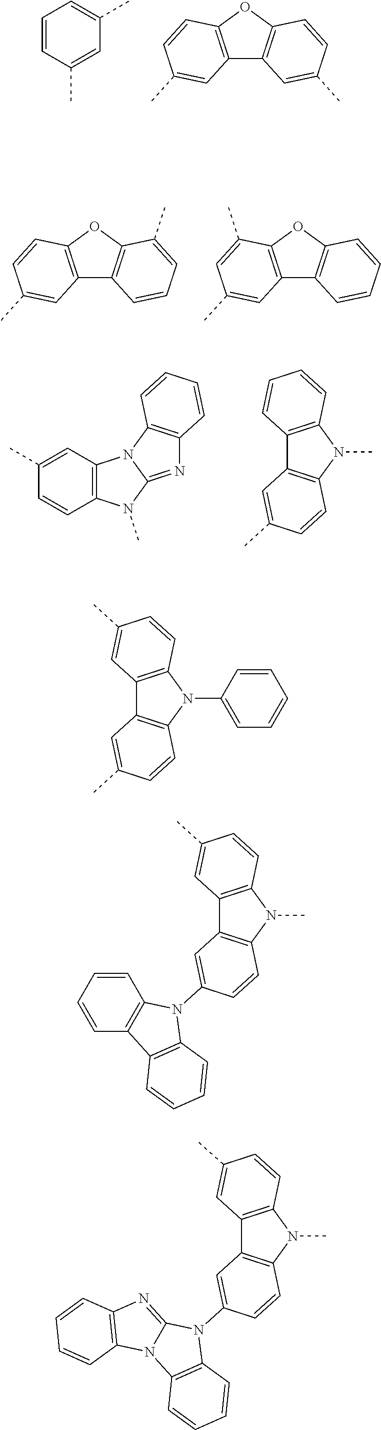

In the formulas (3) and (4), it is preferred that R^{4a} be a group selected from the following group:

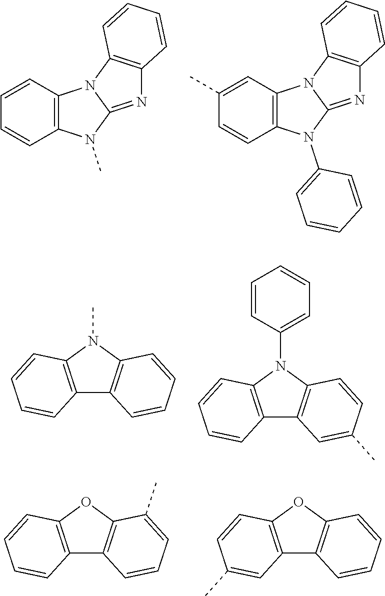

[in the formula, the dotted line indicates an atomic bonding]

In another embodiment, in the formulas (3) and (4), it is preferred that B^{1a} to B^{4a} be a group represented by the above formula and R^{4a} be a group represented by the above formula.

In another embodiment, in the formulas (3) and (4), it is preferred that R²⁶ be selected from the following group:

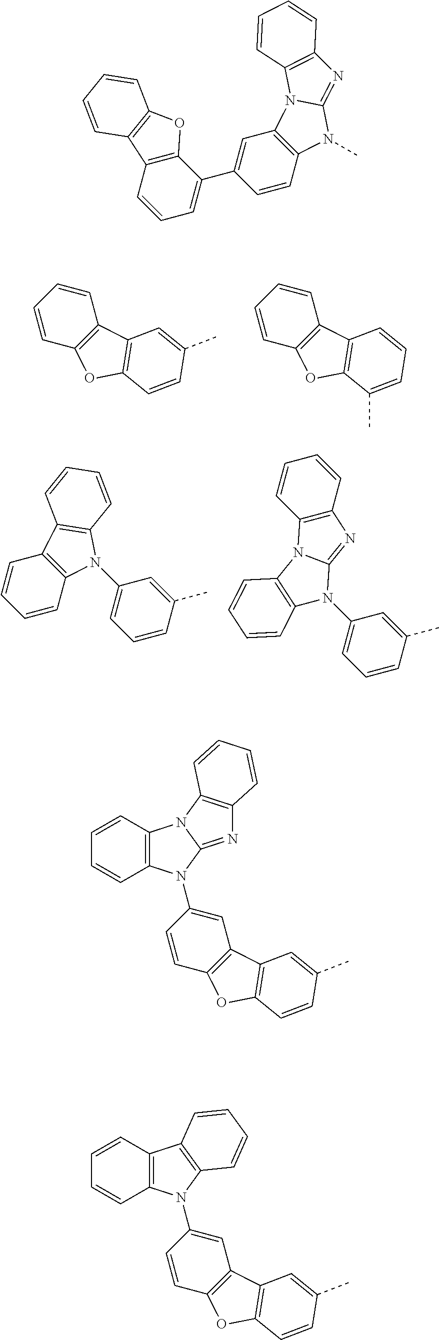

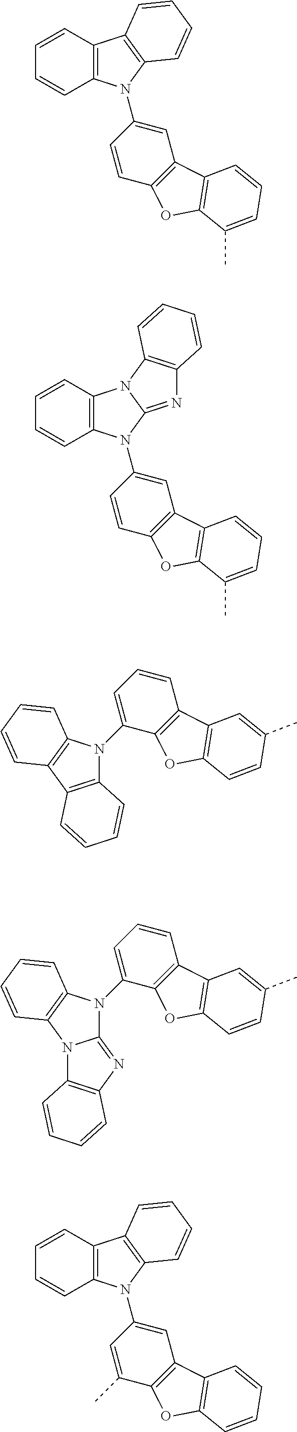

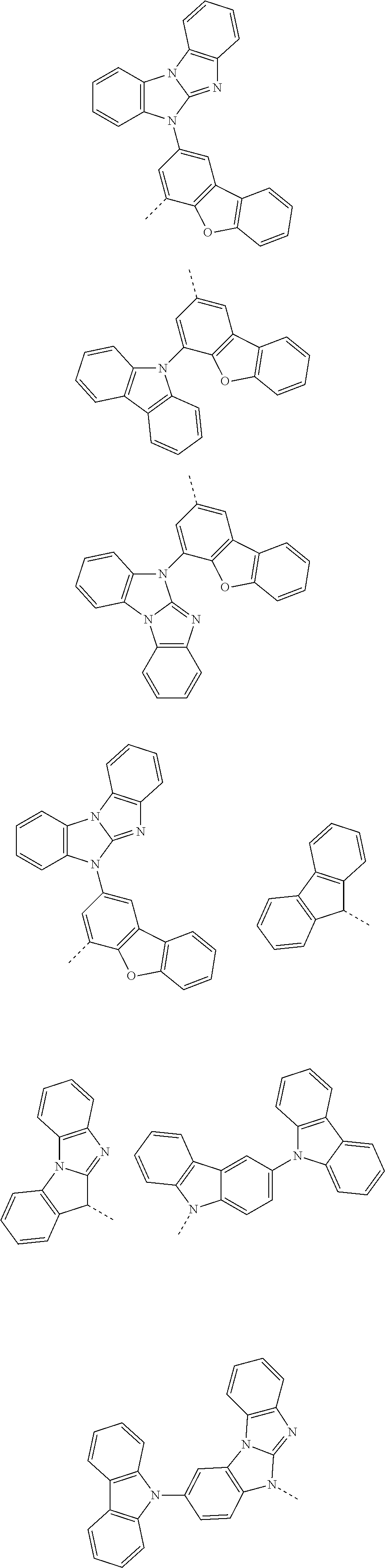

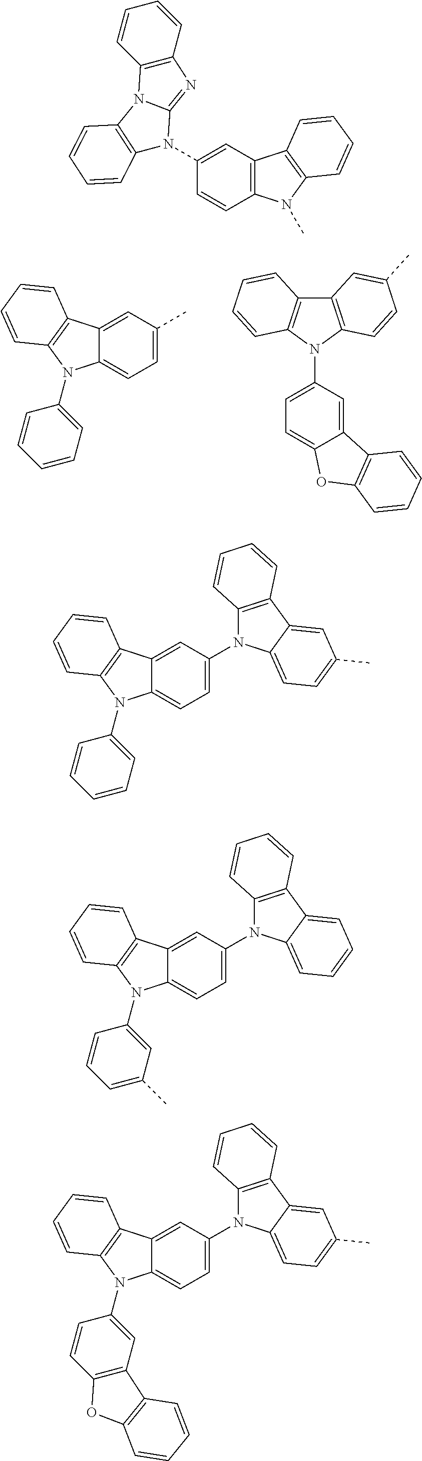

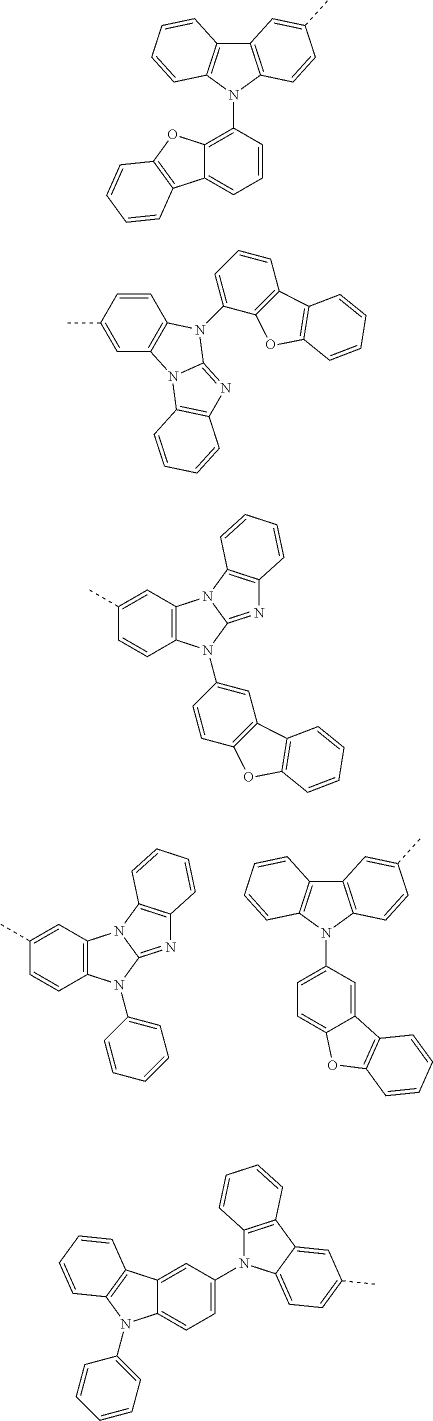

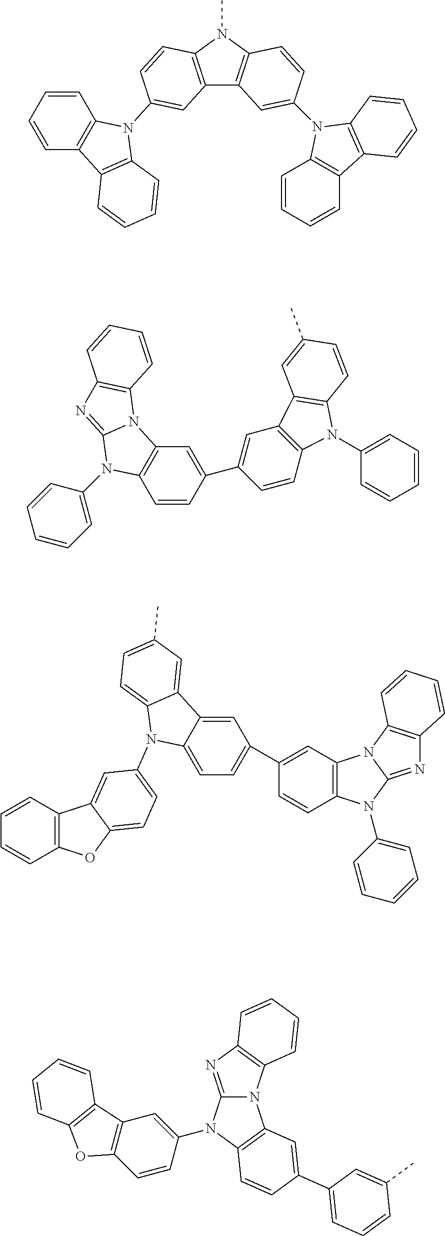

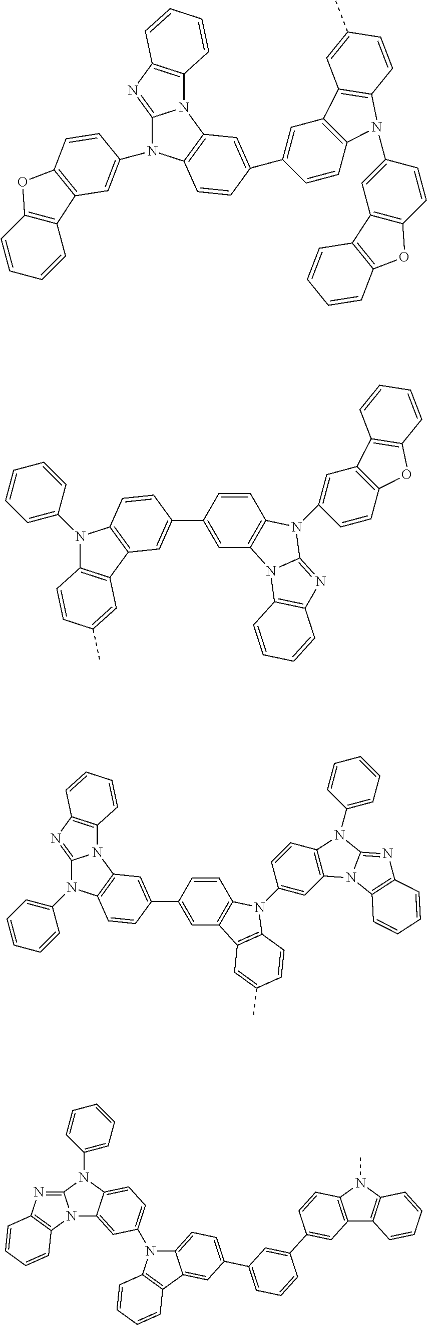

[in the formula, the dotted line indicates an atomic bonding]

In the formula (1), it is preferred that R¹ to R³ be independently a monovalent group formed of a single ring selected from the following group or a monovalent group formed by bonding of two or more rings selected from the following group:

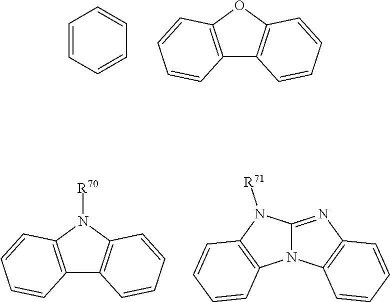

wherein in the formula, R⁷⁰ and R⁷¹ are an atomic bonding, a hydrogen atom, or a substituted or unsubstituted aryl group including 6 to 24 ring carbon atoms or a substituted or unsubstituted heteroaryl group including 5 to 30 ring atoms.

In the formula (1), it is more preferred that R¹ be a group selected from the following group:

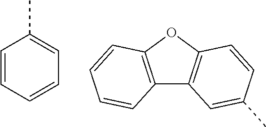

[in the formula, the dotted line indicates an atomic bonding]

R² be a group selected from the following group:

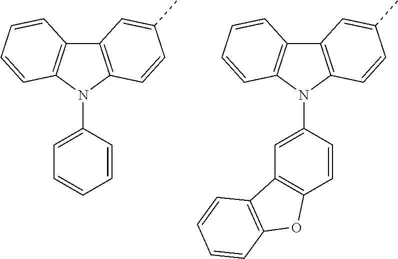

[in the formula, the dotted line indicates an atomic bonding]

and that R³ be a hydrogen atom.

Specific examples of the compound represented by the formula (1) are given below. The compound represented by the formula (1) is not limited thereto.

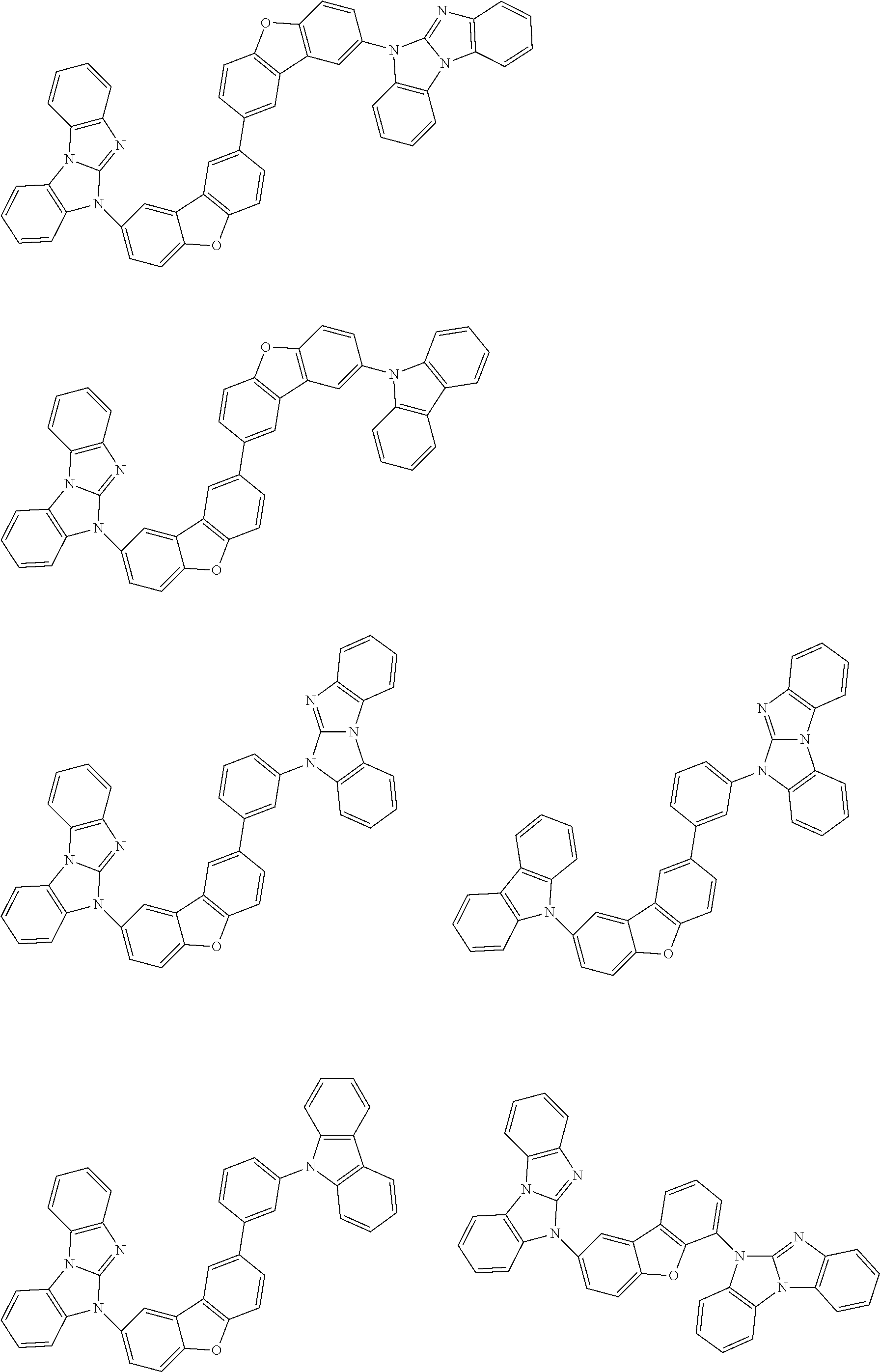

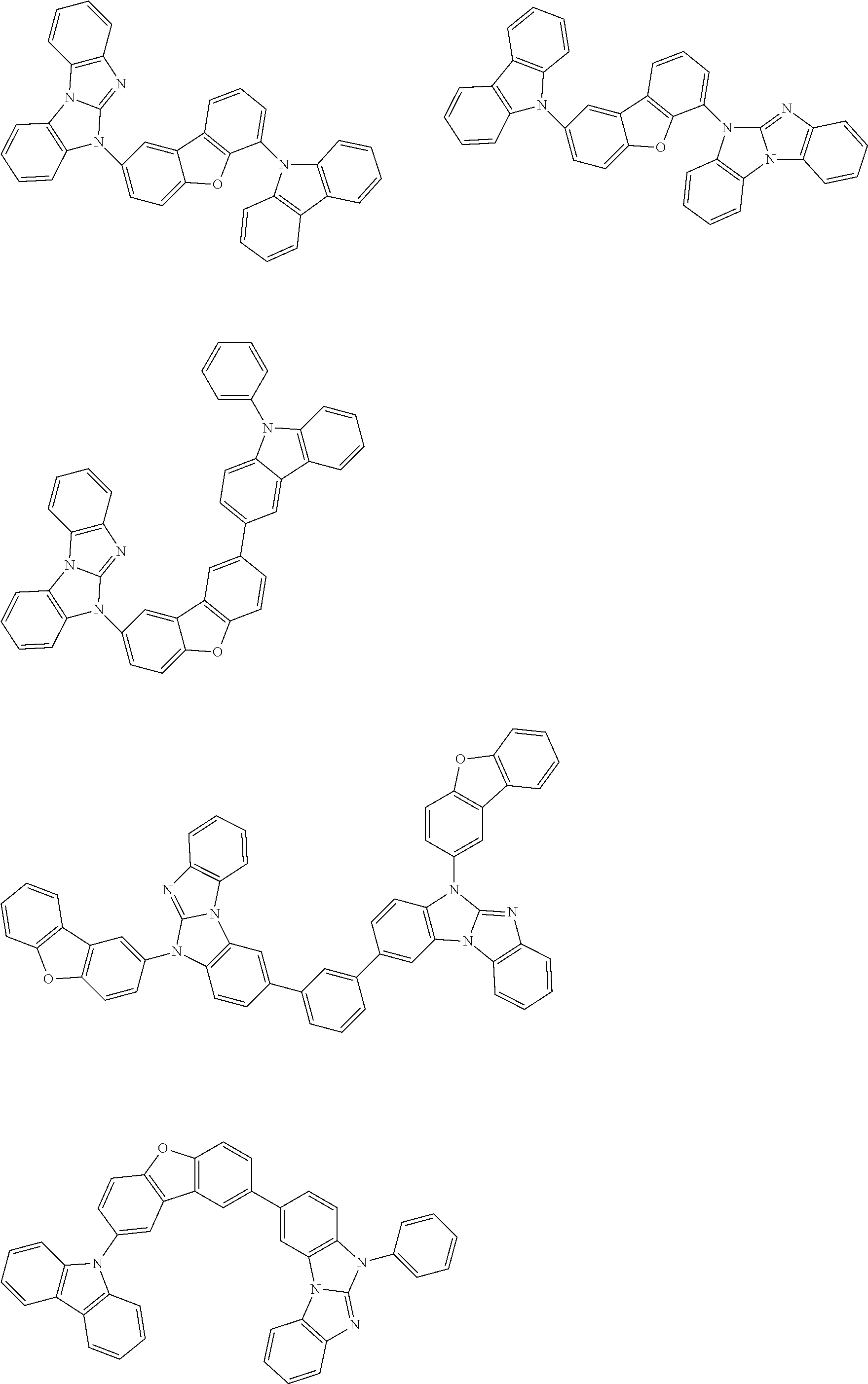 [SMILES: c1ccc2c(c1)nc1n(-c3ccc4oc5c(-n6c7ccccc7c7ccccc76)cccc5c4c3)c3ccccc3n21 ; c1ccc2c(c1)nc1n(-c3cccc4c3oc3ccc(-n5c6ccccc6c6ccccc65)cc34)c3ccccc3n21 ; c1ccc(-n2c3ccccc3c3cc(-c4ccc5oc6ccc(-n7c8ccccc8n8c9ccccc9nc78)cc6c5c4)ccc32)cc1 ; c1cc(-c2ccc3c(c2)n2c4ccccc4nc2n3-c2ccc3oc4ccccc4c3c2)cc(-c2ccc3c(c2)n2c4ccccc4nc2n3-c2ccc3oc4ccccc4c3c2)c1 ; c1ccc(-n2c3ccc(-c4ccc5oc6ccc(-n7c8ccccc8c8ccccc87)cc6c5c4)cc3n3c4ccccc4nc23)cc1]

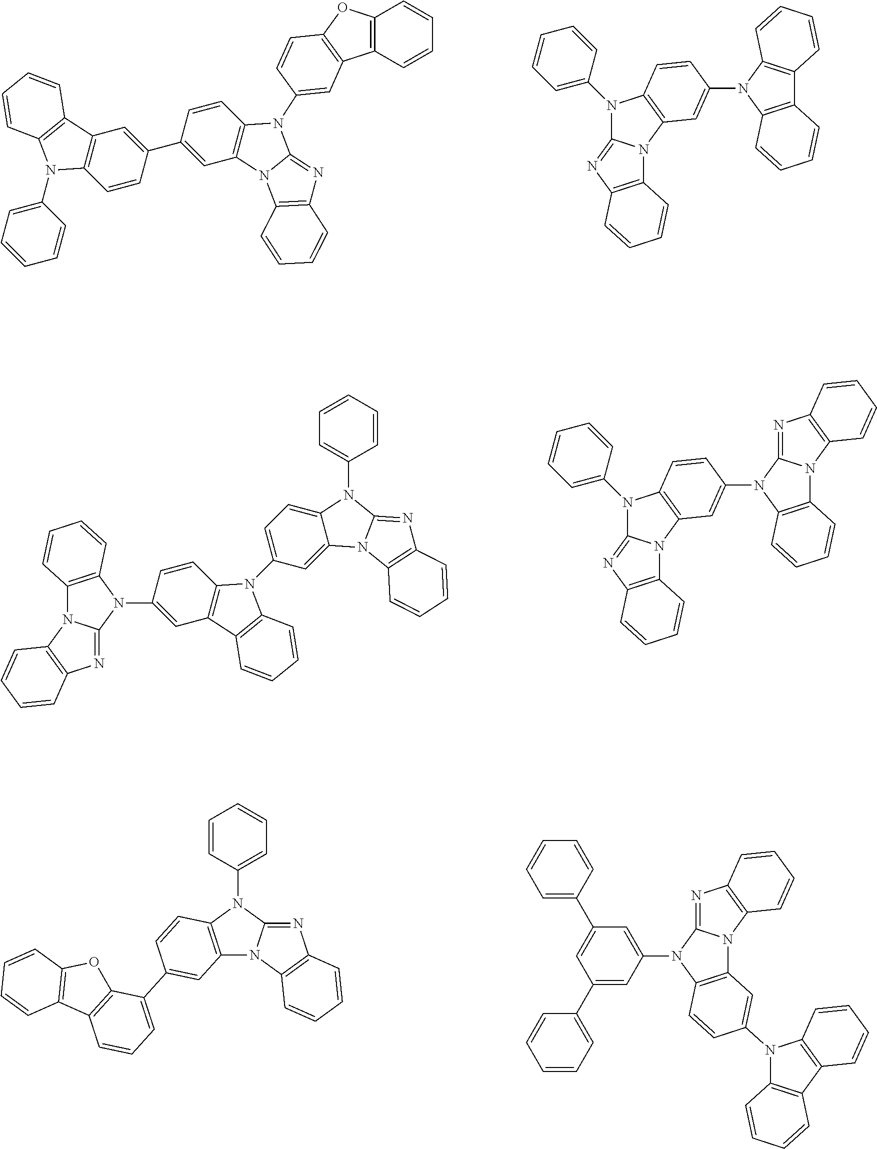 [SMILES: c1ccc(-n2c3ccccc3c3cc(-c4ccc5c(c4)n4c6ccccc6nc4n5-c4ccc5oc6ccccc6c5c4)ccc32)cc1 ; c1ccc(-n2c3ccc(-n4c5ccccc5c5ccccc54)cc3n3c4ccccc4nc23)cc1 ; c1ccc(-n2c3ccc(-n4c5ccccc5c5cc(-n6c7ccccc7n7c8ccccc8nc67)ccc54)cc3n3c4ccccc4nc23)cc1 ; c1ccc(-n2c3ccc(-n4c5ccccc5n5c6ccccc6nc45)cc3n3c4ccccc4nc23)cc1 ; c1ccc(-n2c3ccc(-c4cccc5c4oc4ccccc45)cc3n3c4ccccc4nc23)cc1 ; c1ccc(-c2cc(-c3ccccc3)cc(-n3c4ccc(-n5c6ccccc6c6ccccc65)cc4n4c5ccccc5nc34)c2)cc1]

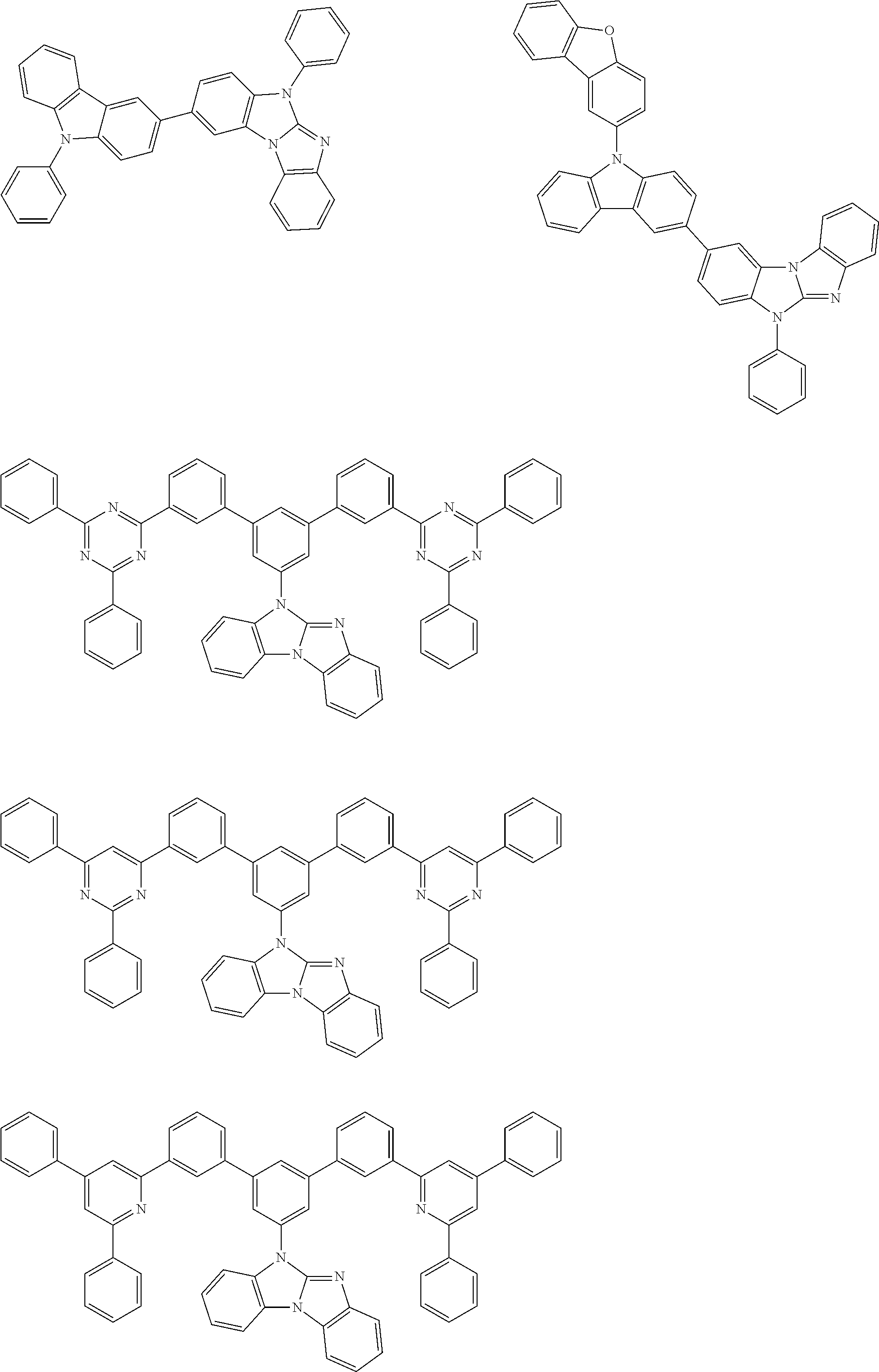

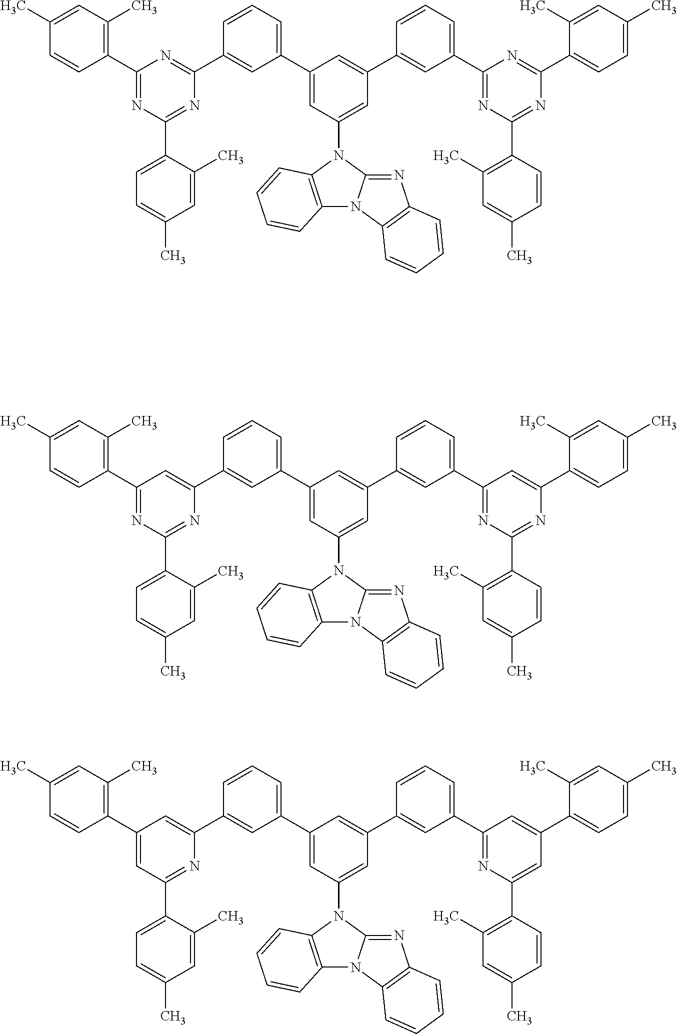

### <Preferable Embodiment of the Compound Represented by the Formula (2)>

In one embodiment of the invention, it is preferred that the compound represented by the formula (2) be a compound represented by the following formula (21):

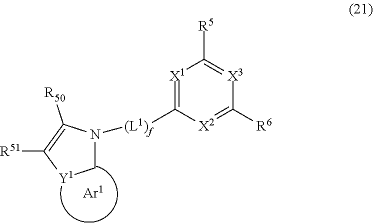

wherein in the formula (21), X¹ to X³, R⁵and R⁶ are as defined in the formula (2),

L¹ is a substituted or unsubstituted arylene group including 6 to 24 ring carbon atom or a substituted or unsubstituted heteroarylene group including 5 to 30 ring atoms,

f is an integer of 0 or 1, and when f is 2 to 4, plural L¹s may be the same or different,

Y¹ is a carbon atom or a nitrogen atom,

Ar¹ is a substituted or unsubstituted aromatic hydrocarbon ring including 6 to 24 ring carbon atoms that shares a carbon atom and Y¹ of an adjacent nitrogen-containing five-membered ring and that is fused to the nitrogen-containing five-membered ring, or a substituted or unsubstituted heterocyclic aromatic ring including 5 to 30 ring atoms that shares a carbon atom and Y¹ of an adjacent nitrogen-containing five-membered ring and that is fused to the nitrogen-containing five-membered ring,

R⁵⁰ and R⁵¹ are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more substituents selected from the group consisting of an alkyl group including 1 to 25 carbon atoms and an aryl group including 6 to 24 ring carbon atoms, a cyano group or a —P(═O)R¹⁴R¹⁵ group, or R⁵⁰ and R⁵¹ are bonded with each other to form a substituted or unsubstituted aromatic ring group including 6 to 24 ring carbon atoms, and

R¹⁴ and R¹⁵are as defined in the formula (2).

It is preferred that the compound represented by the formula (21) be a compound represented by the following formula (22):

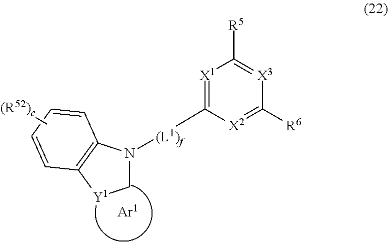

wherein in the formula (22), X¹ to X³, R⁵, R⁶, L¹, f, Y¹ and Ar¹ are as defined in the formula (21);

R⁵² is a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more substituents selected from the group consisting of an alkyl group including 1 to 25 carbon atoms and an aryl group including 6 to 24 ring carbon atoms, a cyano group or a —P(═O) R¹⁴R¹⁵ group; adjacent groups on R⁵² may be bonded with each other to form a ring;

R¹⁴ and R¹⁵ are as defined in the formula (2),

c is an integer of 0 to 4,

when c is 2 to 4, plural R⁵²s may be the same or different, and adjacent R⁵²s may be bonded with each other to form a ring.

It is preferred that the compound represented by the formula (22) be a compound represented by the following formula (23):

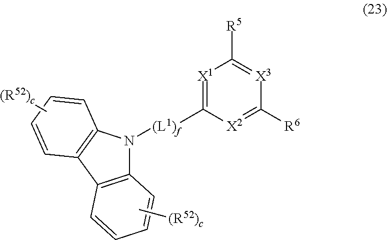

wherein in the formula (23), X¹to X³, R⁵, R⁶, L¹, f, R⁵² and c are as defined in the formula (22).

It is preferred that the compound represented by the formula (23) be a compound represented by the following formula (24):

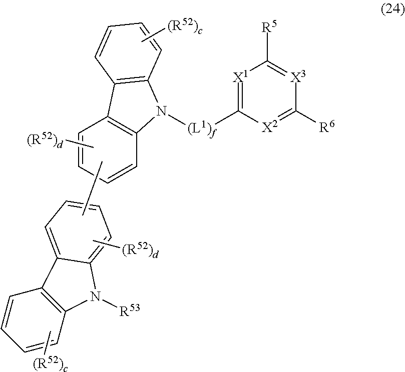

wherein in the formula (24),

X¹ to X³, R⁵, R⁶, L¹, f, R⁵² and c are as defined in the formula (23),

d is an integer of 0 to 3, and when d is 2 or 3, plural R⁵²s may be the same or different;

R⁵³ is a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more substituents selected from the group consisting of an alkyl group including 1 to 25 carbon atoms and an aryl group including 6 to 24 ring carbon atoms, a cyano group or a —P(═O)R¹⁴R¹⁵ group, and adjacent substituents on R⁵³ may be bonded with each other to form a ring.

R¹⁴ and R¹⁵ are as defined in the formula (2).

In another embodiment, it is preferred that the compound represented by the formula (22) be a compound represented by the following formula (25):

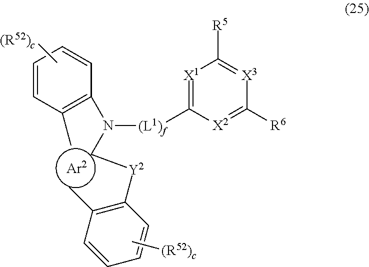

wherein in the formula (25),

X¹ to X³, R⁵, R⁶, L¹, f, R⁵² and c are as defined in the formula (22),

Y² is CR⁵⁴R⁵⁵, NR⁵⁶, an oxygen atom or a sulfur atom,

R⁵⁴ to R⁵⁶ are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more substituents selected from the group consisting of an alkyl group including 1 to 25 carbon atoms and an aryl group including 6 to 24 ring carbon atoms, a cyano group or a —P(═O)R¹⁴R¹⁵ group; and adjacent substituents on R⁵⁴to R⁵⁶ may be bonded with each other to form a ring;

R¹⁴ and R¹⁵ are as defined in the formula (2), and

Ar² is a substituted or unsubstituted aromatic hydrocarbon ring including 6 to 24 ring carbon atoms that shares two carbon atoms of each of adjacent two five-membered rings and that is fused to the two five-membered rings, or a substituted or unsubstituted heterocyclic aromatic ring including 5 to 30 ring atoms that shares two carbon atoms of each of adjacent two five-membered rings and that is fused to the two five-membered rings.

It is preferred that Y² in the formula (25) be CR⁵⁴R⁵⁵ or NR⁵⁶ (wherein R⁵⁴ to R⁵⁶ are as defined in the formula (25)).

It is preferred that the compound represented by the formula (25) be a compound represented by any one of the following formulas (26A) to (26F).

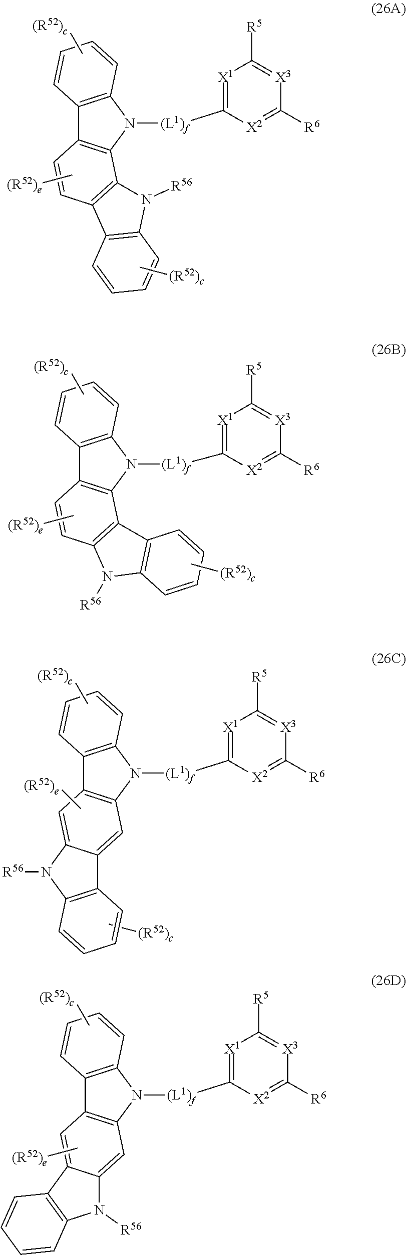

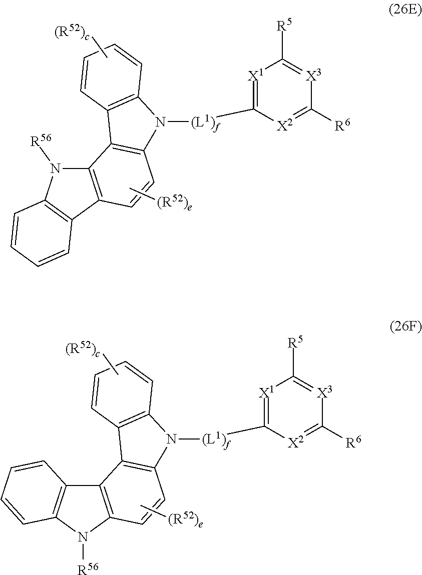

wherein in the formulas (26A) to (26F),

X¹ to X³, R⁵, R⁶, L¹, f, R⁵², R⁵⁶ and c are as defined in the formula (25), and

e is an integer of 0 to 2, and when e is 2, plural R⁵²s may be the same or different.

It is preferred that the compound represented by the formula (25) be a compound represented by any of the following formulas (27A) to (27F):

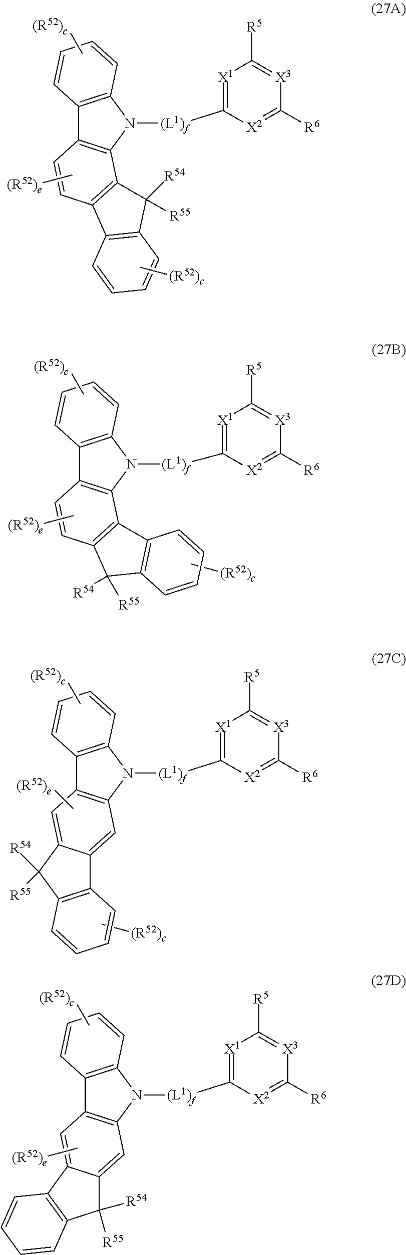

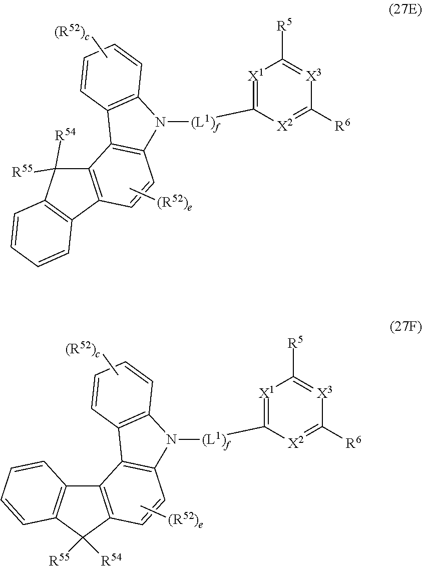

wherein in the formulas (27A) to (27F),

X¹ to X³, R⁵, R⁶, L¹, f, R⁵², R⁵⁴, R⁵⁶ and c are as defined in the formula (25),

e is an integer of 0 to 2, and

when e is 2, plural R⁵²s may be the same or different.

In another embodiment, the compound represented by the formula (22) may be a compound represented by the following formula (28):

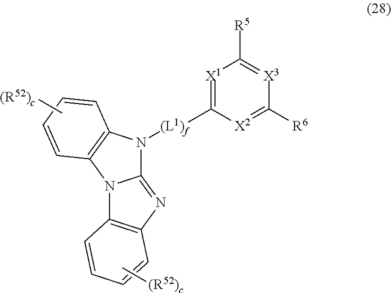

wherein in the formula (28),

X¹ to X³, R⁵, R⁶, L¹, f, R⁵² and c are as defined in the formula (22).

In still another embodiment, it is preferred that the compound represented by the formula (2) be a compound represented by the following formula (30):

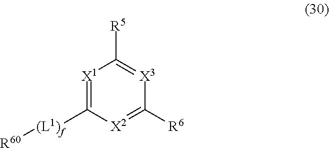

wherein in the formula (30),

X¹ to X³, R⁵ and R⁶ are as defined in the formula (2),

L¹ is a substituted or unsubstituted fused arylene group including 10 to 24 ring carbon atoms or a substituted or unsubstituted fused heteroarylene group including 9 to 30 ring atoms,

f is an integer of 0 or 1, and

R⁶⁰ is a substituted or unsubstituted aryl group including 6 to 24 ring carbon atoms or a substituted or unsubstituted heteroaryl group including 5 to 30 ring atoms.

It is preferred that the substituted or unsubstituted fused aryl group including 10 to 24 ring carbon atoms in R⁶⁰ be a monovalent residue of compounds represented by the following formulas (a1-1) to (a1-3):

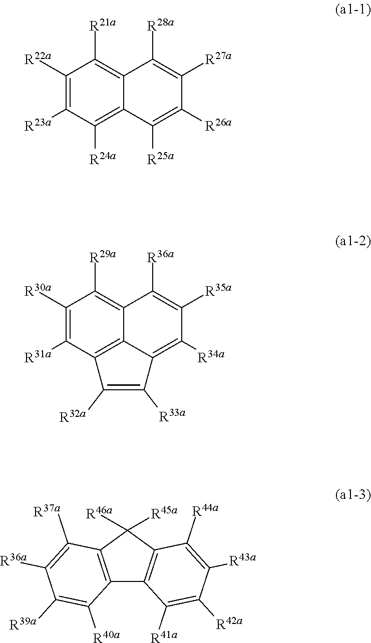

wherein in the formulas (a1-1) to (a1-3),

R^{21a} to R^{46a} are independently a hydrogen atom or a substituent R^{b}, and when plural R^{b}s are present, the plural R^{b}s may be the same or different, and two selected from the plural R^{b}s may be bonded with each other to form a ring.

As the substituent R^{b}, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more substituents selected from the group consisting of an alkyl group including 1 to 25 carbon atoms and an aryl group including 6 to 24 ring carbon atoms, a cyano group or a —P(═O)R¹⁴R¹⁵ group can be given,

R¹⁴ and R¹⁵ are as defined in the formula (2).

As the fused ring-forming residue including 10 to 24 ring carbon atoms in the compound represented by the formula (a1-1), the following fused aromatic rings can be given, for example. Among these, a fused aromatic ring group in which four or more rings are fused is preferable. Specifically, a triphenylenyl group, etc. can be given.

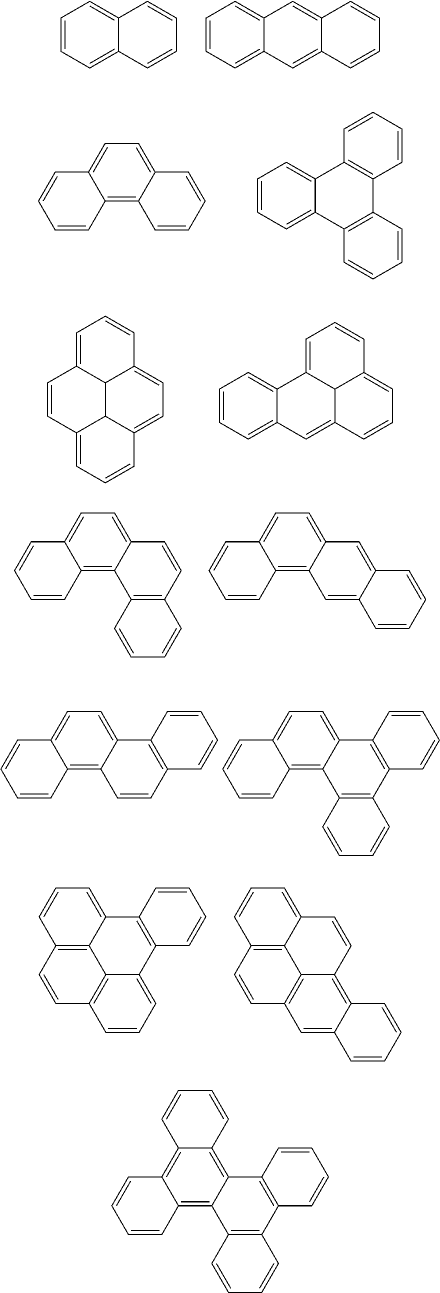

As the fused ring-forming residue including 10 to 24 ring carbon atoms in the compound represented by the formula (a1-2), the following compounds can be given, for example. Among these, a fused ring-forming group in which four or more rings are fused is preferable. Specifically, a fluororanthenyl group, etc. can be given.

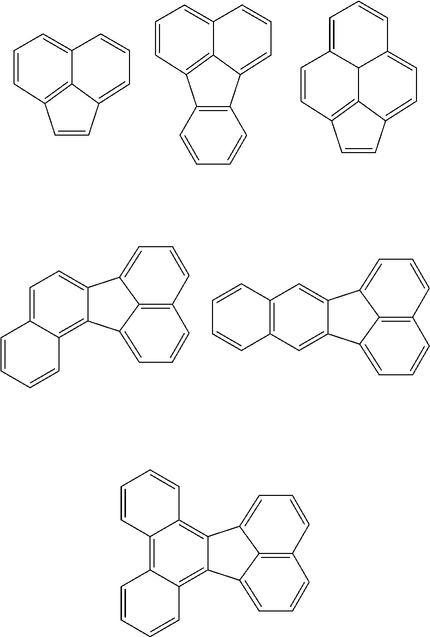

As the fused ring-forming residue including 10 to 24 ring carbon atoms in the compound represented by the formula (a1-3), a fused ring-forming group in which four or more rings are fused is preferable. As specific examples thereof, a benzo[a]fluorenyl group, etc. can be given.

As the substituted or unsubstituted fused heteroaryl group including 9 to 30 ring atoms in R⁶ , among the groups given above as the heteroaryl, one having 9 to 30 ring atoms can be given. Among them, the heteroaryl group is preferably a monovalent residue of a compound represented by the following formula (a2):

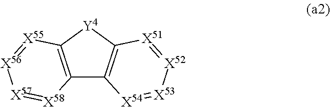

wherein in the formula (a2),

X⁵¹ to X⁵⁸ are independently CH, C(R^{b}) or N,

R^{b} is a substituent, and when plural R^{b}s are present, the plural R^{b}s may be the same or different, and two selected from the plural R^{b}s may be bonded with each other to form a ring,

Y⁴ is an oxygen atom, a sulfur atom, —NR^{d}, or —C(R^{e})(R^{f})—, provided that when Y⁴ is —C(R^{e})(R^{f})—, at least one of X⁵¹ to X⁵⁸ is N, and

R^{d}, R^{e} and R^{f} are independently a hydrogen atom or a substituent R^{b}, and when both of R^{e} and R^{f} are R^{b}, they may be bonded with each other to form a ring.

The substituent R^{b} is as defined above.

It is preferred that the monovalent residue represented by the formula (a2) be a monovalent residue of the compound represented by the following formula (a2-1):

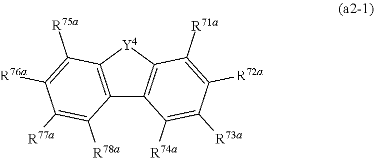

wherein in the formula (a2-1),

Y⁴ is as defined in the formula (a2), and

R^{71a} to R^{71a} are independently a hydrogen atom or a substituent R^{b}, and when plural R^{b}s are present, they may be the same or different, and two selected from the plural R^{b}s may be bonded with each other to form a ring.

In the compound represented by the formula (a2-1), it is preferred that Y⁴ be an oxygen atom, a sulfur atom, NH or C(CH₃)₂, for example.

It is preferred that any one of R^{71a} to R^{71a} form a single bond with L¹.

It is preferred that the compound represented by the formula (30) be a compound represented by the following formula (31):

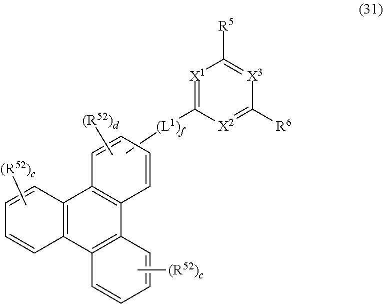

wherein in the formula (31),

X¹ to X³, R⁵, R⁶, L¹ and f are as defined in the formula (30),

R⁵² is a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more substituents selected from the group consisting of an alkyl group including 1 to 25 carbon atoms and an aryl group including 6 to 24 ring carbon atoms, a cyano group or a —P(═O)R¹⁴R¹⁵ group, adjacent substituents on R⁵² may be bonded with each other to form a ring,

R¹⁴ and R¹⁵ are as defined in the formula (2),

c is an integer of 0 to 4 and d is an integer of 0 to 3,

when c is 2 to 4 and d is 2 or 3, plural R⁵²s may be the same or different, and

adjacent R⁵²s may be bonded with each other to form a ring.

In another embodiment, it is preferred that the compound represented by the formula (30) be a compound represented by the following formula (32):

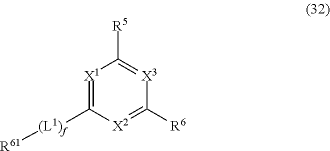

wherein in the formula (32),

X¹ to X³, R⁵, R⁶, L¹ and f are as defined in the formula (30), and

R⁶¹ is a substituted or unsubstituted fused heteroaryl group including 9 to 30 ring atoms that does not comprise a nitrogen atom.

As the fused heteroaryl group including 9 to 30 ring atoms that does not comprise a nitrogen atom, among the groups given above as the heteroaryl, a fused heteroaryl group including 9 to 30 ring atoms and does not comprise a nitrogen atom can be given.

It is preferred that the compound represented by the formula (32) be a compound represented by the following formula (33):

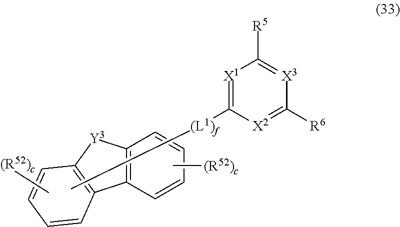

wherein in the formula (33),

X¹ to X³, R⁵, R⁶, L¹ and f are as defined in the formula (32),

R⁵² is a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more substituents selected from the group consisting of an alkyl group including 1 to 25 carbon atoms and an aryl group including 6 to 24 ring carbon atoms, a cyano group or a —P(═O)R¹⁴R¹⁵ group, adjacent substituents on R⁵² may be bonded with each other to form a ring;

R¹⁴ and R¹⁵ are as defined in the formula (2),

c is an integer of 0 to 4,

when c is 2 to 4, plural R⁵²s may be the same or different,

adjacent R⁵²s may be bonded with each other to form a ring, and

Y³ is an oxygen atom or a sulfur atom.

It is preferred that, in the formula (2), R⁵to R⁷ be a monovalent group formed of a single ring selected from the following group or a monovalent group formed by linking of two or more rings selected from the following group:

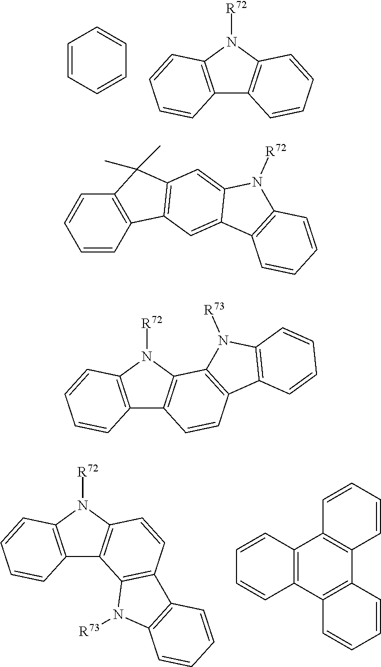

wherein in the formula, R⁷² and R⁷³ are independently an atomic bonding, a hydrogen atom, a substituted or unsubstituted aryl group including 6 to 24 ring carbon atoms or a substituted or unsubstituted heteroaryl group including 5 to 30 ring atoms.

It is preferred that, in the formula (2), X¹ to X³ be N,

R⁵ and R⁶ be a phenyl group, and

R⁷ be a group selected from the following group:

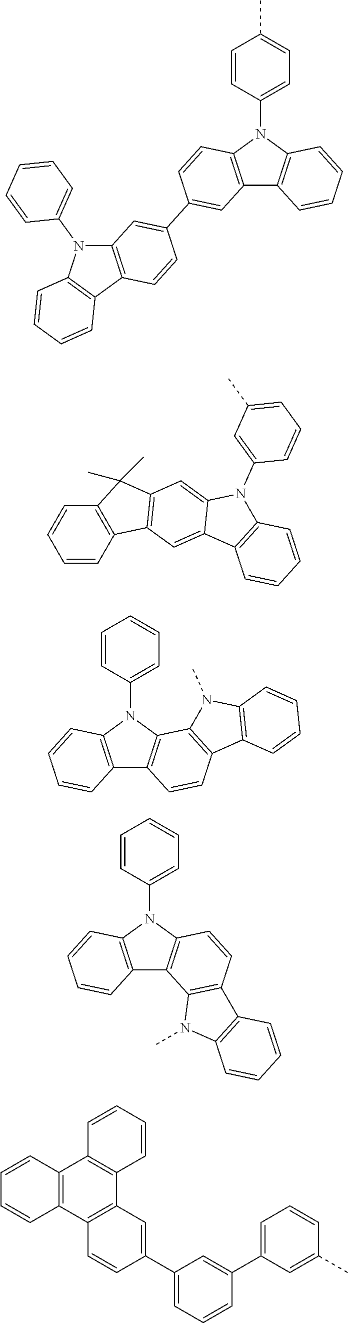

[in the formula, the dotted line indicates an atomic bonding]

Specific examples of the compound represented by the formula (2) are given below. However, the compound represented by the formula (2) is not limited thereto.

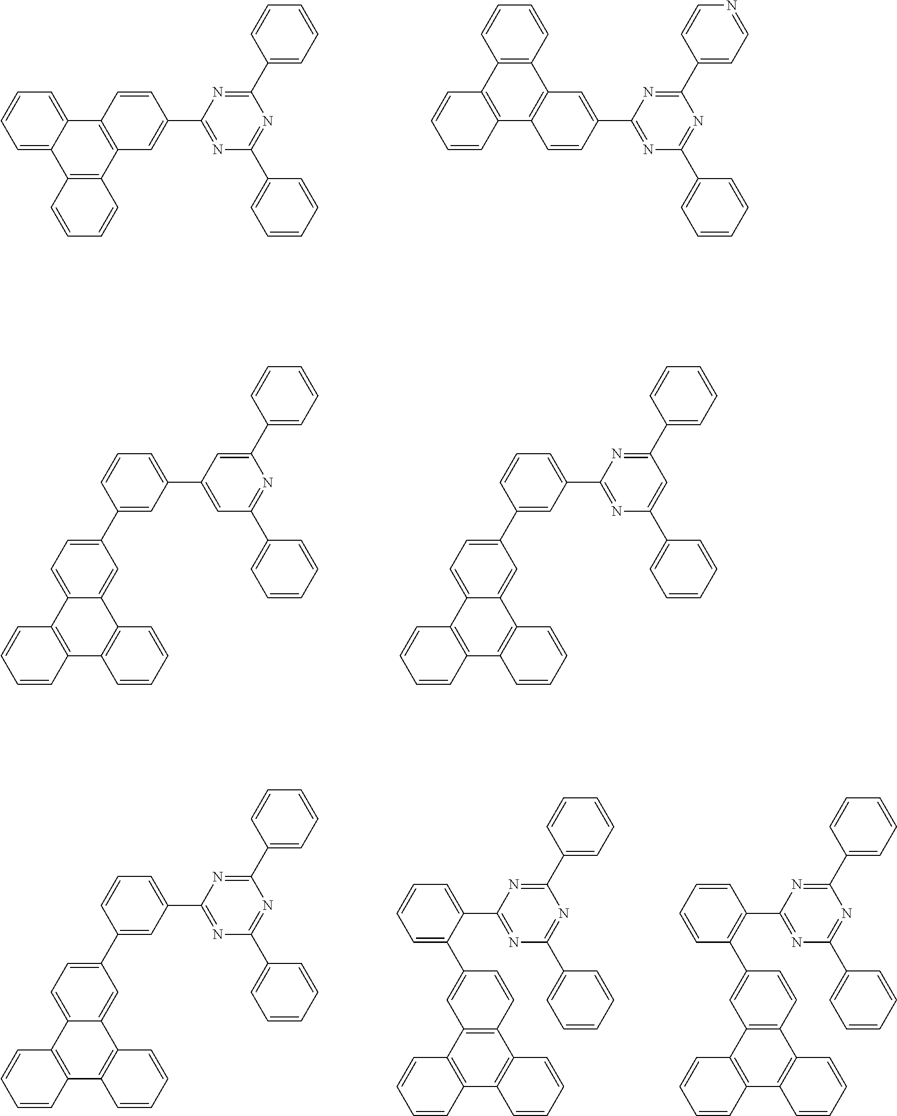

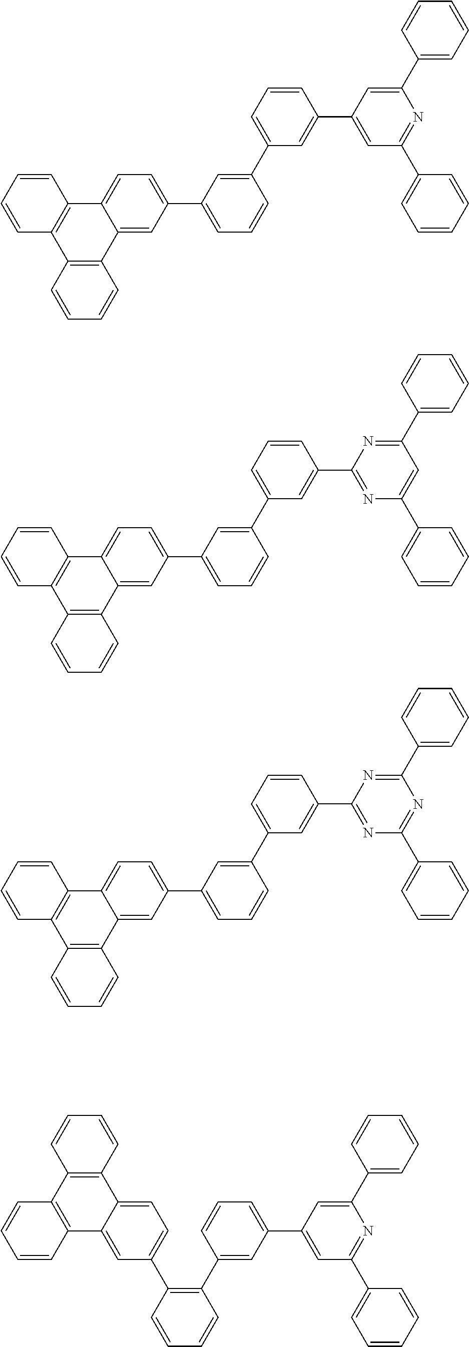 [SMILES: c1ccc(-c2cc(-c3cccc(-c4cccc(-c5ccc6c7ccccc7c7ccccc7c6c5)c4)c3)cc(-c3ccccc3)n2)cc1 ; c1ccc(-c2cc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2cc(-c3cccc(-c4ccccc4-c4ccc5c6ccccc6c6ccccc6c5c4)c3)cc(-c3ccccc3)n2)cc1]

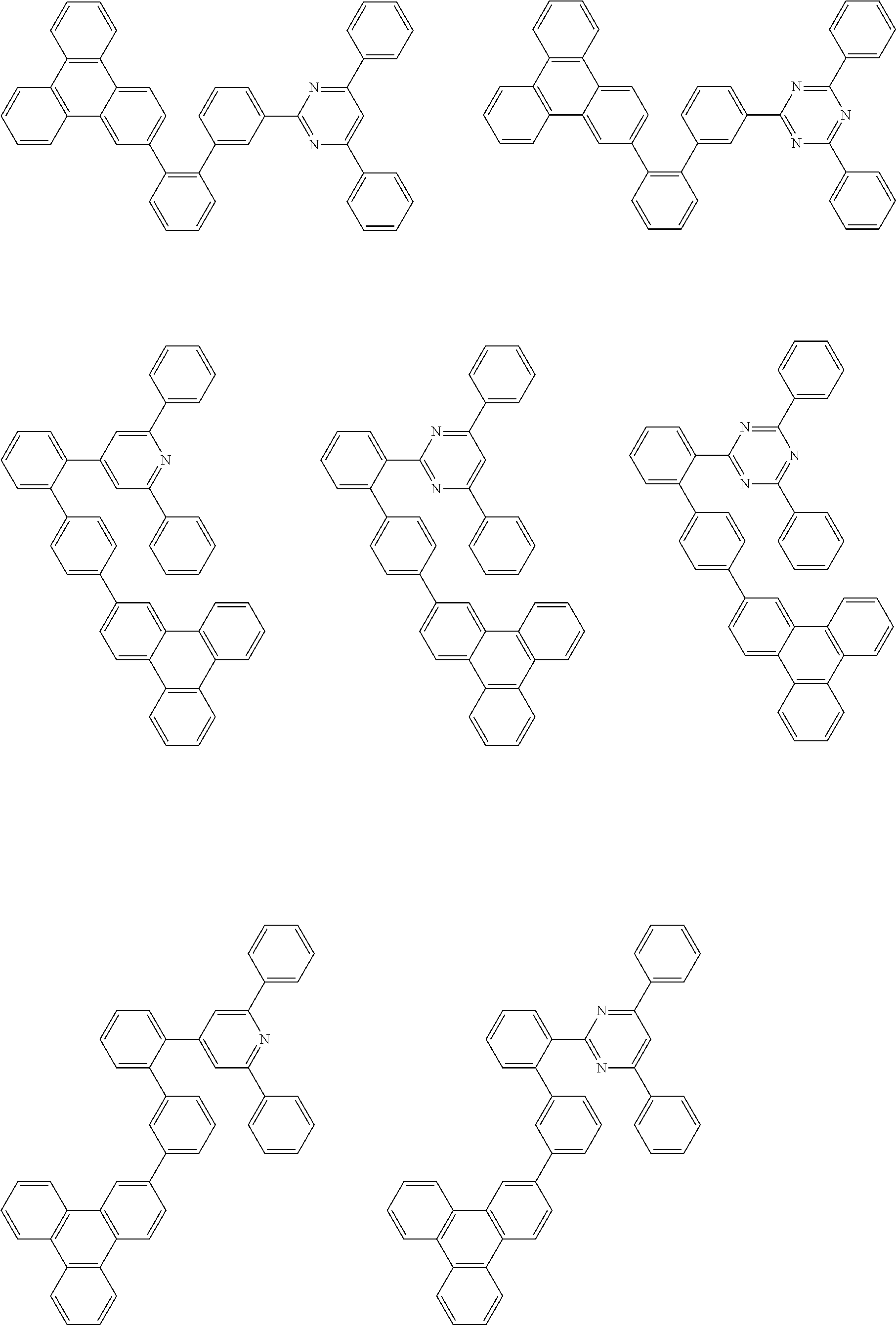

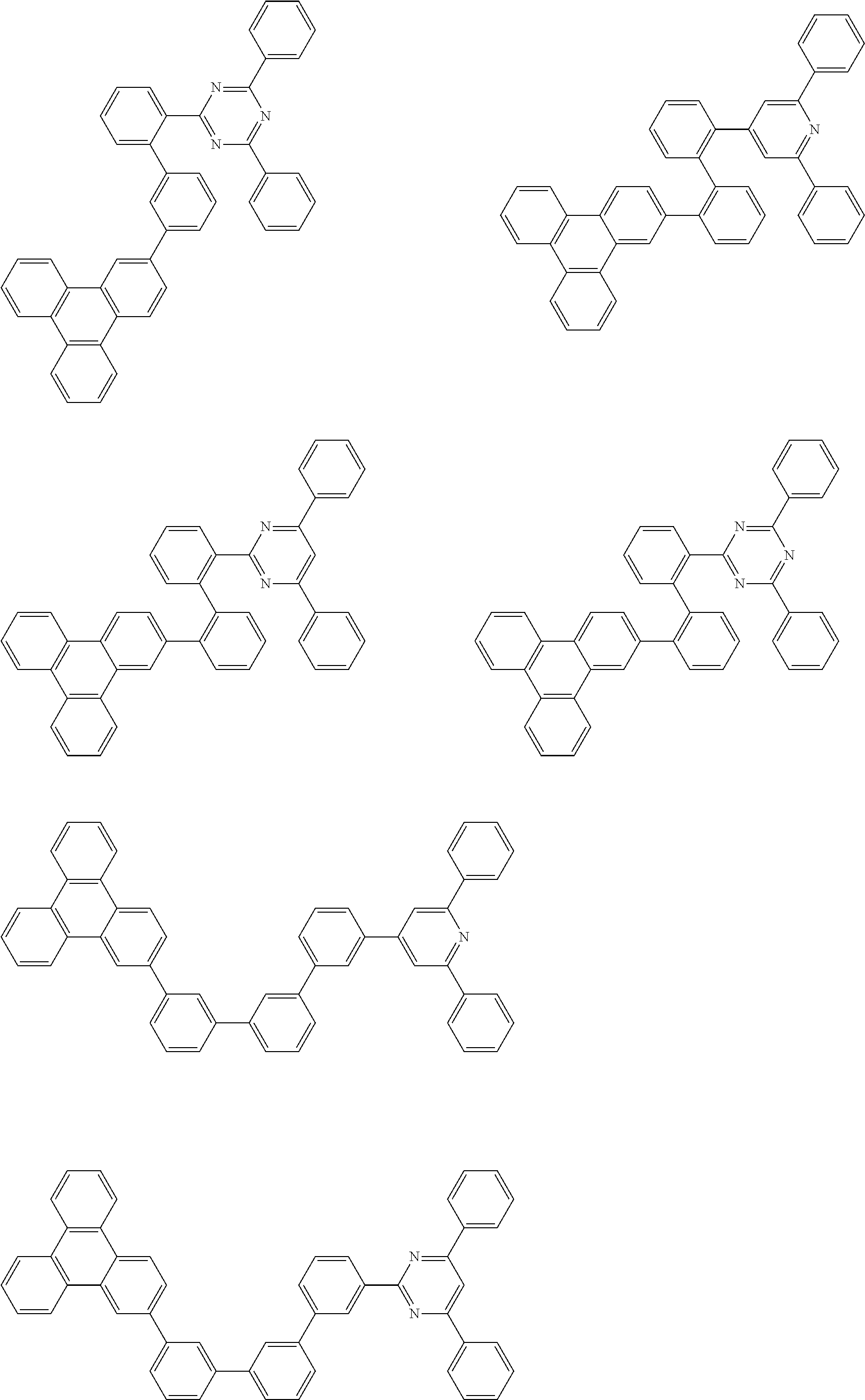

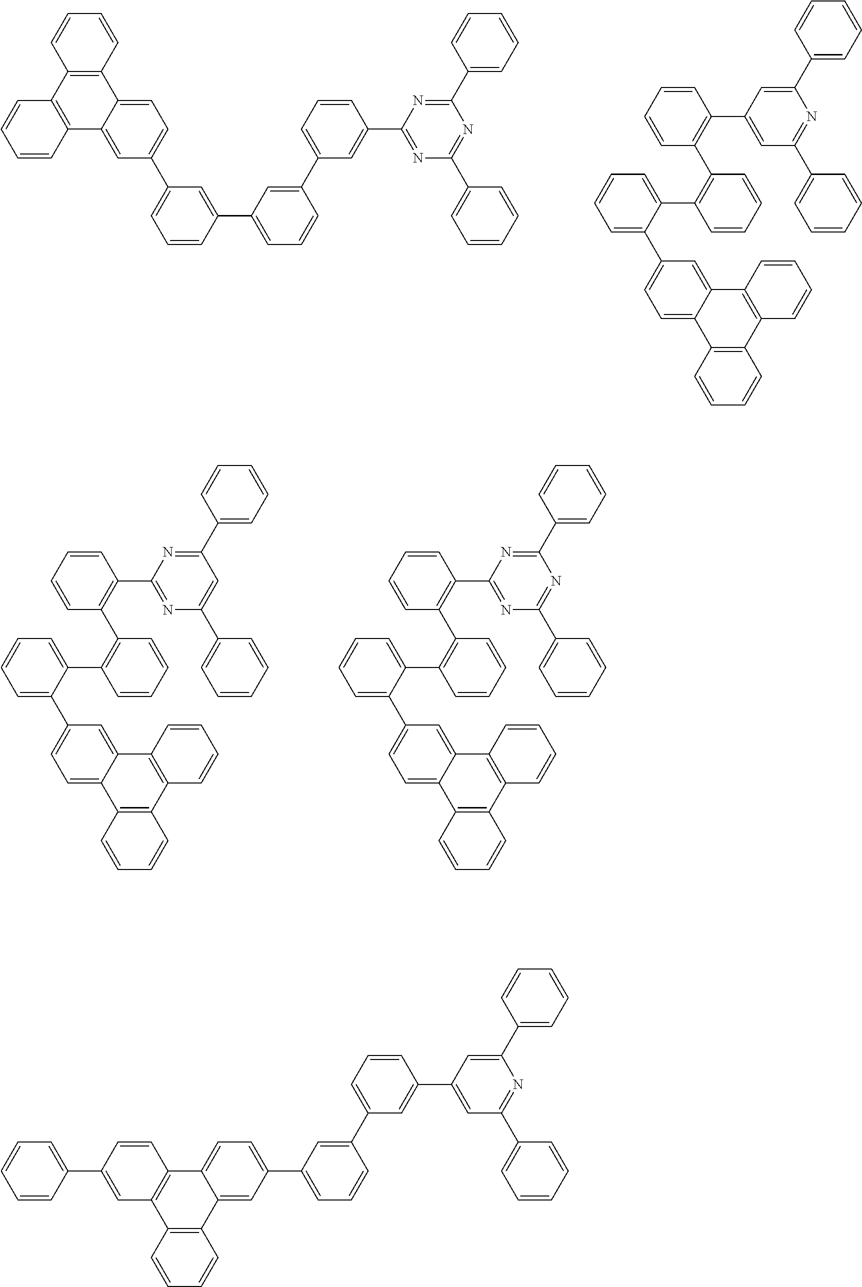 [SMILES: c1ccc(-c2nc(-c3ccccc3)nc(-c3cccc(-c4cccc(-c5cccc(-c6ccc7c8ccccc8c8ccccc8c7c6)c5)c4)c3)n2)cc1 ; c1ccc(-c2cc(-c3ccccc3-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2ccc3c4ccc(-c5cccc(-c6cccc(-c7cc(-c8ccccc8)nc(-c8ccccc8)c7)c6)c5)cc4c4ccccc4c3c2)cc1]

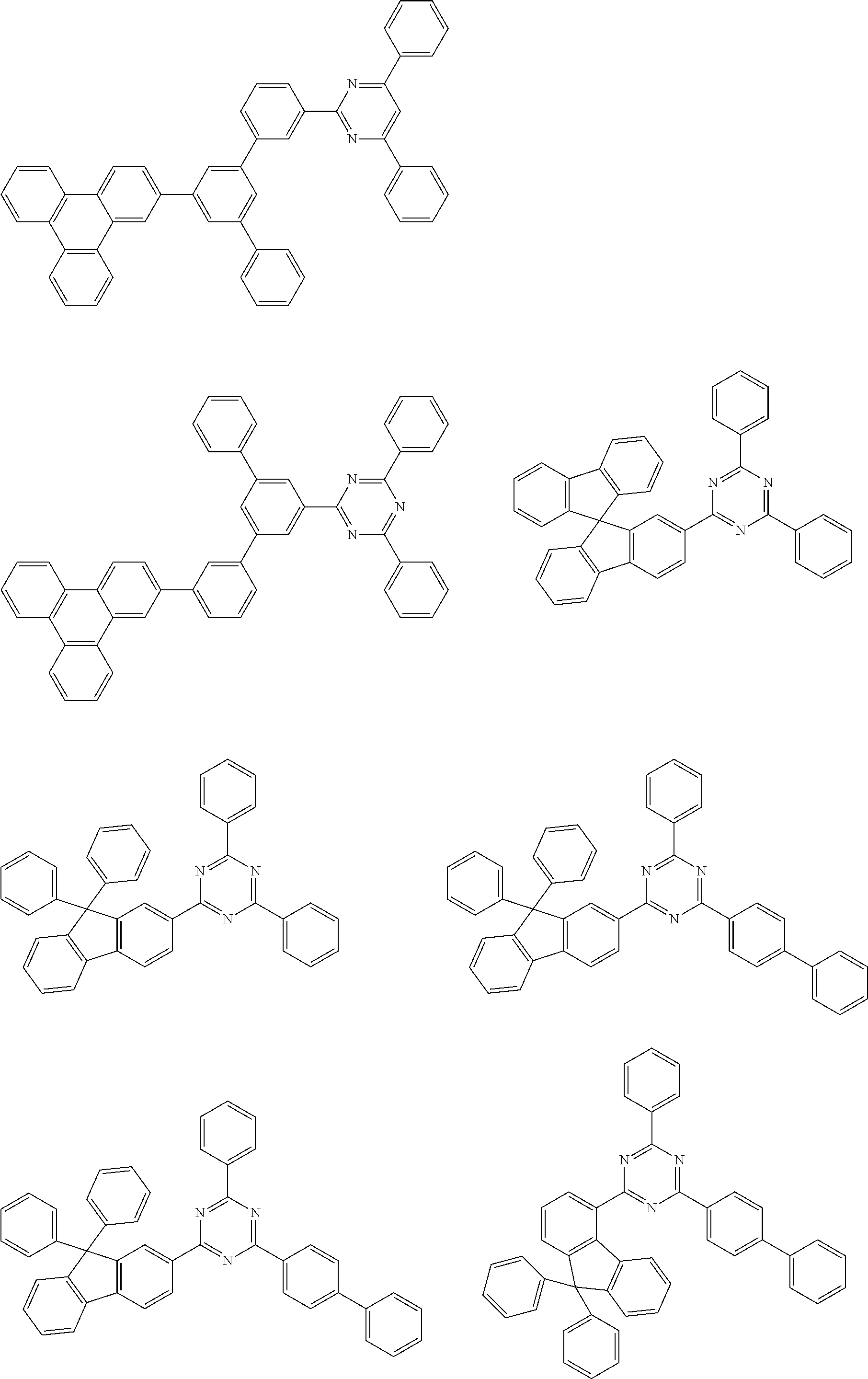

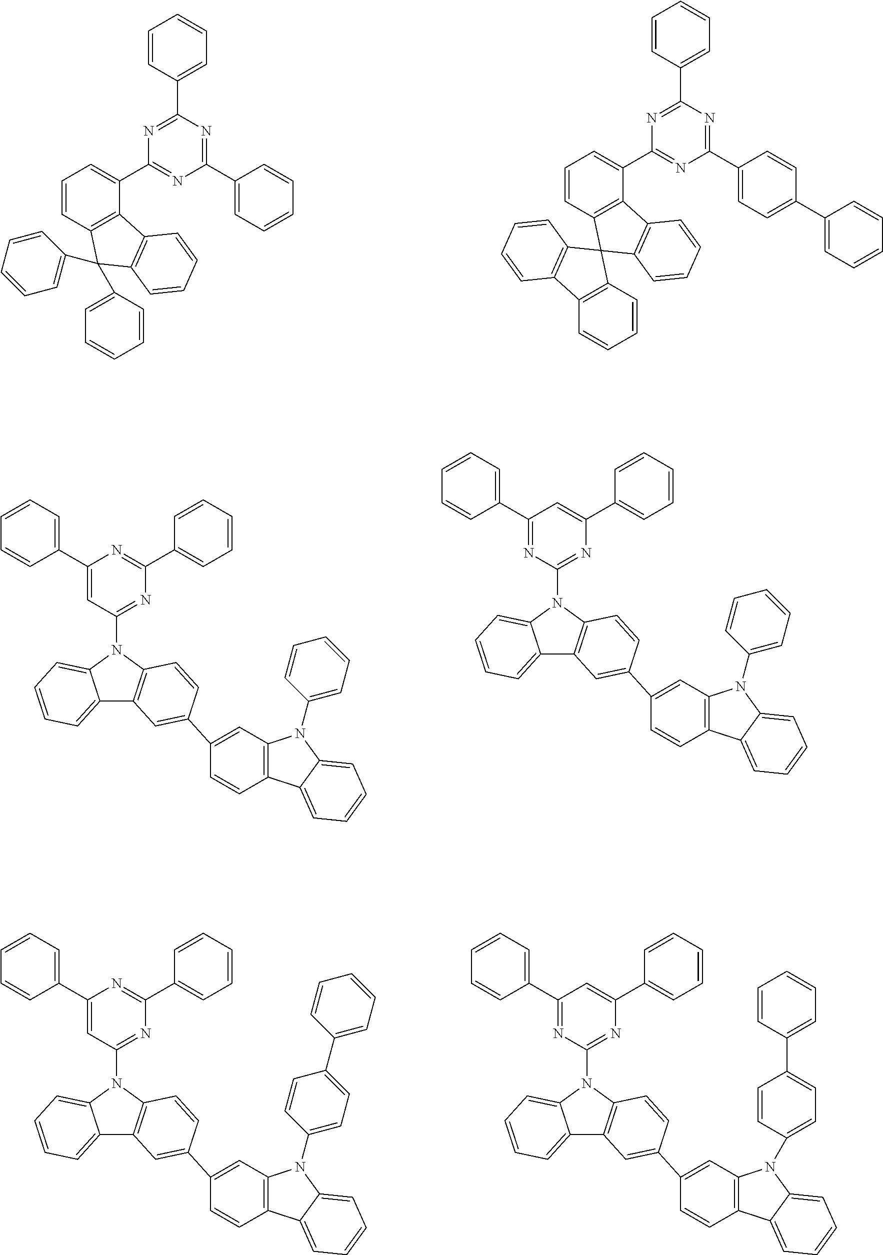

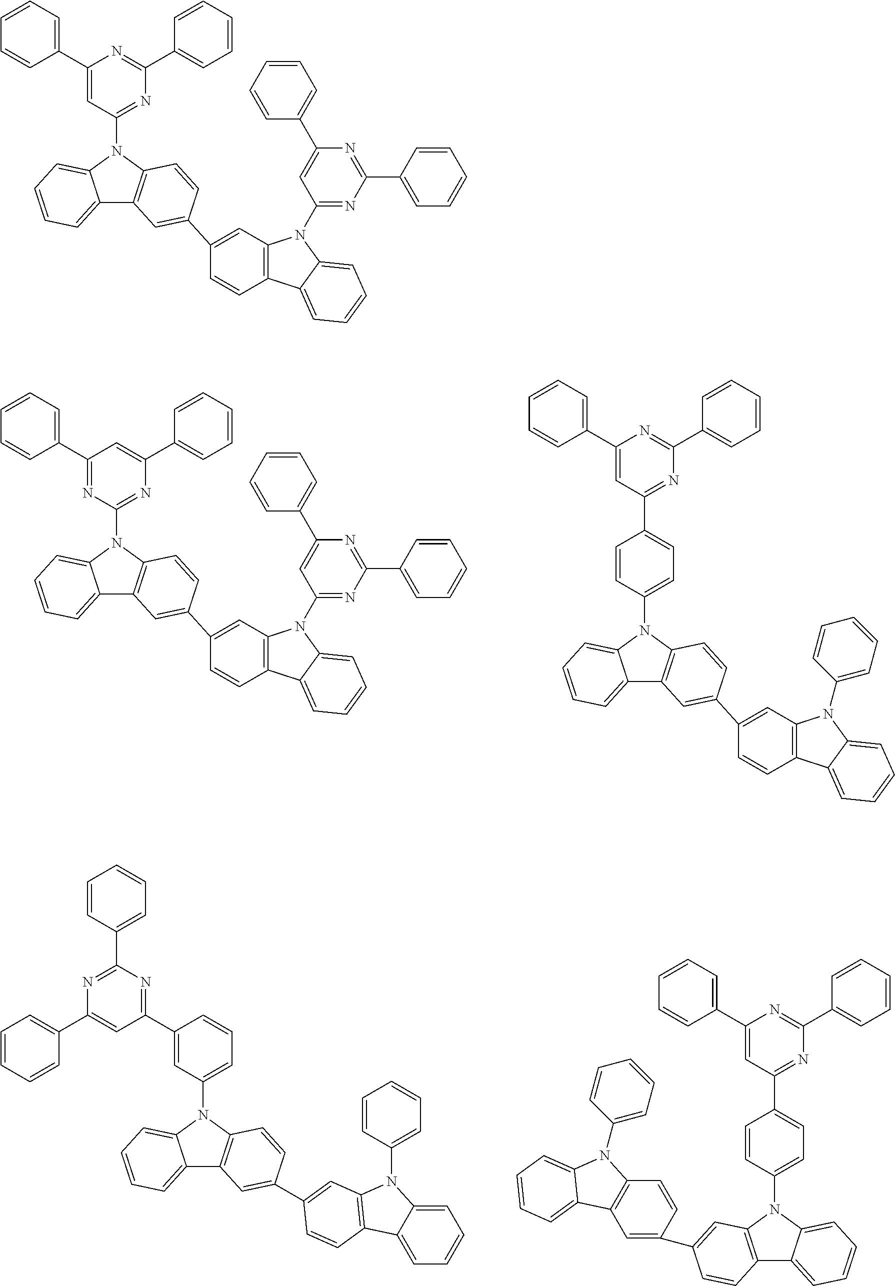

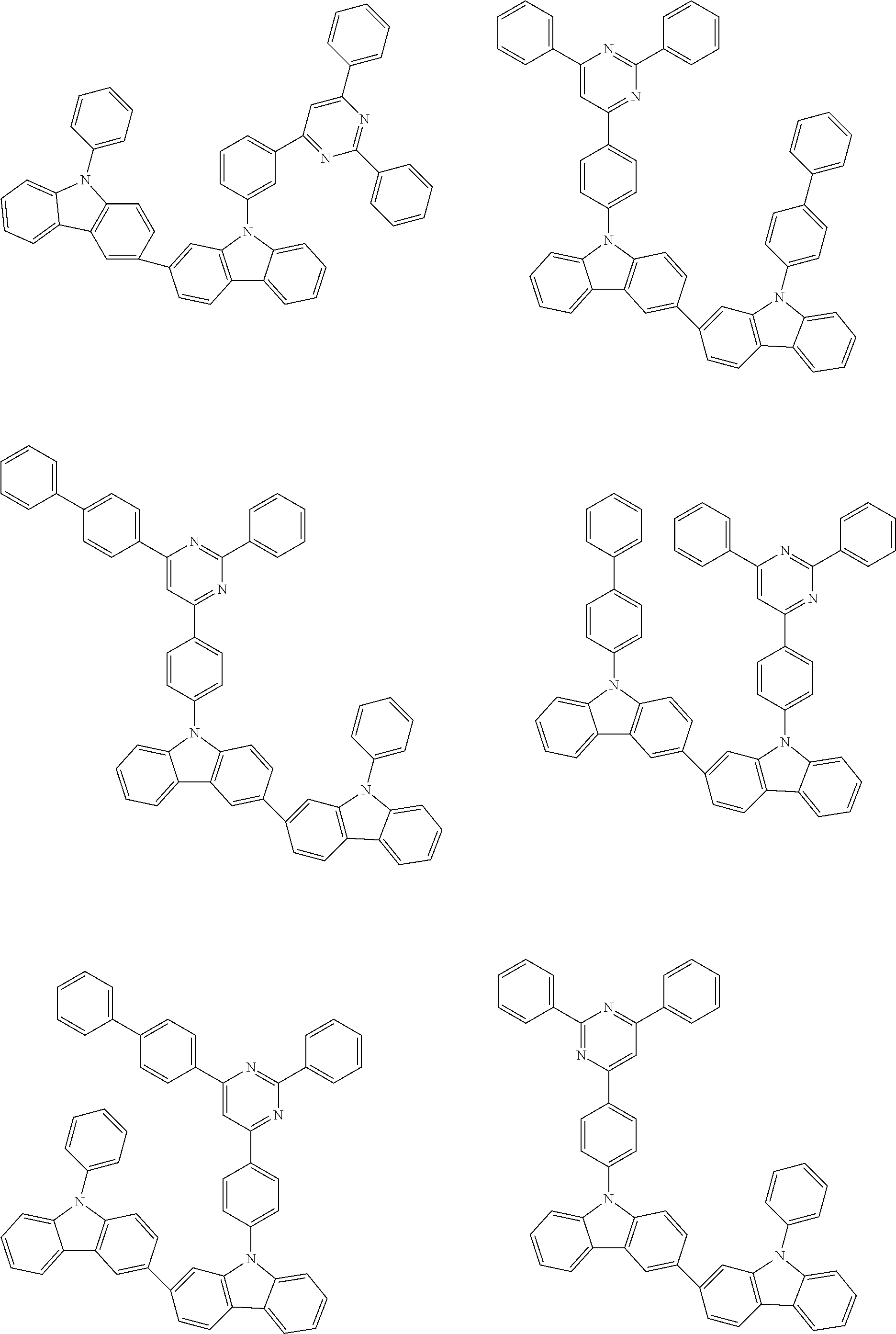

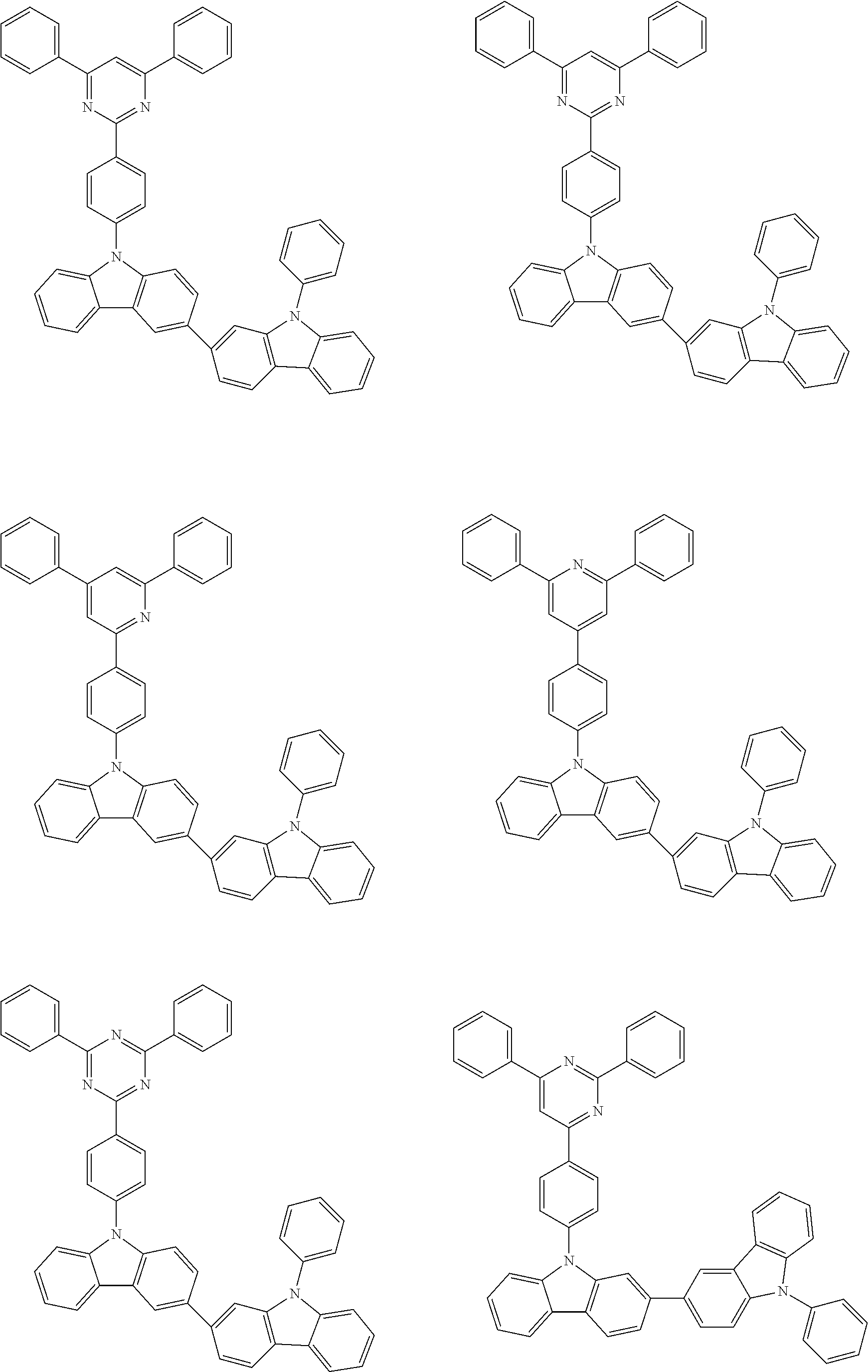

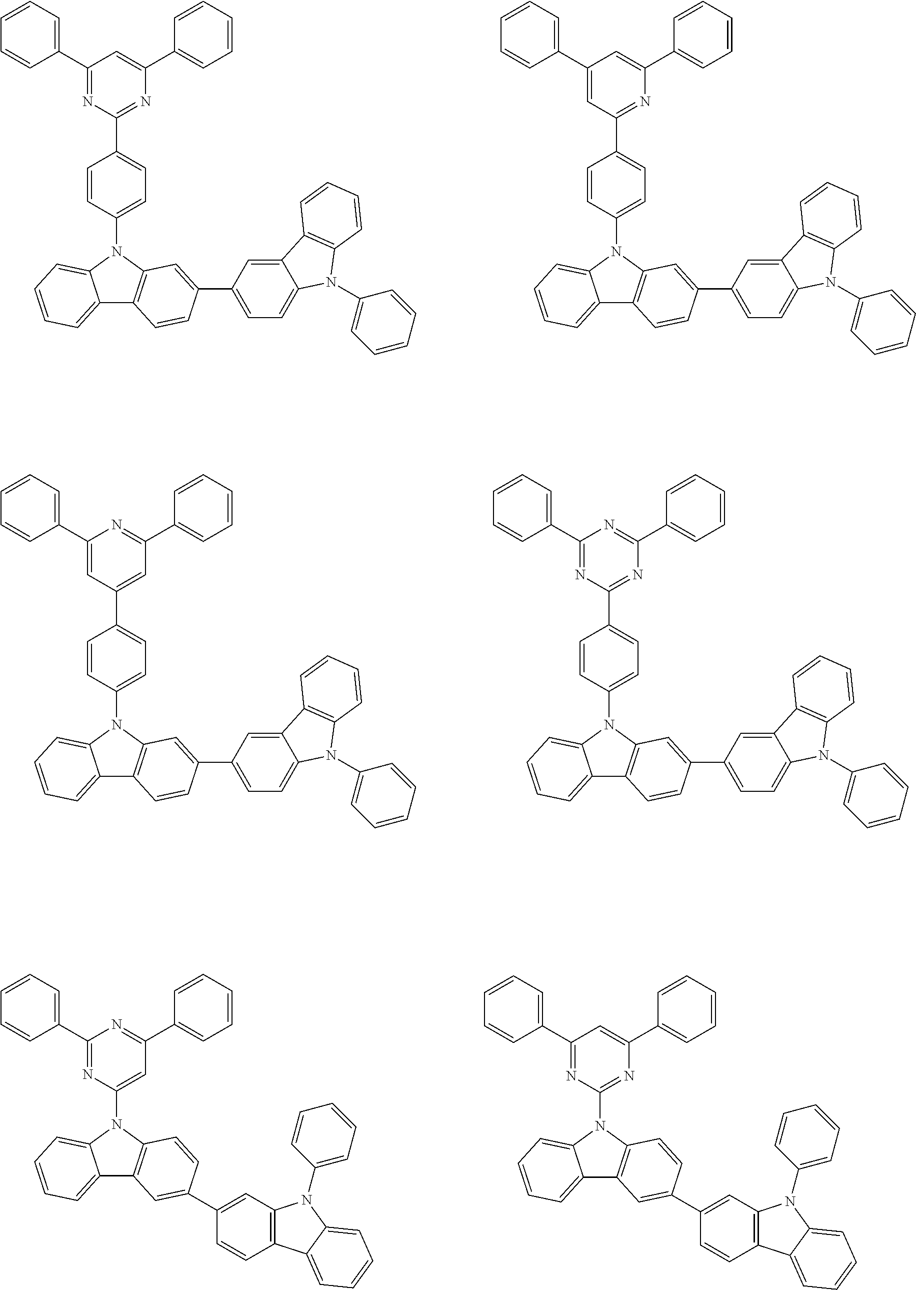

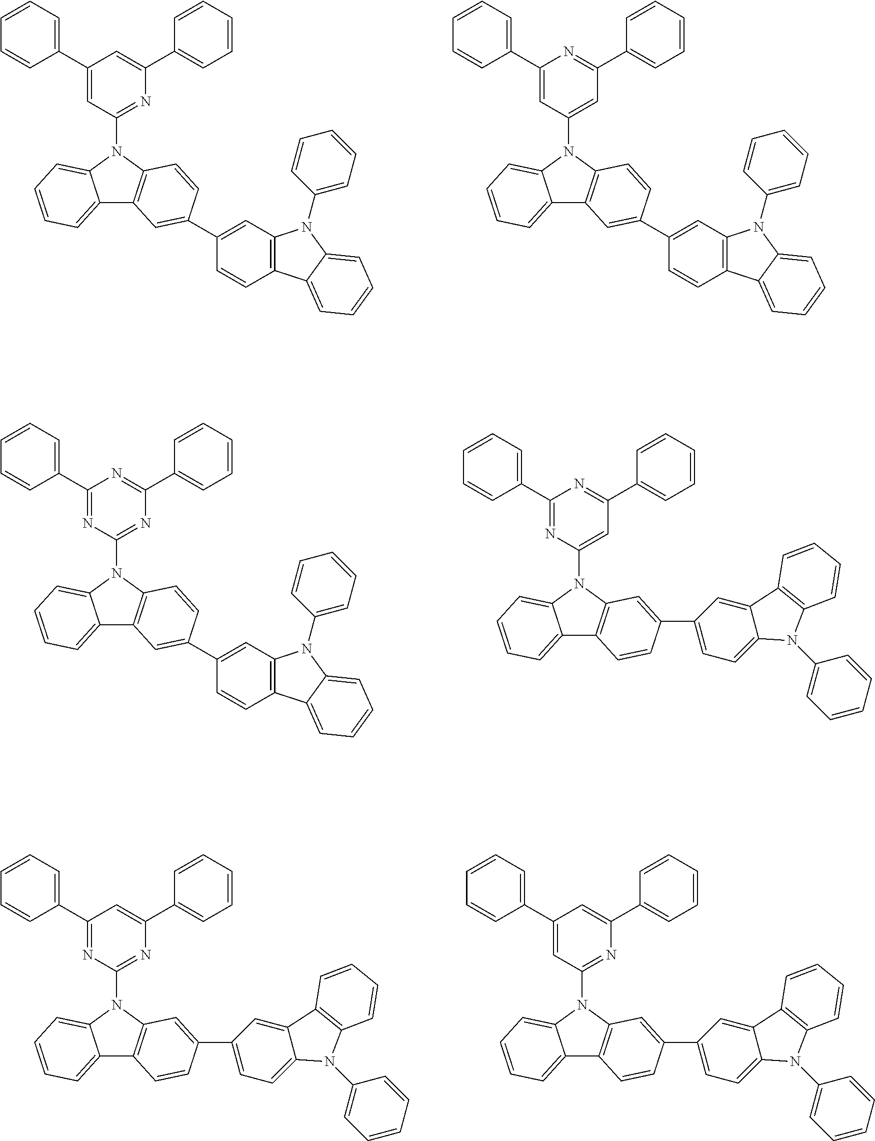

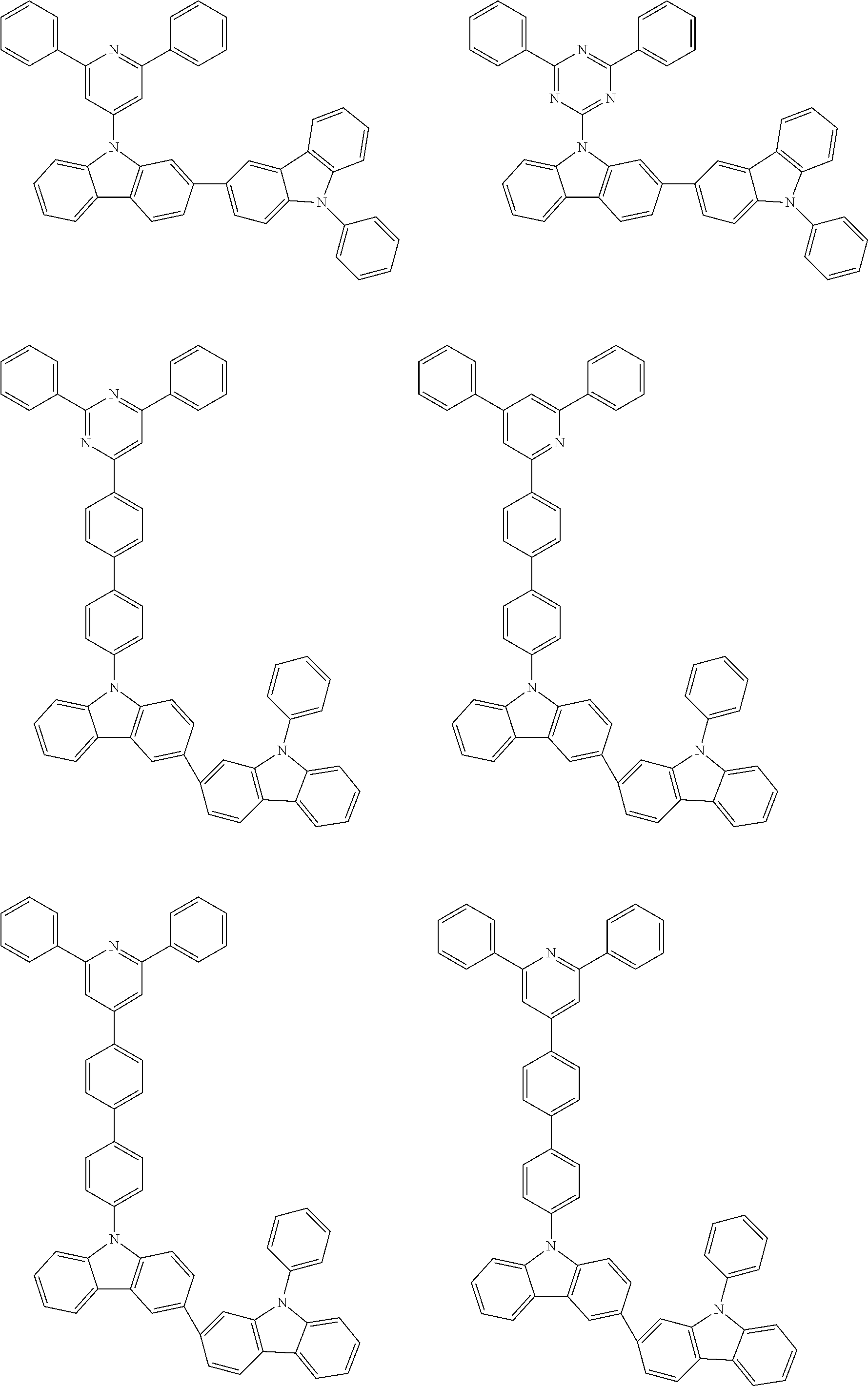

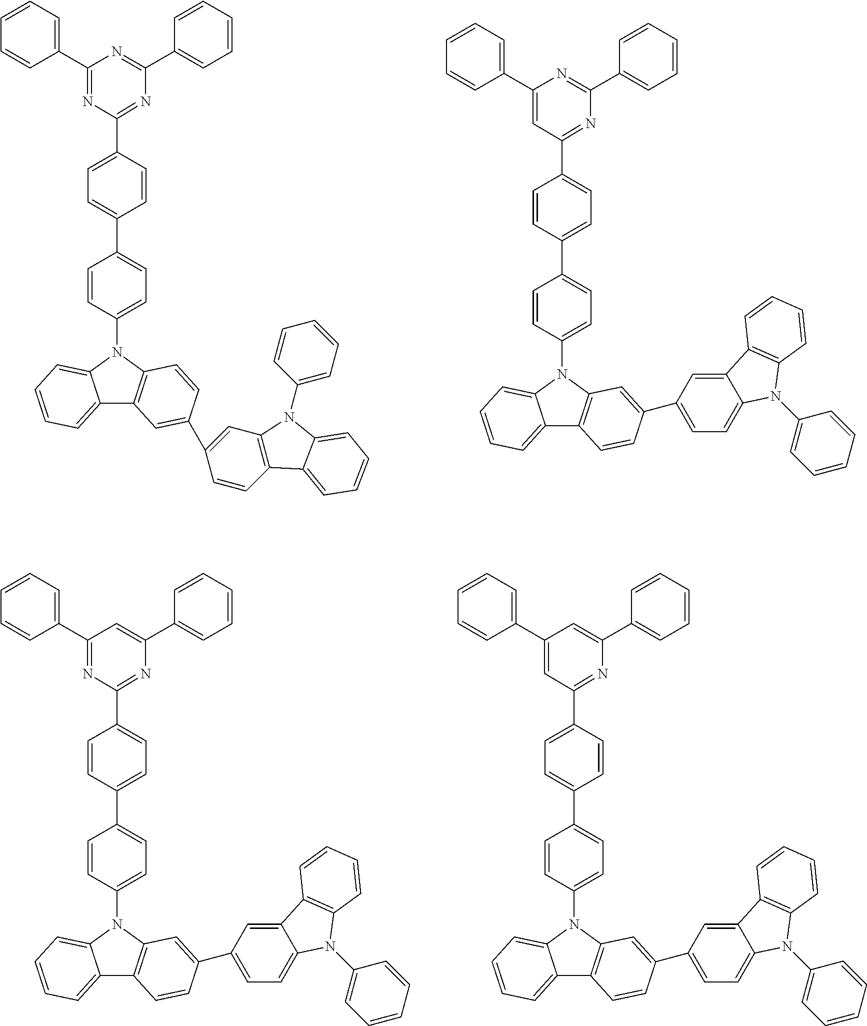 [SMILES: c1ccc(-c2nc(-c3ccccc3)nc(-c3ccc(-c4ccc(-n5c6ccccc6c6cc(-c7ccc8c9ccccc9n(-c9ccccc9)c8c7)ccc65)cc4)cc3)n2)cc1 ; c1ccc(-c2cc(-c3ccc(-c4ccc(-n5c6ccccc6c6ccc(-c7ccc8c(c7)c7ccccc7n8-c7ccccc7)cc65)cc4)cc3)nc(-c3ccccc3)n2)cc1 ; c1ccc(-c2cc(-c3ccccc3)nc(-c3ccc(-c4ccc(-n5c6ccccc6c6ccc(-c7ccc8c(c7)c7ccccc7n8-c7ccccc7)cc65)cc4)cc3)n2)cc1 ; c1ccc(-c2cc(-c3ccccc3)nc(-c3ccc(-c4ccc(-n5c6ccccc6c6ccc(-c7ccc8c(c7)c7ccccc7n8-c7ccccc7)cc65)cc4)cc3)c2)cc1]

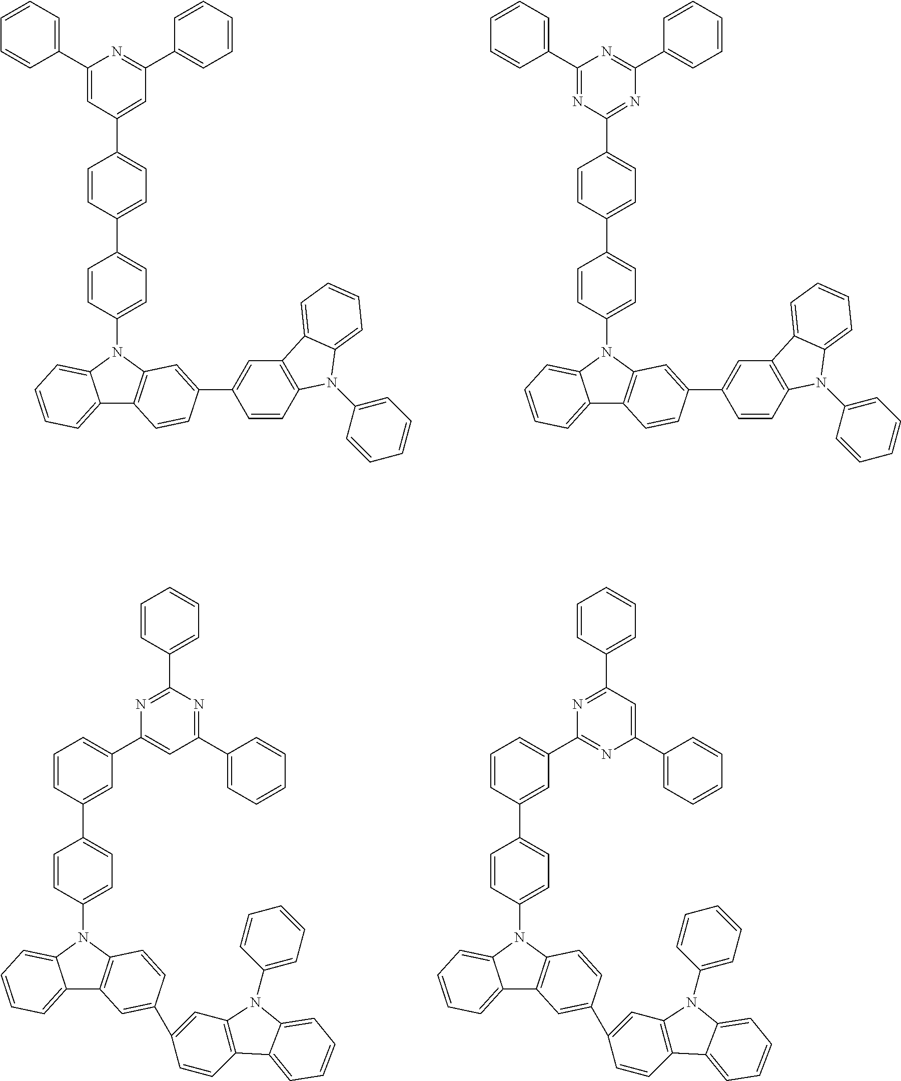 [SMILES: c1ccc(-c2cc(-c3ccc(-c4ccc(-n5c6ccccc6c6ccc(-c7ccc8c(c7)c7ccccc7n8-c7ccccc7)cc65)cc4)cc3)cc(-c3ccccc3)n2)cc1 ; c1ccc(-c2nc(-c3ccccc3)nc(-c3ccc(-c4ccc(-n5c6ccccc6c6ccc(-c7ccc8c(c7)c7ccccc7n8-c7ccccc7)cc65)cc4)cc3)n2)cc1 ; c1ccc(-c2cc(-c3cccc(-c4ccc(-n5c6ccccc6c6cc(-c7ccc8c9ccccc9n(-c9ccccc9)c8c7)ccc65)cc4)c3)nc(-c3ccccc3)n2)cc1 ; c1ccc(-c2cc(-c3ccccc3)nc(-c3cccc(-c4ccc(-n5c6ccccc6c6cc(-c7ccc8c9ccccc9n(-c9ccccc9)c8c7)ccc65)cc4)c3)n2)cc1]

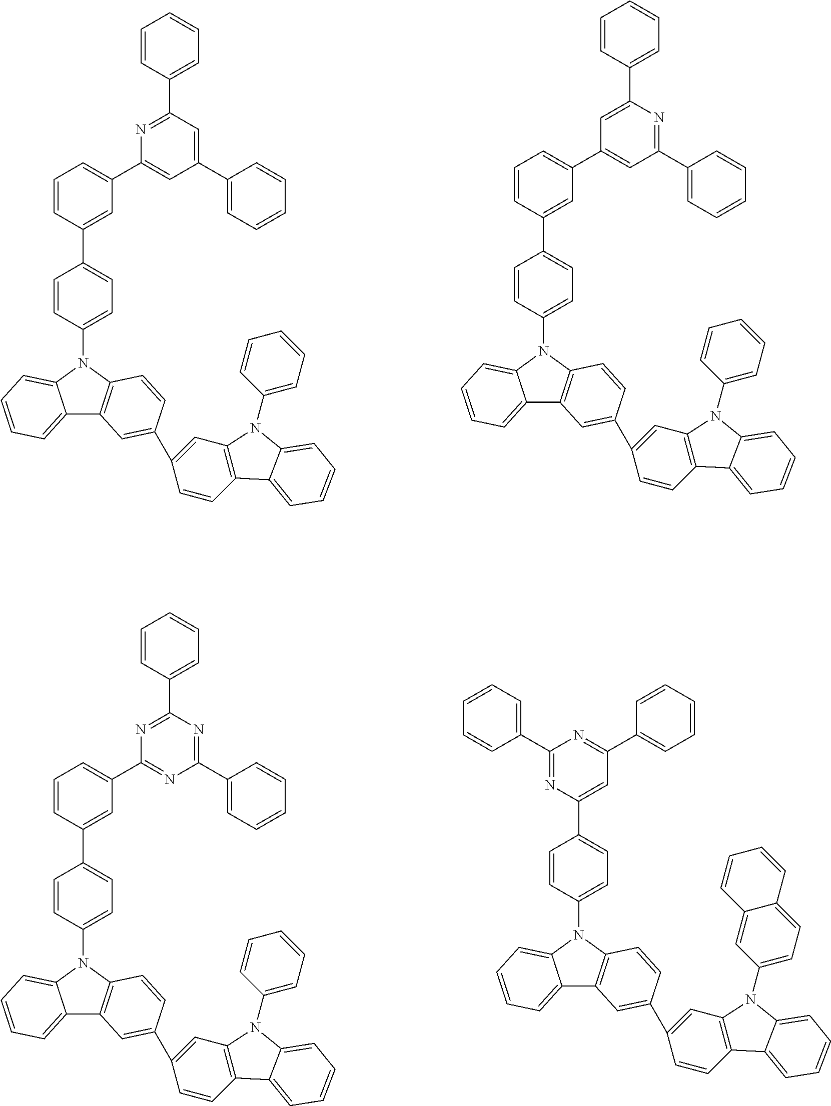 [SMILES: c1ccc(-c2cc(-c3ccccc3)nc(-c3cccc(-c4ccc(-n5c6ccccc6c6cc(-c7ccc8c9ccccc9n(-c9ccccc9)c8c7)ccc65)cc4)c3)c2)cc1 ; c1ccc(-c2cc(-c3cccc(-c4ccc(-n5c6ccccc6c6cc(-c7ccc8c9ccccc9n(-c9ccccc9)c8c7)ccc65)cc4)c3)cc(-c3ccccc3)n2)cc1 ; c1ccc(-c2nc(-c3ccccc3)nc(-c3cccc(-c4ccc(-n5c6ccccc6c6cc(-c7ccc8c9ccccc9n(-c9ccccc9)c8c7)ccc65)cc4)c3)n2)cc1 ; c1ccc(-c2cc(-c3ccc(-n4c5ccccc5c5cc(-c6ccc7c8ccccc8n(-c8ccc9ccccc9c8)c7c6)ccc54)cc3)nc(-c3ccccc3)n2)cc1]

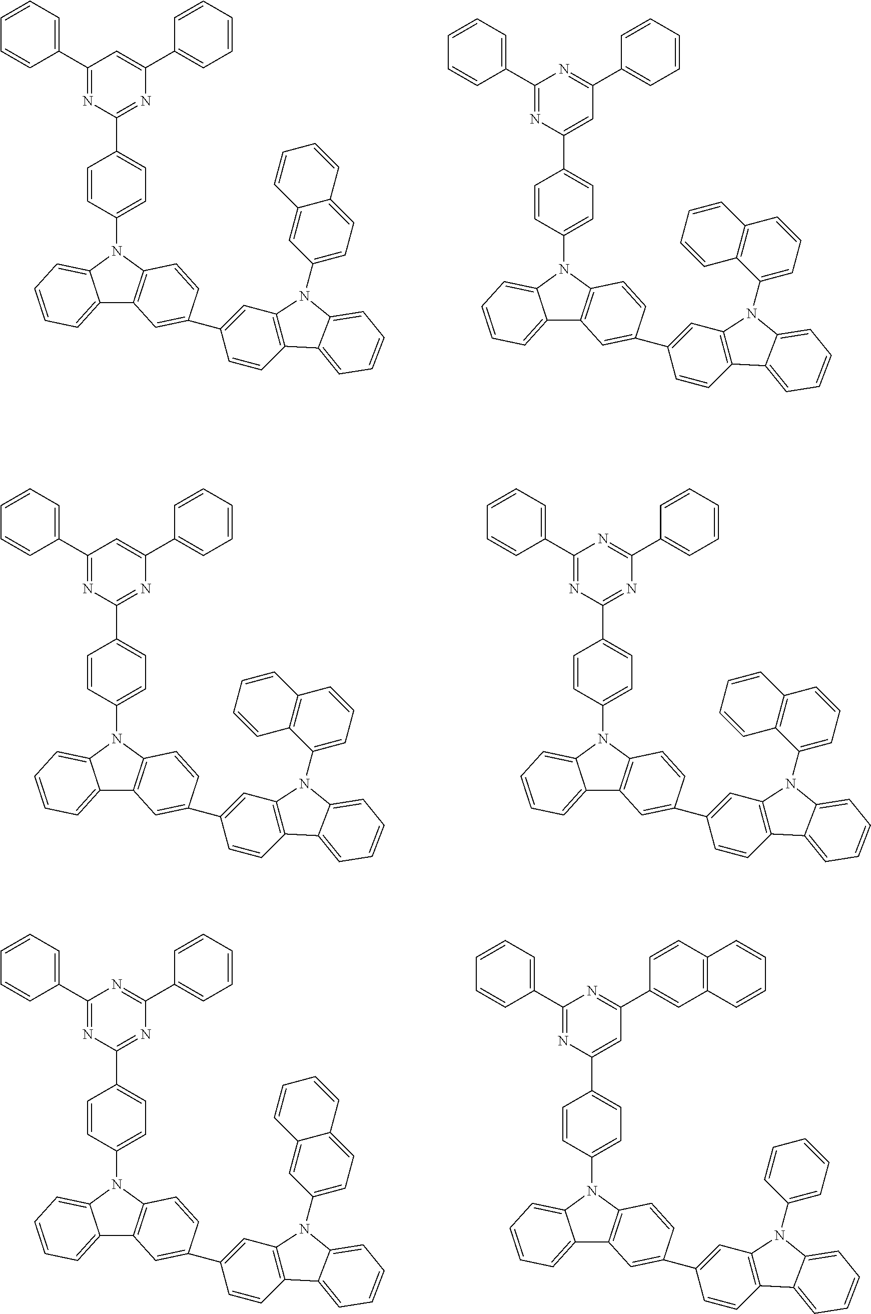

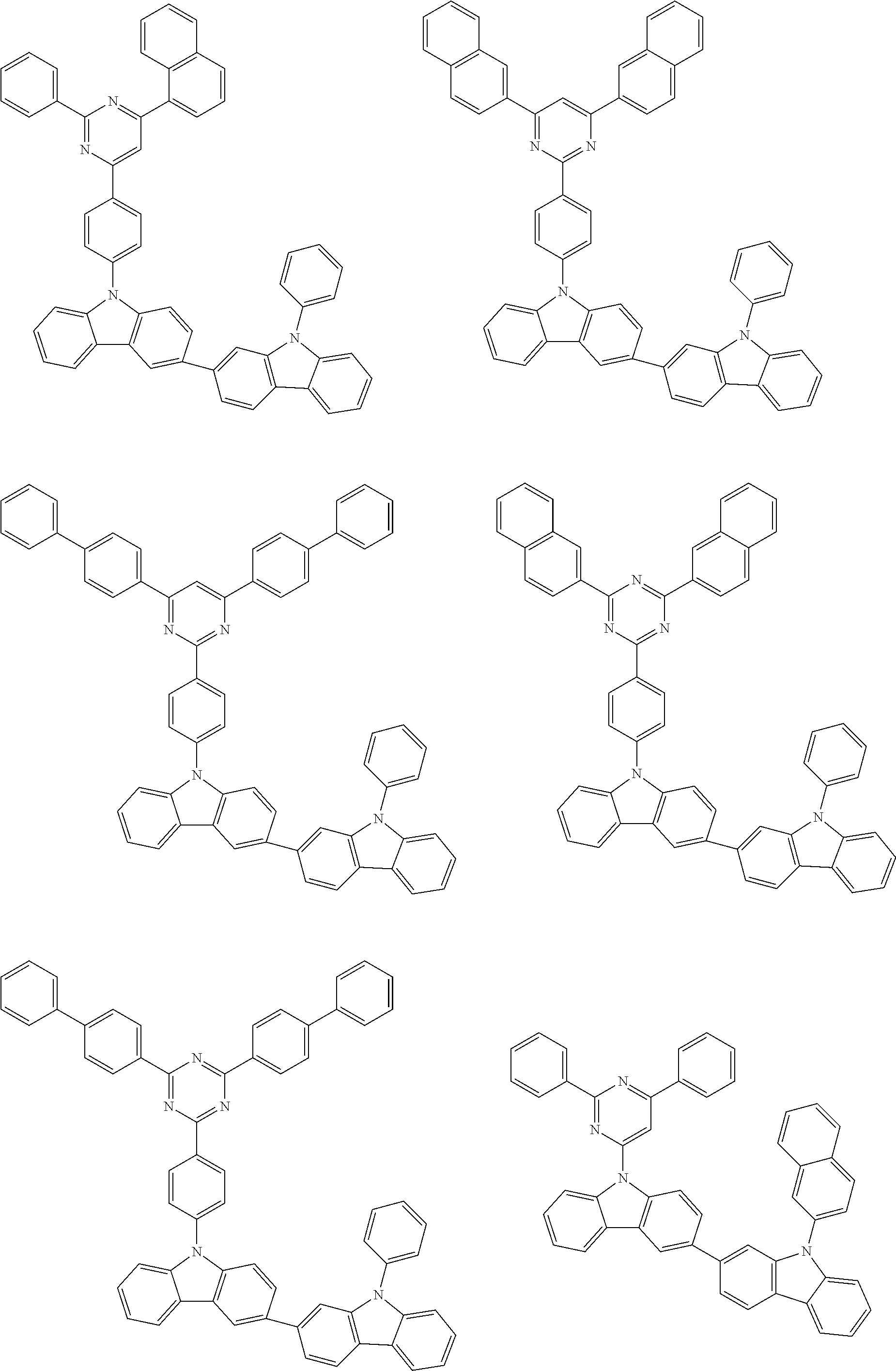

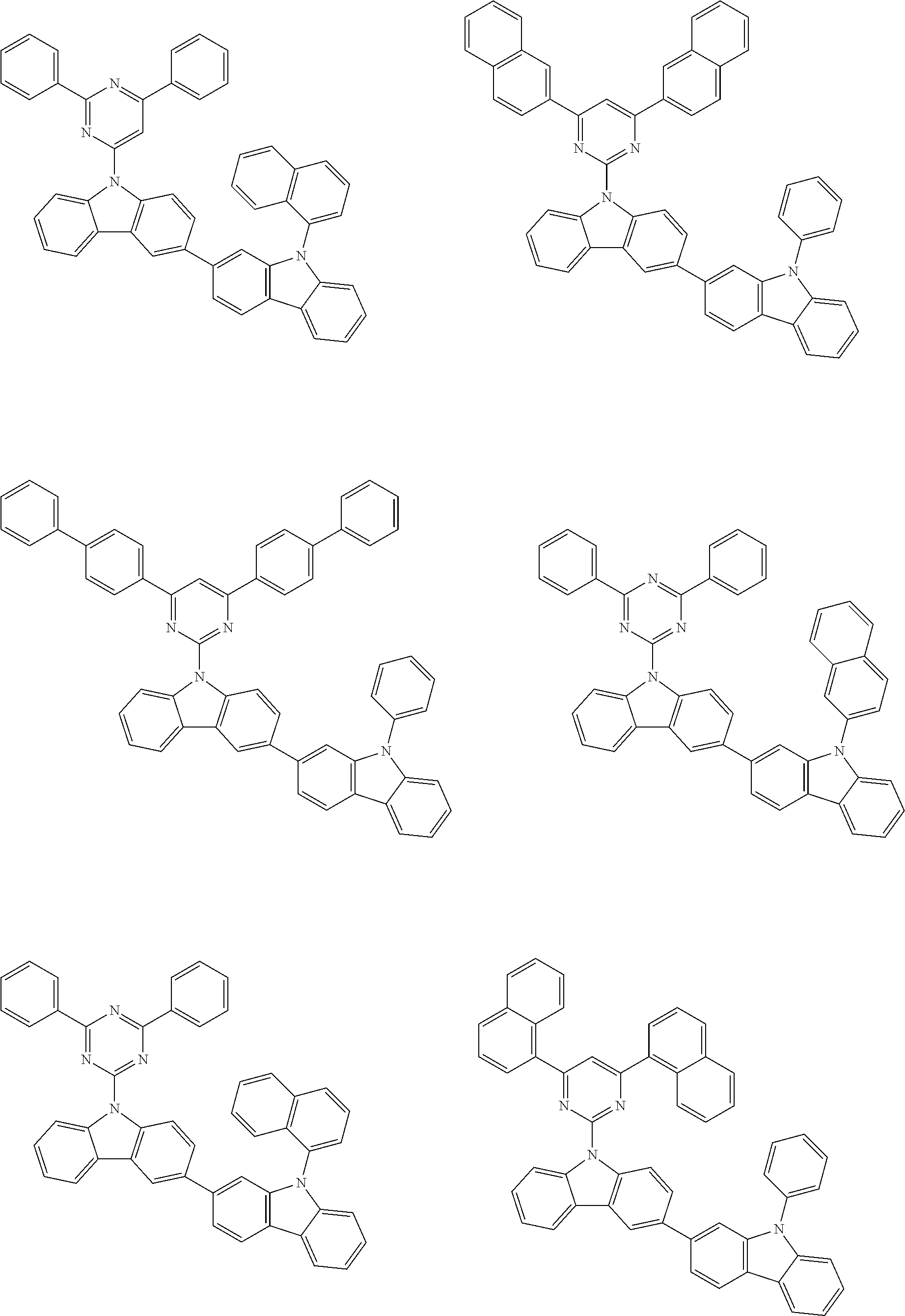

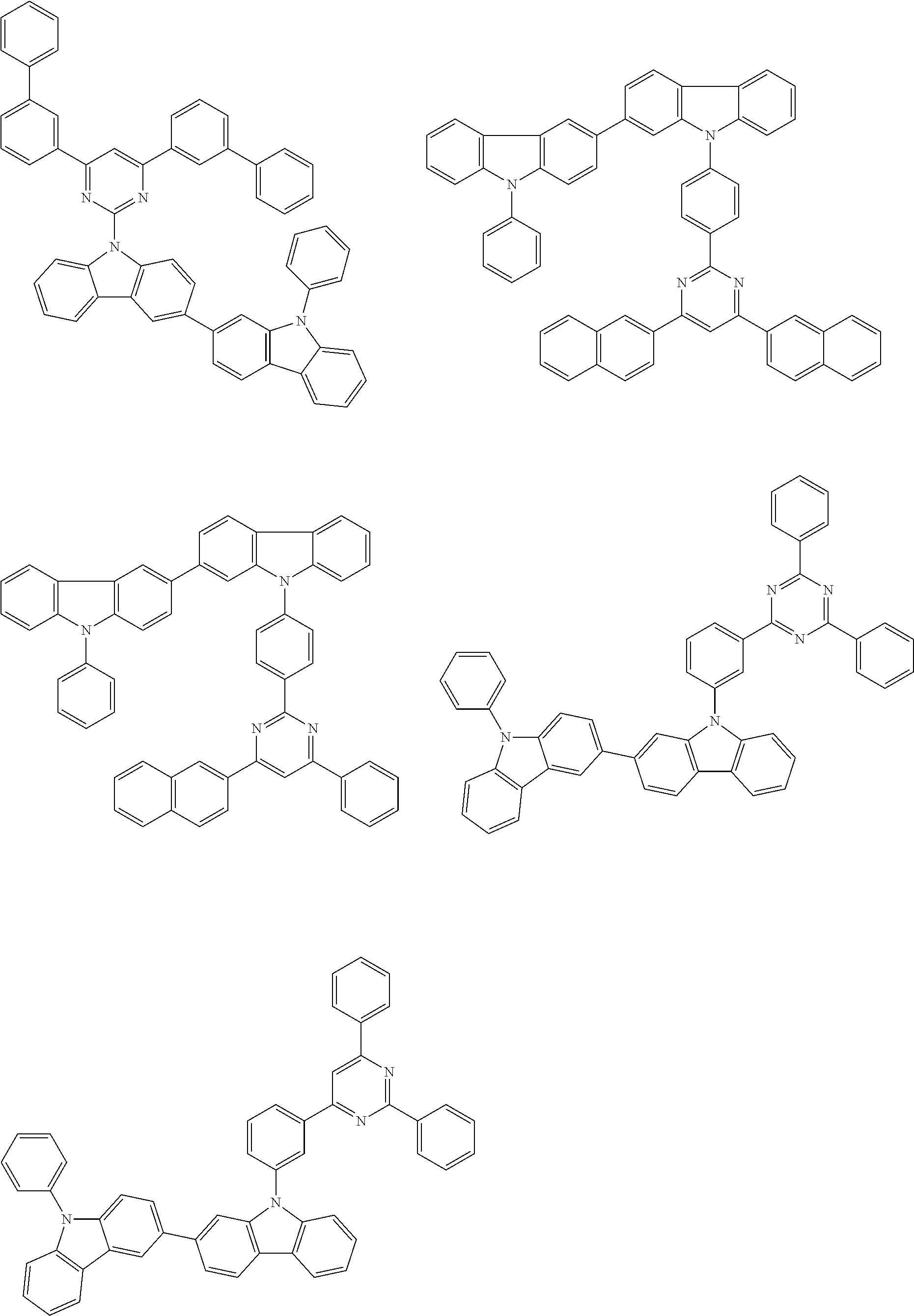

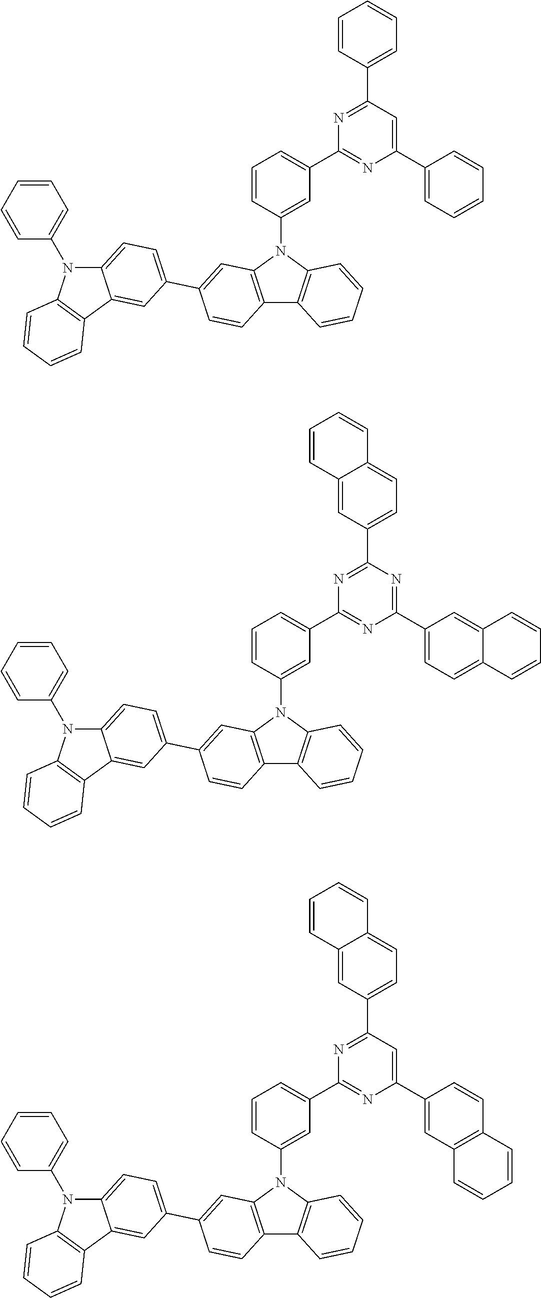 [SMILES: c1ccc(-c2cc(-c3ccccc3)nc(-c3cccc(-n4c5ccccc5c5ccc(-c6ccc7c(c6)c6ccccc6n7-c6ccccc6)cc54)c3)n2)cc1 ; c1ccc(-n2c3ccccc3c3cc(-c4ccc5c6ccccc6n(-c6cccc(-c7nc(-c8ccc9ccccc9c8)nc(-c8ccc9ccccc9c8)n7)c6)c5c4)ccc32)cc1 ; c1ccc(-n2c3ccccc3c3cc(-c4ccc5c6ccccc6n(-c6cccc(-c7nc(-c8ccc9ccccc9c8)cc(-c8ccc9ccccc9c8)n7)c6)c5c4)ccc32)cc1]

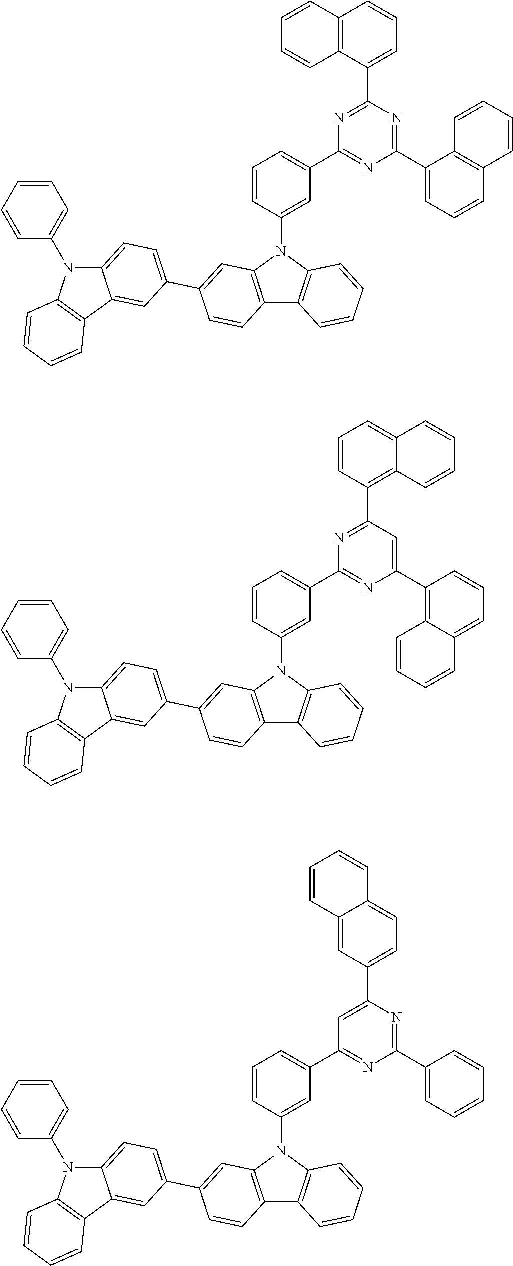 [SMILES: c1ccc(-n2c3ccccc3c3cc(-c4ccc5c6ccccc6n(-c6cccc(-c7nc(-c8cccc9ccccc89)nc(-c8cccc9ccccc89)n7)c6)c5c4)ccc32)cc1 ; c1ccc(-n2c3ccccc3c3cc(-c4ccc5c6ccccc6n(-c6cccc(-c7nc(-c8cccc9ccccc89)cc(-c8cccc9ccccc89)n7)c6)c5c4)ccc32)cc1 ; c1ccc(-c2nc(-c3cccc(-n4c5ccccc5c5ccc(-c6ccc7c(c6)c6ccccc6n7-c6ccccc6)cc54)c3)cc(-c3ccc4ccccc4c3)n2)cc1]

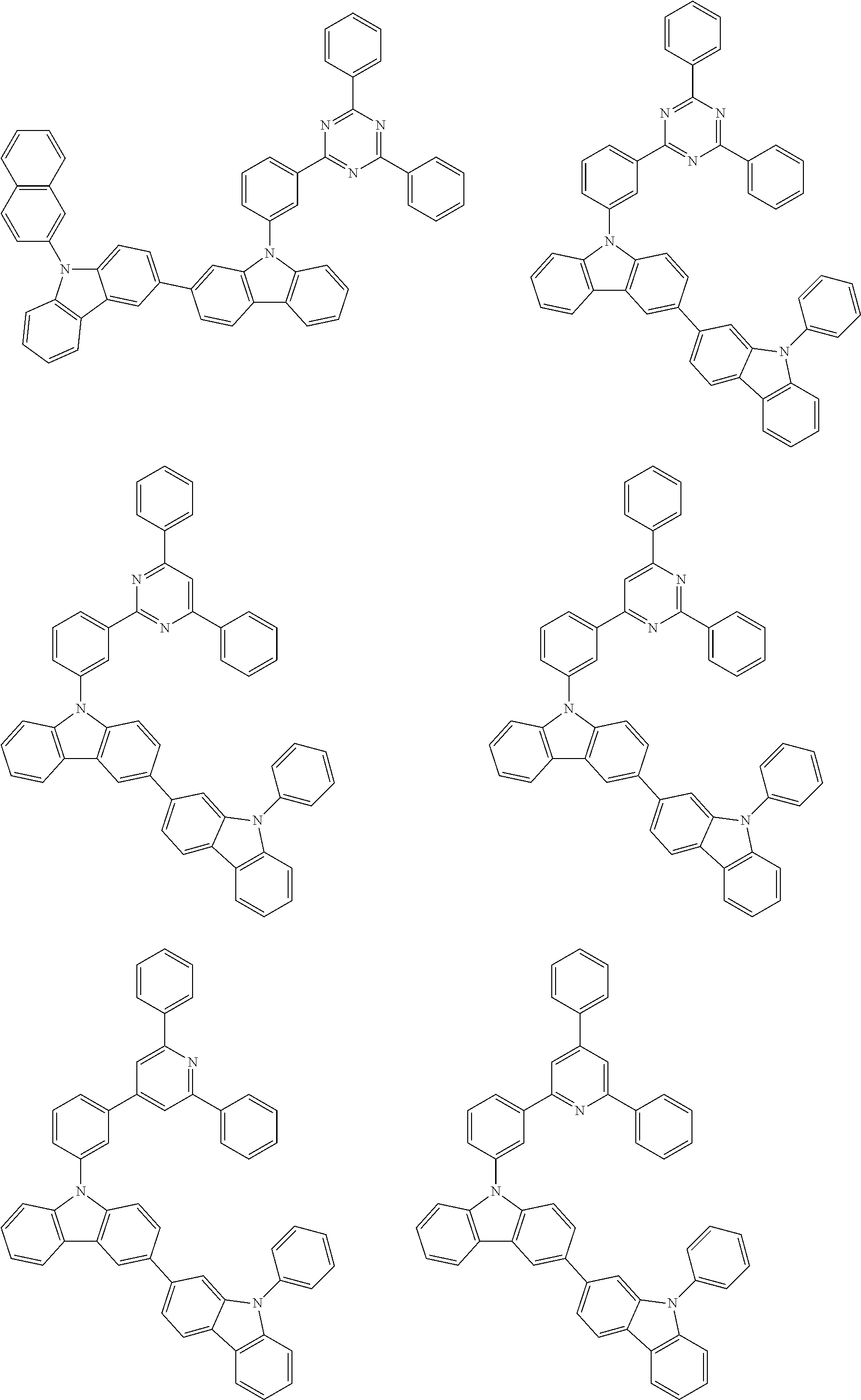

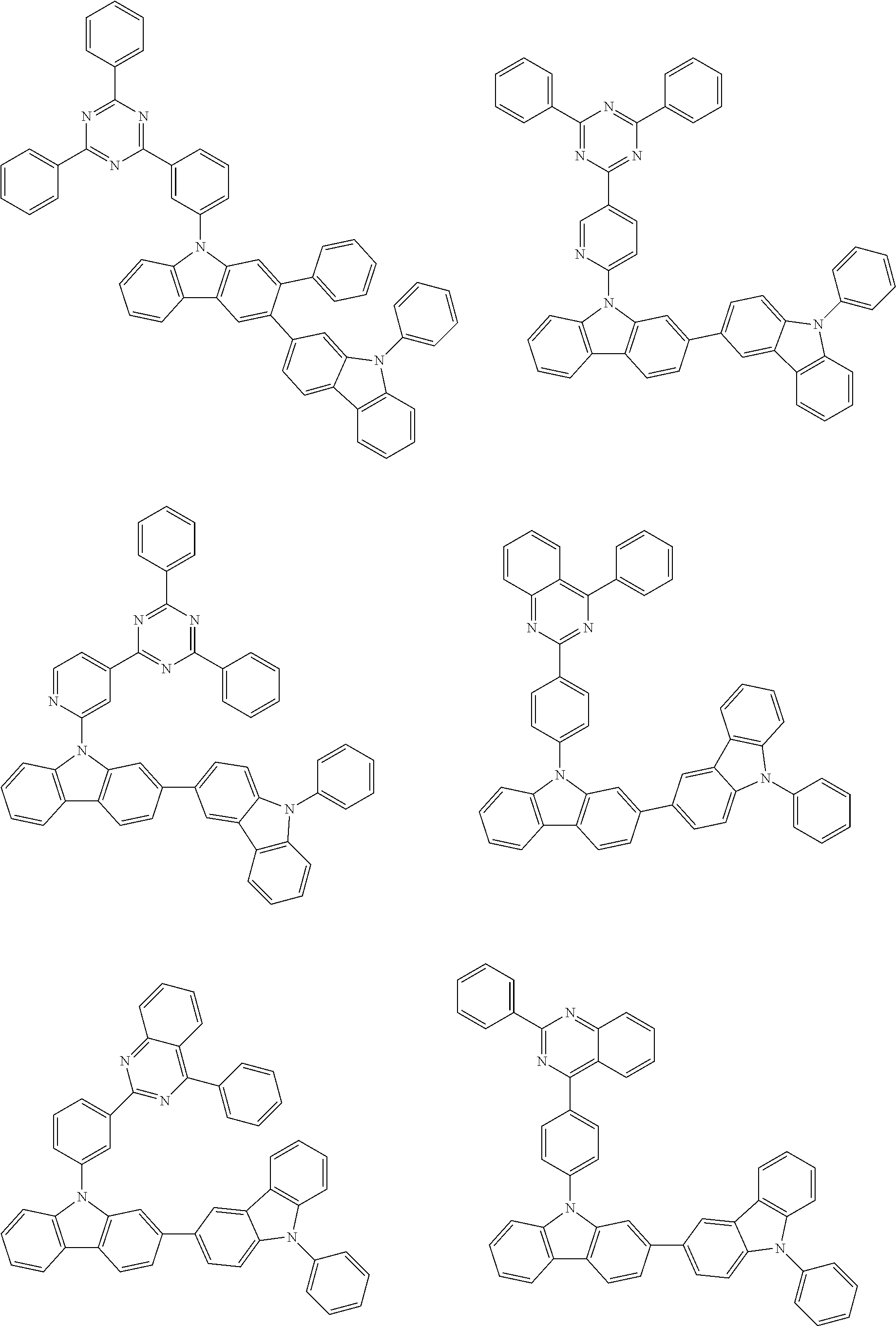

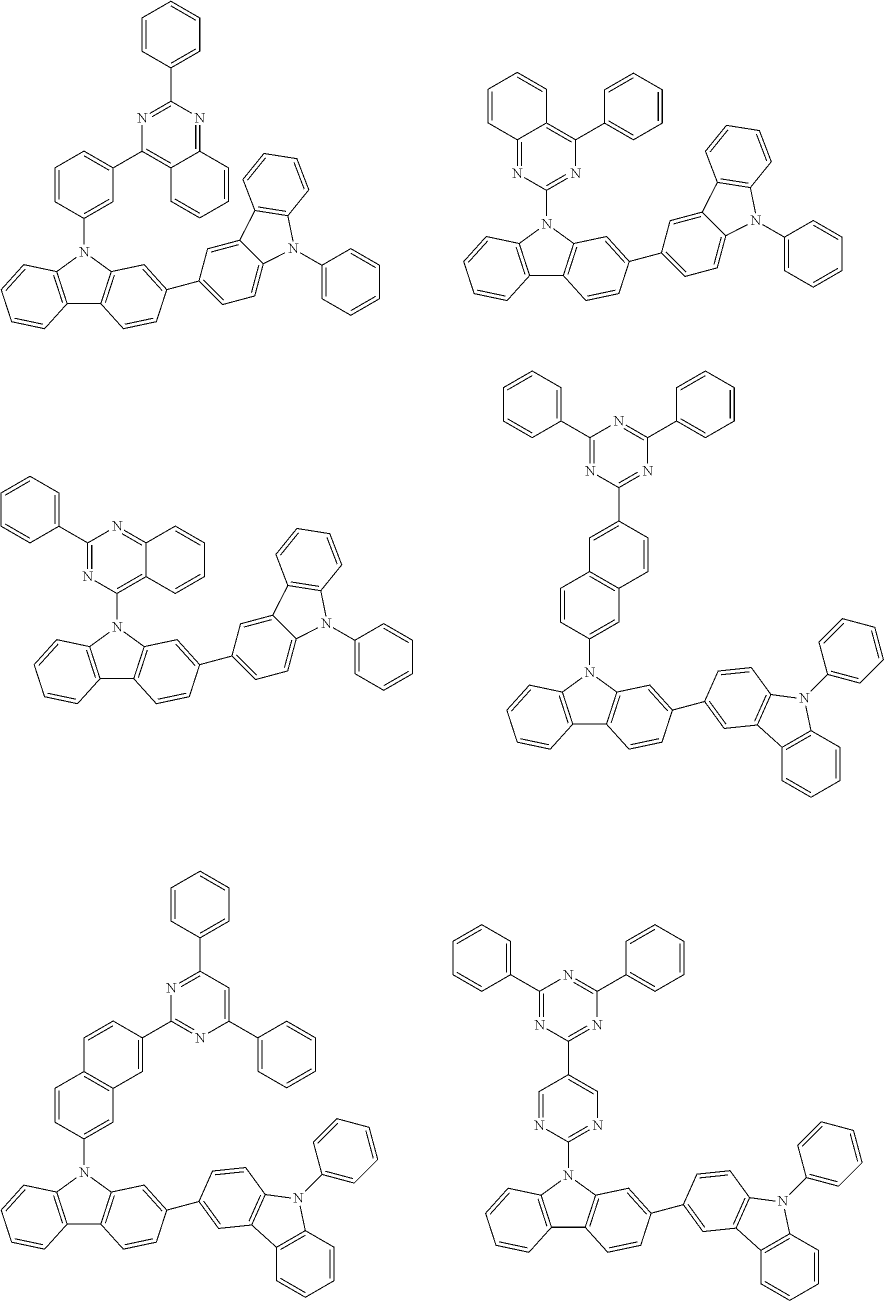

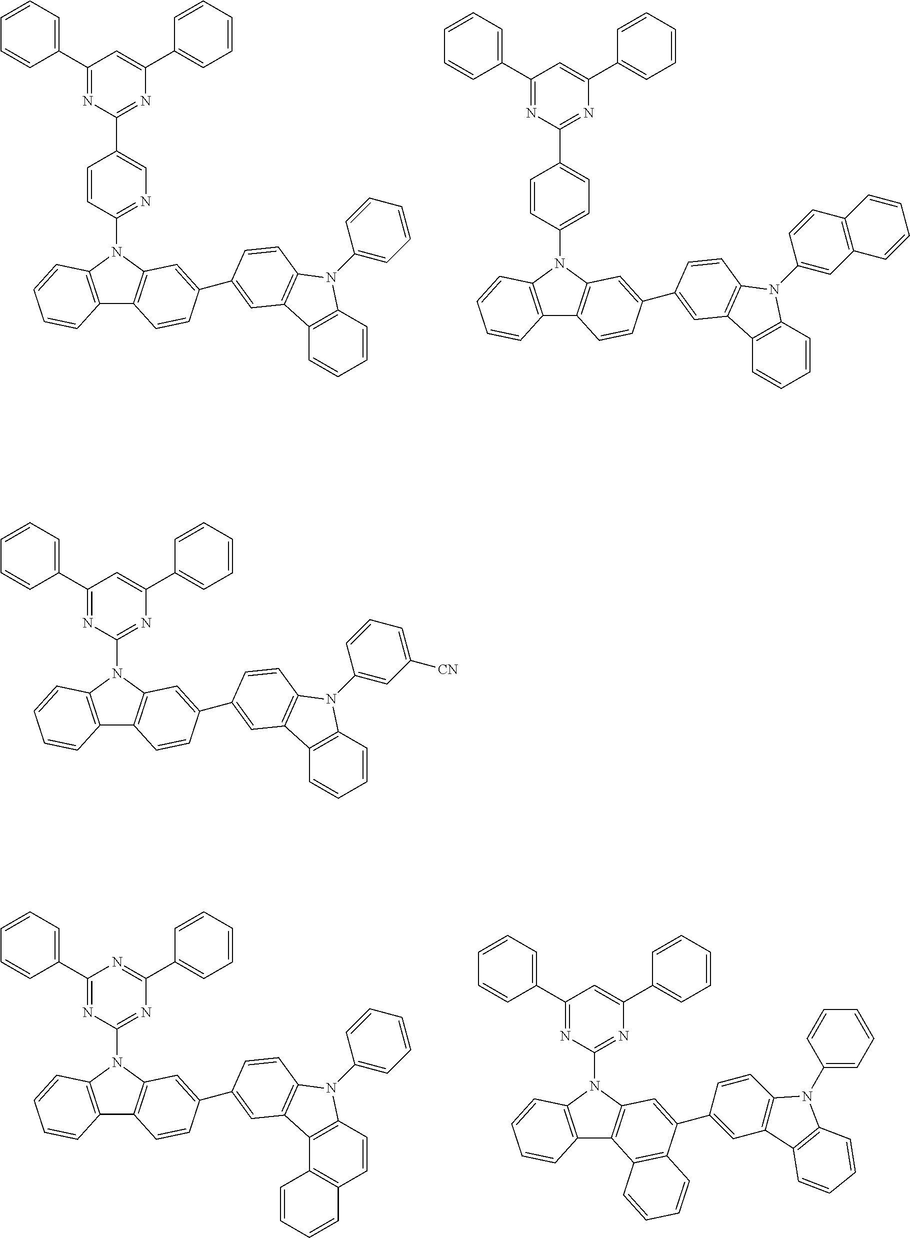 [SMILES: c1ccc(-c2cc(-c3ccccc3)nc(-c3ccc(-n4c5ccccc5c5ccc(-c6ccc7c(c6)c6ccccc6n7-c6ccccc6)cc54)nc3)n2)cc1 ; c1ccc(-c2cc(-c3ccccc3)nc(-c3ccc(-n4c5ccccc5c5ccc(-c6ccc7c(c6)c6ccccc6n7-c6ccc7ccccc7c6)cc54)cc3)n2)cc1 ; N#Cc1cccc(-n2c3ccccc3c3cc(-c4ccc5c6ccccc6n(-c6nc(-c7ccccc7)cc(-c7ccccc7)n6)c5c4)ccc32)c1 ; c1ccc(-c2nc(-c3ccccc3)nc(-n3c4ccccc4c4ccc(-c5ccc6c(c5)c5c7ccccc7ccc5n6-c5ccccc5)cc43)n2)cc1 ; c1ccc(-c2cc(-c3ccccc3)nc(-n3c4ccccc4c4c5ccccc5c(-c5ccc6c(c5)c5ccccc5n6-c5ccccc5)cc43)n2)cc1]

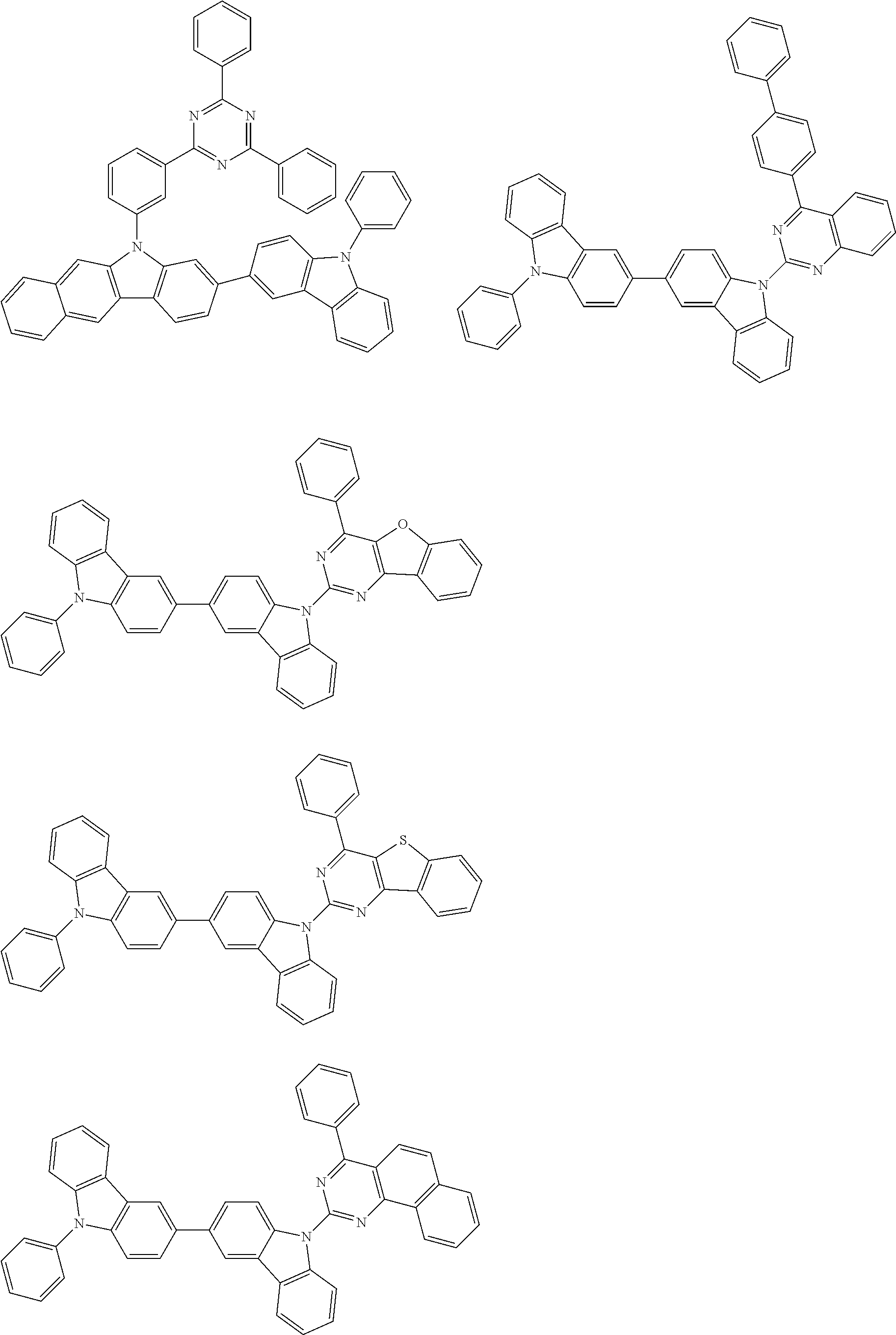

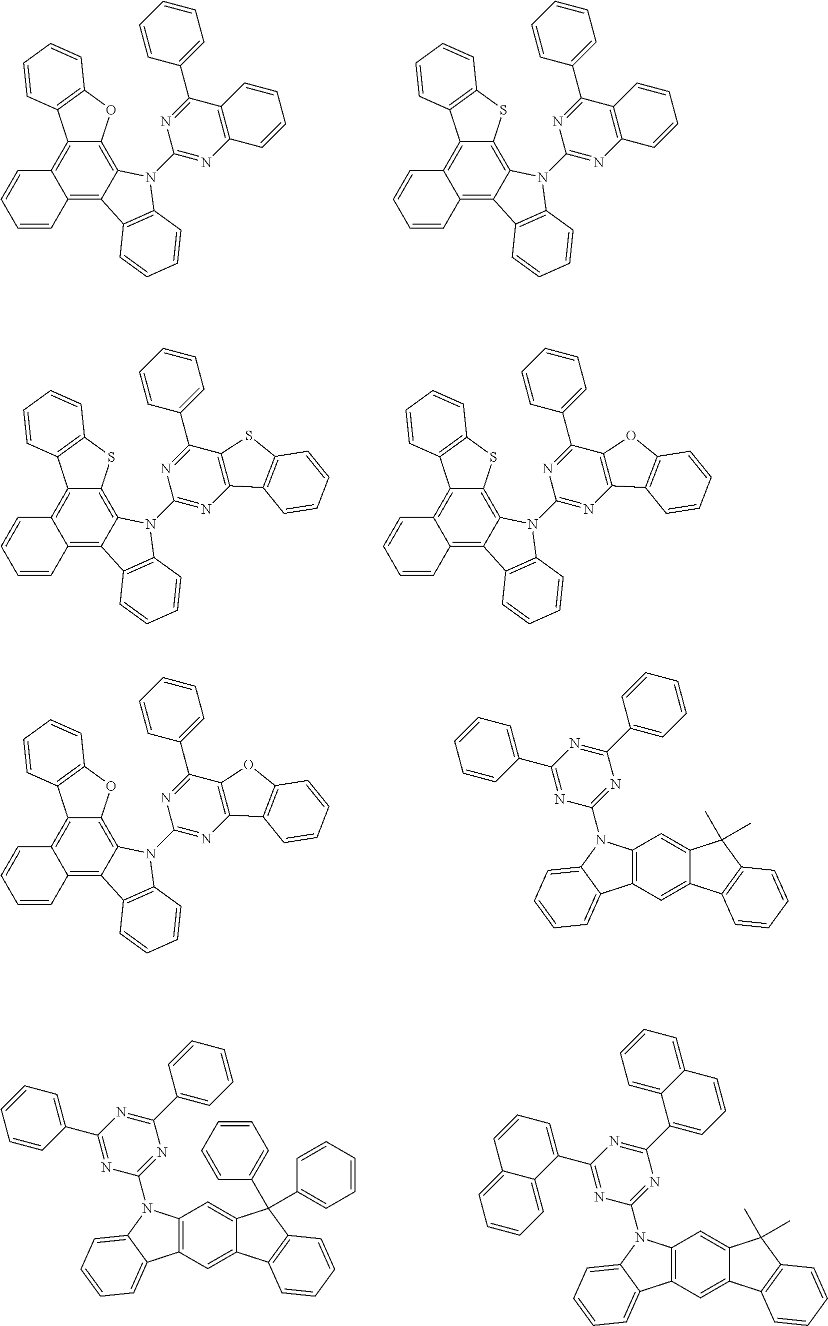

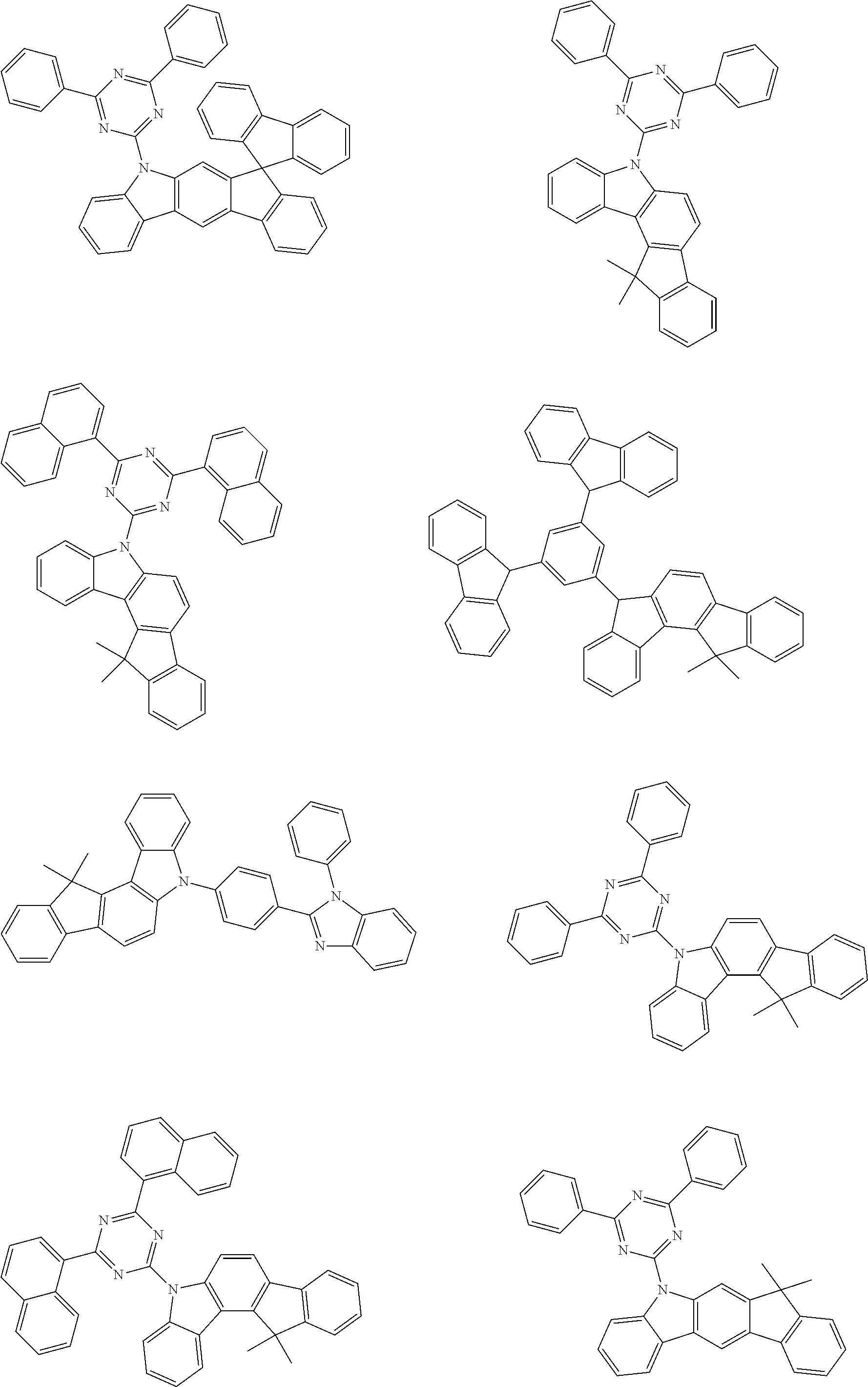

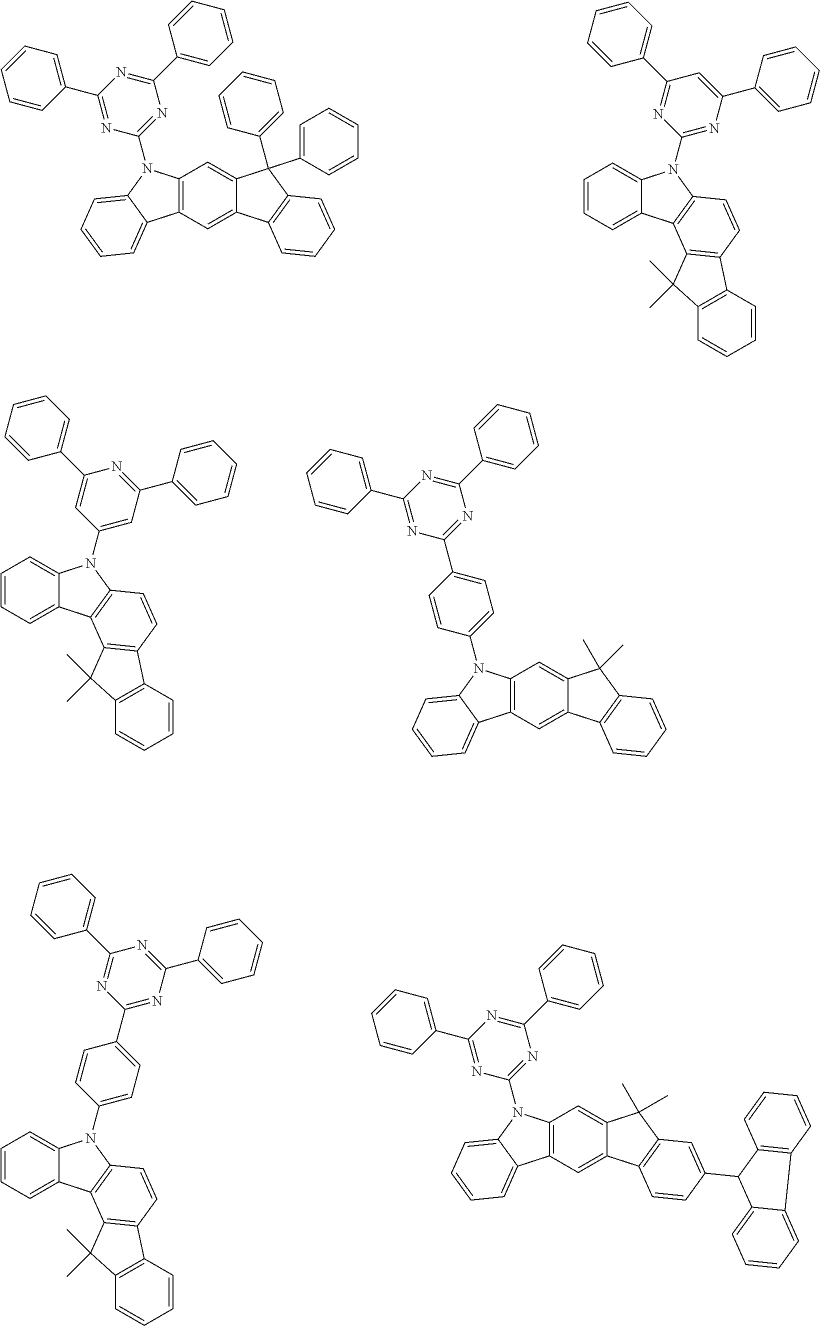 [SMILES: c1ccc(-c2nc(-c3ccccc3)nc(-n3c4ccccc4c4cc5c(cc43)C(c3ccccc3)(c3ccccc3)c3ccccc3-5)n2)cc1 ; CC1(C)c2ccccc2-c2ccc3c(c21)c1ccccc1n3-c1nc(-c2ccccc2)cc(-c2ccccc2)n1 ; CC1(C)c2ccccc2-c2ccc3c(c21)c1ccccc1n3-c1cc(-c2ccccc2)nc(-c2ccccc2)c1 ; CC1(C)c2ccccc2-c2cc3c4ccccc4n(-c4ccc(-c5nc(-c6ccccc6)nc(-c6ccccc6)n5)cc4)c3cc21 ; CC1(C)c2ccccc2-c2ccc3c(c21)c1ccccc1n3-c1ccc(-c2nc(-c3ccccc3)nc(-c3ccccc3)n2)cc1 ; CC1(C)c2cc(C3c4ccccc4-c4ccccc43)ccc2-c2cc3c4ccccc4n(-c4nc(-c5ccccc5)nc(-c5ccccc5)n4)c3cc21]

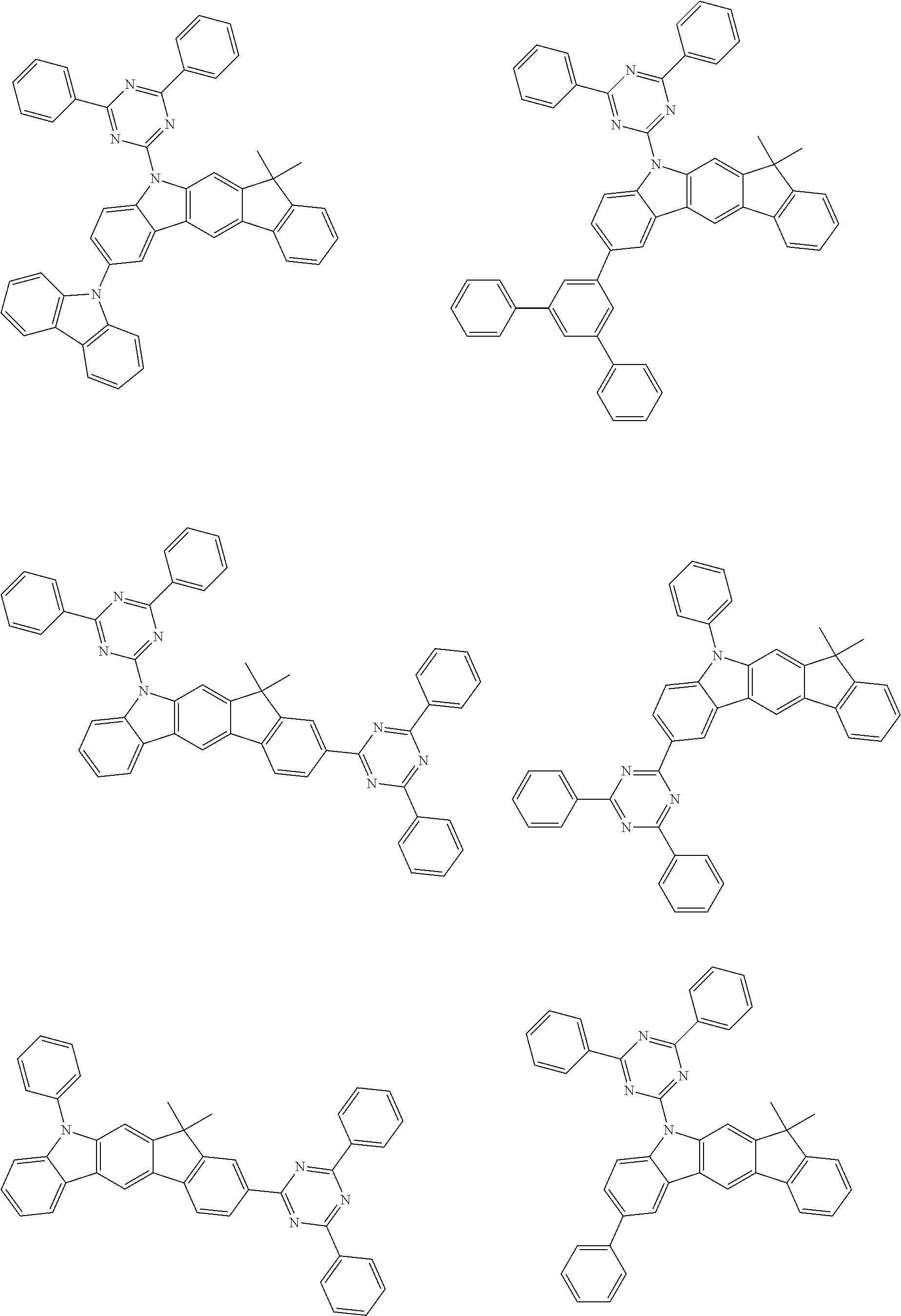

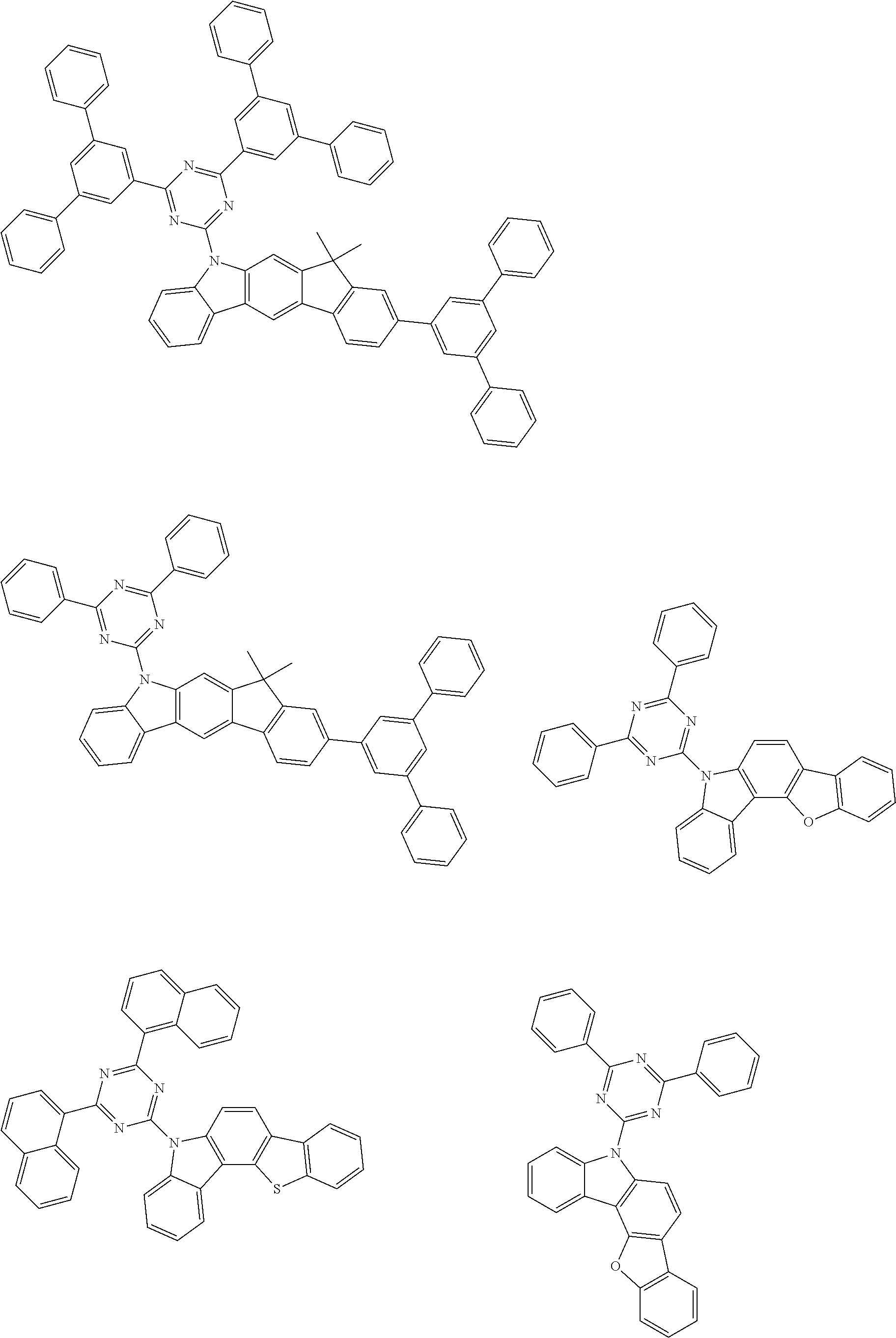 [SMILES: CC1(C)c2cc(-c3cc(-c4ccccc4)cc(-c4ccccc4)c3)ccc2-c2cc3c4ccccc4n(-c4nc(-c5cc(-c6ccccc6)cc(-c6ccccc6)c5)nc(-c5cc(-c6ccccc6)cc(-c6ccccc6)c5)n4)c3cc21 ; CC1(C)c2cc(-c3cc(-c4ccccc4)cc(-c4ccccc4)c3)ccc2-c2cc3c4ccccc4n(-c4nc(-c5ccccc5)nc(-c5ccccc5)n4)c3cc21 ; c1ccc(-c2nc(-c3ccccc3)nc(-n3c4ccccc4c4c5oc6ccccc6c5ccc43)n2)cc1 ; c1ccc2c(-c3nc(-c4cccc5ccccc45)nc(-n4c5ccccc5c5c6sc7ccccc7c6ccc54)n3)cccc2c1 ; c1ccc(-c2nc(-c3ccccc3)nc(-n3c4ccccc4c4c5oc6ccccc6c5ccc43)n2)cc1]

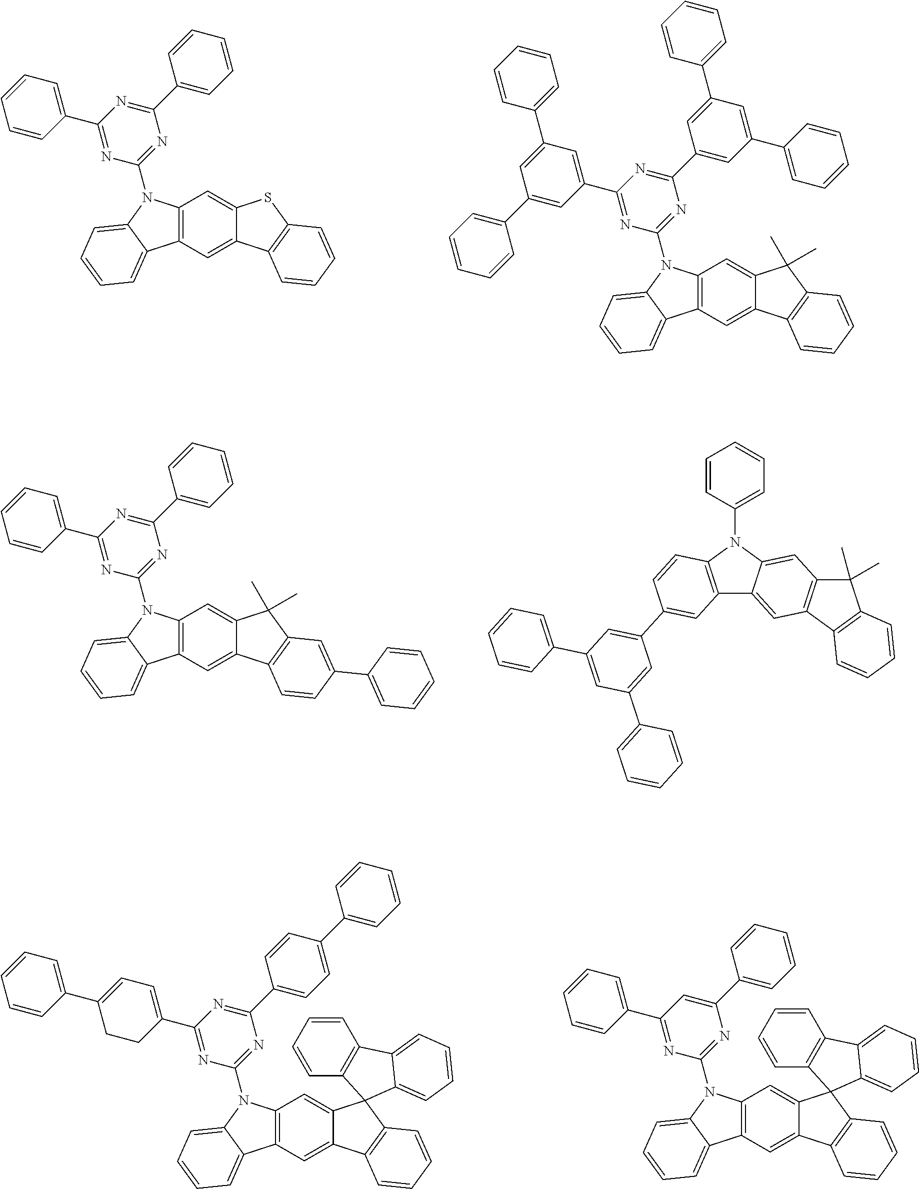

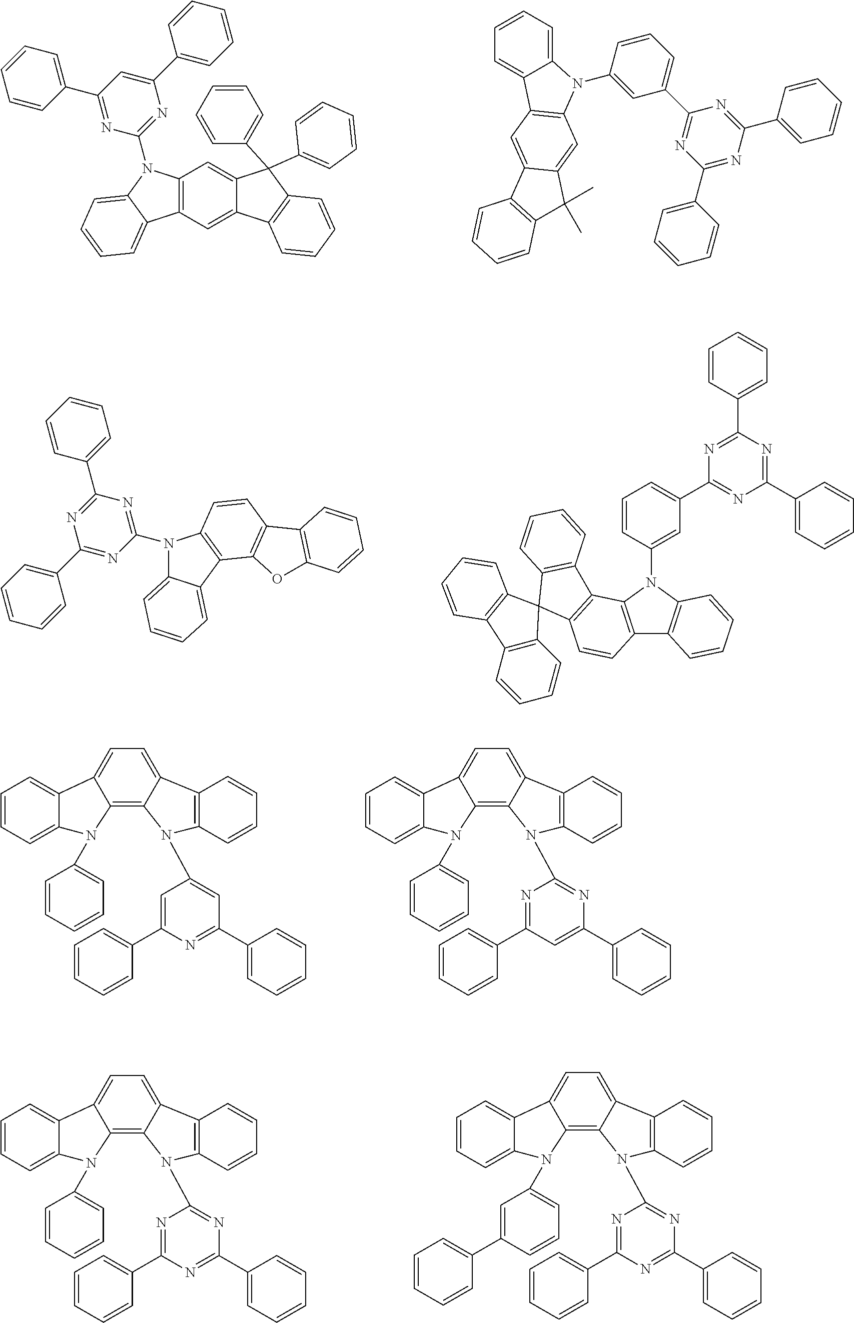

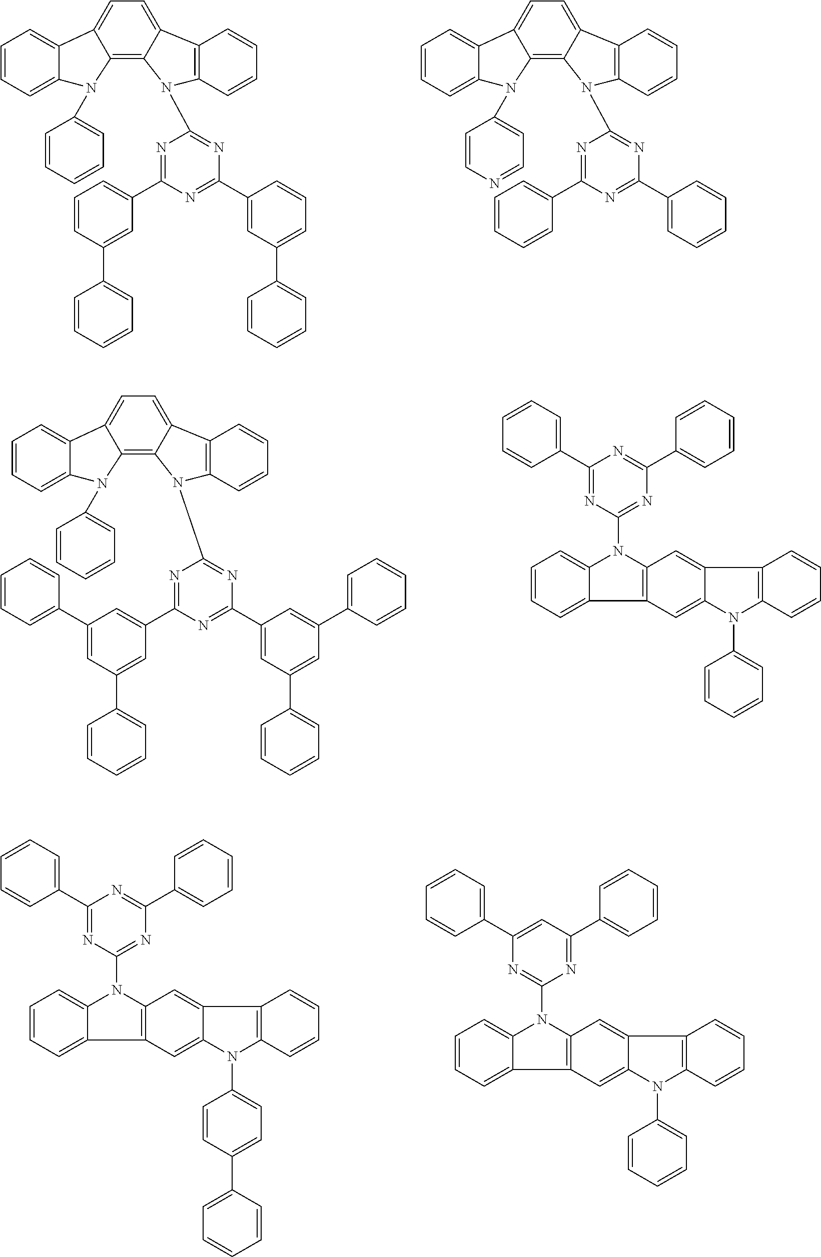

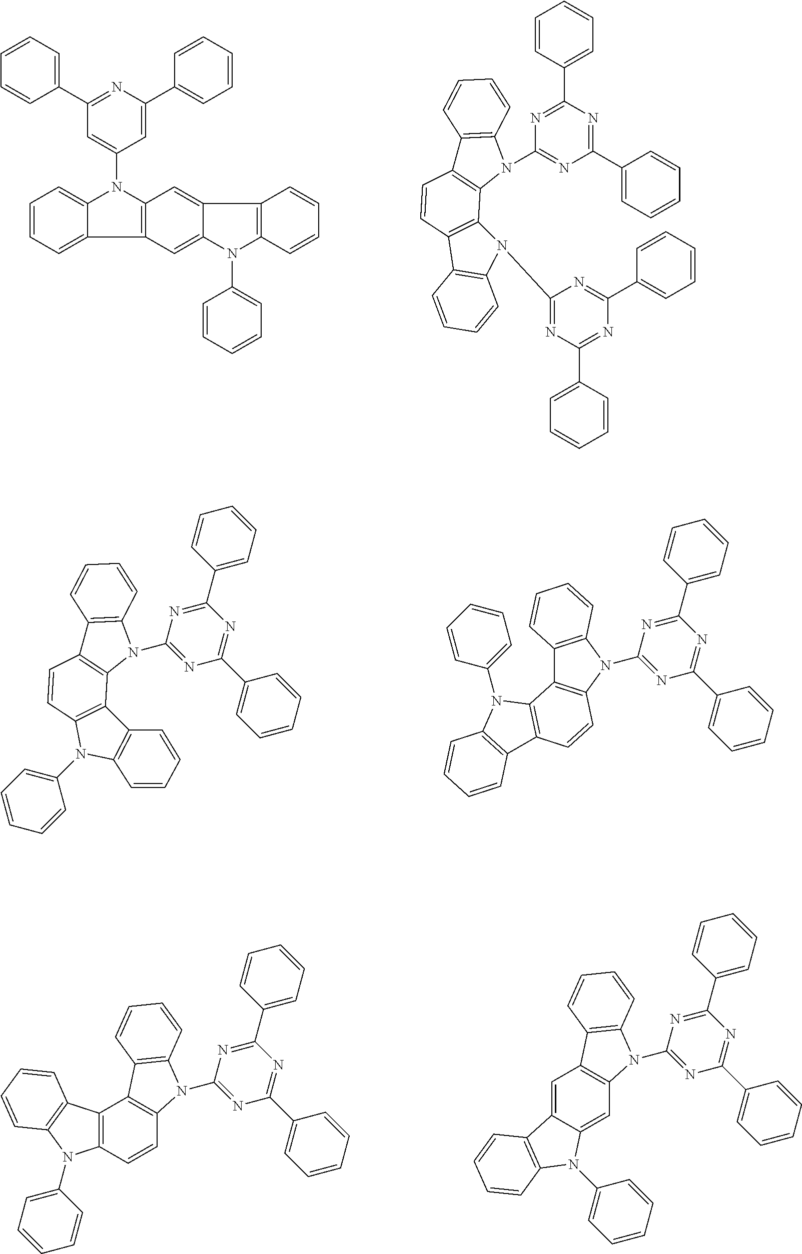 [SMILES: c1ccc(-c2cc(-n3c4ccccc4c4cc5c(cc43)c3ccccc3n5-c3ccccc3)cc(-c3ccccc3)n2)cc1 ; c1ccc(-c2nc(-c3ccccc3)nc(-n3c4ccccc4c4ccc5c6ccccc6n(-c6nc(-c7ccccc7)nc(-c7ccccc7)n6)c5c43)n2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-n3c4ccccc4c4c3ccc3c5ccccc5n(-c5ccccc5)c34)n2)cc1 ; c1ccc(-c2nc(-c3ccccc3)nc(-n3c4ccccc4c4c5c6ccccc6n(-c6ccccc6)c5ccc43)n2)cc1 ; c1ccc(-c2nc(-c3ccccc3)nc(-n3c4ccccc4c4cc5c6ccccc6n(-c6ccccc6)c5cc43)n2)cc1]

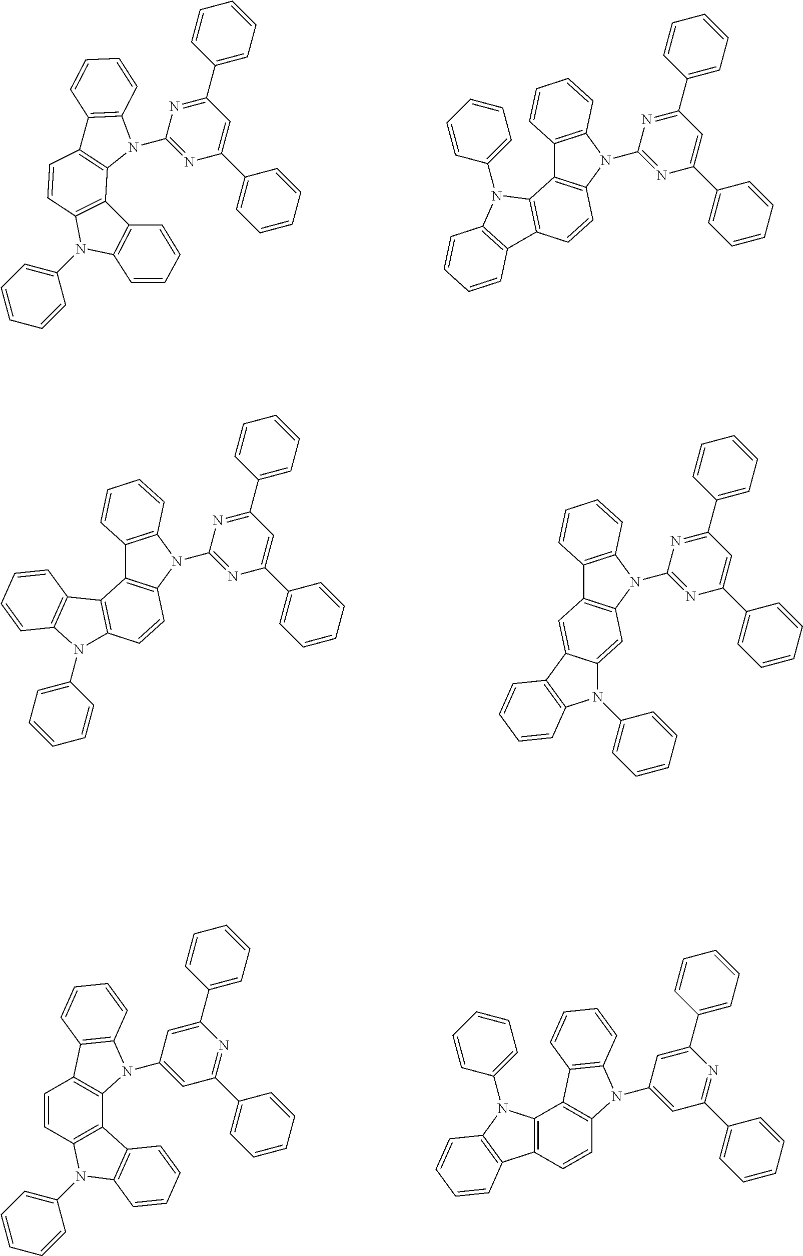 [SMILES: c1ccc(-c2cc(-c3ccccc3)nc(-n3c4ccccc4c4ccc5c(c6ccccc6n5-c5ccccc5)c43)n2)cc1 ; c1ccc(-c2cc(-c3ccccc3)nc(-n3c4ccccc4c4c3ccc3c5ccccc5n(-c5ccccc5)c34)n2)cc1 ; c1ccc(-c2cc(-c3ccccc3)nc(-n3c4ccccc4c4c5c6ccccc6n(-c6ccccc6)c5ccc43)n2)cc1 ; c1ccc(-c2cc(-c3ccccc3)nc(-n3c4ccccc4c4cc5c6ccccc6n(-c6ccccc6)c5cc43)n2)cc1 ; c1ccc(-c2cc(-n3c4ccccc4c4ccc5c(c6ccccc6n5-c5ccccc5)c43)cc(-c3ccccc3)n2)cc1 ; c1ccc(-c2cc(-n3c4ccccc4c4c3ccc3c5ccccc5n(-c5ccccc5)c34)cc(-c3ccccc3)n2)cc1]

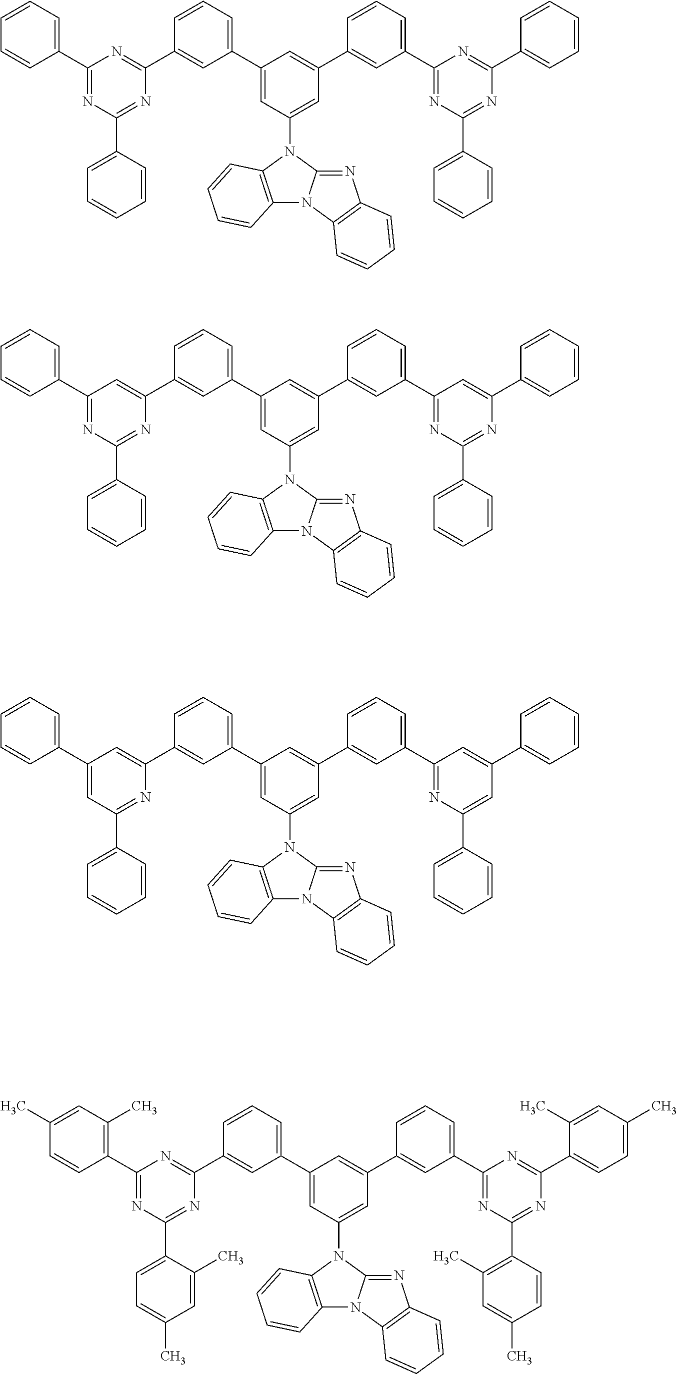

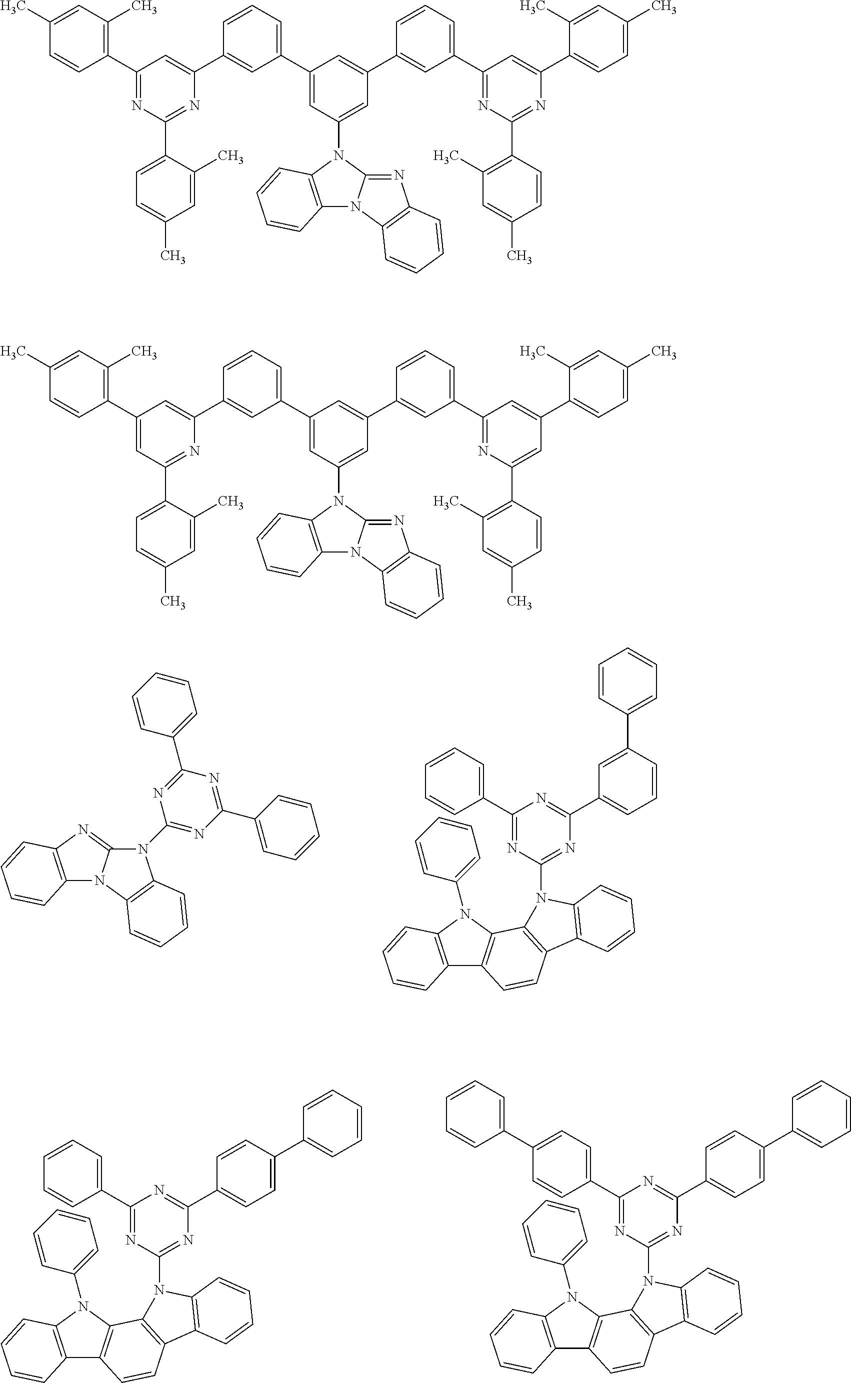

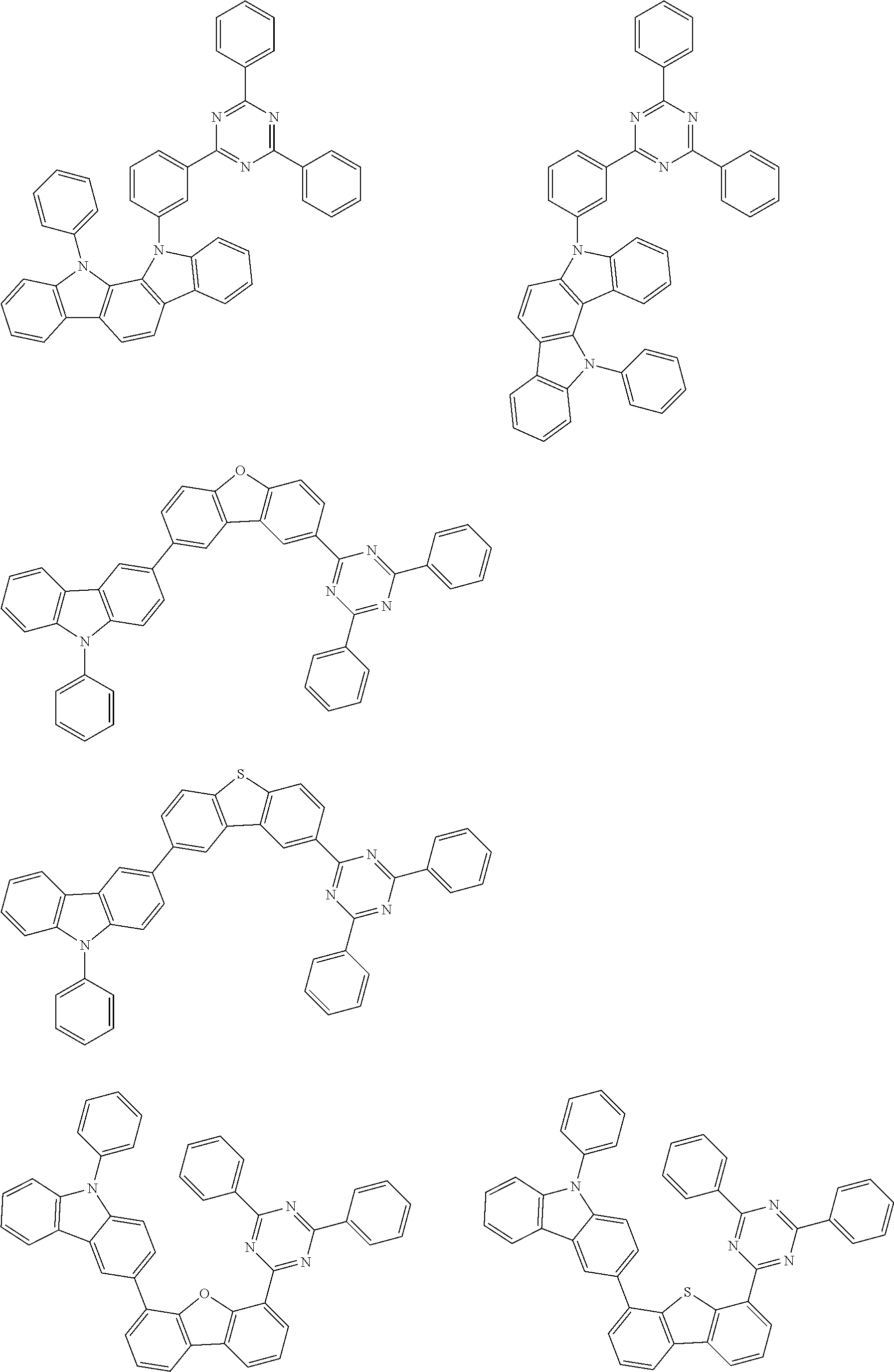

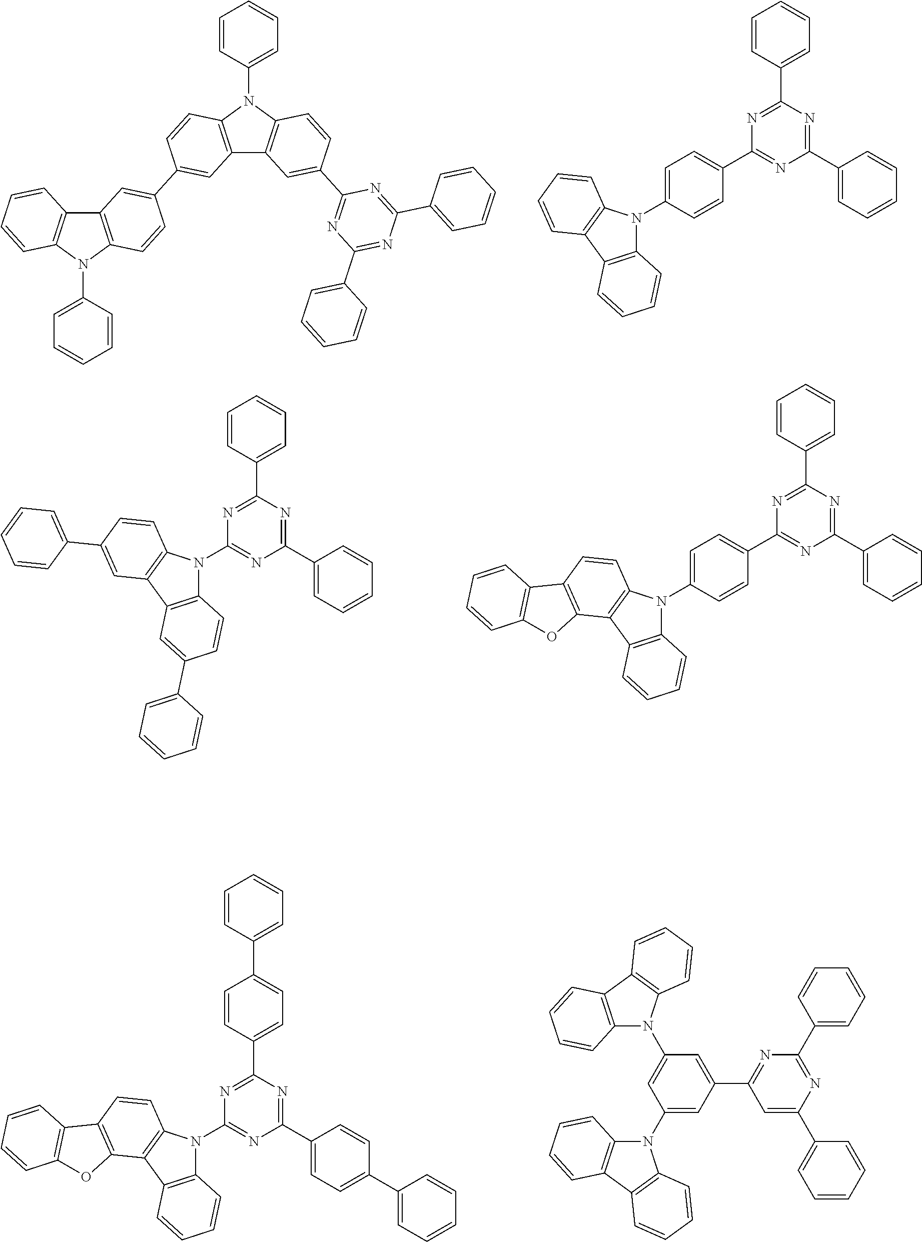

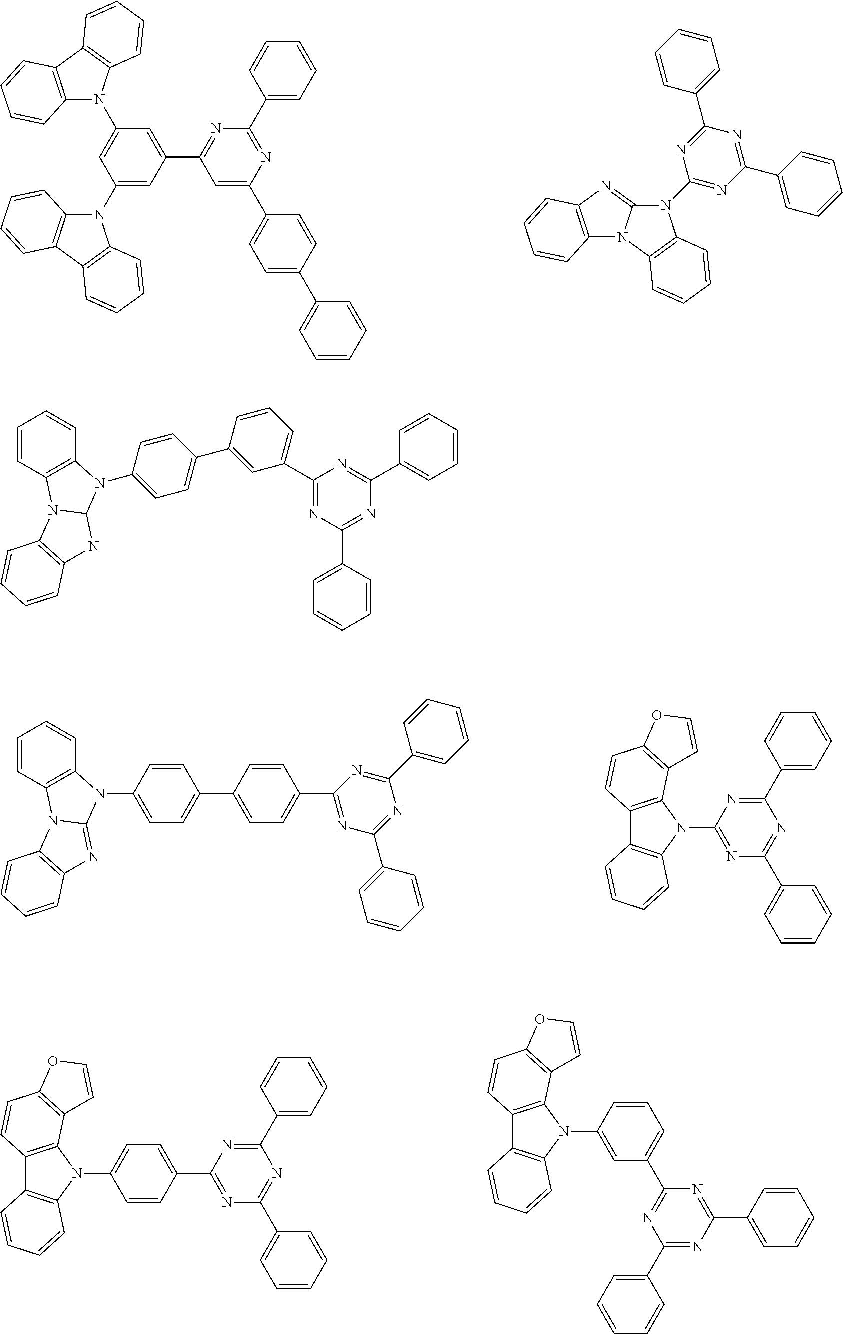

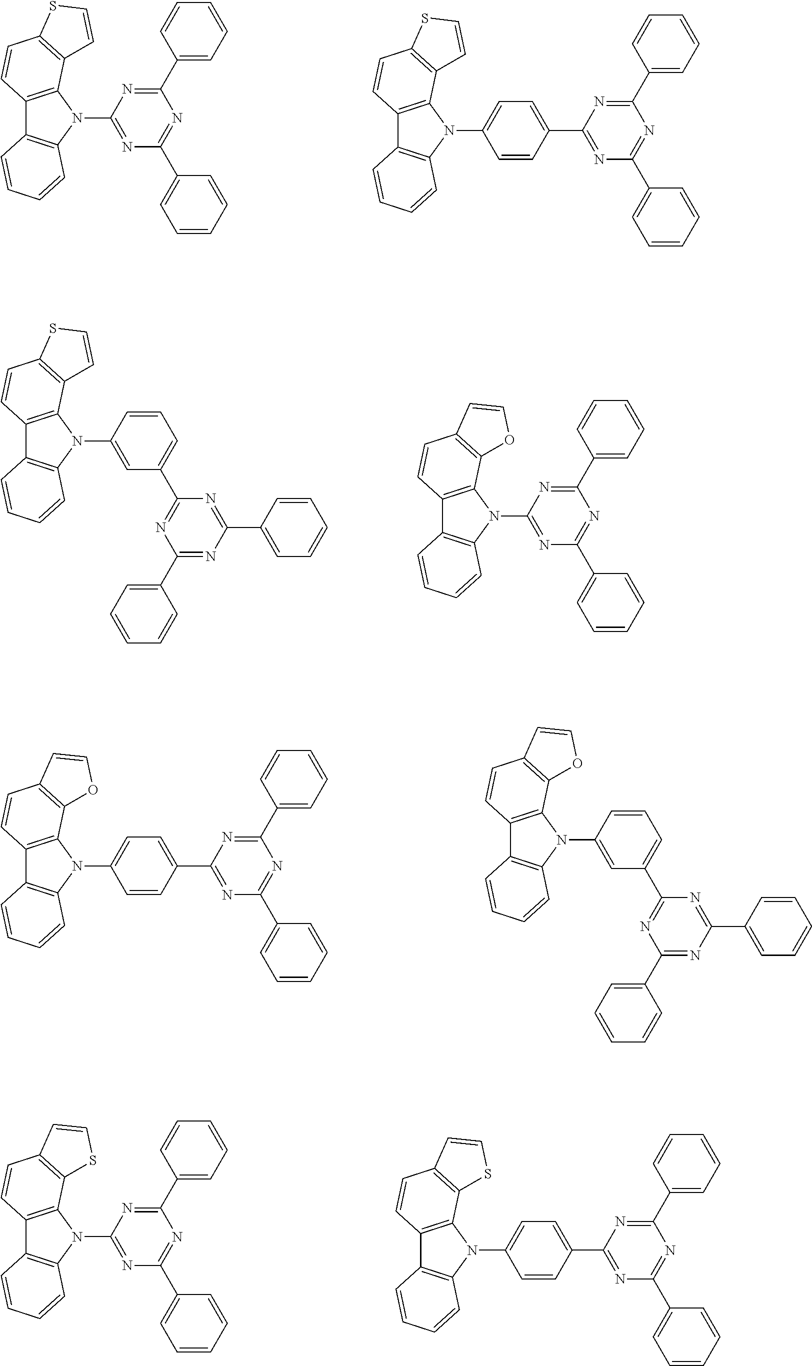

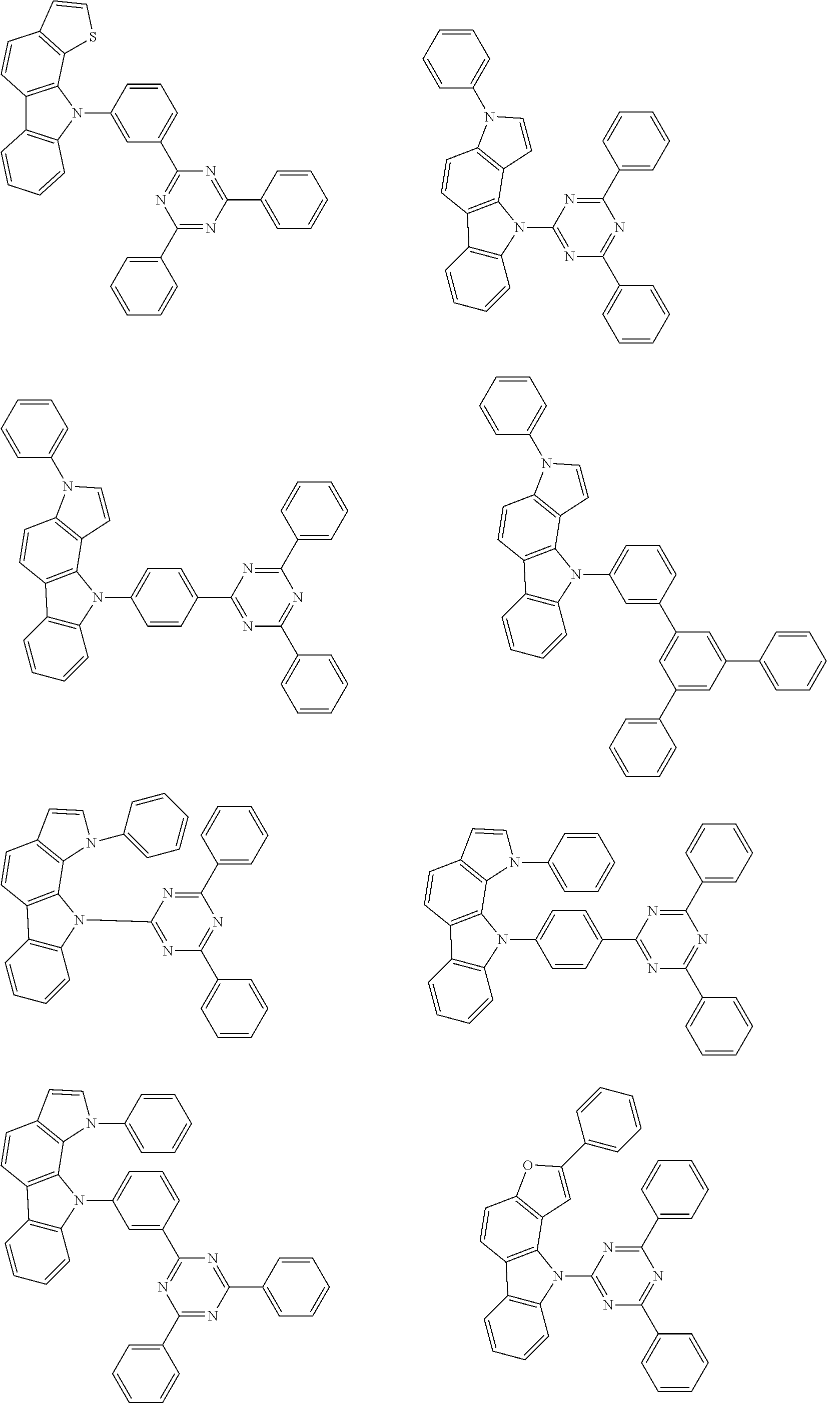

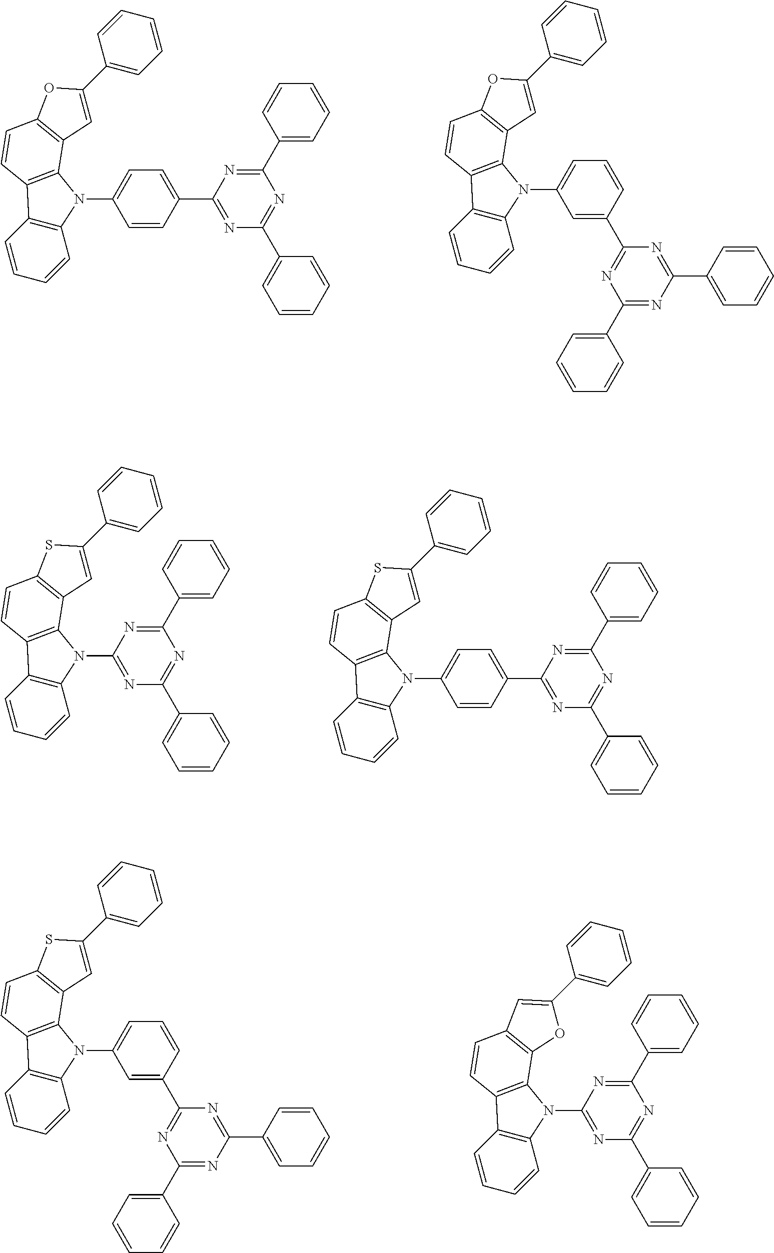 [SMILES: c1ccc(-c2nc(-c3ccccc3)nc(-c3ccc(-n4c5ccccc5c5ccc6oc(-c7ccccc7)cc6c54)cc3)n2)cc1 ; c1ccc(-c2nc(-c3ccccc3)nc(-c3cccc(-n4c5ccccc5c5ccc6oc(-c7ccccc7)cc6c54)c3)n2)cc1 ; c1ccc(-c2nc(-c3ccccc3)nc(-n3c4ccccc4c4ccc5sc(-c6ccccc6)cc5c43)n2)cc1 ; c1ccc(-c2nc(-c3ccccc3)nc(-c3ccc(-n4c5ccccc5c5ccc6sc(-c7ccccc7)cc6c54)cc3)n2)cc1 ; c1ccc(-c2nc(-c3ccccc3)nc(-c3cccc(-n4c5ccccc5c5ccc6sc(-c7ccccc7)cc6c54)c3)n2)cc1 ; c1ccc(-c2nc(-c3ccccc3)nc(-n3c4ccccc4c4ccc5cc(-c6ccccc6)oc5c43)n2)cc1]

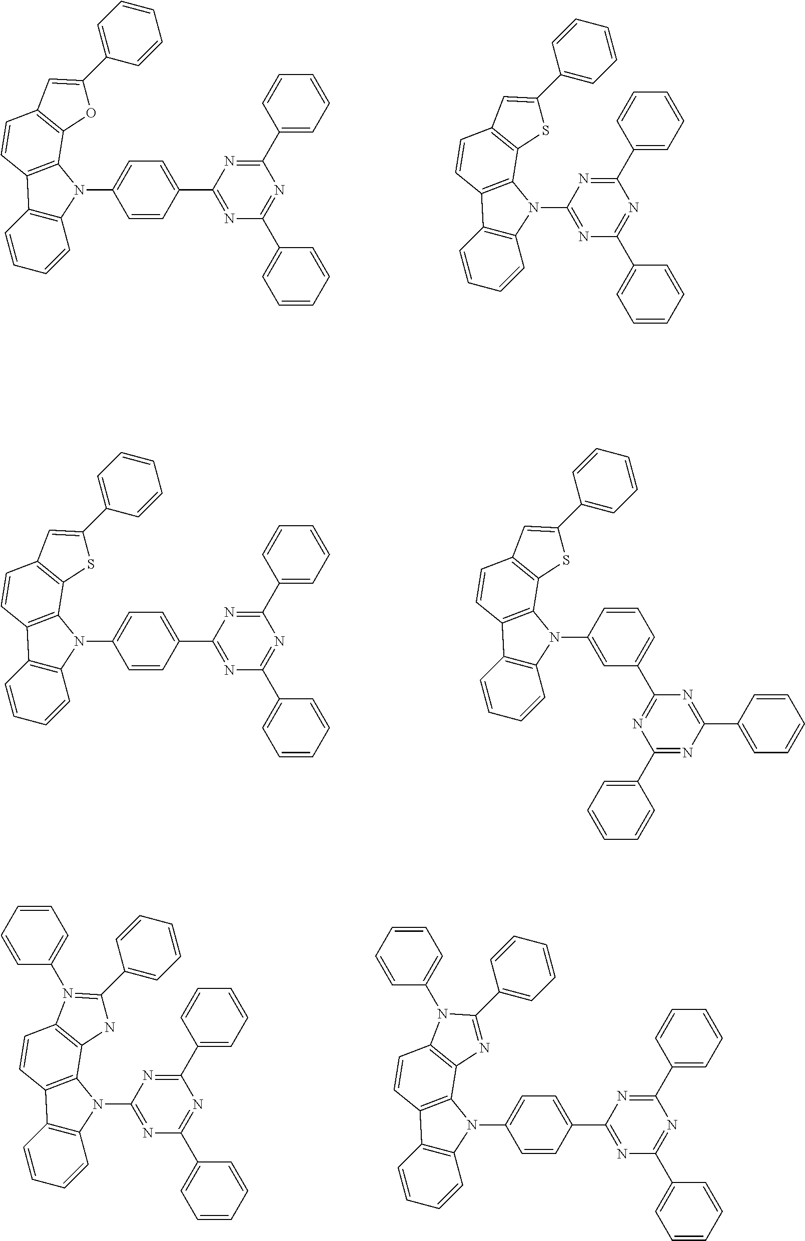

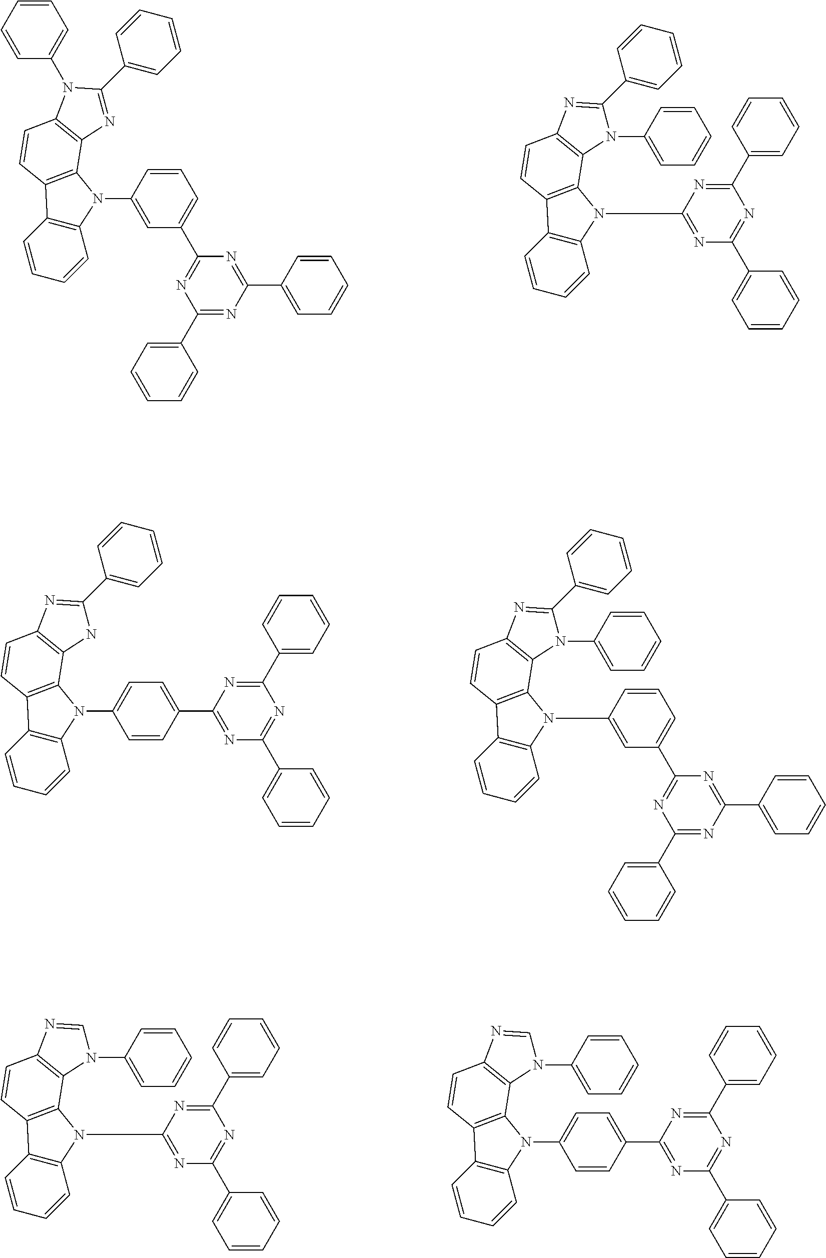

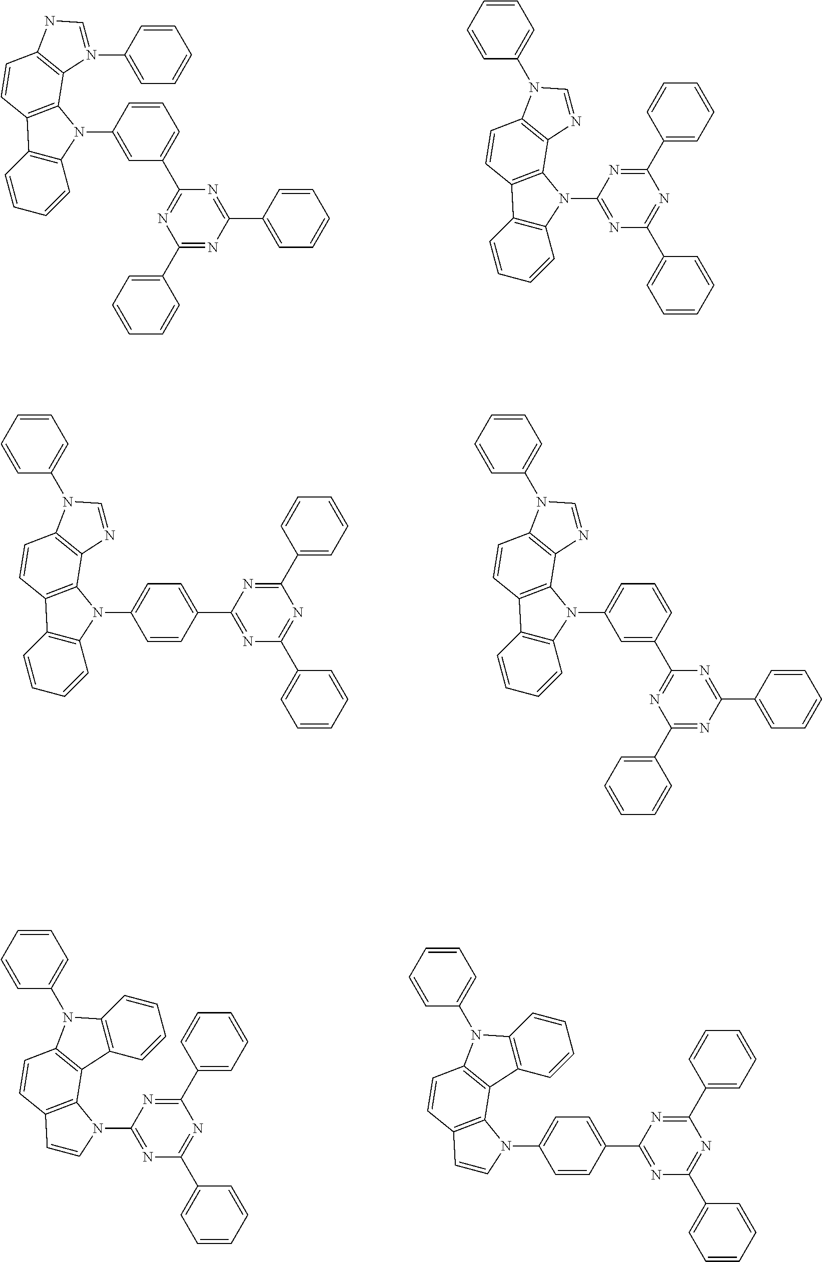

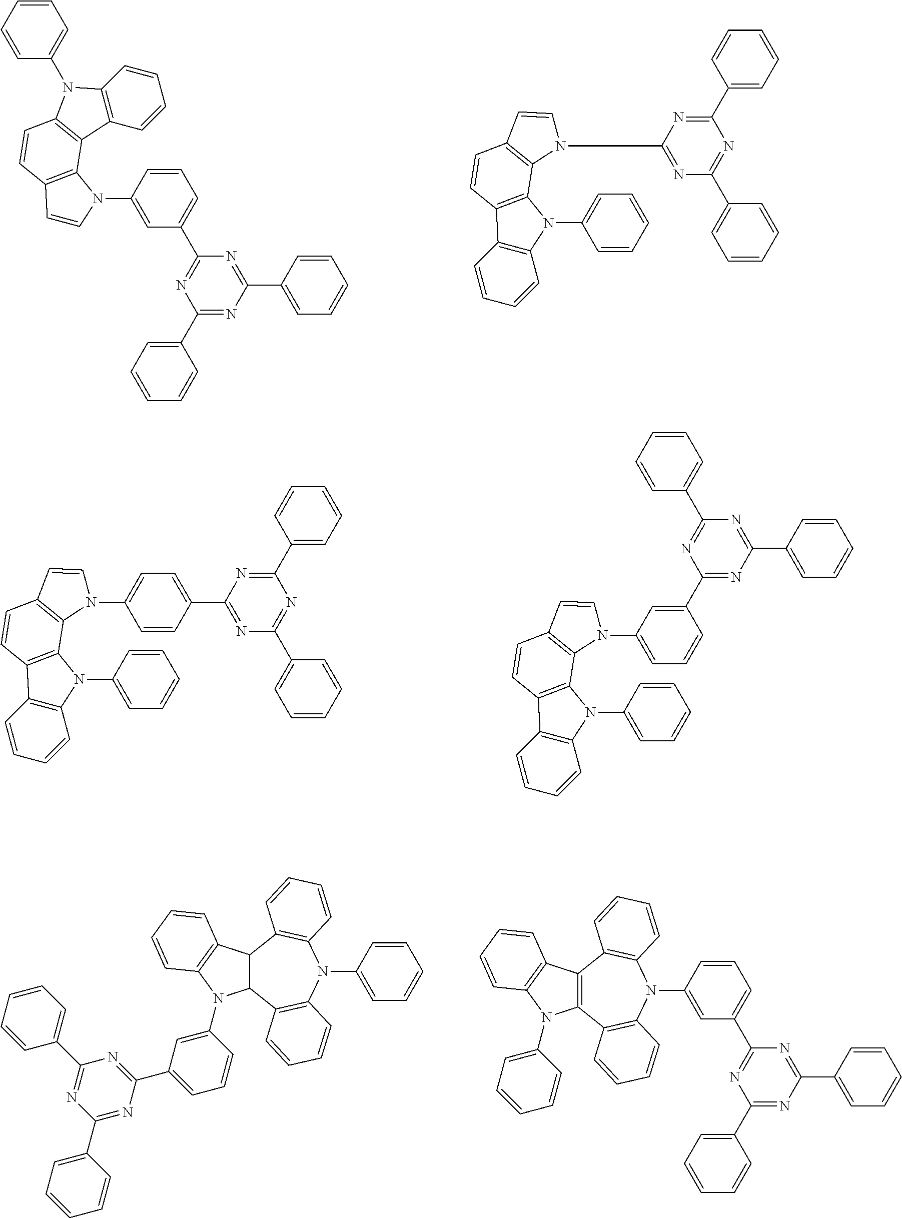 [SMILES: c1ccc(-c2nc(-c3ccccc3)nc(-c3cccc(-n4ccc5ccc6c(c7ccccc7n6-c6ccccc6)c54)c3)n2)cc1 ; c1ccc(-c2nc(-c3ccccc3)nc(-n3ccc4ccc5c6ccccc6n(-c6ccccc6)c5c43)n2)cc1 ; c1ccc(-c2nc(-c3ccccc3)nc(-c3ccc(-n4ccc5ccc6c7ccccc7n(-c7ccccc7)c6c54)cc3)n2)cc1 ; c1ccc(-c2nc(-c3ccccc3)nc(-c3cccc(-n4ccc5ccc6c7ccccc7n(-c7ccccc7)c6c54)c3)n2)cc1 ; c1ccc(-c2nc(-c3ccccc3)nc(-c3cccc(N4c5ccccc5C5c6ccccc6N(c6ccccc6)c6ccccc6C54)c3)n2)cc1 ; c1ccc(-c2nc(-c3ccccc3)nc(-c3cccc(N4c5ccccc5-c5c(n(-c6ccccc6)c6ccccc56)-c5ccccc54)c3)n2)cc1]

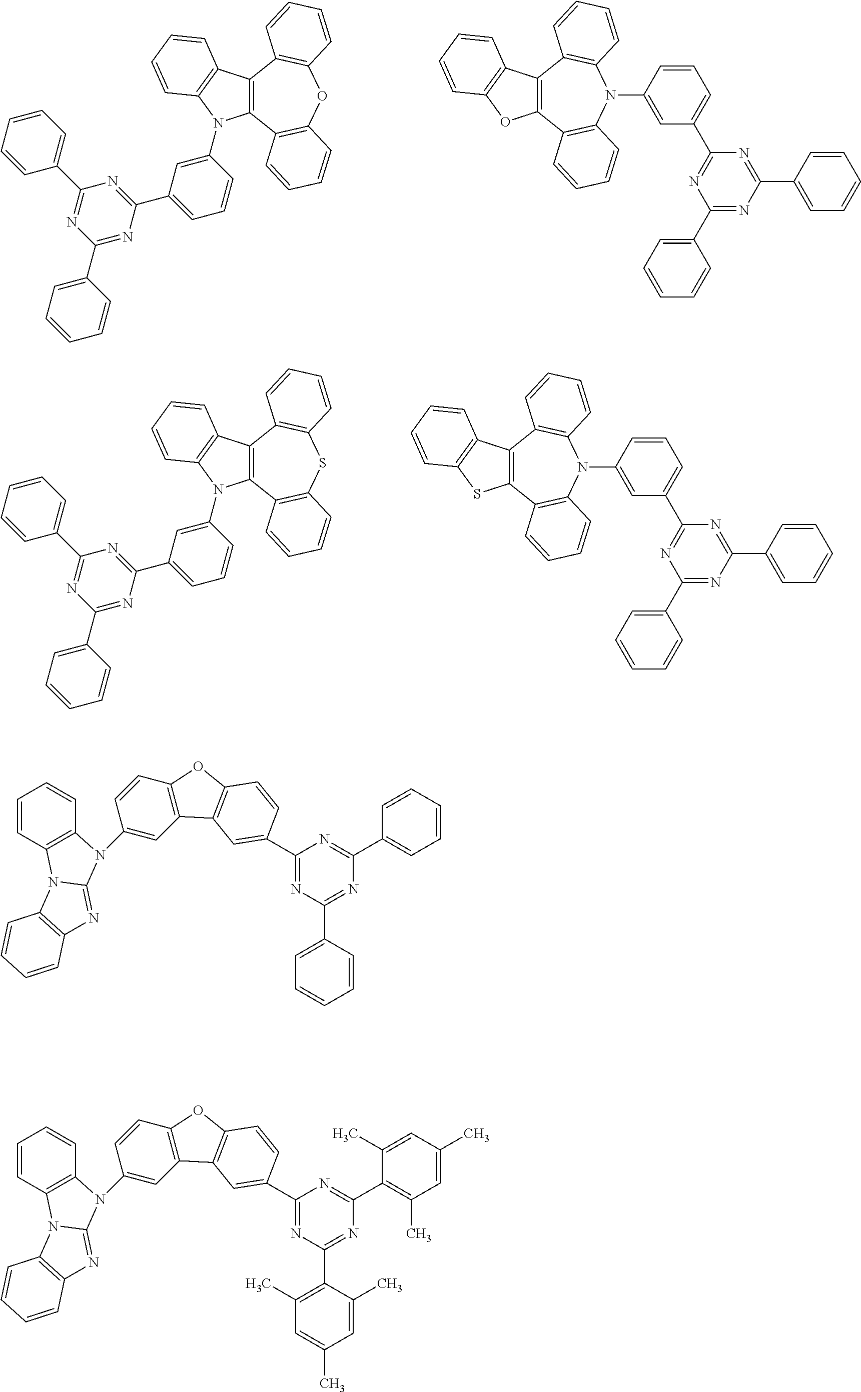

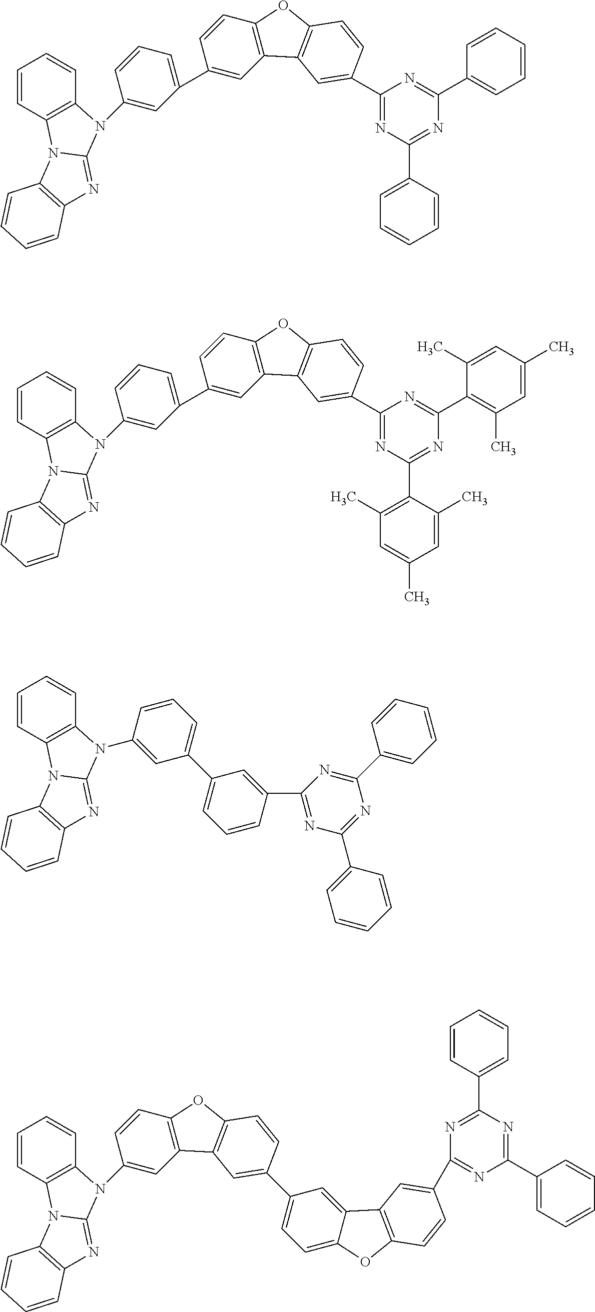 [SMILES: c1ccc(-c2nc(-c3ccccc3)nc(-c3ccc4oc5ccc(-c6cccc(-n7c8ccccc8n8c9ccccc9nc78)c6)cc5c4c3)n2)cc1 ; Cc1cc(C)c(-c2nc(-c3ccc4oc5ccc(-c6cccc(-n7c8ccccc8n8c9ccccc9nc78)c6)cc5c4c3)nc(-c3c(C)cc(C)cc3C)n2)c(C)c1 ; c1ccc(-c2nc(-c3ccccc3)nc(-c3cccc(-c4cccc(-n5c6ccccc6n6c7ccccc7nc56)c4)c3)n2)cc1 ; c1ccc(-c2nc(-c3ccccc3)nc(-c3ccc4oc5ccc(-c6ccc7oc8ccc(-n9c%10ccccc%10n%10c%11ccccc%11nc9%10)cc8c7c6)cc5c4c3)n2)cc1]

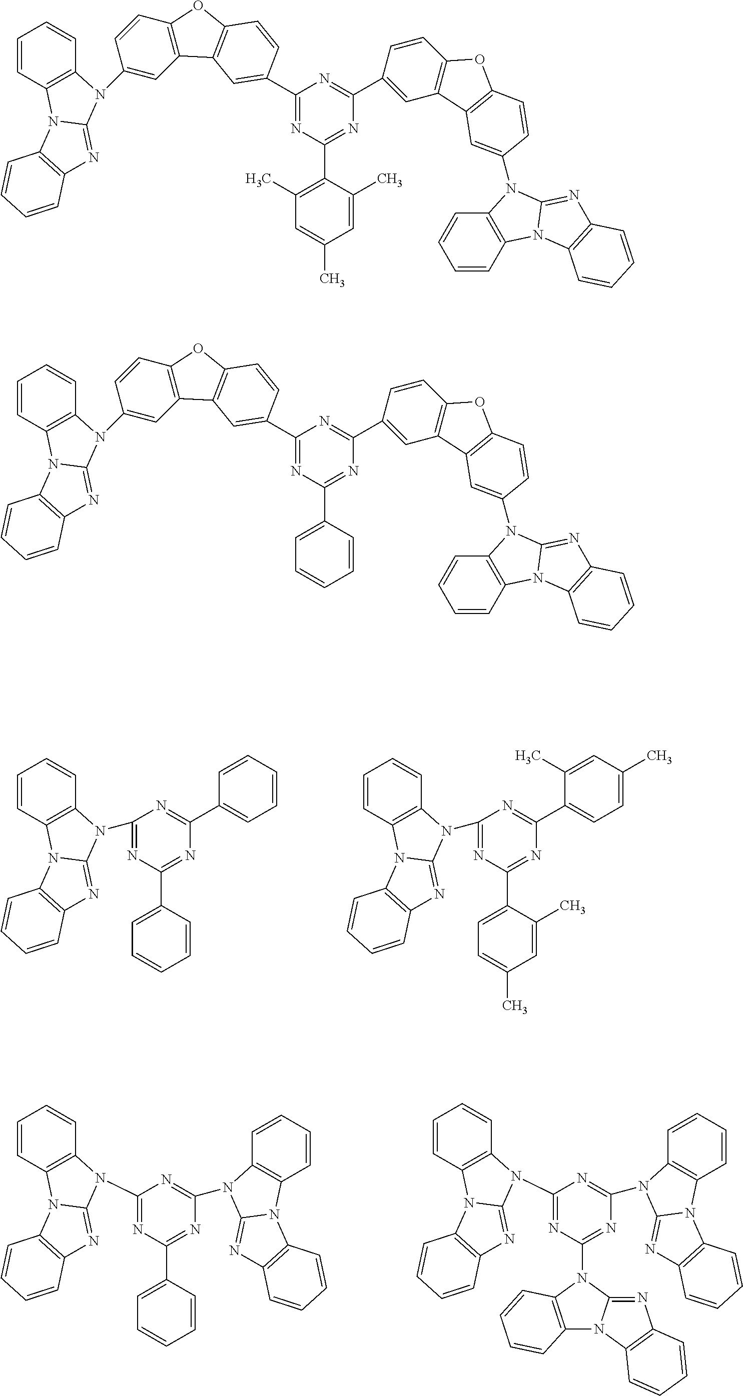

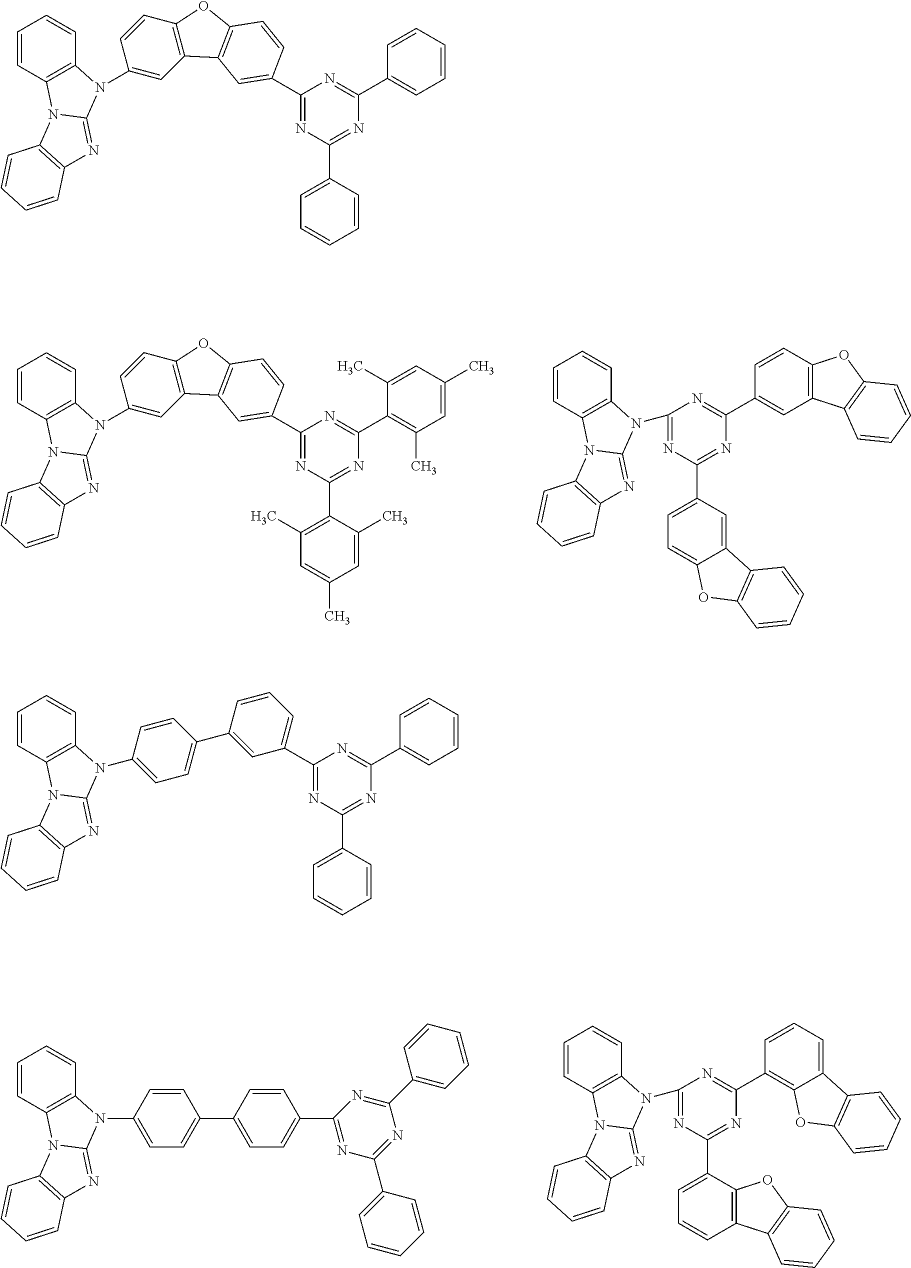

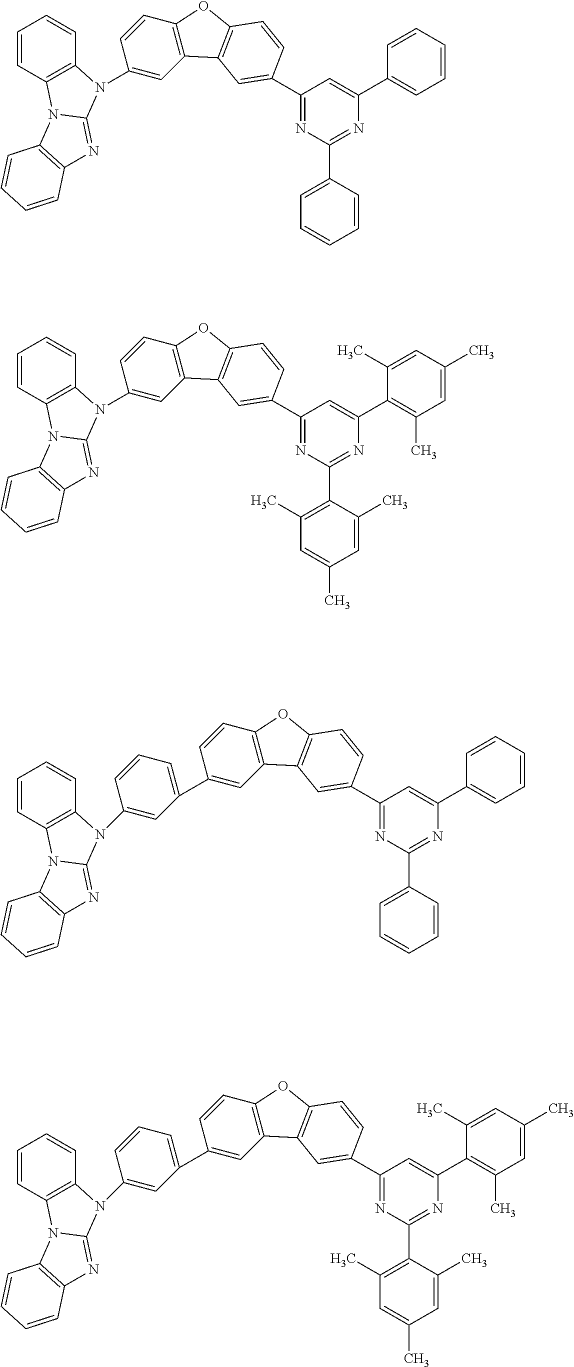 [SMILES: c1ccc(-c2cc(-c3ccc4oc5ccc(-n6c7ccccc7n7c8ccccc8nc67)cc5c4c3)nc(-c3ccccc3)n2)cc1 ; Cc1cc(C)c(-c2cc(-c3ccc4oc5ccc(-n6c7ccccc7n7c8ccccc8nc67)cc5c4c3)nc(-c3c(C)cc(C)cc3C)n2)c(C)c1 ; c1ccc(-c2cc(-c3ccc4oc5ccc(-c6cccc(-n7c8ccccc8n8c9ccccc9nc78)c6)cc5c4c3)nc(-c3ccccc3)n2)cc1 ; Cc1cc(C)c(-c2cc(-c3ccc4oc5ccc(-c6cccc(-n7c8ccccc8n8c9ccccc9nc78)c6)cc5c4c3)nc(-c3c(C)cc(C)cc3C)n2)c(C)c1]

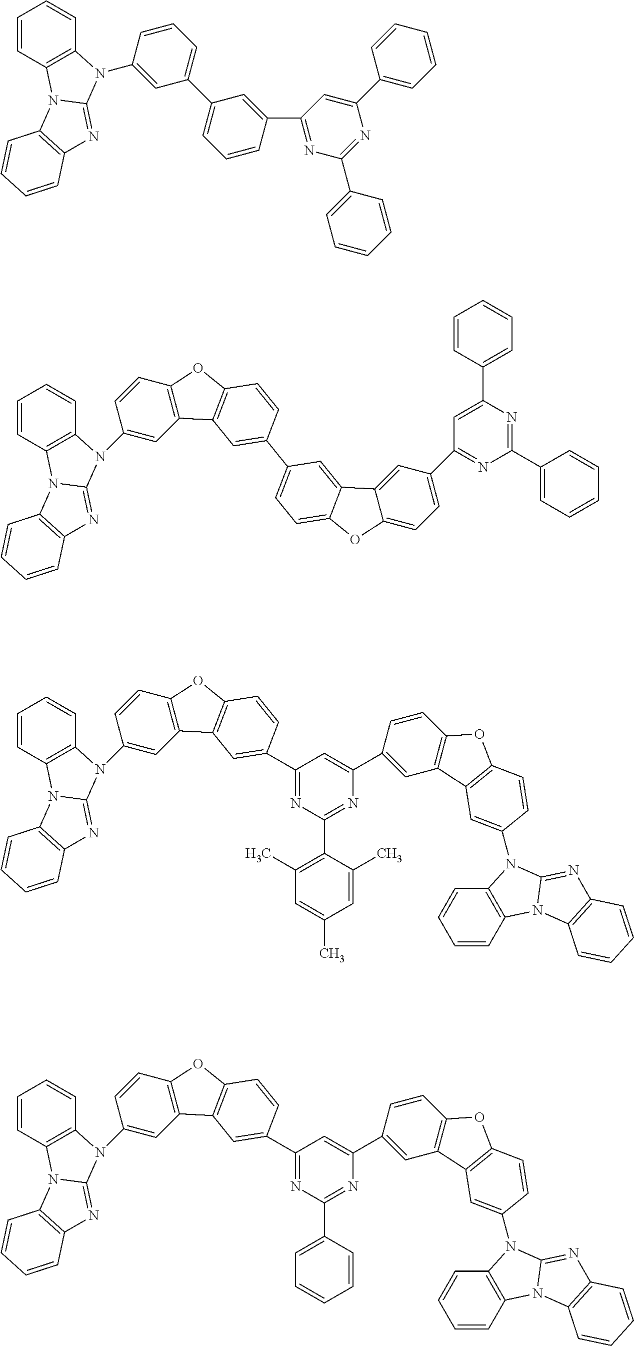 [SMILES: c1ccc(-c2cc(-c3cccc(-c4cccc(-n5c6ccccc6n6c7ccccc7nc56)c4)c3)nc(-c3ccccc3)n2)cc1 ; c1ccc(-c2cc(-c3ccc4oc5ccc(-c6ccc7oc8ccc(-n9c%10ccccc%10n%10c%11ccccc%11nc9%10)cc8c7c6)cc5c4c3)nc(-c3ccccc3)n2)cc1 ; Cc1cc(C)c(-c2nc(-c3ccc4oc5ccc(-n6c7ccccc7n7c8ccccc8nc67)cc5c4c3)cc(-c3ccc4oc5ccc(-n6c7ccccc7n7c8ccccc8nc67)cc5c4c3)n2)c(C)c1 ; c1ccc(-c2nc(-c3ccc4oc5ccc(-n6c7ccccc7n7c8ccccc8nc67)cc5c4c3)cc(-c3ccc4oc5ccc(-n6c7ccccc7n7c8ccccc8nc67)cc5c4c3)n2)cc1]

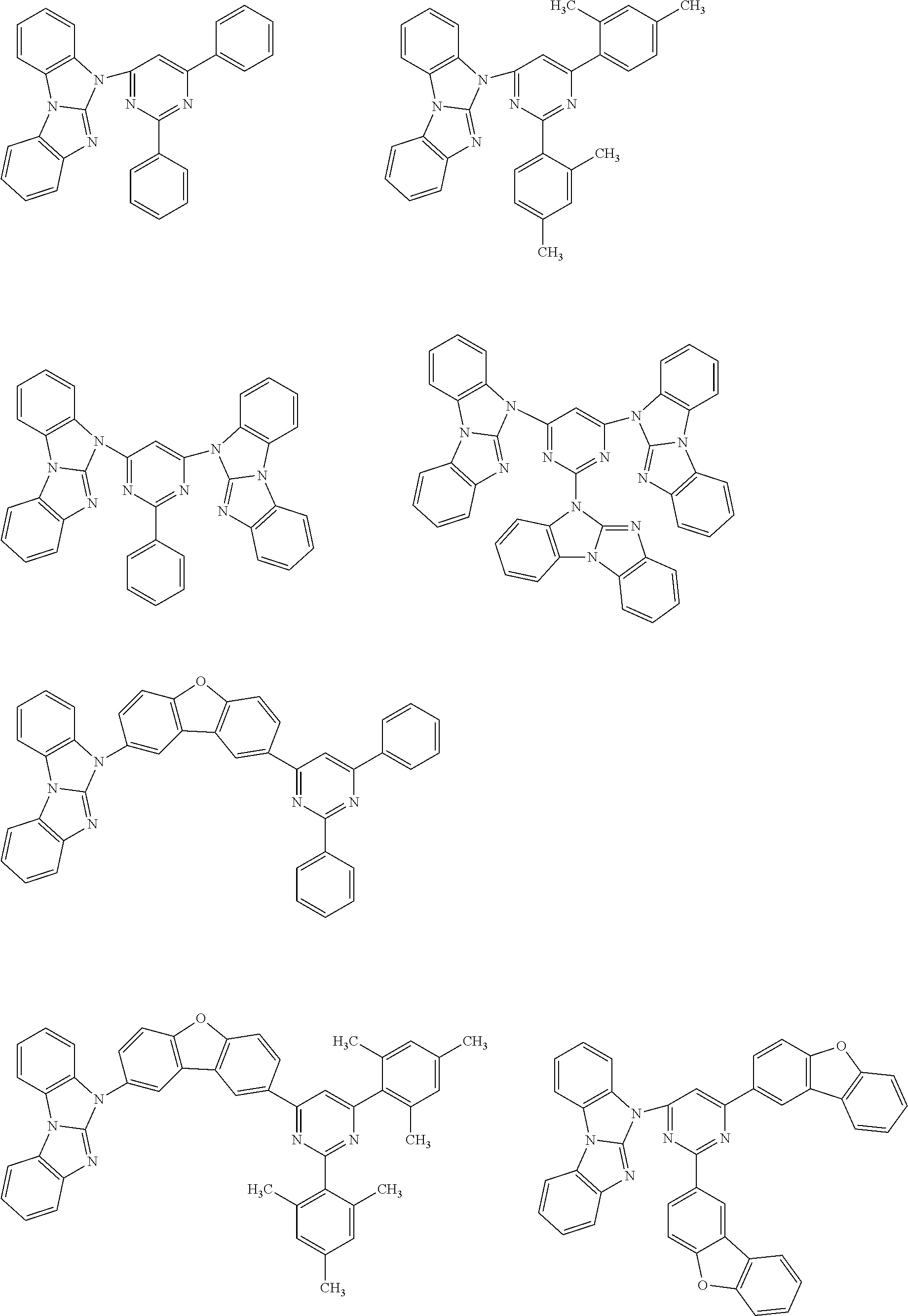

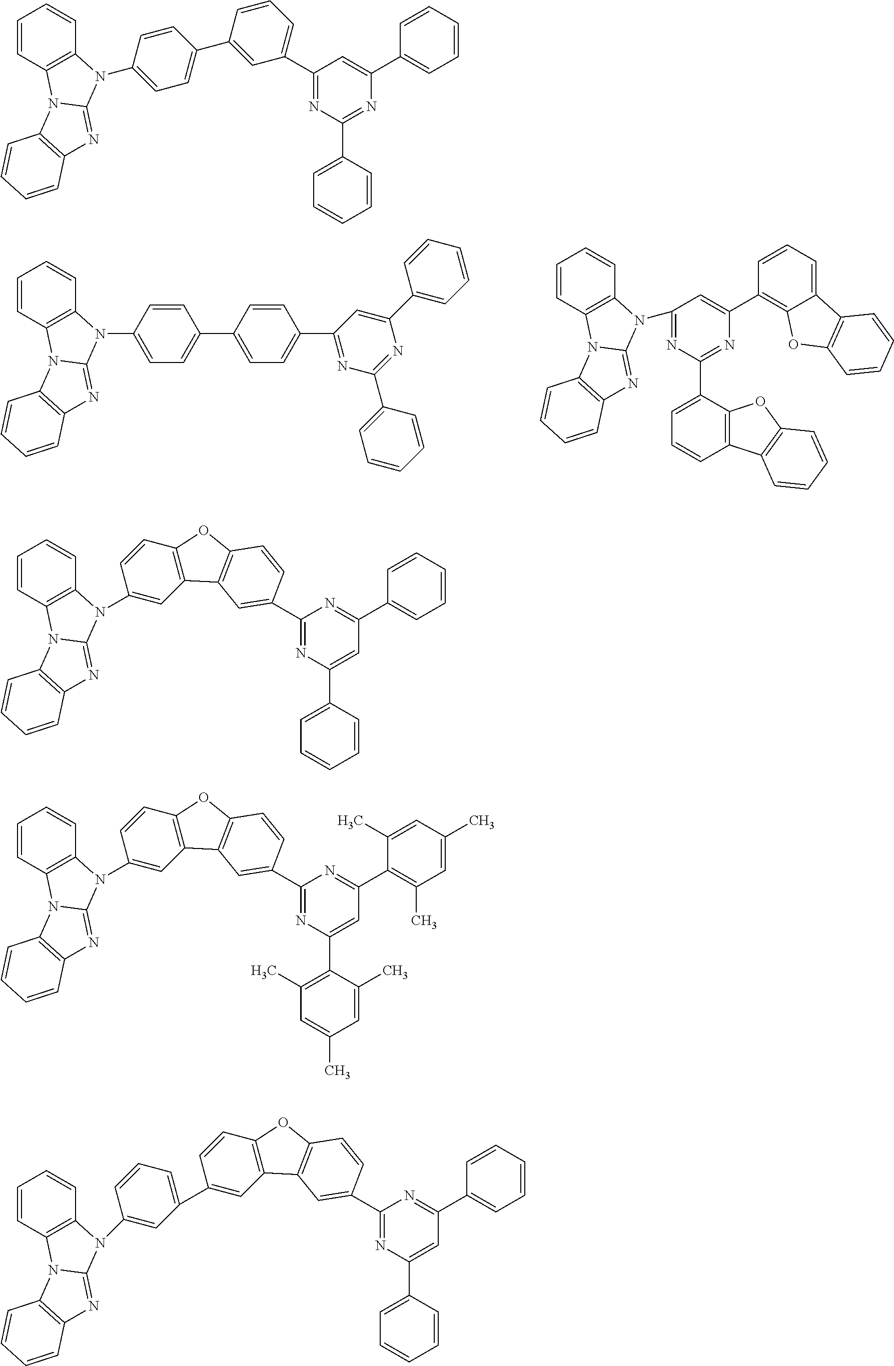

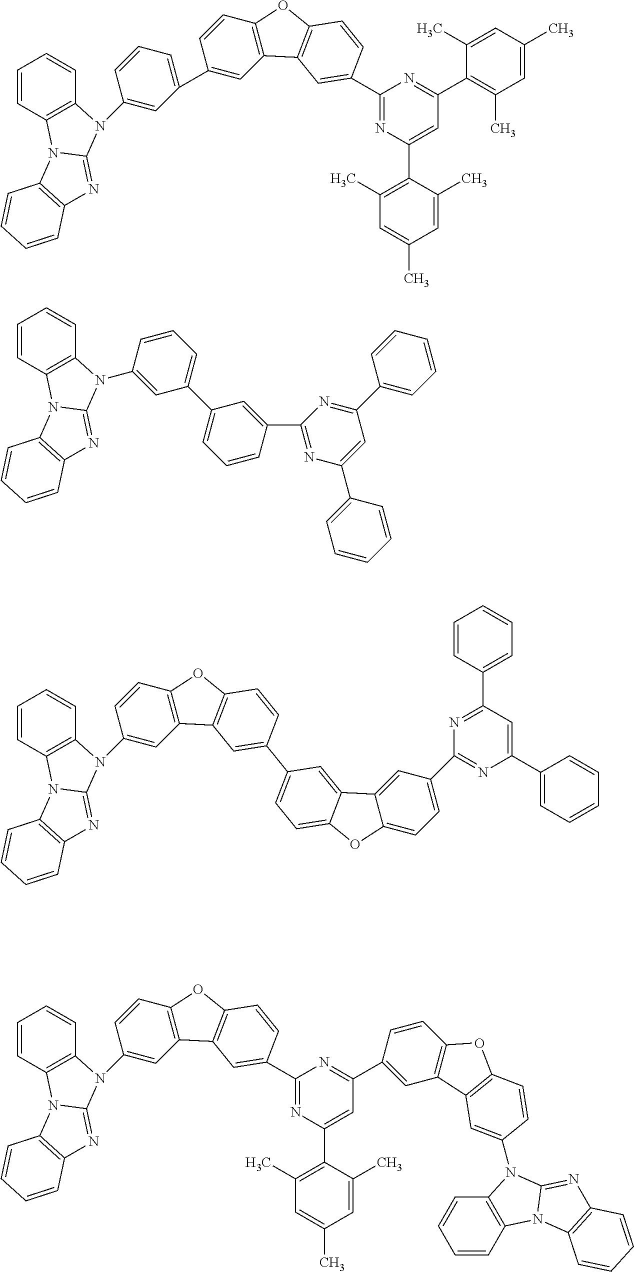 [SMILES: Cc1cc(C)c(-c2cc(-c3c(C)cc(C)cc3C)nc(-c3ccc4oc5ccc(-c6cccc(-n7c8ccccc8n8c9ccccc9nc78)c6)cc5c4c3)n2)c(C)c1 ; c1ccc(-c2cc(-c3ccccc3)nc(-c3cccc(-c4cccc(-n5c6ccccc6n6c7ccccc7nc56)c4)c3)n2)cc1 ; c1ccc(-c2cc(-c3ccccc3)nc(-c3ccc4oc5ccc(-c6ccc7oc8ccc(-n9c%10ccccc%10n%10c%11ccccc%11nc9%10)cc8c7c6)cc5c4c3)n2)cc1 ; Cc1cc(C)c(-c2cc(-c3ccc4oc5ccc(-n6c7ccccc7n7c8ccccc8nc67)cc5c4c3)nc(-c3ccc4oc5ccc(-n6c7ccccc7n7c8ccccc8nc67)cc5c4c3)n2)c(C)c1]

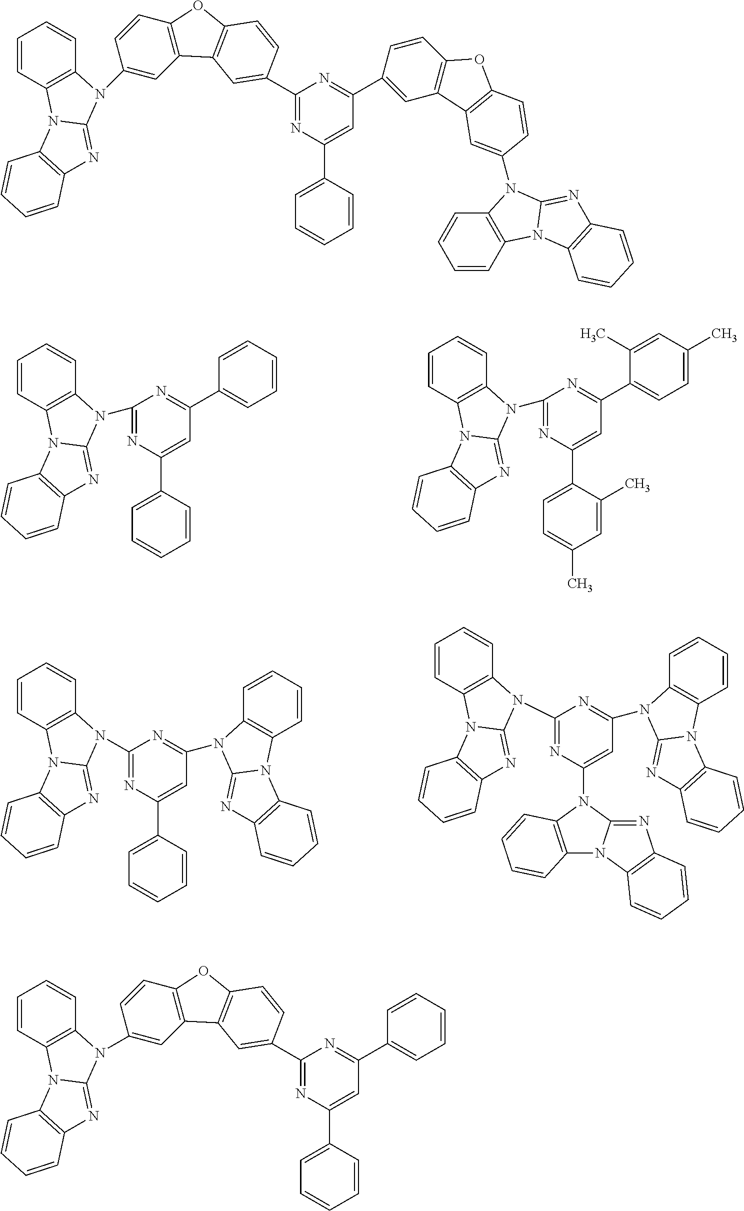 [SMILES: c1ccc(-c2cc(-c3ccc4oc5ccc(-n6c7ccccc7n7c8ccccc8nc67)cc5c4c3)nc(-c3ccc4oc5ccc(-n6c7ccccc7n7c8ccccc8nc67)cc5c4c3)n2)cc1 ; c1ccc(-c2cc(-c3ccccc3)nc(-n3c4ccccc4n4c5ccccc5nc34)n2)cc1 ; Cc1ccc(-c2cc(-c3ccc(C)cc3C)nc(-n3c4ccccc4n4c5ccccc5nc34)n2)c(C)c1 ; c1ccc(-c2cc(-n3c4ccccc4n4c5ccccc5nc34)nc(-n3c4ccccc4n4c5ccccc5nc34)n2)cc1 ; c1ccc2c(c1)nc1n(-c3cc(-n4c5ccccc5n5c6ccccc6nc45)nc(-n4c5ccccc5n5c6ccccc6nc45)n3)c3ccccc3n21 ; c1ccc(-c2cc(-c3ccccc3)nc(-c3ccc4oc5ccc(-n6c7ccccc7n7c8ccccc8nc67)cc5c4c3)n2)cc1]

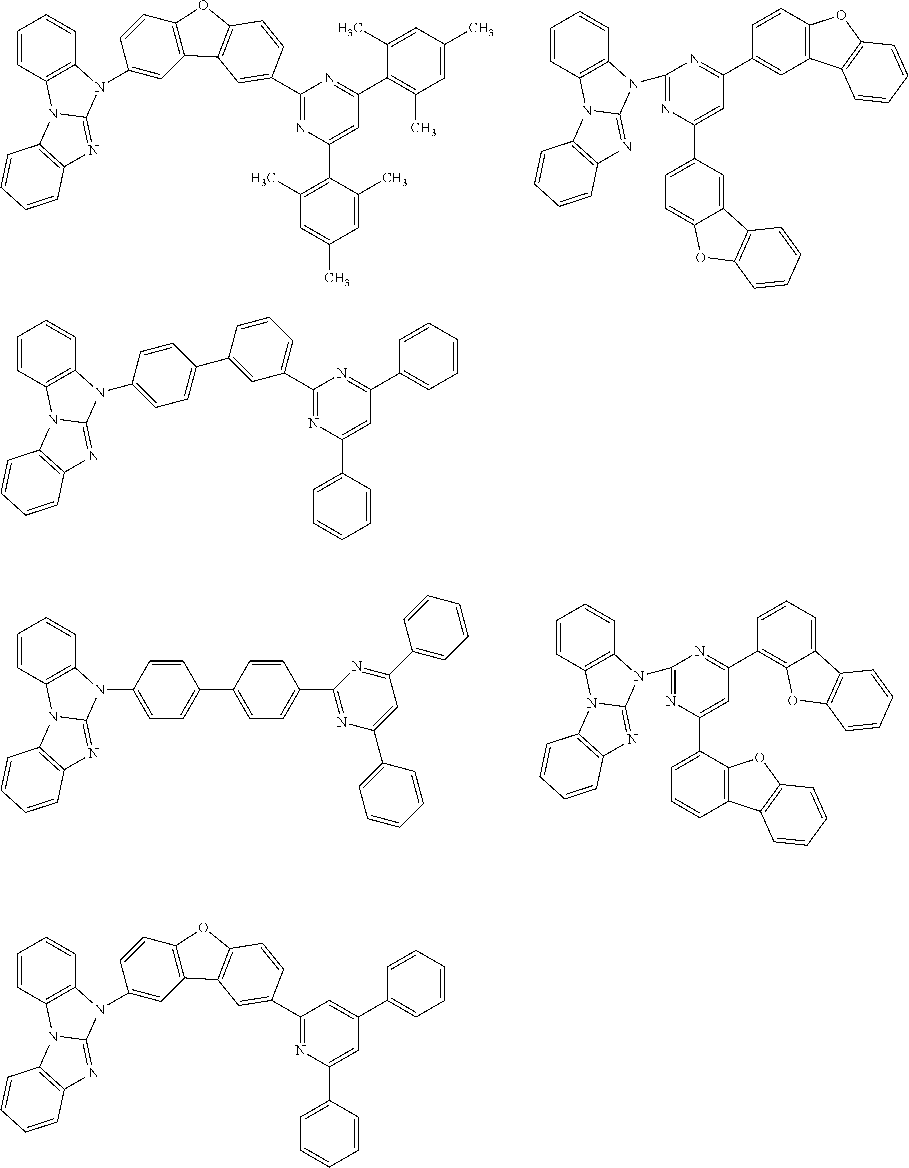

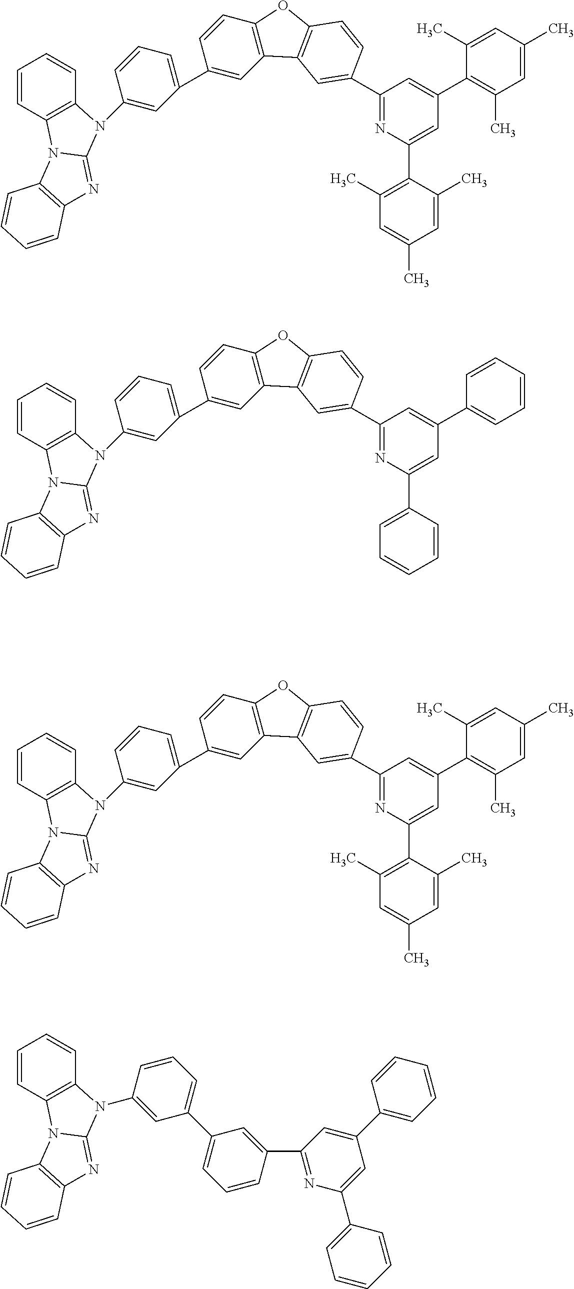 [SMILES: Cc1cc(C)c(-c2cc(-c3ccc4oc5ccc(-c6cccc(-n7c8ccccc8n8c9ccccc9nc78)c6)cc5c4c3)nc(-c3c(C)cc(C)cc3C)c2)c(C)c1 ; c1ccc(-c2cc(-c3ccccc3)nc(-c3ccc4oc5ccc(-c6cccc(-n7c8ccccc8n8c9ccccc9nc78)c6)cc5c4c3)c2)cc1 ; Cc1cc(C)c(-c2cc(-c3ccc4oc5ccc(-c6cccc(-n7c8ccccc8n8c9ccccc9nc78)c6)cc5c4c3)nc(-c3c(C)cc(C)cc3C)c2)c(C)c1 ; c1ccc(-c2cc(-c3ccccc3)nc(-c3cccc(-c4cccc(-n5c6ccccc6n6c7ccccc7nc56)c4)c3)c2)cc1]

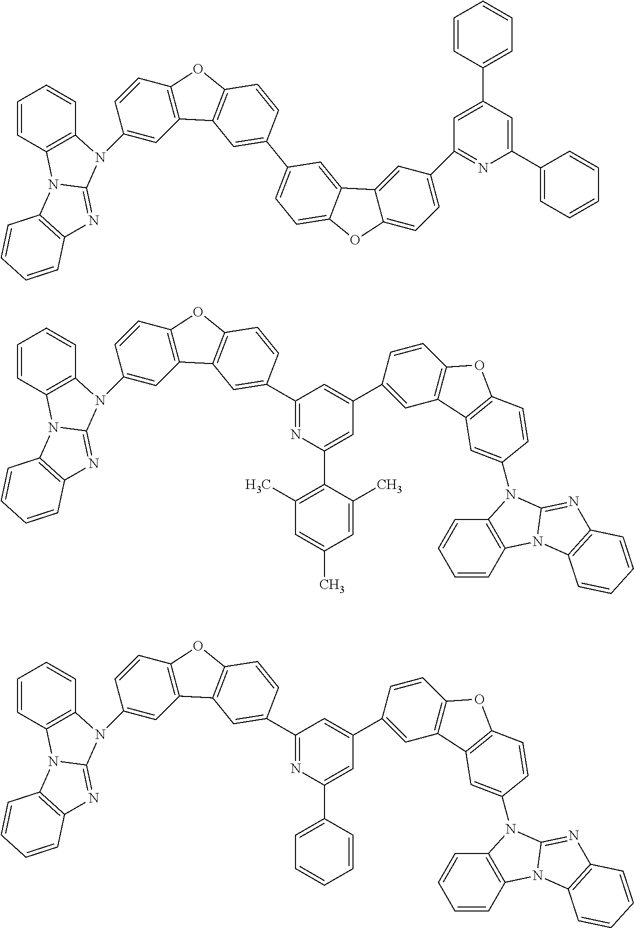 [SMILES: c1ccc(-c2cc(-c3ccccc3)nc(-c3ccc4oc5ccc(-c6ccc7oc8ccc(-n9c%10ccccc%10n%10c%11ccccc%11nc9%10)cc8c7c6)cc5c4c3)c2)cc1 ; Cc1cc(C)c(-c2cc(-c3ccc4oc5ccc(-n6c7ccccc7n7c8ccccc8nc67)cc5c4c3)cc(-c3ccc4oc5ccc(-n6c7ccccc7n7c8ccccc8nc67)cc5c4c3)n2)c(C)c1 ; c1ccc(-c2cc(-c3ccc4oc5ccc(-n6c7ccccc7n7c8ccccc8nc67)cc5c4c3)cc(-c3ccc4oc5ccc(-n6c7ccccc7n7c8ccccc8nc67)cc5c4c3)n2)cc1]

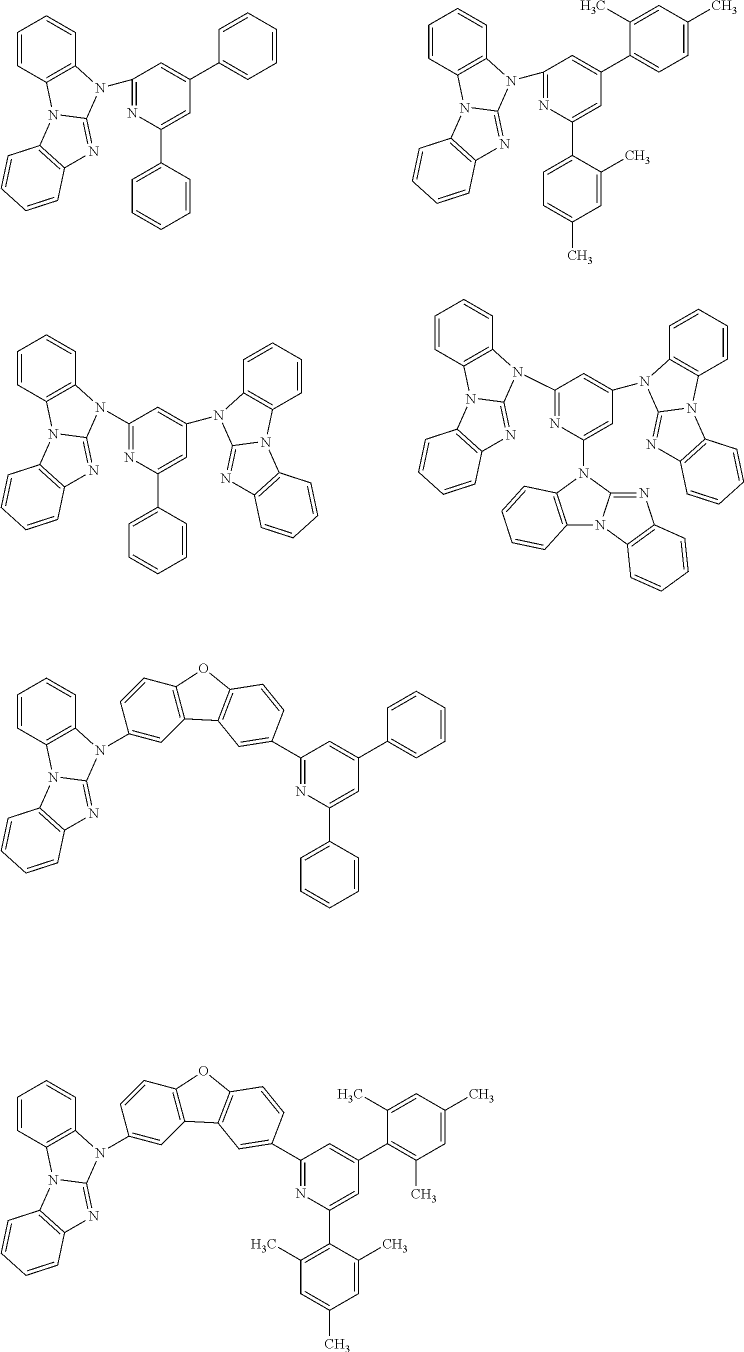 [SMILES: c1ccc(-c2cc(-c3ccccc3)nc(-n3c4ccccc4n4c5ccccc5nc34)c2)cc1 ; Cc1ccc(-c2cc(-c3ccc(C)cc3C)nc(-n3c4ccccc4n4c5ccccc5nc34)c2)c(C)c1 ; c1ccc(-c2cc(-n3c4ccccc4n4c5ccccc5nc34)cc(-n3c4ccccc4n4c5ccccc5nc34)n2)cc1 ; c1ccc2c(c1)nc1n(-c3cc(-n4c5ccccc5n5c6ccccc6nc45)nc(-n4c5ccccc5n5c6ccccc6nc45)c3)c3ccccc3n21 ; c1ccc(-c2cc(-c3ccccc3)nc(-c3ccc4oc5ccc(-n6c7ccccc7n7c8ccccc8nc67)cc5c4c3)c2)cc1 ; Cc1cc(C)c(-c2cc(-c3ccc4oc5ccc(-n6c7ccccc7n7c8ccccc8nc67)cc5c4c3)nc(-c3c(C)cc(C)cc3C)c2)c(C)c1]

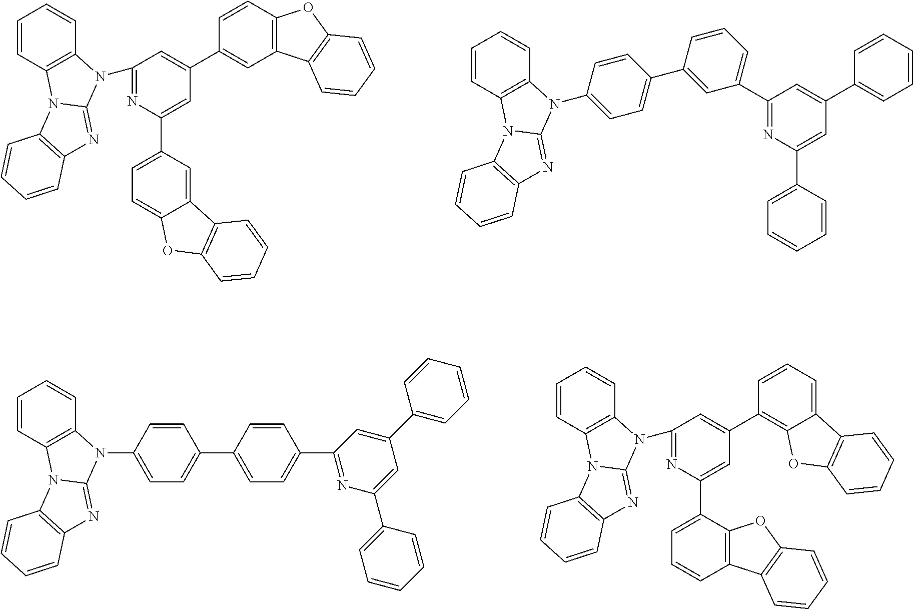 [SMILES: c1ccc2c(c1)nc1n(-c3cc(-c4ccc5oc6ccccc6c5c4)cc(-c4ccc5oc6ccccc6c5c4)n3)c3ccccc3n21 ; c1ccc(-c2cc(-c3ccccc3)nc(-c3cccc(-c4ccc(-n5c6ccccc6n6c7ccccc7nc56)cc4)c3)c2)cc1 ; c1ccc(-c2cc(-c3ccccc3)nc(-c3ccc(-c4ccc(-n5c6ccccc6n6c7ccccc7nc56)cc4)cc3)c2)cc1 ; c1ccc2c(c1)nc1n(-c3cc(-c4cccc5c4oc4ccccc45)cc(-c4cccc5c4oc4ccccc45)n3)c3ccccc3n21]

The organic EL device as one aspect of the invention may further comprise one or more selected from a fluorescent emitting material and a phosphorescent emitting material.

In one embodiment of the invention, it is preferred that the emitting layer comprise a phosphorescent emitting material as an emitting material and that the phosphorescent emitting material be an ortho-metalated complex of a metal atom selected from iridium (Ir), osmium (Os) and platinum (Pt). Preferable phosphorescent emitting materials will be mentioned later.

Between the anode and the emitting layer, a hole-transporting layer may be further provided.

Between the cathode and the emitting layer, an electron-transporting layer may be provided.

The fluorescent emitting material and the phosphorescent emitting material, and materials used in the hole-transporting layer and the electron-transporting layer will be mentioned later.

Each layer of the organic EL device as one aspect of the invention may be formed by a dry film-forming method such as vacuum deposition, sputtering, plasma, or ion plating, or a wet film-forming method such as spin coating, dipping, or flow coating. The thickness of each layer is not particularly limited, but it should be adjusted to be an appropriate thickness. If the thickness of each layer is too large, a high voltage is required to be applied to obtain a certain amount of optical output, so that the efficiency may be deteriorated. If the thickness of each layer is too small, pinholes or the like may be generated, so that a sufficient luminance may not be obtained when an electric field is applied. The thickness of each layer is normally 5 nm to 10 μm, and further preferably 10 nm to 0.2 μm.

Hereinbelow, materials or the like of each element constituting the organic EL device of the invention will be explained below.

### (Substrate)

The substrate is used as a base of an emitting element. As the substrate, glass, quarts, plastic or the like can be used, for example. A flexible substrate may be used. A flexible substrate is a substrate that can be bent. For example, a plastic substrate made of polycarbonate or polyvinyl chloride or the like can be given.

### (Anode)

For an anode formed on the substrate, it is preferable to use a metal, an alloy, an electrically conductive compound having a large work function (specifically, 4.0 eV or more), a mixture thereof or the like. Specifically, indium oxide-tin oxide (ITO: Indium Tin Oxide), indium oxide-tin oxide containing silicon or silicon oxide, indium oxide-zinc oxide, tungsten oxide, indium oxide containing zinc oxide, graphene or the like can be given, for example. In addition, gold (Au), platinum (Pt) or a nitride of a metal material (e.g. titanium nitride) or the like can be given.

### (Hole-Injecting Layer)

A hole-injecting layer is a layer that contains a substance having high hole-injection property. As the substance having high hole-injection property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, or a polymer compound (oligomer, dendrimer, polymer, etc.) or the like can be used.

### (Hole-Transporting Layer)

A hole-transporting layer is a layer that contains a substance having high hole-transporting property. In the hole-transporting layer, aromatic amine compounds, carbazole derivatives, anthracene derivatives and the like can be used. Polymer compounds such as poly(N-vinylcarbazole) (abbreviation: PVK) or poly(4-vinyltriphenylamine) (abbreviation: PVTPA) can also be used. As long as it has high property of transporting holes rather than electrons, other substances than those mentioned above can be used. The layer containing the substance having high hole-transporting property may be not only a single layer but also a layer obtained by stacking two or more layers composed of the above-mentioned substances.

### (Guest Material of Emitting Layer)

An emitting layer is a layer that contains a substance having high emitting property, and various materials can be used for the emitting layer. For example, as the substance having high emitting property, a fluorescent compound that emits fluorescence or a phosphorescent compound that emits phosphorescence can be used. A fluorescent compound is a compound that can emit light from the singlet excited state, and a phosphorescent compound is a compound that can emit light from the triplet excited state. These compounds are often referred to as a dopant or a dopant material.

As the blue fluorescent emitting material that can be used in the emitting layer, pyrene derivatives, styrylamine derivatives, chrysene derivatives, fluoranthene derivatives, fluorene derivatives, diamine derivatives, triarylamine derivatives or the like can be used. Specific examples thereof include N,N′-bis[4-(9H-carbazol-9-yl)phenyl]-N,N′-diphenylstilbene-4,4′-diamine (abbreviation: YGA2S), 4-(9H-carbazolyl-9-yl)-4′-(10-phenyl-9-anthryl)triphenylamine (abbreviation: YGAPA), 4-(10-phenyl-9-anthryl)-4′-(9-phenyl-9H-carbazol-3-yl)triphenylamine (abbreviation: PCBAPA) or the like.

As the green fluorescent emitting material that can be used in the emitting layer, an aromatic amine derivative or the like can be used. Specific examples thereof include N-(9,10-diphenyl-2-anthryl)-N,9-diphenyl-9H-carbazole-3-amine (abbreviation: 2PCAPA), N-[9,10-bis(1,1′-biphenyl-2-yl)-2-anthryI]-N,9-diphenyl-9H-carbazole-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryI]-N,N′,N′-triphenyl-1,4-phenylenediamine (abbreviation: 2DPABPhA), N-[9,10-bis(1,1′-biphenyl-2-yl)]-N-[4-(9H-carbazole-9-yl)phenyl]-N-phenylanthracen-2-amine (abbreviation: 2YGABPhA), N,N,9-triphenylanthracene-9-amine (abbreviation: DPhAPhA) or the like.

As the red fluorescent emitting material that can be used in the emitting layer, tetracene derivatives, diamine derivatives or the like can be used. Specific examples thereof include N,N,N′,N′-tetrakis(4-methylphenyl) tetracene-5,11-diamine (abbreviation: p-mPhTD), 7,14-diphenyl-N,N,N′,N′-tetrakis (4-methylphenyl)acenaphtho[1,2-a]fluoranthene-3,10-diamine (abbreviation: p-mPhAFD) or the like.

In one aspect of the invention, it is preferred that the fluorescent emitting material contain at least one selected from an anthracene derivative, a fluoranthene derivative, a styrylamine derivative and an arylamine derivative.

As the blue phosphorescent emitting material that can be used in the emitting layer, a metal complex such as an iridium complex, an osmium complex and a platinum complex can be used. Specific examples include bis[2-(4′,6′-difluorophenyl)pyridinato-N,C2′]tetrakis(1-pyrazolyl)(borate) iridium(III) (abbreviation: FIr₆), bis[2-(4′,6′-difluorophenyl)pyridinato-N,C2′](picolinato) iridium (III)(abbreviation: Flrpic), bis[2-(3′,5′-bistrifluoromethylphenyl)pyridinato-N,C2′](picolinato) iridium (III)(abbreviation: Ir(CF₃ppy)₂(pic)), bis[2-(4′,6′-difluorophenyl)pyridinato-N,C2′](acetylacetonato) iridium (III)(abbreviation: Flracac) or the like.

As the green phosphorescent emitting material that can be used in the emitting layer, an iridium complex or the like can be used. Tris(2-phenylpyridinato-N,C2′)iridium (III)(abbreviation: Ir(ppy)₃), bis(2-phenylpyridinato-N,C2′)(acetylacetonato) iridium (III)(abbreviation: Ir(ppy)₂(acac)), bis(1,2-diphenyl-1H-benzoimidazolate) (acetylacetonato) iridium (III)(abbreviation: Ir(pbi)₂(acac)), bis(benzo[h]quinolinato)(acetylacetonato) iridium (III)(abbreviation: Ir(bzq)₂(acac)) or the like can be given.

As the red phosphorescent emitting material that can be used in the emitting layer, a metal complex such as an iridium complex, a platinum complex, a terbium complex and a europium complex is used. Specific examples thereof include an organic metal complex such as bis[2-(2′-benzo[4,5-α]thienyl)pyridinato-N,C3′](acetylacetonato) iridium (III) (abbreviation: Ir(btp)₂(acac)), bis(1-phenylisoquinolinato-N,C2′)(acetylacetonato) iridium (III) (abbreviation: Ir(piq)₂(acac)), (acetyl acetonato) bis[2,3-bis(4-fluorophenyl)quinoxalinato]iridium (III) (abbreviation: Ir(Fdpq)₂(acac)), 2,3,7,8,12,13,17,18-octaethyl-21H,23H-porphyline platinum (II) (abbreviation: PtOEP) or the like.

Further, since a rare earth metal complex such as tris(acetyl acetonato)(monophenanthroline) terbium(III) (abbreviation: Tb(acac)₃(Phen)), tris(1,3-diphenyl-1,3-propanedionate) (monophenanthroline) europium (III) (abbreviation: Eu(DBM)₃(Phen)), tris[1-(2-thenoyl)-3,3,3-trifluoroacetonato](monophenanthroline)europium (III) (abbreviation: Eu(TTA)₃(Phen)) emits light from rare earth metal ions (electron transition between different multiplicities), it can be used as a phosphorescent compound.

In one aspect of the invention, the phosphorescent emitting material is preferably an ortho-metalated complex of a metal element selected from iridium (Ir), osmium (Os) and platinum (Pt).

A phosphorescent emitting material that is an ortho-metalated complex of a metal element selected from iridium (Ir), osmium (Os) and platinum (Pt) is preferably a complex represented by the following formula (α):

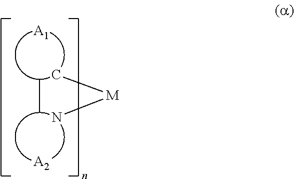

In the formula (α), M is at least one metal selected from osmium, iridium and platinum, and n is the valence of the metal.

The ring A₁ is a substituted or unsubstituted aryl group including 6 to 24 ring carbon atoms or a heteroaryl group including 5 to 30 ring atoms, and the ring A₂ is a substituted or unsubstituted heteroaryl group including 5 to 30 ring atoms that contains nitrogen as the atom constituting a hetero ring.

As the aryl group including 6 to 24 ring carbon atoms in the ring A₁ in the formula (α), the aryl group in the above-mentioned formula (1) can be given.

As the heteroaryl group including 5 to 30 ring atoms in the ring A₁ and the ring A₂ in the formula (α), the aryl group in the above-mentioned formula (1) can be given.

The substituent which the ring A₁ and the ring A₂ in the formula (α) can include is the same as those in the above-mentioned formula (1).

Further, the complex represented by the formula (α) is preferably a complex represented by the following formula (T) or (U):

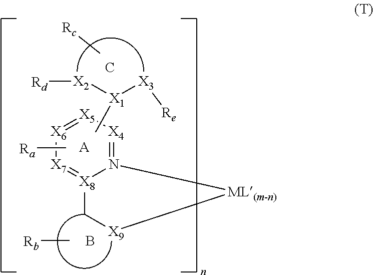

In the formula (T), M is a metal and the ring B and the ring C are independently an aryl group or a heteroaryl group including 5 or 6 ring atoms.

The ring A-ring B is a bonding pair of the aryl group or the heteroaryl group, and it is coordinated at the metal M through the nitrogen atom in the ring A and the sp² hybridized atom in the ring B.

The ring A-ring C is a bonding pair of the aryl group or the heteroaryl group.

Rₐ, R_{b} and R_{c} are independently any one selected from a hydrogen atom, a substituted or unsubstituted alkyl group including 1 to 25 carbon atoms, a substituted or unsubstituted alkoxy group including 1 to 25 carbon atoms, a substituted or unsubstituted amino group, a substituted or unsubstituted alkenyl group including 2 to 25 carbon atoms, a substituted or unsubstituted alkynyl group including 2 to 25 carbon atoms, a substituted or unsubstituted aralkyl group including 7 to 50 carbon atoms, a substituted or unsubstituted aryl group including 6 to 24 ring carbon atoms and a substituted or unsubstituted heteroaryl group including 5 to 30 ring atoms. They are independently one to four.

X₁ to X₉ are independently a carbon atom or a nitrogen atom.

R_{d} and Rₑ are independently any one selected from the group consisting of a hydrogen atom, a substituted or unsubstituted alkyl group including 1 to 25 carbon atoms, a substituted or unsubstituted alkoxy group including 1 to 25 carbon atoms, a substituted or unsubstituted amino group, a substituted or unsubstituted alkenyl group including 2 to 25 carbon atoms, a substituted or unsubstituted alkynyl group including 2 to 25 carbon atoms, a substituted or unsubstituted aralkyl group including 7 to 50 carbon atoms, a substituted or unsubstituted aryl group including 6 to 24 ring carbon atoms and a substituted or unsubstituted heteroaryl group including 5 to 30 ring atoms, and at least one of R_{c}, R_{d} and Rₑ that is bonded with the ring C is not a hydrogen atom.

m shows an oxidization state of the metal M, and n is one or more. L′ is a monoanionic bidentate ligand.

In the formula (T), as examples of M, osmium, iridium, platinum or the like can be given, with iridium being preferable.

As the aryl group including 5 or 6 ring atoms represented by the ring B and the ring C, an aryl group in the formula (1) mentioned above can be given.

As the heteroaryl group including 5 or 6 ring atoms represented by the ring B and the ring C, the heteroaryl group mentioned above can be given.

As the substituted or unsubstituted alkyl group including 1 to 25 carbon atoms, the substituted or unsubstituted alkoxy group including 1 to 25 carbon atoms, the substituted or unsubstituted aralkyl group including 7 to 50 carbon atoms, the substituted or unsubstituted aryl group including 6 to 24 ring carbon atoms and the substituted or unsubstituted heteroaryl group including 5 to 30 ring atoms represented by R₁, R₂, Rₐ, R_{b} and R_{c}, the same groups as those mentioned above can be given.

As the substituted or unsubstituted alkenyl group including 2 to 25 carbon atoms and the substituted or unsubstituted alkynyl group including 2 to 25 carbon atoms represented by R₁, R₂, Rₐ, R_{b} and R_{c}, the same groups as those mentioned above can be given.

As the monoanionic bidentate ligand represented by L′, a ligand represented by the following formula (L′) can be given.

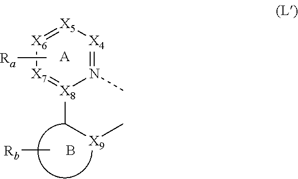

In the formula (L′), X₄ to X₉, Rₐ and R_{b} are as defined in X₄ to X₉, Rₐ and R_{b} in the formula (T), and preferable aspects are also the same.

Through a solid line extending from X₉ to the outside of the ring B and a dotted line extending from the nitrogen atom of the ring A to the outside of the ring A, the ligand represented by the formula (L′) is coordinated on the metal M represented by the formula (T):

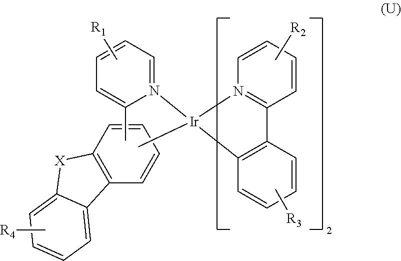

In the formula (U), X is any selected from NR, an oxygen atom, a sulfur atom, BR and a selenium atom, and R is a hydrogen atom or a substituted or unsubstituted alkyl group including 1 to 25 carbon atoms.

R₁, R₂, R₃ and R₄ are independently any selected from a hydrogen atom, a substituted or unsubstituted alkyl group including 1 to 25 carbon atoms and a substituted or unsubstituted aryl group including 6 to 24 ring carbon atoms. They are independently one to four.

In the formula (U), as the alkyl group including 1 to 25 carbon atoms represented by R, R₁, R₂, R₃ and R₄, the same groups as those mentioned above can be given, and preferable aspects are also the same.

As the aryl group including 6 to 24 ring carbon atoms represented by R₁, R₂, R₃ and R₄, the same groups as those mentioned above can be given, and preferable aspects are also the same.

As the complex represented by the formula (T) or (U), the following compounds are preferable. The complex is not particularly limited to those.

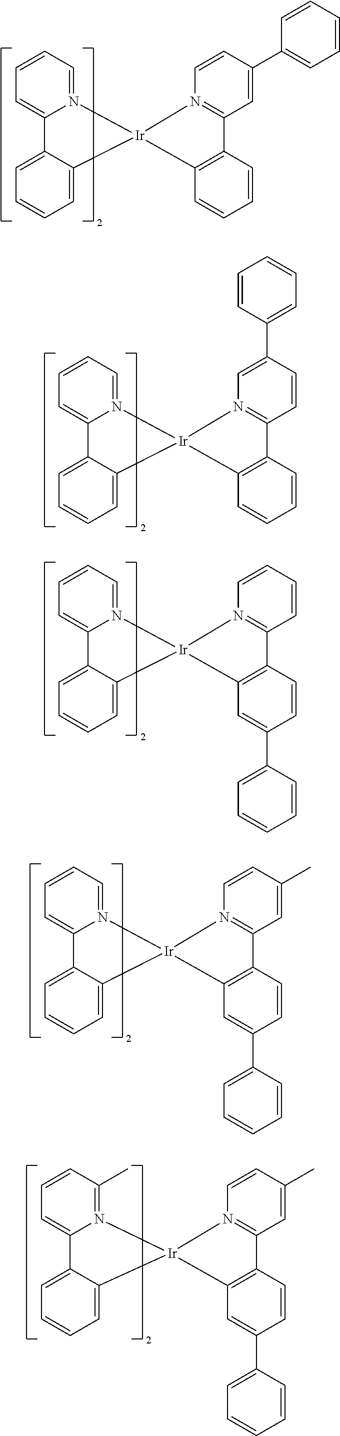

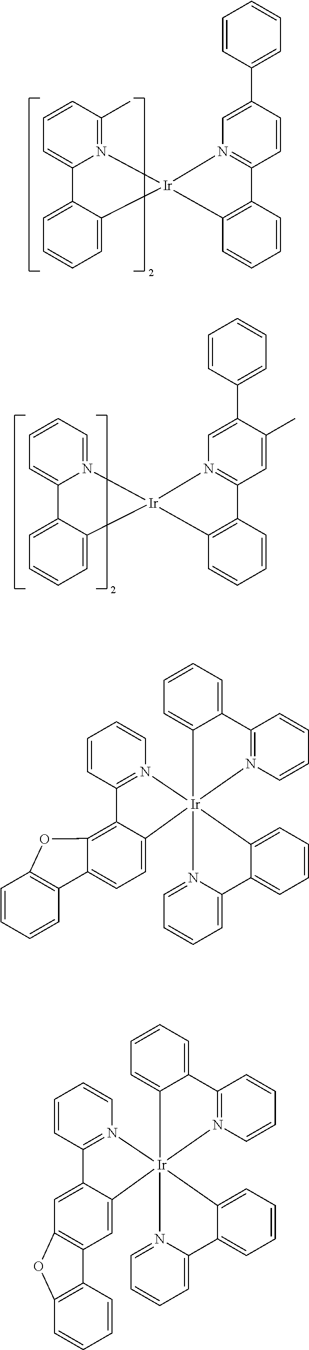

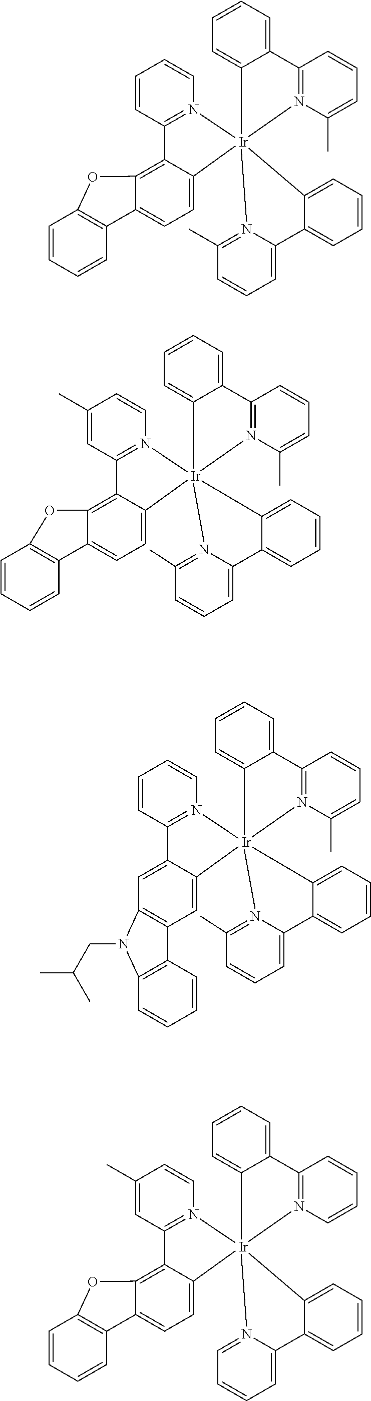

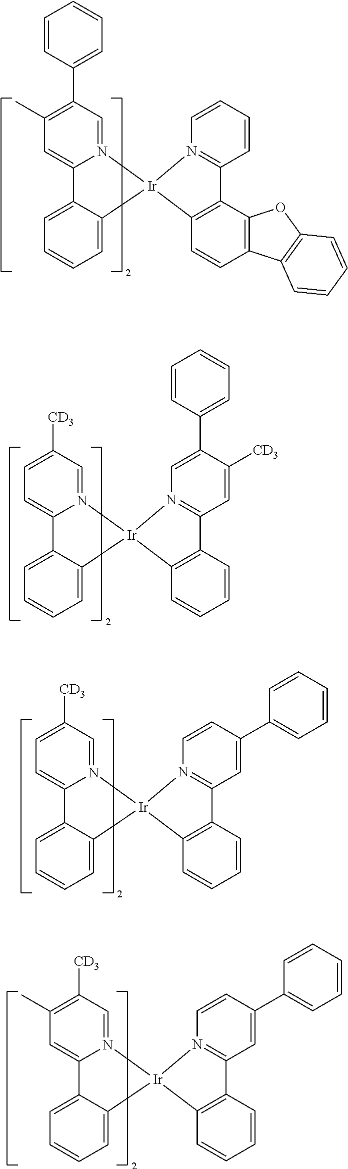

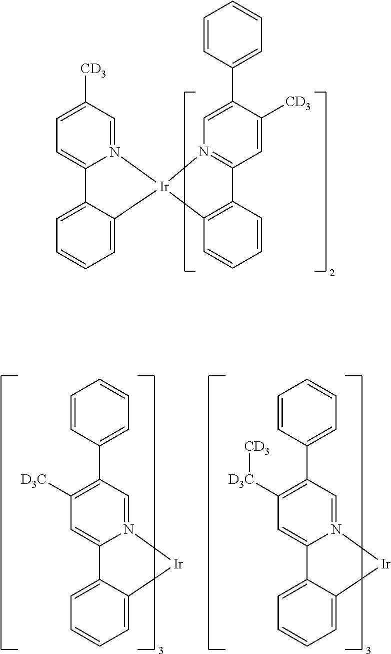

As the complex represented by the formula (α), other than the complex represented by the formula (T) or (U), a complex represented by the following formula (V), (X), (Y) or (Z) can also be used.

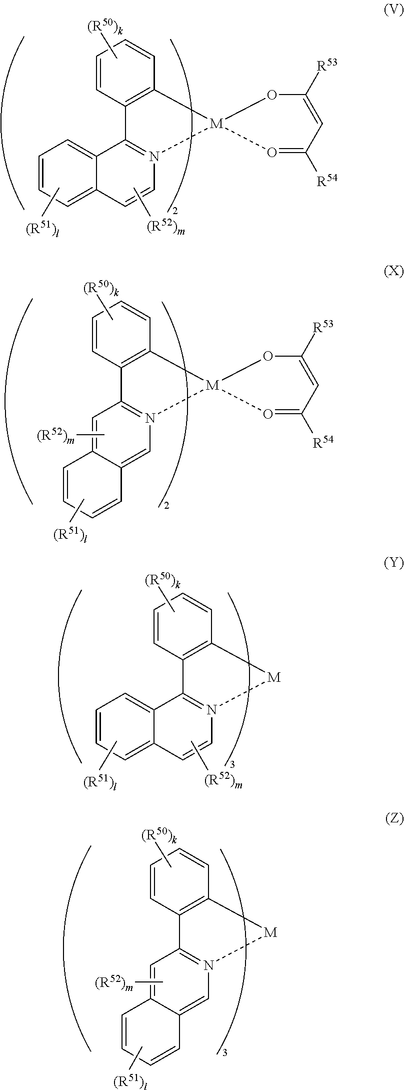

In the formula (V), (X), (Y) or (Z), R⁵⁰ to R⁵⁴ are a hydrogen atom or a substituent, k is an integer of 1 to 4, I is an integer of 1 to 4, and m is an integer of 1 to 2. M is Ir, Os or Pt.

As the substituent represented by R⁵⁰to R⁵⁴, the same substituents as those mentioned above can be given.

The complex represented by the formula (V) is preferably a complex represented by the following formula (V-1), and the complex represented by the formula (X) is preferably a complex represented by the following formula (X-1) or (X-2). In the following formulas (V-1), (X-1) and (X-2), R⁵⁰, k and M are the same as R⁵⁰, k and M mentioned above.

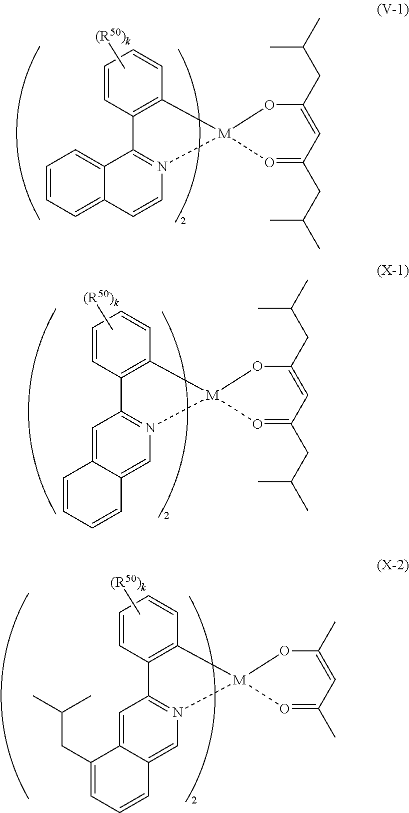

The specific examples of the complex represented by the formulas (V), (Y) or (Z) are shown below, but not particularly limited thereto.

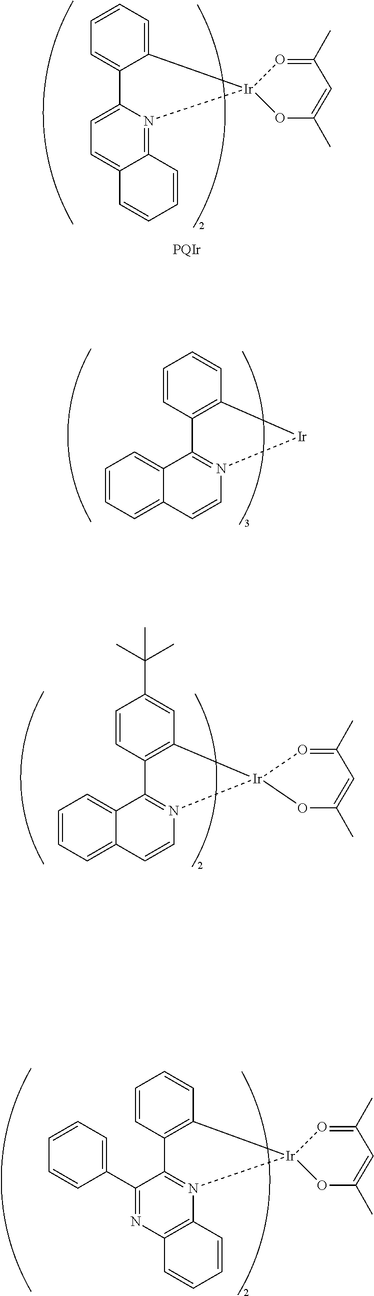

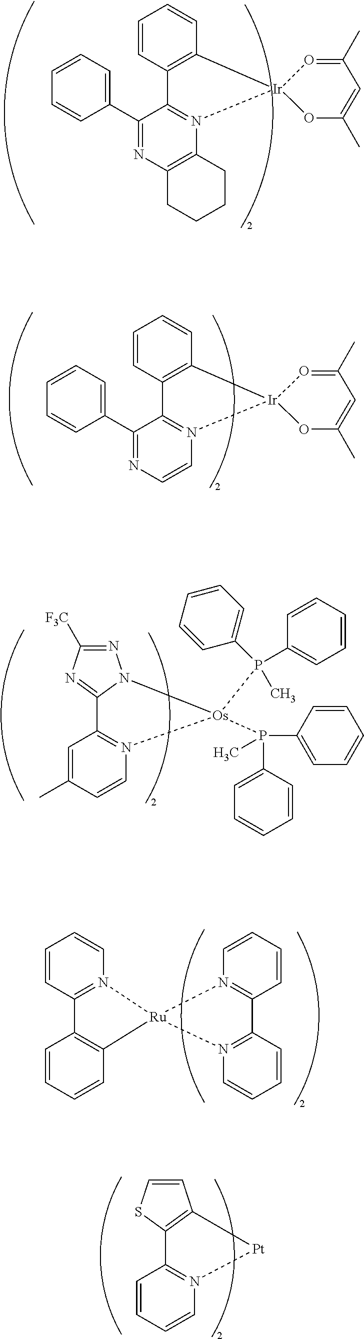

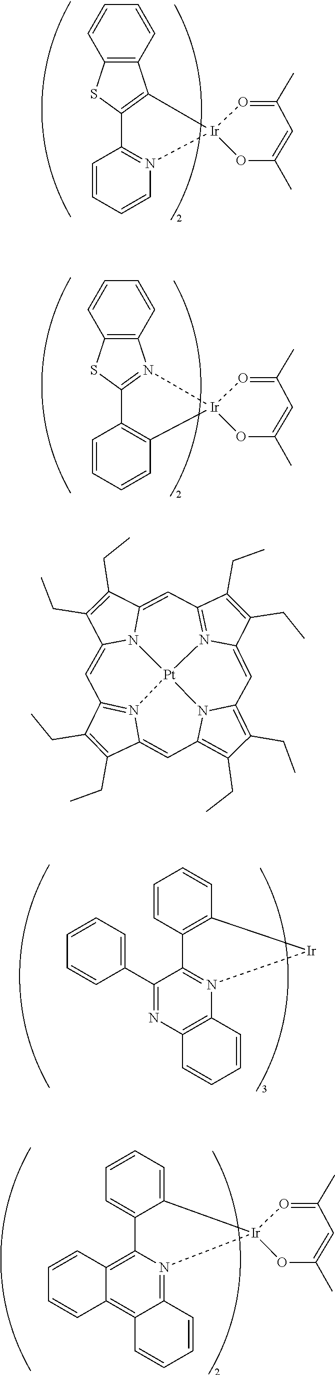

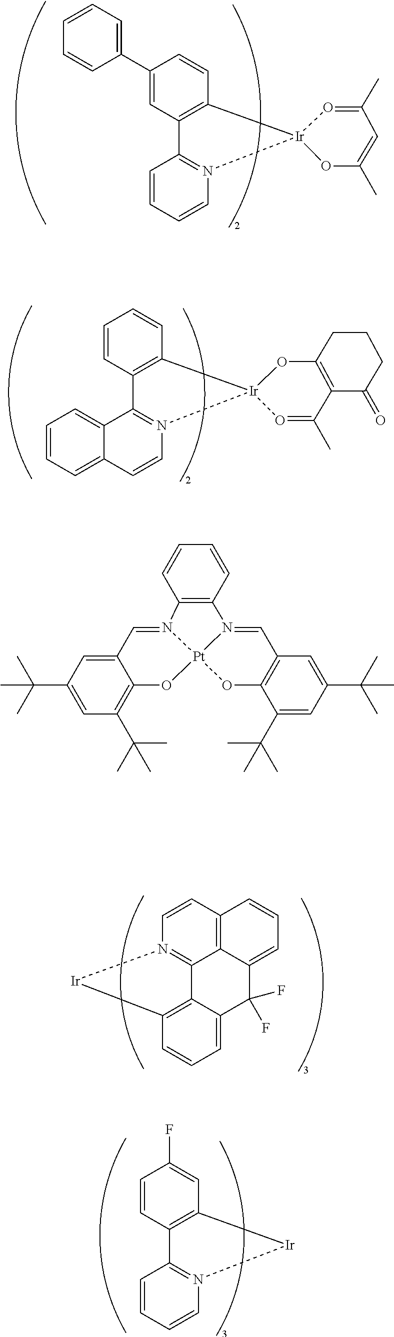

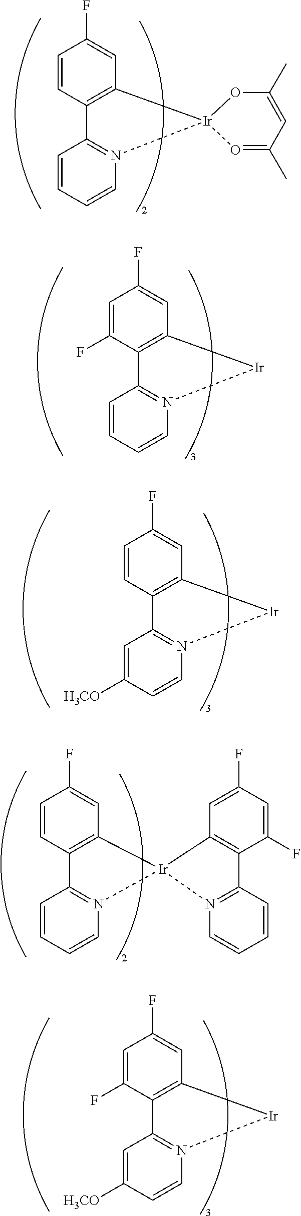

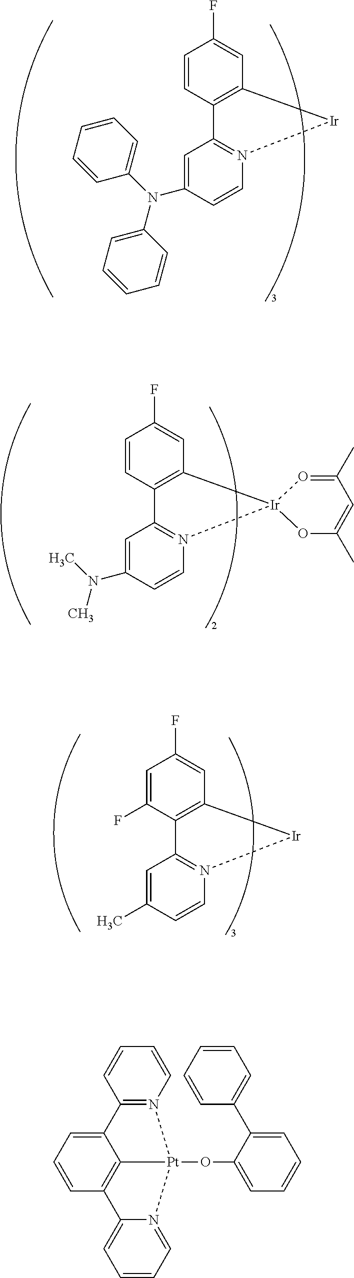

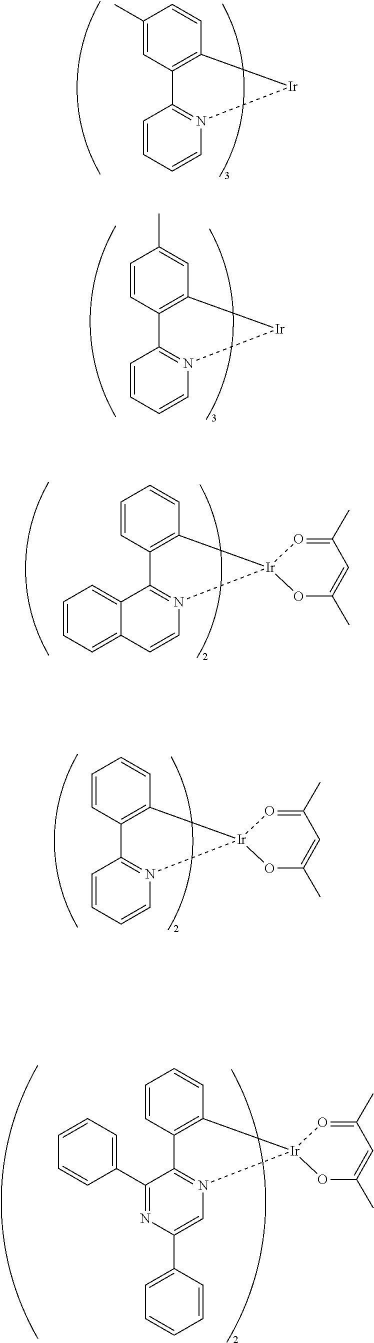

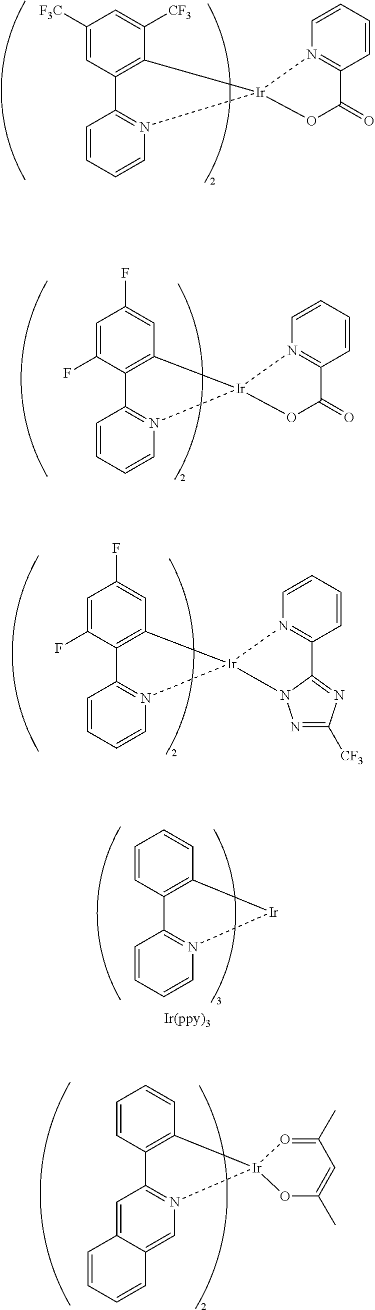

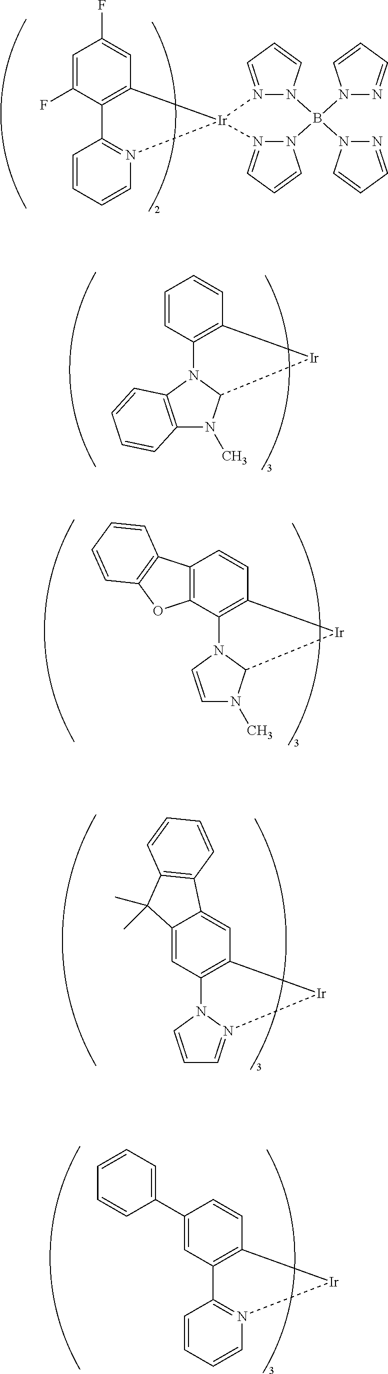

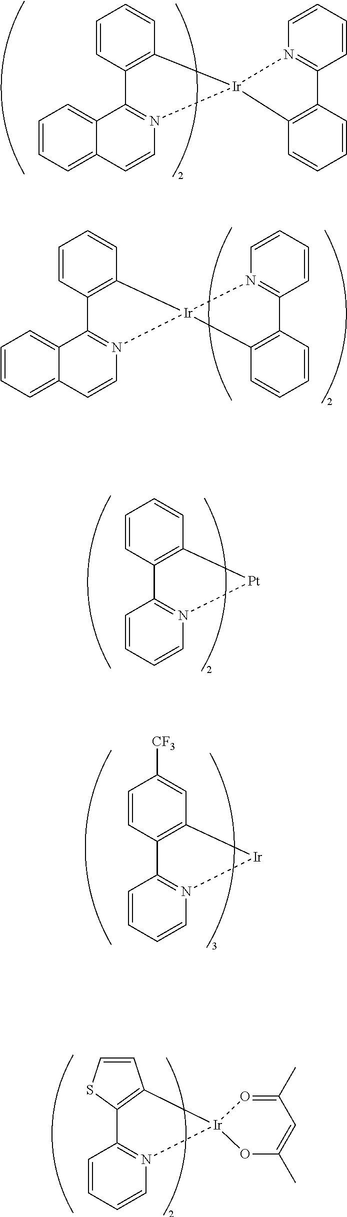

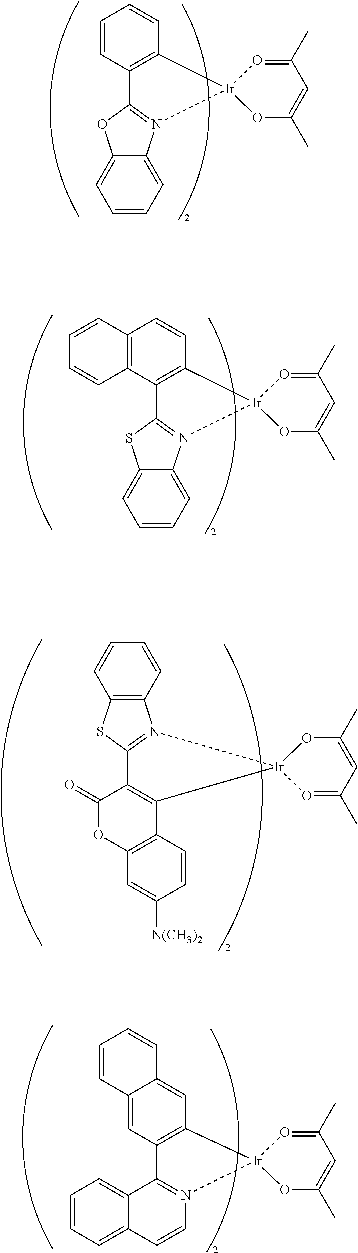

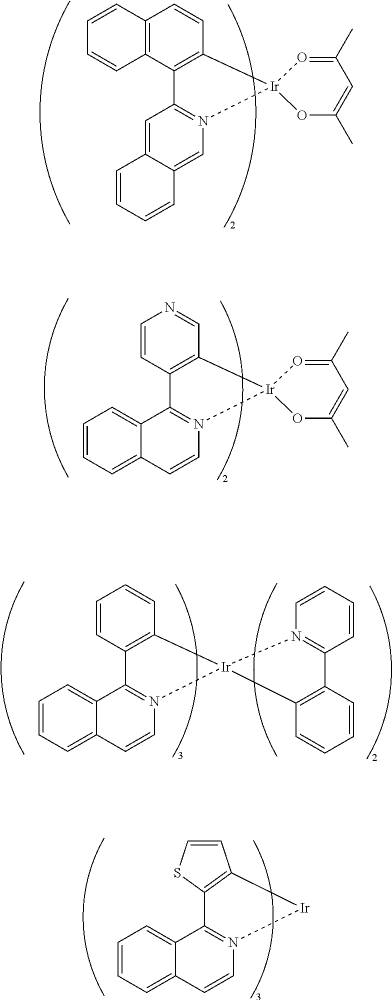

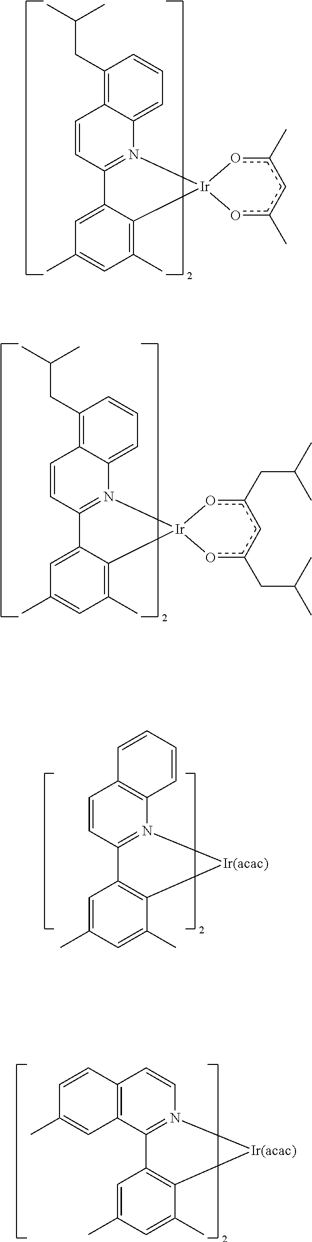

As the phosphorescent emitting material, an iridium complex represented by the following formula (β) is also preferable.

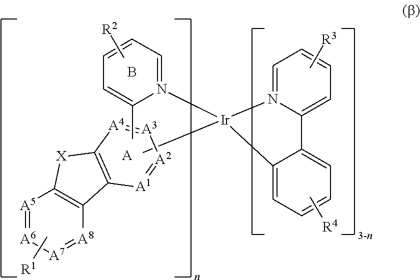

In the formula (β), A¹ to A⁸ include carbon or nitrogen, and at least one of A¹ to A⁸ is nitrogen, the ring B is bonded with the ring A by a C—C bond, and iridium (Ir) is bonded with the ring A by an Ir—C bond. It is preferred that only one of A¹ to A⁸ be nitrogen, and it is further preferred that only one of A⁵ to A⁸ be nitrogen.

X is O, S or Se, with O being preferable.

R¹ to R⁴ are independently a mono-, di-, tri-, or tetra-substituted or unsubstituted. Adjacent R¹ to R⁴ may be bonded with each other to form a ring. R¹ to R⁴ are independently selected from the group consisting of hydrogen, deuterium, halogen, a substituted or unsubstituted alkyl including 1 to 25 carbon atoms, a substituted or unsubstituted cycloalkyl including 3 to 25 ring carbon atoms, a substituted or unsubstituted heteroalkyl including 2 to 25 atoms, a substituted or unsubstituted arylalkyl including 7 to 50 carbon atoms, a substituted or unsubstituted alkoxy including 1 to 25 carbon atoms, a substituted or unsubstituted aryloxy including 6 to 24 ring carbon atoms, a substituted or unsubstituted amino, silyl substituted by one or more groups selected from the group consisting of an alkyl group including 1 to 25 carbon atoms and an aryl group including 6 to 24 ring carbon atoms, a substituted or unsubstituted alkenyl including 2 to 25 carbon atoms, cycloalkenyl including 3 to 25 ring carbon atoms, heteroalkenyl including 3 to 25 atoms, alkynyl including 2 to 25 carbon atoms, aryl including 6 to 24 ring carbon atoms, heteroaryl including 5 to 30 ring atoms, acyl, carbonyl substituted by one or more groups selected from the group consisting of an alkyl group including 1 to 25 carbon atoms and aryl group including 6 to 24 ring carbon atoms, carboxylic acid, ester, nitrile, isonitrile, tolyl, sulfanyl, sulfinyl, sulfonyl, phosphino and a combination thereof. R¹ to R⁴ are preferably selected from hydrogen, deuterium, an alkyl group including 1 to 25 carbon atoms and a combination thereof. It is preferred that R² and/or R³ be an alkyl group including 1 to 25 carbon atoms. It is further preferred that the alkyl group be deuterated or partially deuterated.

n is an integer of 1 to 3, with 1 being preferable.

The iridium complex represented by the formula (β) is preferably an iridium complex represented by the following formula (β-1):

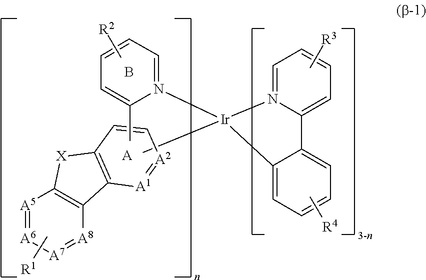

A¹, A², A⁵ to A⁸, X, R¹to R⁴, and n in the formula (β-1) are the same as those in the formula (β).

The specific examples of the iridium complex represented by the formula (β) or the formula (β-1) are shown below, but the iridium complex is not limited thereto.

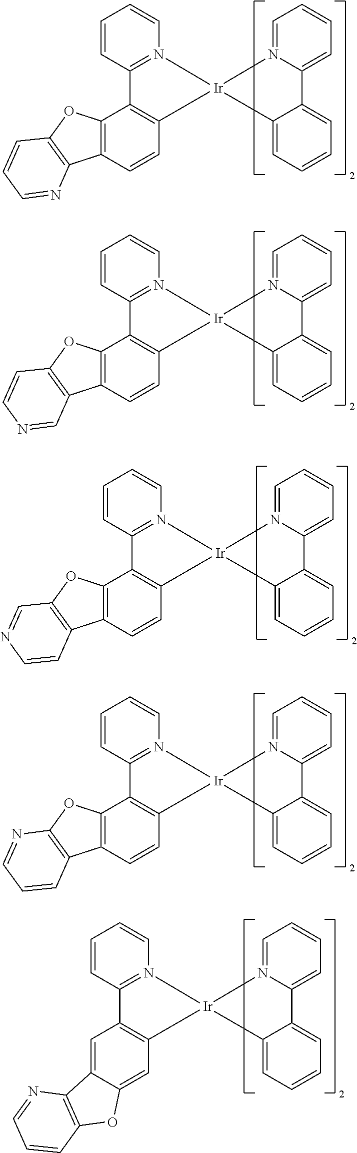

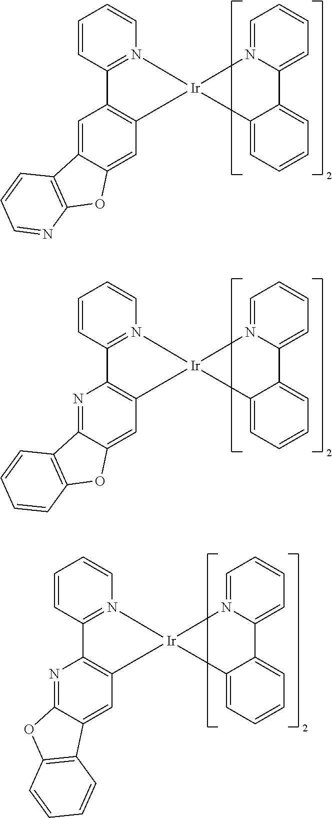

The iridium complex represented by the formula (β) is also preferably an iridium complex represented by the following formula (β-2):

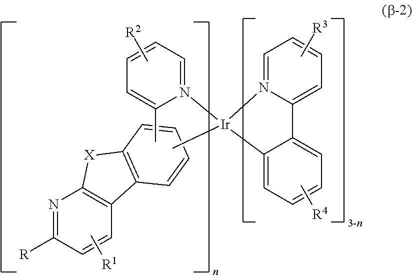

In the formula (β-2), R¹ to R⁴, X and n are the same as in the formula (β).

R is selected from the group consisting of a substituted or unsubstituted alkyl including 1 to 25 carbon atoms, a substituted or unsubstituted cycloalkyl including 3 to 25 ring carbon atoms and a combination thereof. R is preferably a substituted or unsubstituted alkyl including 1 to 25 carbon atoms or a substituted or unsubstituted cycloalkyl including 3 to 25 ring carbon atoms.

The specific examples of the iridium complex represented by the formula (β-2) are shown below, but the iridium complex is not limited thereto.

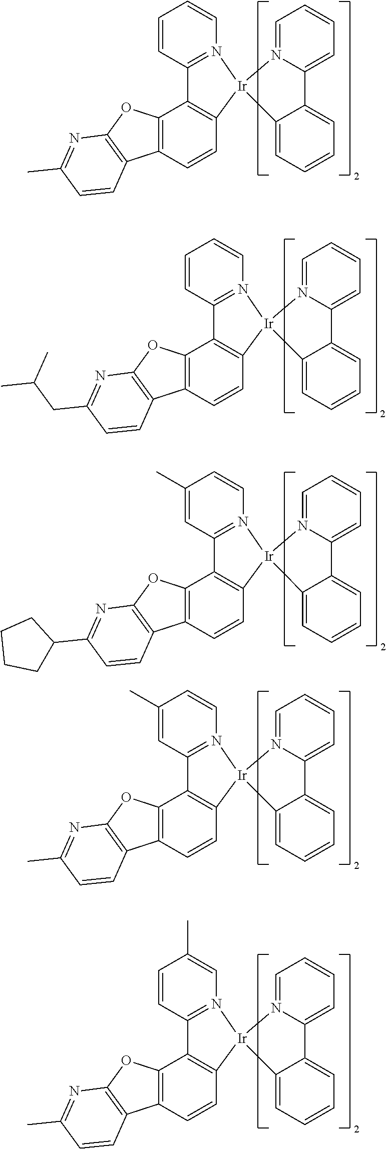

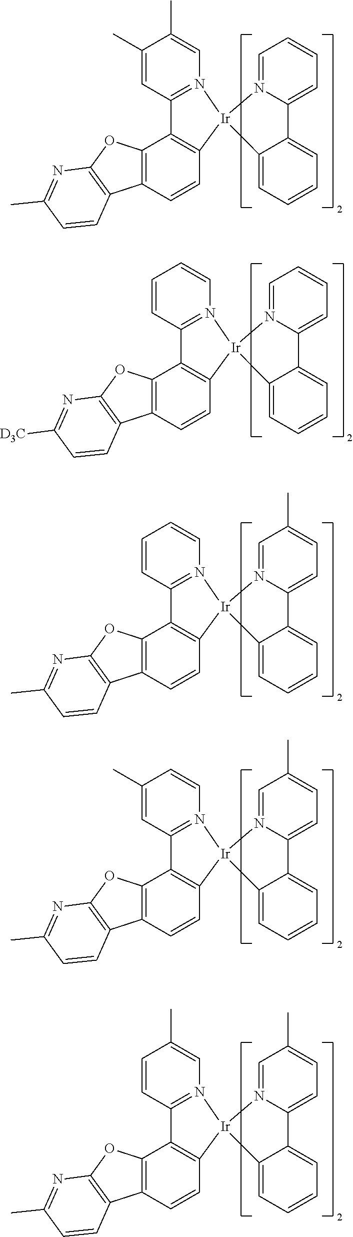

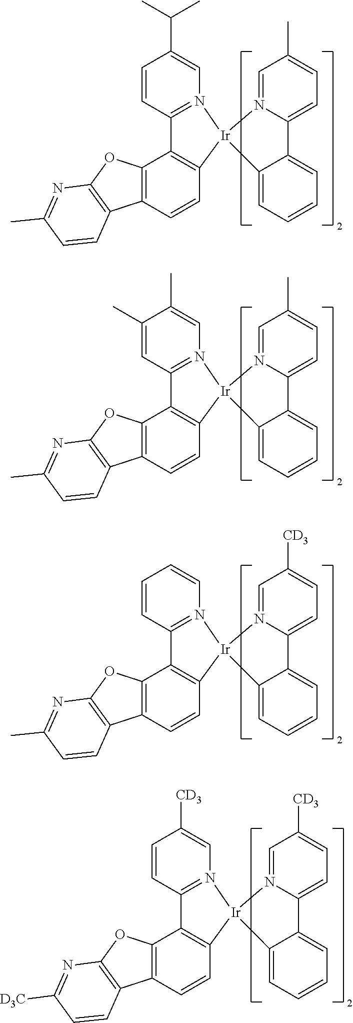

### (Host Material of Emitting Layer)

The emitting layer may have a structure in which the above-mentioned substance having high emitting property (guest material) is dispersed in other substances (host material). As the substance for dispersing the substance having high emitting property, various substances can be used in addition to the compound represented by the formula (1) and the compound represented by the formula (2). It is preferable to use a substance having a higher lowest unoccupied molecular orbital (LUMO level) than that of the substance having high emitting property, and having a lower highest unoccupied molecular orbital (HOMO level) than that of the substance having high emitting property.

As the substance for dispersing a substance having high emitting property (host material), 1) metal complex such as an aluminum complex, a beryllium complex or a zinc complex, 2) heterocyclic compound such as an oxadiazole derivative, a benzimidazole derivative, a phenanthroline derivative or the like, 3) fused aromatic compound such as a carbazole derivative, an anthracene derivative, a phenanthrene derivative, a pyrene derivative or a chrysene derivative, and 3) aromatic amine compound such as a triarylamine derivative or a fused polycyclic aromatic amine derivative can be used.

### (Electron-Transporting Layer)

An electron-transporting layer is a layer containing a substance having high electron-transporting property. In the electron-transporting layer, 1) metal complex such as an aluminum complex, a beryllium complex or a zinc complex; 2) a heteroaromatic compound such as an imidazole derivative, a benzimidazole derivative, an azine derivative, a carbazole derivative and a phenanthroline derivative, and 3) a polymer compound can be used.

### (Electron-Injecting Layer)

An electron-injecting layer is a layer containing a substance having high-electron injection property. In the electron-injecting layer, an alkali metal or an alkaline earth metal such as lithium (Li), lithium fluoride (LiF), cesium fluoride (CsF), calcium fluoride (CaF₂), lithium oxide (LiOₓ); or a compound of those can be used.

### (Cathode)

For a cathode, it is preferable to use a metal having a small work function (specifically, 3.8 eV or less), an alloy, an electrically conductive compound, a mixture of those or the like. As specific examples of the cathode material, an element belonging to Group 1 or Group 2 of the periodic table of the elements, i.e., a rare earth metal such as an alkali metal such as lithium (Li) or cesium (Cs), an alkaline earth metal such as magnesium (Mg), alloys containing those (e.g. MgAg, AlLi), and an alloy containing those or the like can be given.
B. Electronic Appliance

An electronic appliance according to one aspect of the invention is provided with the organic electroluminescence device as one aspect of the invention.

The organic electroluminescence device as one aspect of the invention can be used in various electronic appliances. For example, it can be used in a planar luminous body such as a flat panel display of a wall-hanging TV, a backlight of a copier, a printer and a crystal liquid display, or a light source of instruments, a displaying board, sign lighting or the like. Further, the compound of the invention can be used not only in an organic EL device but also in the field of an electrophotographic photoreceptor, a photoelectric conversion element, a solar cell, an image sensor or the like.

### EXAMPLES

The invention will be explained in more detail with reference to the Examples and the Comparative Examples, which should not be construed as limiting the scope of the invention.

### Example 1

A glass substrate with an ITO transparent electrode (anode) having a dimension of 25 mm×75 mm×1.1 mm (manufactured by GEOMATIC Co., Ltd.) was subjected to ultrasonic cleaning in isopropyl alcohol for 5 minutes and then to UV ozone cleaning for 30 minutes. The thickness of the ITO electrode was 77 nm.

The cleaned glass substrate with transparent electrode lines was mounted on a substrate holder in a vacuum deposition apparatus. First, the following electron-acceptor compound C-1 was deposited on the surface on which the transparent electrode lines had been formed so as to cover the transparent electrode, whereby a 5 nm-thick C-1 film was formed.

On this C-1 film, as the first hole-transporting material, the following aromatic amine derivative (compound X-1) was deposited, whereby a first hole-transporting layer having a thickness of 100 nm was formed.

Subsequent to the formation of the first hole-transporting layer, as the second hole-transporting material, the following aromatic amine derivative (compound X-2) was deposited, whereby a second hole-transporting layer having a thickness of 60 nm was formed.

Then, on this second hole-transporting layer, as the host material, the following compound 1-1(host 1), the following compound 2-1 (host 2) and the following compound Ir(ppy)₃ as the phosphorescent dopant material were co-deposited, whereby a 40 nm-thick emitting layer was formed. In the emitting layer, the concentration of the compound Ir(ppy)₃was 5.0 mass %, the concentration of the compound 1-1 was 47.5 mass % and the concentration of the compound 2-1 was 47.5 mass %. This co-deposition film functions as the emitting layer.

Subsequent to the formation of the emitting layer, the following compound ET1 was deposited in a film thickness of 30 nm. This compound ET film functions as an electron-transporting layer.

Then, LiF was formed into a 1 nm-thick film as an electron-injecting electrode (cathode) at a film-forming speed of 0.1 Å/min. On this LiF film, metal Al was deposited, whereby a metal cathode was formed in a film thickness of 80 nm.

The organic EL device of Example 1 had a following layer configuration.

ITO(77 nm)/Compound C-1(5 nm)/Compound X-1(100 nm)/Compound X-2(60 nm)/Compound 1-1:Compound 2-1:Ir(ppy)₃(40 nm, 47.5 mass %, 47.5 mass %, 5.0 mass %)/ET1(30 nm)/LiF(1 nm)/Al(80 nm)

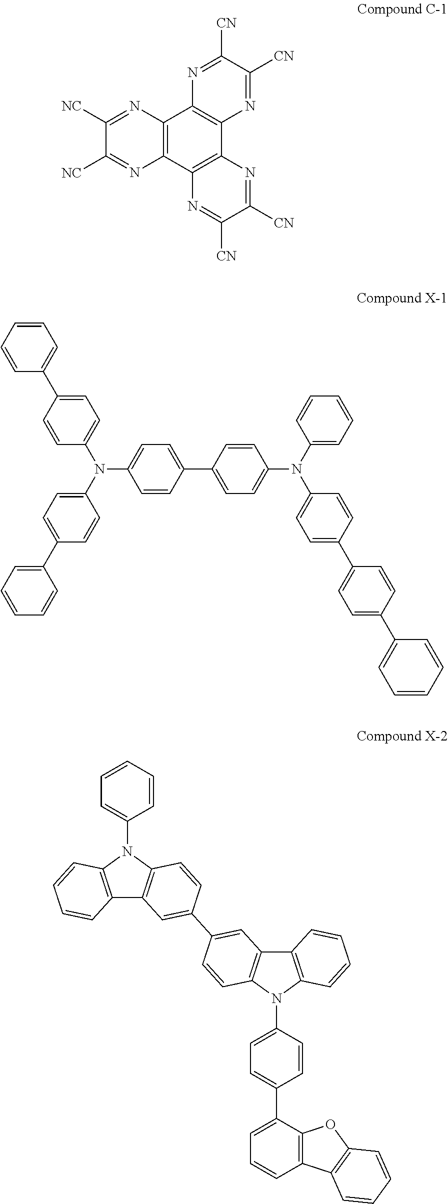 [SMILES: N#Cc1nc2c3nc(C#N)c(C#N)nc3c3nc(C#N)c(C#N)nc3c2nc1C#N ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1]

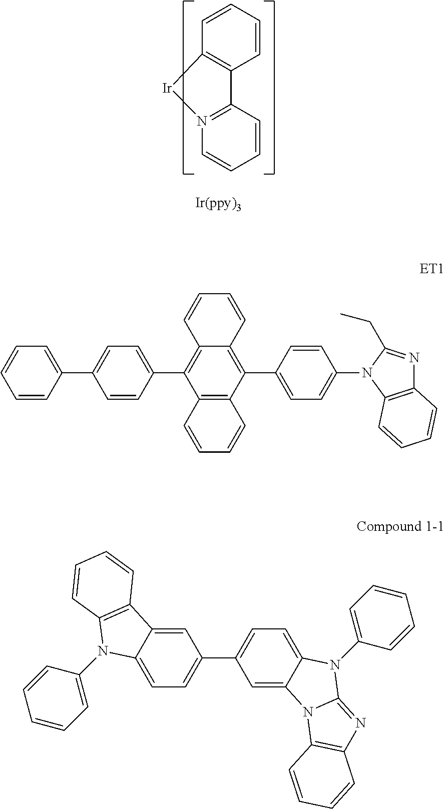

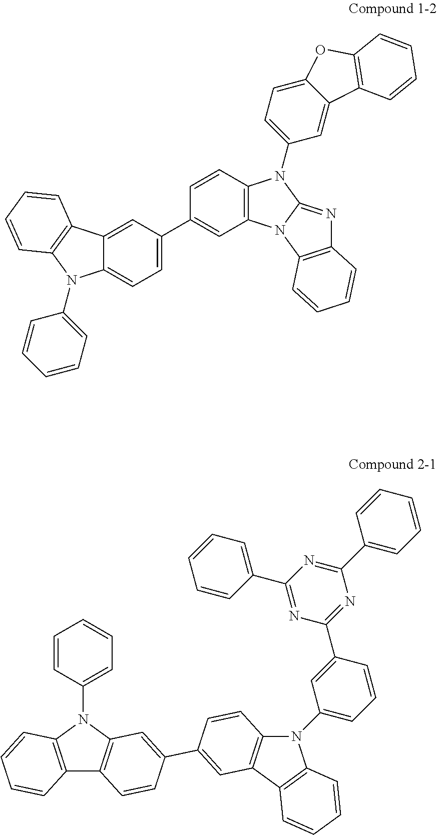 [SMILES: c1ccc(-n2c3ccccc3c3cc(-c4ccc5c(c4)n4c6ccccc6nc4n5-c4ccc5oc6ccccc6c5c4)ccc32)cc1 ; c1ccc(-c2nc(-c3ccccc3)nc(-c3cccc(-n4c5ccccc5c5cc(-c6ccc7c8ccccc8n(-c8ccccc8)c7c6)ccc54)c3)n2)cc1]

### <Evaluation of Organic EL Device>

For the obtained organic EL devices, the following evaluations were conducted. The results of evaluation are shown in Table 1.
(1) Driving Voltage (V)

A voltage (unit: V) when electric current was passed between the ITO transparent electrode and the metal Al cathode such that the current density became 10 mA/cm² was measured.
(2) External Quantum Efficiency (EQE; %)

A spectral radiance spectrum when a voltage was applied to the device such that the current density became 10 mA/cm² was measured by means of a spectroradiometer “CS-1000” (product name, manufactured by Konica Minolta Japan, Ltd.).

From the resulting spectral radiance spectrum, an external quantum efficiency (EQE) (unit: %) was calculated on the assumption that lambassian radiation was conducted.
(3) Lifetime (LT₈₀; Time)

A continuous electric current test (DC) was conducted with the initial current density being set as 50 mA/cm². A period of time taken for which the luminance was reduced to 80% as compared with the luminance at the time of starting the test was measured, and the measurement time was taken as lifetime (LT80).

### Example 2 and Comparative Example 1

Organic EL devices were fabricated and evaluated in the same manner as in Example 1, except that the host 1 was changed to the compounds shown in the following table 1. The results are shown in Table 1.

TABLE 1

| | | | V | EQE | LT₈₀ |
|---|---|---|---|---|---|
| | Host 1 | Host 2 | [V] | [%] | [h] |
| Ex. 1 | Compound 1-1 | Compound 2-1 | 3.53 | 17 | 100 |
| Ex. 2 | Compound 1-2 | Compound 2-1 | 3.47 | 17 | 100 |
| Comp. Ex. 1 | Compound X-2 | Compound 2-1 | 3.81 | 17 | 100 |

### Example 3

A glass substrate with an ITO transparent electrode (anode) having a dimension of 25 mm×75 mm×1.1 mm (manufactured by GEOMATIC Co., Ltd.) was subjected to ultrasonic cleaning in isopropyl alcohol for 5 minutes and then to UV ozone cleaning for 30 minutes. The thickness of the ITO electrode was 130 nm.

The cleaned glass substrate with transparent electrode lines was mounted on a substrate holder in a vacuum deposition apparatus. First, the following compound HA-1 was deposited on the surface on which the transparent electrode lines had been formed so as to cover the transparent electrode, whereby a 5 nm-thick HA-1 film was formed to form a hole-injecting layer.

Subsequently, on this hole-injecting layer, the compound HT-1 was deposited to form a 130 nm-thick HT-1 film, whereby a first hole-transporting layer was formed.

Then, on this first hole-transporting layer, compound HT-2 was deposited to form a 20 nm-thick HT-2 film, whereby a second hole-transporting layer was formed.

Then, on this hole-transporting layer, compound 1-1 (host 1), compound 2-2 (host 2) and a phosphorescent dopant material Ir(ppy)₃ were formed into a film by co-deposition, whereby a 40 nm-thick emitting layer was formed. The concentration of the first host and the second host was 47.5 mass %:47.5 mass % and the concentration of the phosphorescent dopant material Ir(ppy)₃ was 5 mass %.

Subsequent to the formation of the emitting layer, compound ET-1 and 8-quinolinolate lithium (Liq) were co-deposited in a mass ratio of 50:50, whereby a 25 nm-thick electron-transporting layer was formed.

On this electron-transporting layer, Liq was co-deposited to form a 1 nm-thick electron-injecting layer.

On this electron-injecting layer, metal Al was deposited to form a 80 nm-thick metal cathode.

The organic EL device according to Example 3 was fabricated in this way, and then evaluated in the same manner as in Example 1. The results are shown in Table 2.

The organic EL device of Example 3 had the following layer configuration: ITO(130 nm)/Compound HA-1(5 nm)/Compound HT-1(130 nm)/Compound HT-2 (20nm)/Compound 1-1:Compound 2-1: Ir(ppy)₃(40 nm, 47.5 mass %, 47.5 mass %, 5.0 mass %)/ET-1:Liq (25 nm, 50 mass %, 50 mass %)/Liq (1 nm)/Al(80 nm)

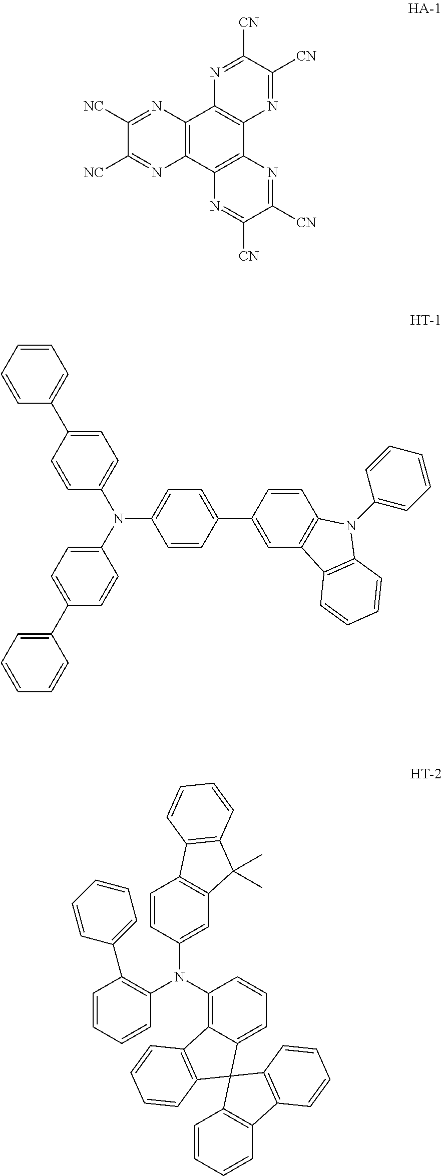 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccccc3-c3ccccc3)c3cccc4c3-c3ccccc3C43c4ccccc4-c4ccccc43)cc21]

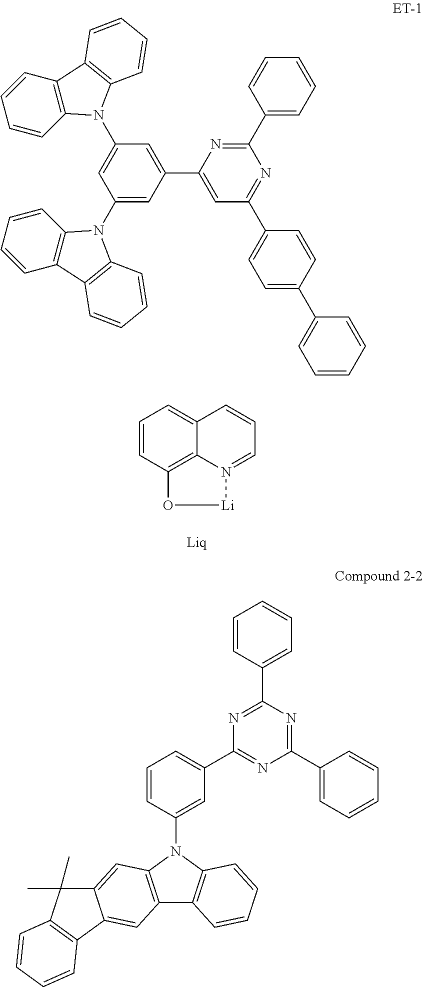

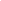

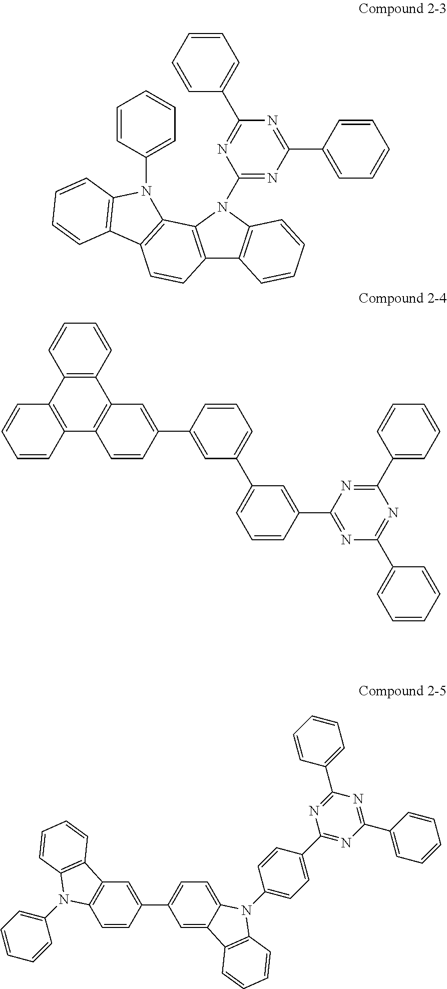 [SMILES: c1ccc(-c2nc(-c3ccccc3)nc(-n3c4ccccc4c4ccc5c6ccccc6n(-c6ccccc6)c5c43)n2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

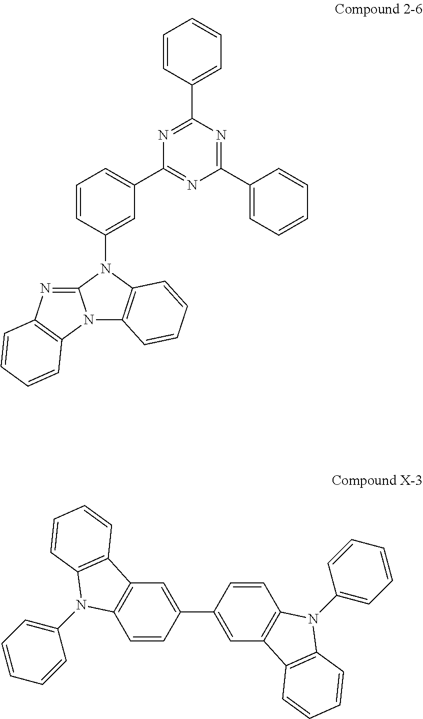 [SMILES: c1ccc(-c2nc(-c3ccccc3)nc(-c3cccc(-n4c5ccccc5n5c6ccccc6nc45)c3)n2)cc1 ; c1ccc(-n2c3ccccc3c3cc(-c4ccc5c(c4)c4ccccc4n5-c4ccccc4)ccc32)cc1]

### Examples 4 to 12 and Comparative Examples 2 to 8

Organic EL devices were fabricated in the same manner as in Example 3, except that the host 1 and the host 2 were changed to the compounds shown in the following table 2. The organic EL devices were evaluated in the same manner as in Example 1. The results are shown in Table 2.

TABLE 2

| | | | V | EQE | LT₈₀ |
|---|---|---|---|---|---|
| | Host 1 | Host 2 | [V] | [%] | [h] |
| Ex. 3 | Compound 1-1 | Compound 2-2 | 4.01 | 17.3 | 200 |
| Ex. 4 | Compound 1-2 | Compound 2-2 | 4.00 | 17.1 | 210 |
| Comp. Ex. 2 | Compound X-3 | Compound 2-2 | 4.21 | 17.2 | 200 |
| Ex. 5 | Compound 1-1 | Compound 2-3 | 3.90 | 16.8 | 210 |
| Ex. 6 | Compound 1-2 | Compound 2-3 | 3.92 | 16.9 | 200 |
| Comp. Ex. 3 | Compound X-3 | Compound 2-3 | 4.10 | 16.5 | 200 |
| Ex. 7 | Compound 1-1 | Compound 2-4 | 4.33 | 18.1 | 110 |
| Ex. 8 | Compound 1-2 | Compound 2-4 | 4.30 | 18.0 | 100 |
| Comp. Ex. 4 | Compound X-3 | Compound 2-4 | 4.62 | 18.1 | 100 |
| Ex. 9 | Compound 1-1 | Compound 2-5 | 4.02 | 17.8 | 220 |
| Ex. 10 | Compound 1-2 | Compound 2-5 | 4.01 | 17.9 | 210 |
| Comp. Ex. 5 | Compound X-3 | Compound 2-5 | 4.23 | 17.8 | 210 |
| Ex. 11 | Compound 1-1 | Compound 2-6 | 4.32 | 17.1 | 90 |
| Ex. 12 | Compound 1-2 | Compound 2-6 | 4.34 | 17.0 | 80 |
| Comp. Ex. 6 | Compound X-3 | Compound 2-6 | 4.53 | 16.9 | 80 |
| Comp. Ex. 7 | Compound 1-1 | — | 6.12 | 2.4 | 10 |
| Comp. Ex. 8 | Compound X-3 | — | 6.35 | 1.8 | 10 |

From the results shown in Tables 1 and 2, the organic EL device according to one aspect of the invention can be driven at a lower voltage while maintaining the luminous efficiency and the device life that are equivalent to that of the conventional organic EL device.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The specification of the Japanese patent applications claiming the priority under the Paris Convention to the invention is incorporated herein by reference in its entirety.

1. An organic electroluminescence device comprising an anode, a cathode and an emitting layer between the anode and the cathode,
wherein the emitting layer comprises as a host material a compound represented by the following formula (1) and a compound represented by the following formula (2): 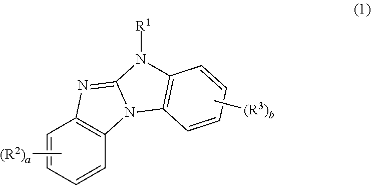
wherein in the formula (1),
R¹ to R³ are independently a group represented by —(B¹)ₒ—(B²)ₚ—(B³)_{q}—(B⁴)ᵣ—R⁴,
o, p, q and r are independently 0 or 1,
B¹ to B⁴ are independently a substituted or unsubstituted arylene group including 6 to 24 ring carbon atoms or a substituted or unsubstituted heteroarylene group including 5 to 30 ring atoms,
R⁴ is a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of an alkyl group including 1 to 25 carbon atoms and an aryl group including 6 to 24 ring carbon atoms, a cyano group or a —P(═O)R⁹R¹⁰ group,
R⁹ and R¹⁰ are independently a substituted or unsubstituted alkyl group including 1 to 25 carbon atoms, a substituted or unsubstituted aryl group including 6 to 18 ring carbon atoms or a substituted or unsubstituted heteroaryl group including 5 to 18 ring atoms, and
a and b are independently an integer of 0 to 4, 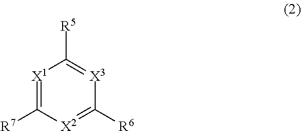
wherein in the formula (2),
X¹ to X³ are independently CR¹¹ or N,
R⁵ to R⁷ and R¹¹ are independently —(B⁵)ₛ—(B⁶)ₜ—(B⁷)ᵤ—(B⁸)ᵥ—R⁸,
s, t, u and v are independently 0 or 1,
B⁵ to B⁸ are independently a substituted or unsubstituted arylene group including 6 to 24 ring carbon atoms or a substituted or unsubstituted heteroarylene group including 5 to 30 ring atoms,
R⁸ is a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of an alkyl group including 1 to 25 carbon atoms and an aryl group including 6 to 24 ring carbon atoms, a cyano group or a —P(═O)R¹⁴R¹⁵ group,
adjacent substituents on R⁸ may be bonded with each other to form a ring, and
R¹⁴ and R¹⁵ are independently a substituted or unsubstituted alkyl group including 1 to 25 carbon atoms, a substituted or unsubstituted aryl group including 6 to 18 ring carbon atoms or a substituted or unsubstituted heteroaryl group including 5 to 18 ring atoms.

2. The organic electroluminescence device according to claim 1, wherein the compound represented by the formula (1) is a compound represented by the following formula (3): 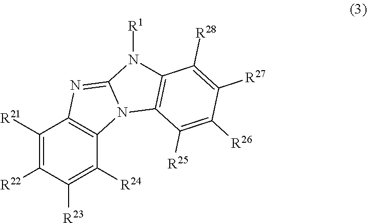
wherein in the formula (3),
R¹ is as defined in the formula (1), and
R²¹ to R²⁸ are independently a hydrogen atom, a substituted or unsubstituted alkyl group including 1 to 25 carbon atoms, a substituted or unsubstituted aryl group including 6 to 24 ring carbon atoms or a substituted or unsubstituted heteroaryl group including 5 to 30 ring atoms.

3. The organic electroluminescence device according to claim 2, wherein the compound represented by the formula (3) is a compound represented by the following formula (4): 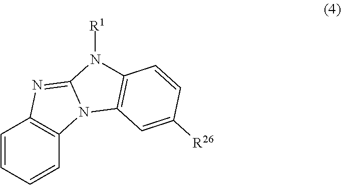
wherein in the formula (4), R¹ and R²⁶ are as defined in the formula (3).

4. The organic electroluminescence device according to claim 1, wherein the compound represented by the formula (2) is a compound represented by the following formula (21): 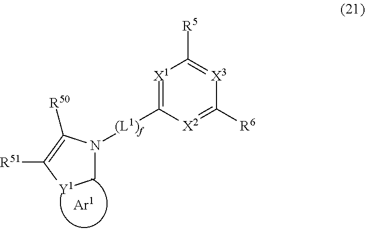
wherein in the formula (21),
X¹ to X³, R⁵ and R⁶ are as defined in the formula (2),
L¹ is a substituted or unsubstituted arylene group including 6 to 24 ring carbon atoms or a substituted or unsubstituted heteroarylene group including 5 to 30 ring atoms,
f is an integer of 0 or 1,
Y¹ is a carbon atom or a nitrogen atom,
Ar¹ is a substituted or unsubstituted aromatic hydrocarbon ring including 6 to 24 ring carbon atoms that shares a carbon atom and Y¹ of an adjacent nitrogen-containing five-membered ring and that is fused to the nitrogen-containing five-membered ring, or a substituted or unsubstituted heterocyclic aromatic ring including 5 to 30 ring atoms that shares a carbon atom and Y¹ of an adjacent nitrogen-containing five-membered ring and that is fused to the nitrogen-containing five-membered ring,
R⁵⁰ and R⁵¹ are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of an alkyl group including 1 to 25 carbon atoms and an aryl group including 6 to 24 ring carbon atoms, a cyano group or a —P(═O)R¹⁴R¹⁵ group, or R⁵⁰ and R⁵¹ are bonded with each other to form a substituted or unsubstituted aromatic ring group including 6 to 24 ring carbon atoms, and
R¹⁴ and R¹⁵ are as defined in the formula (2).

5. The organic electroluminescence device according to claim 4, wherein the compound represented by the formula (21) is a compound represented by the following formula (22): 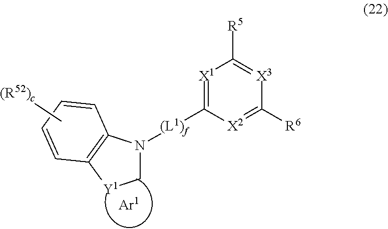
wherein in the formula (22),
X¹ to X³, R⁵, R⁶, L¹, f, Y¹ and Ar¹ are as defined in the formula (21),
R⁵² is a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of an alkyl group including 1 to 25 carbon atoms and an aryl group including 6 to 24 ring carbon atoms, a cyano group or a —P(═O)R¹⁴R¹⁵ group,
adjacent substituents on R⁵² may be bonded with each other to form a ring,
R¹⁴ and R¹⁵ are as defined in the formula (2),
c is an integer of 0 to 4,
when c is 2 to 4, the plural R⁵²s may be the same or different, and
adjacent R⁵²s may be bonded with each other to form a ring.

6. The organic electroluminescence device according to claim 5, wherein the compound represented by the formula (22) is a compound represented by the following formula (23): 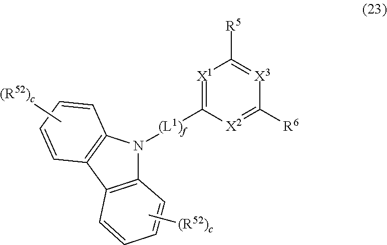
wherein in the formula (23),
X¹ to X³, R⁵, R⁶, L¹, f, R⁵² and c are as defined in the formula (22).

7. The organic electroluminescence device according to claim 6, wherein the compound represented by the formula (23) is a compound represented by the following formula (24): 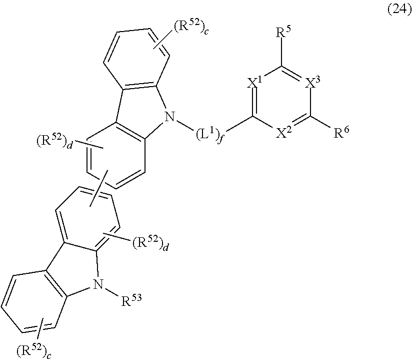
wherein in the formula (24),
X¹ to X³, R⁵, R⁶, L¹, f, R⁵² and c are as defined in the formula (23),
d is an integer of 0 to 3, when d is 2 or 3, plural R⁵²s may be the same or different,
R⁵³ is a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of an alkyl group including 1 to 25 carbon atoms and an aryl group including 6 to 24 ring carbon atoms, a cyano group or a —P(═O)R¹⁴R¹⁵ group,
adjacent substituents on R⁵³ may be bonded with each other to form a ring, and
R¹⁴ and R¹⁵ are as defined in the formula (2).

8. The organic electroluminescence device according to claim 5, wherein the compound represented by the formula (22) is a compound represented by the following formula (25): 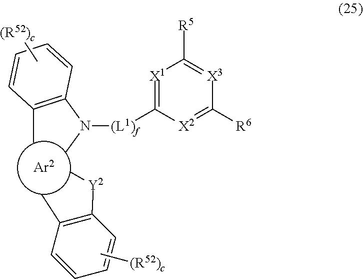
wherein in the formula (25),
X¹ to X³, R⁵, R⁶, L¹, f, R⁵² and c are as defined in the formula (22),
Y² is CR⁵⁴R⁵⁵, NR⁵⁶, an oxygen atom or a sulfur atom,
R⁵⁴ to R⁵⁶ are independently a hydrogen atom, a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of an alkyl group including 1 to 25 carbon atoms and an aryl group including 6 to 24 ring carbon atoms, a cyano group or a —P(═O)R¹⁴R¹⁵ group,
adjacent substituents on R⁵⁴ to R⁵⁶ may be bonded with each other to form a ring,
R¹⁴ and R¹⁵ are as defined in the formula (2), and
Ar² is a substituted or unsubstituted aromatic hydrocarbon ring including 6 to 24 ring carbon atoms that shares two carbon atoms of each of adjacent two five-membered rings and that is fused to the two five-membered rings or a substituted or unsubstituted heterocyclic aromatic ring including 5 to 30 ring atoms that shares two carbon atoms of each of two adjacent five-membered rings and that is fused to the two five-membered rings.

9. The organic electroluminescence device according to claim 8, wherein the compound represented by the formula (25) is a compound represented by any one of the following formulas (26A) to (26F); 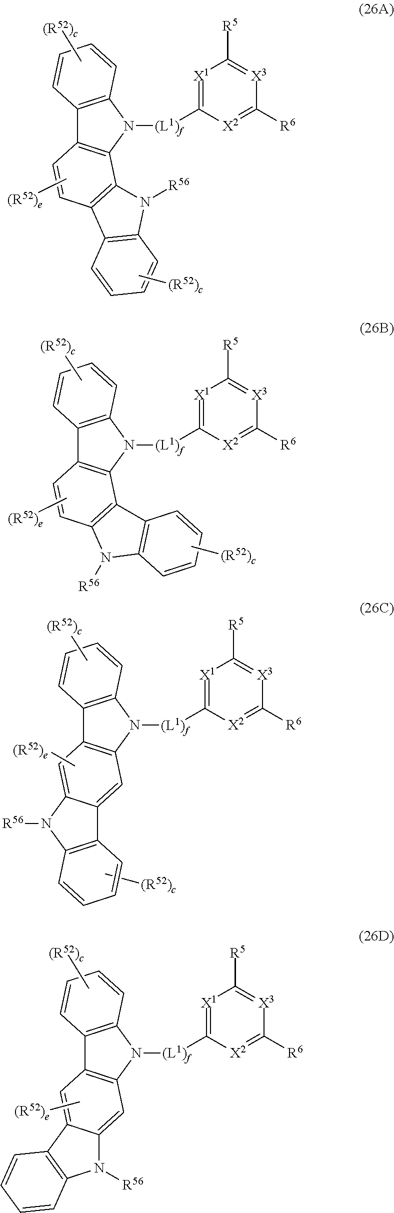 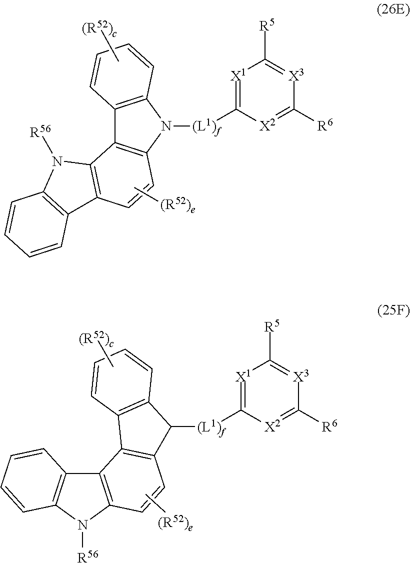
wherein in the formulas (26A) to (26F),
X¹ to X³, R⁵, R⁶, L¹, f, R⁵², R⁵⁶ and c are as defined in the formula (25),
e is an integer of 0 to 2, and
when e is 2, plural R⁵²s may be the same or different.

10. The organic electroluminescence device according to claim 8, wherein the compound represented by the formula (25) is represented by any one of the following formulas (27A) to (27F): 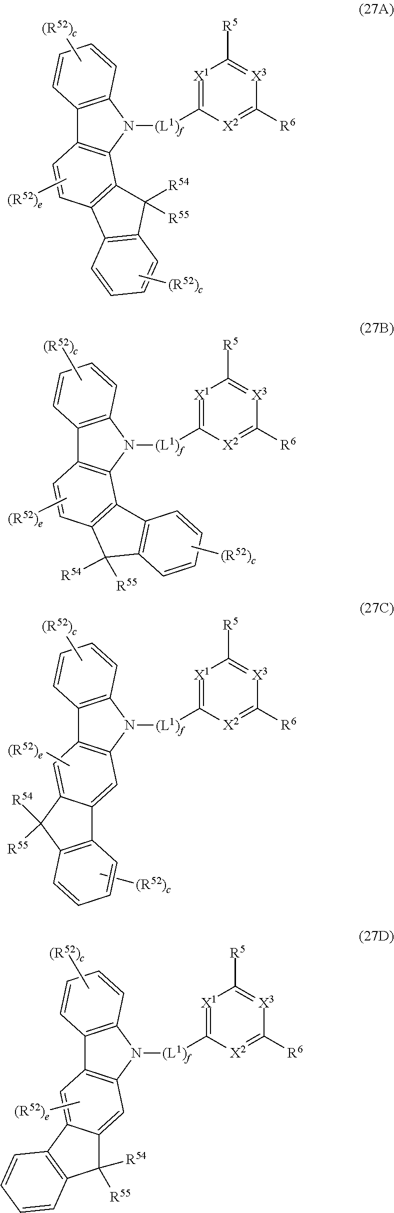 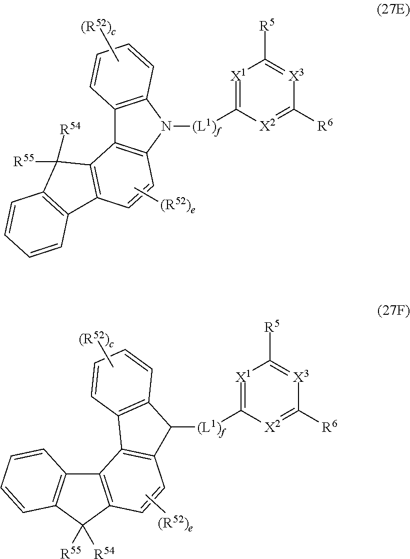
wherein in the formulas (27A) to (27F),
X¹ to X³, R⁵, R⁶, L¹, f, R⁵², R⁵⁴, R⁵⁵ and c are as defined in the formula (25),
e is an integer of 0 to 2, and
when e is 2, plural R⁵²s may be the same or different.

11. The organic electroluminescence device according to claim 1, wherein the compound represented by the formula (2) is a compound represented by the following formula (30): 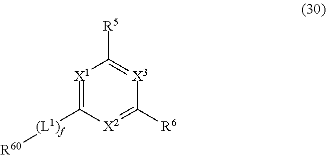
wherein in the formula (30),
X¹ to X³, R⁵ and R⁶ are as defined in the formula (2),
L¹ is a substituted or unsubstituted fused arylene group including 10 to 24 ring carbon atoms or a substituted or unsubstituted fused heteroarylene group including 9 to 30 ring atoms,
f is an integer of 0 or 1, and
R⁶⁰ is a substituted or unsubstituted aryl group including 6 to 24 ring carbon atoms or a substituted or unsubstituted heteroaryl group including 5 to 30 ring atoms.

12. The organic electroluminescence device according to claim 11, wherein the compound represented by the formula (30) is a compound represented by the following formula (31): 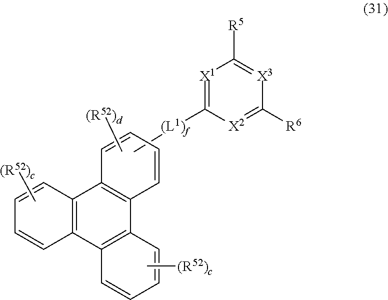
wherein in the formula (31),
X¹ to X³, R⁵, R⁶, L¹ and f are as defined in the formula (30),
R⁵² is a halogen atom, a substituted or unsubstituted alkyl group including 1 to 25 carbon atoms, a substituted or unsubstituted alkenyl group including 2 to 25 carbon atoms, a substituted or unsubstituted alkynyl group including 2 to 25 carbon atoms, a substituted or unsubstituted cycloalkyl group including 3 to 25 ring carbon atoms, a substituted or unsubstituted alkoxy group including 1 to 25 carbon atoms, a substituted or unsubstituted aryl group including 6 to 24 ring carbon atoms, a substituted or unsubstituted heteroaryl group including 5 to 30 ring atoms, a substituted or unsubstituted aryloxy group including 6 to 24 ring carbon atoms, a substituted or unsubstituted alkylthio group including 1 to 25 carbon atoms, a substituted or unsubstituted arylthio group including 6 to 24 ring carbon atoms, a silyl group substituted by one or more groups selected from the group consisting of an alkyl group including 1 to 25 carbon atoms and an aryl group including 6 to 24 ring carbon atoms, a cyano group or a —P(═O)R¹⁴R¹⁵ group,
adjacent substituents on R⁵² may be bonded with each other to form a ring,
R¹⁴ and R¹⁵ are as defined in the formula (2), and
c is an integer of 0 to 4,
d is an integer of 0 to 3,
when c is 2 to 4 and d is 2 or 3, plural R⁵²s may be the same or different, and
adjacent R⁵²s may be bonded with each other to form a ring.

13. The organic electroluminescence device according to claim 11, wherein the compound represented by the formula (30) is a compound represented by the following formula (32): 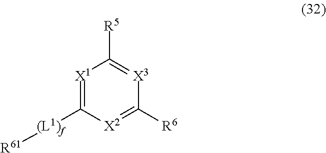
wherein in the formula (32),
X¹ to X³, R⁵, R⁶, L¹ and f are as defined in the formula (30), and
R⁶¹ is a substituted or unsubstituted fused heteroaryl group including 9 to 30 ring atoms that does not include a nitrogen atom.

14. The organic electroluminescence device according to claim 1, wherein the emitting layer further comprises one or more selected from a fluorescent emitting material and a phosphorescent emitting material.

15. The organic electroluminescence device according to claim 14, wherein the emitting layer comprises a phosphorescent emitting material as an emitting material and the phosphorescent emitting material is an ortho-metalated complex of a metal atom selected from iridium (Ir), osmium (Os) and platinum (Pt).

16. The organic electroluminescence device according to claim 1, which further has a hole-transporting layer.

17. The organic electroluminescence device according to claim 1, which further has an electron-transporting layer.

18. An electronic appliance provided with the organic electroluminescence device according to claim 1.
